(19) United States
(12) Patent Application Publication
(10) Pub. No.: US 2021/0261561 A1
(43) Pub. Date: Aug. 26, 2021

(54) BICYCLIC HETEROCYCLE DERIVATIVES HAVING SELECTIVE BACE1 INHIBITORY ACTIVITY

(71) Applicant: SHIONOGI & CO., LTD., Osaka-shi, Osaka (JP)

(72) Inventors: Tatsuhiko UENO, Osaka (JP); Kouki FUCHINO, Osaka (JP); Kazuki TAKAHARA, Osaka (JP); Frederik ROMBOUTS, Beerse (BE); Dries VAN DEN BOSSCHE, Beerse (BE); Michel SURKYN, Beerse (BE); Michel DE CLEYN, Beerse (BE)

(21) Appl. No.: 17/049,818

(22) PCT Filed: Apr. 22, 2019
(86) PCT No.: PCT/JP2019/017054

§ 371 Date: Oct. 22, 2020

(30) Foreign Application Priority Data
Apr. 23, 2018 (JP) 2018-082264
Apr. 27, 2018 (JP) 2018-086435

(51) Int. Cl. C07D 491/056; C07D 519/00; A61P 25/28

(52) U.S. Cl. CPC C07D 491/056; A61P 25/28; C07D 519/00

(57) ABSTRACT

The present invention provides a compound which has an effect of inhibiting amyloid ß production, especially an effect of inhibiting selective BACE1, and which is useful as a therapeutic or prophylactic agent for diseases induced by production, secretion and/or deposition of amyloid ß proteins.

A compound of the formula (IA) or the like, wherein
-A₁- is alkylene optionally substituted with one or more halogen;
R² is substituted or unsubstituted alkyl or the like;
R³ and R⁴ are each independently a hydrogen atom, halogen, alkyl or haloalkyl or the like;
R⁵ is a hydrogen atom or halogen;
A₄ is N or CR⁶ wherein R⁶ is a hydrogen atom, halogen, or substituted or unsubstituted alkyl;
A₅ is NR⁷ or CR⁸R⁹;
A₆ is CR¹⁸ or N;
R¹⁸ is a hydrogen atom;
R⁷ is substituted or unsubstituted alkyl;
R⁸ and R⁹ are each independently a hydrogen atom, halogen, alkyl or haloalkyl or the like; and
Ring B is bicyclic ring;
or a pharmaceutically acceptable salt thereof.

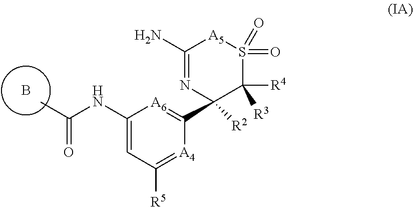

### TECHNICAL FIELD

The present invention relates to a compound which has amyloid ß production inhibitory activity, and is useful as an agent for treating or preventing disease induced by production, secretion and/or deposition of amyloid ß proteins.

### BACKGROUND ART

In the brain of Alzheimer's patient, the peptide composed of about 40 amino acids residue as is called amyloid ß protein, that accumulates to form insoluble specks (senile specks) outside nerve cells is widely observed. It is concerned that these senile specks kill nerve cells to cause Alzheimer's disease, so the therapeutic agents for Alzheimer's disease, such as decomposition agents of amyloid ß protein and amyloid vaccine, are under investigation.

Secretase is an enzyme which cleaves a protein called amyloid ß precursor protein (APP) in cell and produces amyloid ß protein. The enzyme which controls the production of N terminus of amyloid ß protein is called as ß-secretase (beta-site APP-cleaving enzyme 1, BACE1). It is thought that inhibition of this enzyme leads to reduction of producing amyloid ß protein and that the therapeutic or prophylactic agent for Alzheimer's disease will be created due to the inhibition.

Patent Documents 1 to 34 and Non-Patent Documents 1 to 7 disclose compounds which are useful as therapeutic agent for Alzheimer's disease, Alzheimer's relating symptoms, diabetes or the like, but each of substantially disclosed compounds has a structure different from the compounds of the present invention.

### CITATION LIST

### Patent Literature

### [PTL 1]

WO2017/112901

### [PTL 2]

WO2014/096377

### [PTL 3]

US2016/0213645

### [PTL 4]

WO2016/040903

### [PTL 5]

WO2015/120364

### [PTL 6]

WO2015/051239

### [PTL 7]

WO2016/025364

### [PTL 8]

US2014/0271911

### [PTL 9]

WO2014/096377

### [PTL 10]

WO2014/093190

### [PTL 11]

WO2014/089149

### [PTL 12]

WO2011/044181

### [PTL 13]

U.S. Pat. No. 9,273,042

### [PTL 14]

US2015/0344500

### [PTL 15]

WO2015/038446

### [PTL 16]

WO2015/017407

### [PTL 17]

WO2014/150344

### [PTL 18]

WO2014/120658

### [PTL 19]

WO2014/099794

### [PTL 20]

WO2014/059185

### [PTL 21]

WO2013/174781

### [PTL 23]

WO2013/028670

### [PTL 24]

WO2012/139425

### [PTL 25]

JP2017/071603

### [PTL 26]

WO2015/156421

### [PTL 27]

JP2014/101354

### [PTL 28]

WO2014/065434

### [PTL 29]

WO2014/001228

### [PTL 30]

WO2013/041499

### [PTL 31]

US2013/0072478

### [PTL 32]

JP2012/250933

### [PTL 33]

WO2012/107371

### [PTL 34]

WO2011/071135

### Non-Patent Literature

### [NPL 1]

Methods and Principles in Medicinal Chemistry, Volume: 67, Issue: Fragment-Based Drug Discovery, Pages: 329-353

### [NPL 2]

Journal of Medicinal Chemistry, Volume: 59, Issue: 23, Pages: 10435-10450

### [NPL 3]

Pharmacological Reports, Volume: 68, Issue: 1, Pages: 127-138

### [NPL 4]

BioDrugs, Volume: 30, Issue: 3, Pages: 173-194, (MK-8931)

### [NPL 5]

Nature Reviews Drug Discovery, Volume: 15, Issue: 3, Pages: 151

### [NPL 6]

AAPS Journal, Volume: 18, Issue: 2, Pages: 519-527,

### [NPL 7]

Frontiers in Aging Neuroscience, Volume: 6, 165/1-165/9, 9 pp.,

### SUMMARY OF INVENTION

### Technical Problem

The present invention provides compounds which have reducing effects to produce amyloid ß protein, especially selective BACE1 inhibitory activity, and are useful as an agent for treating disease induced by production, secretion and/or deposition of amyloid ß protein.

### Advantageous Effects of Invention

The compound of the present invention has BACE1 selective inhibitory activity and is useful as an agent for treating and/or preventing disease induced by production, secretion or deposition of amyloid ß proteins such as Alzheimer dementia.

### Solution to Problem

The present invention, for example, provides the inventions described in the following items.

(1) A compound of Formula (IA), (IB) or (IC):

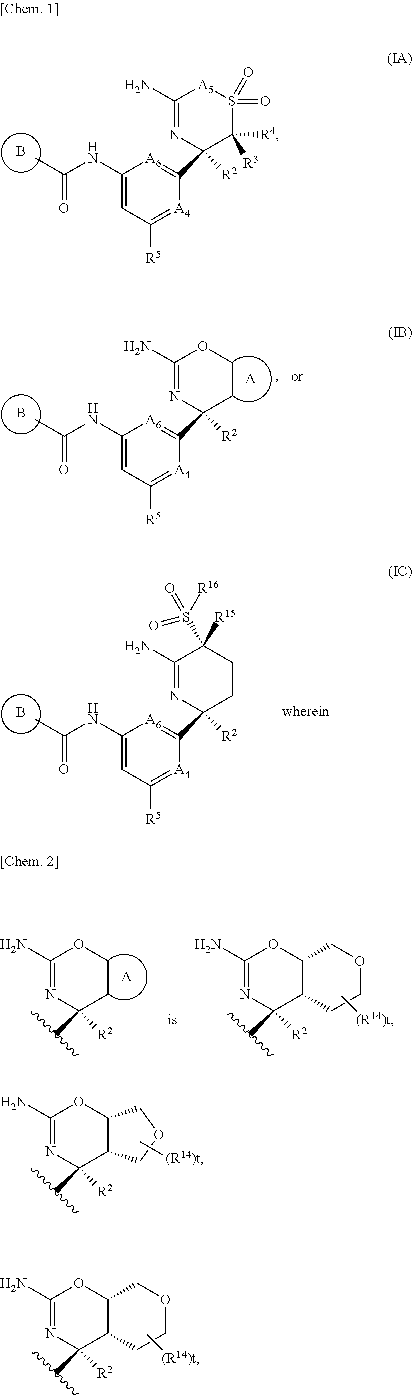

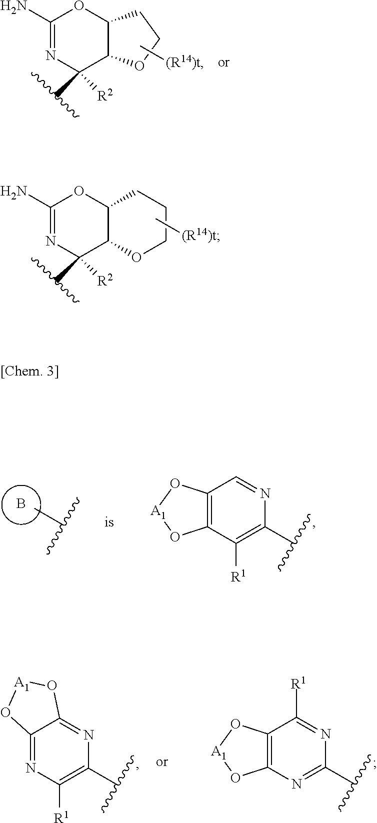

-A₁- is alkylene optionally substituted with one or more halogen;
R¹ is a hydrogen atom, halogen, alkyl, haloalkyl or amino;
R² is substituted or unsubstituted alkyl;
R³ and R⁴ are each independently a hydrogen atom, halogen, alkyl or haloalkyl;
R⁵ is a hydrogen atom or halogen;
A₄ is N or CR⁶ wherein R⁶ is a hydrogen atom, halogen or substituted or unsubstituted alkyl;
A₆ is CR¹⁸ or N;
R¹⁸ is a hydrogen atom;
A₄ and A₆ are not simultaneously both N;
A₅ is NR⁷ or CR⁸R⁹;
R⁷ is substituted or unsubstituted alkyl;
R⁸ and R⁹ are each independently a hydrogen atom, halogen, alkyl or haloalkyl;
R², R³, R⁴, R⁸ and R⁹ may be any one of (i) to (iv):
(i) R² and one of R³ and R⁴ may be taken together with the carbon atoms to which they are attached to form a carbocycle or a heterocycle;
(ii) one of R³ and R⁴ and one of R⁸ and R⁹ may form alkylene wherein each carbon atom in the alkylene may be replaced with an oxygen atom or a nitrogen atom; the carbon atom(s) in the alkylene is each independently substituted with one or more group(s) selected from R^{a}; and the nitrogen atom(s) in the alkylene is each substituted with one or more group(s) selected from R^{b}; R^{a} is a hydrogen atom, halogen, hydroxy, cyano, or substituted or unsubstituted alkyl; R^{b} is a hydrogen atom or substituted or unsubstituted alkyl;
(iii) R³ and R⁴ may be taken together with the carbon atom to which they are attached to form a carbocycle or a heterocycle; and
(iv) R⁸ and R⁹ may be taken together with the carbon atom to which they are attached to form a carbocycle or a heterocycle;
R¹⁴ is each independently alkyl optionally substituted with one or more group(s) selected from halogen, cyano, alkyloxy, haloalkyloxy, and non-aromatic carbocyclyl; or heteroaryl optionally substituted with one or more alkyl;
two R¹⁴s attached to a same carbon atom may be taken together with the carbon atom to which they are attached to form a 3- to 5-membered non-aromatic carbocycle optionally substituted with one or more group(s) selected from halogen, alkyl and haloalkyl;
t is an integer from 0 to 3;
R¹⁵ is alkyl optionally substituted with one or more group(s) selected from halogen; and
R¹⁶ is substituted or unsubstituted alkyl or non-aromatic carbocyclyl;
or a pharmaceutically acceptable salt thereof.
(1)′ A compound of Formula (IA-2)′

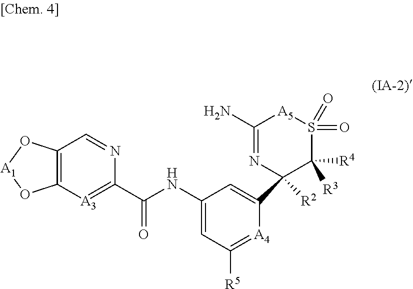

wherein
A₁ is alkylene optionally substituted with fluoro;
A₃ is N or CR¹ wherein R¹ is a hydrogen atom, halogen, alkyl or haloalkyl;
R² is substituted or unsubstituted alkyl;
R³ and R⁴ are each independently a hydrogen atom, halogen, alkyl or haloalkyl;
R⁵ is a hydrogen atom or halogen;
A₄ is N or CR⁶ wherein R⁶ is a hydrogen atom, halogen, or substituted or unsubstituted alkyl;
A₅ is NR⁷ or CR⁸R⁹;
R⁷ is substituted or unsubstituted alkyl;
R⁸ and R⁹ are each independently a hydrogen atom, halogen, alkyl or haloalkyl;
R² and one of R³ and R⁴ may be taken together with an adjacent atom to form carbocycle or heterocycle;
one of R³ and R⁴ and one of R⁸ and R⁹ may form alkylene wherein each carbon atom in the alkylene may be replaced with an oxygen atom or a nitrogen atom; the carbon atom(s) in the alkylene is each independently substituted with the substituent selected from R^{a}; and the nitrogen atom(s) in the alkylene is each substituted with the substituent selected from R^{b};
R^{a} is a hydrogen atom, halogen, hydroxy, cyano, or substituted or unsubstituted alkyl;
R^{b} is a hydrogen atom, or substituted or unsubstituted alkyl;
R³ and R⁴ may be taken together with an adjacent atom to form carbocycle or heterocycle;
R⁸ and R⁹ may be taken together with an adjacent atom to form carbocycle or heterocycle;
or a pharmaceutically acceptable salt.
(2) The compound according to the item (1) or (1)′, wherein the compound is represented by the Formula (IA-2):

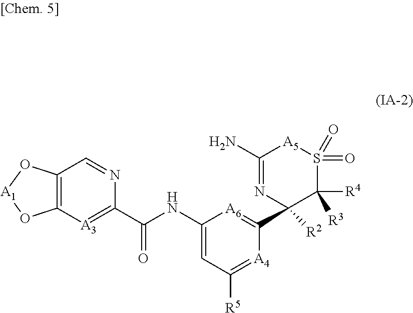

wherein
A₃ is N or CR¹; and the other symbols are the same as defined in the above item (1), or a pharmaceutically acceptable salt thereof.
(2-2) The compound according to the item (1), wherein the compound is represented by the Formula (IB-2):

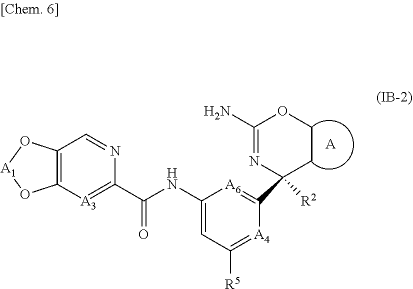

wherein each symbol is the same as defined in the above item (1), or a pharmaceutically acceptable salt thereof.
(2-2)′ The compound according to the items (1) or (2-2), wherein

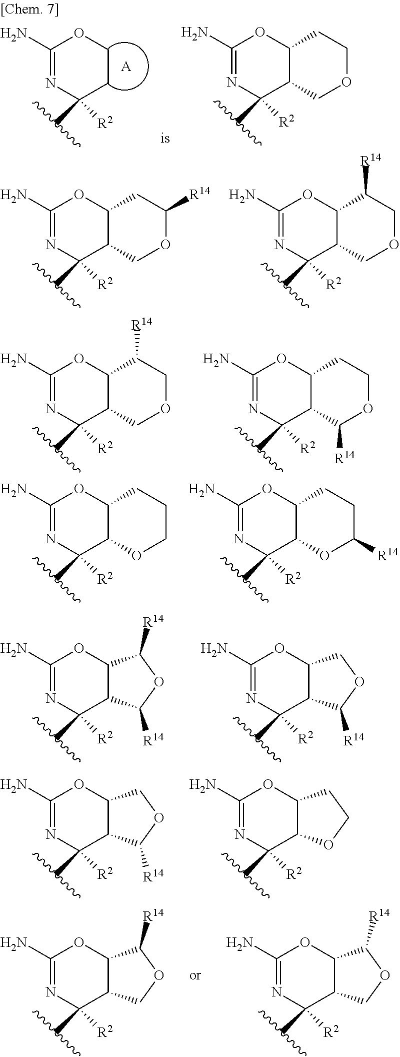

wherein each symbol is the same as defined in the above item (1),
or a pharmaceutically acceptable salt thereof.
(2-2)″ The compound according to the item (1) or (2-2), wherein

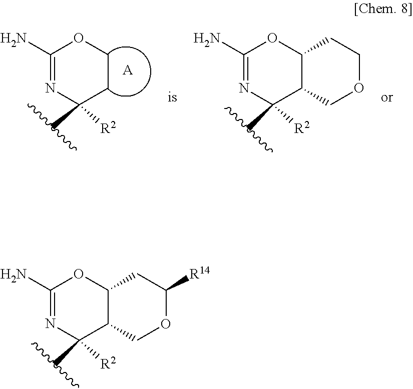

wherein each symbol is the same as defined in the above item (1),
or a pharmaceutically acceptable salt thereof.
(2-2)′″ The compound according to any one of the items (1), (2-2), (2-2)′, or (2-2)″, wherein R¹⁴ is each independently alkyl optionally substituted with halogen, or a pharmaceutically acceptable salt thereof.
(2-3) The compound according to the item (1), wherein the compound is represented by the Formula (IC-2):

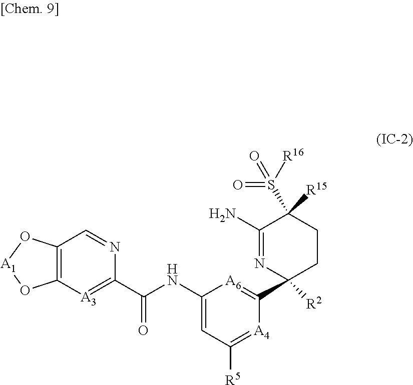

wherein each symbol is the same as defined in the above item (1),
or a pharmaceutically acceptable salt thereof.
(3) The compound according to any one of the items (1), (2), (2-2), (2-2)′, (2-2)″, (2-2)′″, (2-3) and (1)′, wherein -A₁- is selected from the group consisting of

### (i) —CH₂—,

(ii) —CH₂—CH₂—,
(iii) —CH₂—CH₂—CH₂—,

### (iv) —CD₂-,

(v) —CD₂-CD₂-,
(vi) —CD₂-CD₂-CD₂-,
(vii) —CF₂—,
(viii) —CF₂—CH₂—,
(ix) —CH₂—CF₂—,
(x) —CF₂—CH₂—CH₂—,
(xi) —CH₂—CF₂—CH₂—,
(xii) CH₂—CH₂—CF₂—,
(xiii) —CHF—,
(xiv) —CHF—CH₂—,

### (xv) —CH₂—CHF—,

(xvi) —CHF—CH₂—CH₂—,
(xvii) —CH₂—CHF—CH₂—,
(xviii) —CH₂—CH₂—CHF—,
(xix) —CH(Me)-,

### (xx) —CH(Me)-CH₂—,

(xxi) —CH₂—CH(Me)-.
(xxii) —CH(Me)-CH₂—CH₂—
(xxiii) —CH₂—CH(Me)-CH₂—, and
(xxiv) —CH₂—CH₂—CH(Me)-;
or a pharmaceutically acceptable salt.
(3-2) The compound according to any one of the items (1) to (3), (2-2), (2-2)′, (2-2)″,
(2-2)′″, (2-3) and (1)′, wherein -A₁- is selected from the group consisting of
(ii) —CH₂—CH₂—,
(iii) —CH₂—CH₂—CH₂—,
(v) —CD₂-CD₂-,
(vii) —CF₂—,
(viii) —CF₂—CH₂—,
(ix) —CH₂—CF₂—,
(xiv) —CHF—CH₂—,

### (xv) —CH₂—CHF—,

### (xx) —CH(Me)-CH₂—, and

(xxi) —CH₂—CH(Me)-;
or a pharmaceutically acceptable salt.
(3-3) The compound according to any one of the items (1) to (3), (2-2), (2-2)′, (2-2)″, (2-2)′″, (2-3), (3-2) and (1)′, wherein -A₁- is selected from the group consisting of
(ii) —CH₂—CH₂—,
(vii) —CF₂—,
(ix) —CH₂—CF₂—, and
(xiv) —CHF—CH₂—, or a pharmaceutically acceptable salt thereof.
(4) The compound according to any one of the items (1) to (3), (2-2), (2-2)′, (2-2)″, (2-2)′″, (2-3), (3-2), (3-3) and (1)′, wherein -A₁- is selected from the group consisting of

### (iv) —CD₂-,

(v) —CD₂-CD₂-,
(vi) —CD₂-CD₂-CD₂-,
(vii) —CF₂—,
(viii) —CF₂—CH₂—,
(ix) —CH₂—CF₂—,
(x) —CF₂—CH₂—CH₂—,
(xi) —CH₂—CF₂—CH₂—,
(xii) CH₂—CH₂—CF₂—,
(xiii) —CHF—,
(xiv) —CHF—CH₂—,

### (xv) —CH₂—CHF—,

(xvi) —CHF—CH₂—CH₂—,
(xvii) —CH₂—CHF—CH₂—, and
(xviii) —CH₂—CH₂—CHF—,
or a pharmaceutically acceptable salt thereof.
(5) The compound according to any one of the items (1) to (4), (2-2), (2-2)′, (2-2)″, (2-2)′″, (2-3), (3-2), (3-3) and (1)′, wherein

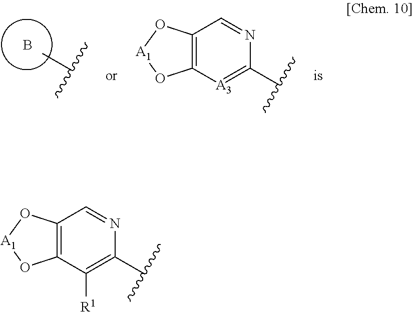

wherein R¹ is a hydrogen atom, fluoro, chloro, or methyl; or a pharmaceutically acceptable salt thereof. (5)′ The compound according to any one of the items (1) to (4), (2-2), (2-2)′, (2-2)″, (2-2)′″, (2-3), (3-2), (3-3), (1)′ and (5)′, wherein A₃ is CR¹ wherein R¹ is a hydrogen atom or chloro; or a pharmaceutically acceptable salt thereof.
(6) The compound according to any one of the items (1) to (5), (2-2), (2-2)′, (2-2)″, (2-2)′″, (2-3), (3-2), (3-3), (1)′ and (5)′, wherein

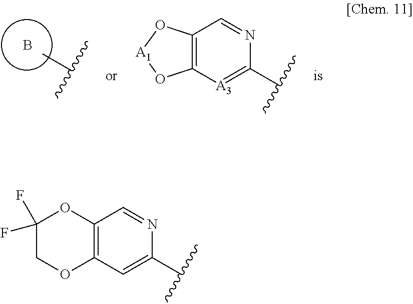

or a pharmaceutically acceptable salt thereof.
(7) The compound according to any one of the items (1) to (6), (2-2), (2-2)′, (2-2)″, (2-2)′″, (2-3), (3-2), (3-3), (1)′ and (5)′, wherein R² is methyl optionally substituted with fluoro; or a pharmaceutically acceptable salt thereof.
(7)′ The compound according to any one of the items (1) to (7), (2-2), (2-2)′, (2-2)″, (2-2)′″, (2-3), (3-2), (3-3), (1)′ and (5)′, wherein R² is methyl; or a pharmaceutically acceptable salt thereof.
(8) The compound according to any one of the items (1) to (7), (2-2), (2-2)′, (2-2)″, (2-2)′″, (2-3), (3-2), (3-3), (1)′, (5)′ and (7)′, wherein R³ and R⁴ are a hydrogen atom; or a pharmaceutically acceptable salt thereof.
(8-2) The compound according to any one of the items (1) to (8), (2-2), (2-2)′, (2-2)″, (2-2)′″, (2-3), (3-2), (3-3), (1)′, (5)′ and (7)′, wherein R² and one of R³ and R⁴ are taken together with an adjacent atom to form carbocycle or heterocycle; or a pharmaceutically acceptable salt thereof.
(8-3) The compound according to any one of the items (1) to (8), (2-2), (2-2)′, (2-2)″, (2-2)′″, (2-3), (3-2), (3-3), (5-2), (8-2), (1)′, (5)′ and (7)′, wherein one of R³ and R⁴ and one of R⁸ and R⁹ are taken together with an adjacent atom to form carbocycle or heterocycle; or a pharmaceutically acceptable salt thereof.
(9) The compound according to any one of the items (1) to (8), (2-2), (2-2)′, (2-2)″, (2-2)′″, (2-3), (3-2), (3-3), (5-2), (8-2), (8-3), (1)′, (5)′ and (7)′, wherein R⁵ is a hydrogen atom; or a pharmaceutically acceptable salt thereof.
(10) The compound according to any one of the items (1) to (9), (2-2), (2-2)′, (2-2)″, (2-2)′″, (2-3), (3-2), (3-3), (5-2), (8-2), (8-3), (1)′, (5)′ and (7)′, wherein A₄ is CR⁶ wherein R⁶ is halogen; or a pharmaceutically acceptable salt thereof.
(10)′ The compound according to any one of the items (1) to (10), (2-2), (2-2)′, (2-2)″, (2-2)′″, (2-3), (3-2), (3-3), (5-2), (8-2), (8-3), (1)′, (5)′ and (7)′, wherein A₆ is CR¹⁸ and R¹⁸ is a hydrogen atom
or a pharmaceutically acceptable salt thereof.
(11) The compound according to any one of the items (1) to (10), (2-2), (2-2)′, (2-2)″, (2-2)′″, (2-3), (3-2), (3-3), (5-2), (8-2), (8-3), (1)′, (5)′, (10)′, and (7)′, wherein A₅ is NR⁷; or a pharmaceutically acceptable salt thereof.
(12) The compound according to any one of the items (1) to (11), (2-2), (2-2)′, (2-2)″, (2-2)′″, (2-3), (3-2), (3-3), (5-2), (8-2), (8-3), (1)′, (5)′, (10)′, and (7)′, wherein R⁷ is methyl; or a pharmaceutically acceptable salt thereof.
(13) The compound according to any one of the items (1) to (12), (2-2), (2-2)′, (2-2)″, (2-2)′″, (2-3), (3-2), (3-3), (5-2), (8-2), (8-3), (1)′, (5)′, (10)′, and (7)′, wherein A₅ is CR⁸R⁹; or a pharmaceutically acceptable salt thereof.
(14) The compound according to any one of the items (1) to (13), (2-2), (2-2)′, (2-2)″, (2-2)′″, (2-3), (3-2), (3-3), (5-2), (8-2), (8-3), (1)′, (5)′, (10)′, and (7)′, wherein R⁸ and R⁹ are methyl; or a pharmaceutically acceptable salt thereof.
(14)′ The compound according to the item any one of the items (1) to (14), (2-2), (2-2)′, (2-2)″, (2-2)′″, (2-3), (3-2), (3-3), (5-2), (8-2), (8-3), (1)′, (5)′, (10)′, and (7)′, selected from the group consisting of Compound I-001, I-004, I-009, I-011, I-012, I-023, I-024, I-026, I-027, and I-029; or a pharmaceutically acceptable salt thereof.
(14-2) The compound according to the item any one of the items (1) to (14), (2-2), (2-2)′, (2-2)″, (2-2)′″, (2-3), (3-2), (3-3), (5-2), (8-2), (8-3), (1)′, (5)′ (10)′, and (14)′, selected from the group consisting of Compound I-012, I-035, and I-043; or a pharmaceutically acceptable salt thereof.
(15) A pharmaceutical composition comprising the compound according to any one of the items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof.
(16) The pharmaceutical composition having BACE1 inhibitory activity comprising the compound according to the item (15), or a pharmaceutically acceptable salt thereof.
(17) The pharmaceutical composition according to the items (15) or (16), for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia.
(18) A compound according to any one of the items (1) to (14), (2-2), (2-2)′, (2-2)″, (2-2)′″, (2-3), (3-2), (3-3), (5-2), (8-2), (8-3), (14-2), (1)′, (5)′ (10)′, and (14)′, or a pharmaceutically acceptable salt thereof for use in a method for inhibiting BACE1 activity.
(19) A compound according to any one of the items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof for use in treating or preventing Alzheimer dementia, mild cognitive impairment or prodromal Alzheimer's disease, for use in preventing the progression of Alzheimer dementia, mild cognitive impairment or prodromal Alzheimer's disease, or for use in preventing the progression in a patient asymptomatic at risk for Alzheimer dementia.
(20) A method for inhibiting BACE1 activity comprising administering the compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof.
(21) A method for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia comprising administering the compound according to any one of items (1) to (14), (2-2), (2-2)′, (2-2)″, (2-2)′″, (2-3), (3-2), (3-3), (5-2), (8-2), (8-3), (14- 2), (1)′, (5)′, (7)′, (10)′, and (14)′, or a pharmaceutically acceptable salt thereof.
(22) A BACE1 inhibitor comprising the compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof.
(22-2) Use of the compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′ and (5-3), or a pharmaceutically acceptable salt thereof for manufacturing a medicament for inhibiting BACE1 activity.
(23) The pharmaceutical composition according to the item (15) or (16) for treating or preventing a disease induced by production, secretion or deposition of amyloid ß proteins.
(24) A method for treating or preventing diseases induced by production, secretion or deposition of amyloid ß proteins comprising administering the compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof.
(25) A compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof for use in treating or preventing diseases induced by production, secretion or deposition of amyloid ß proteins.
(26) Use of the compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof for manufacturing a medicament for treating or preventing diseases induced by production, secretion or deposition of amyloid ß proteins.
(27) The pharmaceutical composition according to the item (15) or (16), for treating or preventing Alzheimer dementia.
(28) A method for treating or preventing Alzheimer dementia comprising administering the compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof.
(29) A compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof for use in treating or preventing Alzheimer dementia.
(30) Use of the compound according to any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof for manufacturing a medicament for treating or preventing Alzheimer dementia.
(31) A pharmaceutical composition comprising the compound of any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof, for a pediatric or geriatric patient.
(32) A pharmaceutical composition consisting of a combination of the compound of any one of items (1) to (14), (2-2), (2-2)′, (2-2)″, (2-2)′″, (2-3), (3-2), (3-3), (5-2), (8-2), (8-3), (14-2), (1)′, (5)′, (7)′, (10)′, and (14)′, or a pharmaceutically acceptable salt thereof and acetylcholinesterase inhibitor, NMDA antagonist, or other medicament for Alzheimer dementia.
(33) A pharmaceutical composition comprising the compound of any one of items (1) to (14), (2-2), (2-2)′, (2-2)″, (2-2)′″, (2-3), (3-2), (3-3), (5-2), (8-2), (8-3), (14- 2), (1)′, (5)′, (7)′, (10)′, and (14)′, or a pharmaceutically acceptable salt thereof, for a combination therapy with acetylcholinesterase inhibitor, NMDA antagonist, or other medicament for Alzheimer dementia.

### DESCRIPTION OF EMBODIMENTS

Hereinafter, the present invention is described with reference to embodiments. It should be understood that, throughout the present specification, the expression of a singular form includes the concept of its plural form unless specified otherwise. Accordingly, it should be understood that an article in singular form (for example, in the English language, “a,” “an,” “the,” and the like) includes the concept of its plural form unless specified otherwise. Furthermore, it should be understood that the terms used herein are used in a meaning normally used in the art unless specified otherwise. Thus, unless defined otherwise, all technical and scientific terms used herein have the same meaning as those generally understood by those skilled in the art in the field to which the present invention pertains. If there is a contradiction, the present specification (including definitions) precedes.

Each meaning of terms used herein is described below. Both when used alone and in combination unless otherwise noted, each term is used in the same meaning.

In the specification, the term of “consisting of” means having only components.

In the specification, the term of “comprising” means not restricting with components and not excluding undescribed factors.

In the specification, the “halogen” includes fluorine, chlorine, bromine, and iodine. Fluorine and chlorine are preferable. Fluorine is more preferable.

In the specification, the “alkyl” includes linear or branched alkyl of a carbon number of 1 to 15, for example, a carbon number of 1 to 10, for example, a carbon number of 1 to 6, for example, a carbon number of 1 to 4, preferably carbon number of 1 to 3, and more preferably carbon number of 1 or 2. Examples include methyl, ethyl, n-propyl, isopropyl, n-butyl, isobutyl, sec-butyl, tert-butyl, n-pentyl, isopentyl, neopentyl, hexyl, isohexyl, n-heptyl, isoheptyl, n-octyl, isooctyl, n-nonyl and n-decyl. Examples are methyl, ethyl, n-propyl, isopropyl, n-butyl, isobutyl, sec-butyl, tert-butyl and n-pentyl.

In one embodiment, “alkyl” is methyl, ethyl, n-propyl, isopropyl or tert-butyl.

The term of “haloalkyl” includes a group wherein one or more hydrogen atoms attached to one or more carbon atoms of the above “alkyl” are replaced with one or more above “halogen”. Examples are monofluoromethyl, monofluoroethyl, monofluoropropyl, difluoromethyl, difluoroethyl, difluoropropyl, trifluoromethyl, trifluoroethyl, trifluoropropyl, pentafluoropropyl, monochloromethyl, monochloroethyl, monochloropropyl, dichloromethyl, dichloroethyl, dichloropropyl, trichloromethyl, trichloroethyl, trichloropropyl, pentachloropropyl, 1-fluoroethyl, 2-fluoroethyl, 1,1-difluoroethyl, 2,2-difluoroethyl, 2,2,2-trifluoroethyl, 1-chloroethyl, 2-chloroethyl, 1,1-dichloroethyl, 2,2-dichloroethyl, 2,2,2-trichloroethyl, 1,2-dibromoethyl, 1,1,1-trifluoropropan-2-yl and 2,2,3,3,3-pentafluoropropyl. Examples are monofluoromethyl, difluoromethyl, trifluoromethyl, 1-fluoroethyl, 1,1-difluoroethyl, and 2,2-difluoroethyl. Examples are monofluoromethyl, difluoromethyl, 1-fluoroethyl, 1,1-difluoroethyl and 2,2-difluoroethyl.

The term “alkylene” include a linear or branched divalent carbon chain of a carbon number of 1 to 15, for example, a carbon number of 1 to 10, for example, a carbon number of 1 to 6, and for example a carbon number of 1 to 3. Examples are methylene, dimethylene, and trimethylene.

One or more hydrogens of the alkylene in a compound of formula (IA), (IB), or (IC) can be replaced with an isotope of hydrogen ²H (deuterium).

The term of “carbocycle” includes non-aromatic carbocycle and aromatic carbocycle.

The term of “non-aromatic carbocycle” includes saturated carbocycle or unsaturated non-aromatic carbocycle which is monocyclic or which consists of two or more rings. A “non-aromatic carbocycle” of two or more rings includes a fused cyclic group wherein a non-aromatic monocyclic carbocycle or a non-aromatic carbocycle of two or more rings is fused with a ring of the above “aromatic carbocycle”.

In addition, the “non-aromatic carbocycle” also includes a cyclic group having a bridge or a cyclic group to form a spiro ring as follows:

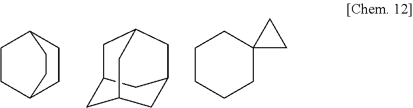

The term “non-aromatic monocyclic carbocycle” includes a group having 3 to 16 carbon atoms, for example, 3 to 12 carbon atoms, for example, 3 to 8 carbon atoms, and for example, 3 to 5 carbon atoms. Examples are cyclopropane, cyclobutane, cyclopentane, cyclohexane, cycloheptane, cyclooctane, cyclononane, cyclodecane, cyclopropenane, cyclobutenane, cyclopentenane, cyclohexenane, cycloheptenane and cyclohexadienane. For example, cyclopropane.

Examples of non-aromatic carbocycle consisting of two or more rings include a group having 6 to 14 carbon atoms, and examples are indane, indenane, acenaphthalene, tetrahydronaphthale and fluorenone.

The term of “aromatic carbocycle” includes an aromatic hydrocarbon ring which is monocyclic or which consists of two or more rings. Examples are an aromatic hydrocarbon group of a carbon number of 6 to 14, and specific examples are benzene, naphthalene, anthracene and phenanthrene.

In one embodiment, “aromatic carbocycle” is benzene.

In one embodiment, “carbocycle” is cyclopropane, cyclobutane and cyclopentane.

The term of “heterocycle” includes non-aromatic heterocycle and aromatic heterocycle.

The term of “non-aromatic heterocycle” includes a non-aromatic group which is monocyclic, or which consists of two or more rings, containing one or more of heteroatoms selected independently from oxygen, sulfur and nitrogen atoms.

A “non-aromatic heterocycle” of two or more rings includes a fused cyclic group wherein non-aromatic monocyclic heterocycle or non-aromatic heterocycle of two or more rings is fused with a ring of the above “aromatic carbocycle”, “non-aromatic carbocycle” and/or “aromatic heterocycle”.

In addition, the “non-aromatic heterocycle” also includes a cyclic ring having a bridge or a cyclic group to form a spiro ring as follows:

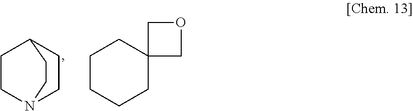

The term “non-aromatic monocyclic heterocycle” includes a 3- to 8-membered ring, and for example, 4-, 5- or 6-membered ring. Examples are dioxane, thiirane, oxirane, oxetane, oxathiolane, azetidine, thiane, thiazolidine, pyrrolidine, pyrroline, imidazolidine, imidazoline, pyrazolidine, pyrazoline, piperidine, piperazine, morpholinyl, morpholine, thiomorpholine, dihydropyridine, tetrahydropyridine, tetrahydrofuran, tetrahydropyrane, dihydrothiazoline, tetrahydrothiazoline, tetrahydroisothiazoline, dihydrooxazine, hexahydroazepine, tetrahydrodiazepine, tetrahydropyridazine, hexahydropyrimidine, dioxolane, dioxazine, aziridine, dioxoline, oxepane, thiolane, thiine and thiazine.

Examples of non-aromatic heterocycle of two or more rings includes a 9 to 14-membered group, and examples are indoline, isoindoline, chromane and isochromane.

The term of “aromatic heterocycle” includes an aromatic ring which is monocyclic, or which consists of two or more rings, containing one or more of heteroatoms selected independently from oxygen, sulfur and nitrogen atoms.

An “aromatic heterocycle” of two or more rings includes a fused cyclic group wherein aromatic monocyclic heterocyclyl or non-aromatic heterocycle consisting of two or more rings is fused with a ring of the above “aromatic carbocycle”.

The term “aromatic monocyclic heterocycle” includes a 5- to 8-membered group, and for example, 5- to 6-membered ring. Examples are pyrrole, imidazole, pyrazole, pyridine, pyridazine, pyrimidine, pyrazine, triazole, triazine, tetrazole, furane, thiophene, isoxazole, oxazole, oxadiazole, isothiazole, thiazole and thiadiazole.

Examples of aromatic bicyclic heterocycle includes a 9- to 10-membered ring, and examples are indoline, isoindoline, indazoline, indolizine, quinoline, isoquinoline, cinnoline, phthalazine, quinazoline, naphthyridine, quinoxaline, purine, pteridine, benzimidazole, benzisoxazole, benzoxazole, benzoxadiazole, benzisothiazole, benzothiazole, benzothiadiazole, benzofurane, isobenzofurane, benzothiophene, benzotriazole, imidazopyridine, triazolopyridine, imidazothiazole, pyrazinopyridazine, oxazolopyridine and thiazolopyridine.

Examples of aromatic heterocycle of three or more rings includes a 13 to 14-membered group, and examples are carbazole, acridine, xanthene, phenothiazine, phenoxathiine phenoxazine and dibenzofurane.

In one embodiment, “heterocycle” is 1,4-oxathiane.

The term “R² and one of R³ and R⁴ may be taken together with an adjacent atom to form carbocycle or heterocycle” and “R² and one of R³ and R⁴ may be taken together with the carbon atoms to which they are attached to form a carbocycle or a heterocycle” include

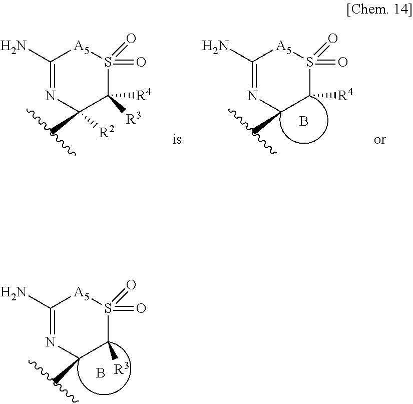

wherein ring B is substituted or unsubstituted carbocycle or substituted or unsubstituted heterocyle.

The term “one of R³ and R⁴ and one of R⁸ and R⁹ may form alkylene;” includes

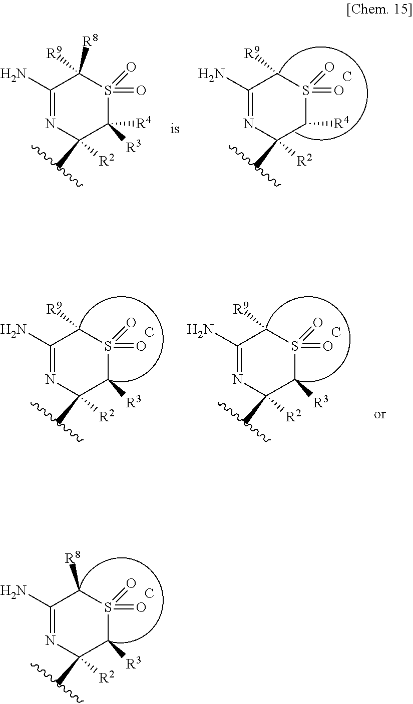

and one of the carbon atoms that consist of the alkylene may be replaced with an oxygen atom or a nitrogen atom; the carbon atoms that consist of the alkylene are each independently substituted with the substituent selected from R^{a}, and the nitrogen atom that consists of the alkylene is substituted with the substituent selected from R^{b};
R^{a} is a hydrogen atom, halogen, hydroxy, cyano, or substituted or unsubstituted alkyl;
R^{b} is a hydrogen atom, substituted or unsubstituted alkyl.

The term “R⁸ and R⁹ may be taken together with an adjacent atom to form carbocycle or heterocycle” and “R⁸ and R⁹ may be taken together with the carbon atom to which they are attached to form a carbocycle or a heterocycle” include

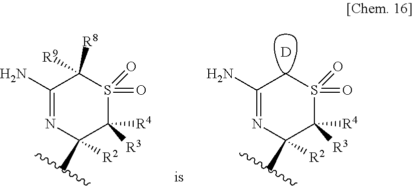

wherein ring D is substituted or unsubstituted carbocycle or substituted or unsubstituted heterocyle.

The term “R³ and R⁴ may be taken together with an adjacent atom to form carbocycle or heterocycle” and “R³ and R⁴ may be taken together with the carbon atom to which they are attached to form a carbocycle or a heterocycle” include

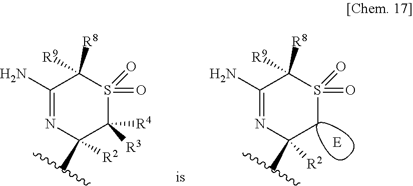

Examples of substituents of “substituted or unsubstituted alkyl” are one or more groups selected from the following substituent group α.

The substituent group α is a group consisting of halogen, hydroxy, alkyloxy, haloalkyloxy, alkyloxyalkyloxy, carboxy, amino, and cyano.

The substituents of “substituted or unsubstituted alkyl” are, for example, halogen, cyano and the like.

Examples of the substituent of “substituted or unsubstituted carbocycle”, or “substituted or unsubstituted heterocycle” include a group selected from the substituent group α.

The substituents of “substituted or unsubstituted alkyl” in R² are for example, halogen and the like.

The substituents of “substituted or unsubstituted alkyl” in R¹⁴ are for example, halogen, alkyloxy and the like.

Specific embodiments of each symbol of the formula (IA), (IB), (IC), (IA-2), (IB-2) and (IC-2) are illustrated below. All combination of these embodiments are examples of the compounds of formulas (IA), (IB), (IC), (IA-2)′, (IA-2), (IB-2) and (IC-2).

-A₁- is alkylene optionally substituted with one or more halogen.
-A₁- is selected from the group consisting of:

### (i) —CH₂—,

(ii) —CH₂—CH₂—,
(iii) —CH₂—CH₂—CH₂—,

### (iv) —CD₂-,

(v) —CD₂-CD₂-,
(vi) —CD₂-CD₂-CD₂-,
(vii) —CF₂—,
(viii) —CF₂—CH₂—,
(ix) —CH₂—CF₂—,
(x) —CF₂—CH₂—CH₂—,
(xi) —CH₂—CF₂—CH₂—,
(xii) —CH₂—CH₂—CF₂—,
(xiii) —CHF—,
(xiv) —CHF—CH₂—,

### (xv) —CH₂—CHF—,

(xvi) —CHF—CH₂—CH₂—,
(xvii) —CH₂—CHF—CH₂—,
(xviii) —CH₂—CH₂—CHF—,
(xix) —CH(Me)-,

### (xx) —CH(Me)-CH₂—,

(xxi) —CH₂—CH(Me)-.
(xxii) —CH(Me)-CH₂—CH₂—
(xxiii) —CH₂—CH(Me)-CH₂—, and
(xxiv) —CH₂—CH₂—CH(Me)-.
-A₁- is selected from the group consisting of:

### (iv) —CD₂-,

(v) —CD₂-CD₂-,
(vi) —CD₂-CD₂-CD₂-,
(vii) —CF₂—,
(viii) —CF₂—CH₂—,
(ix) —CH₂—CF₂—,
(x) —CF₂—CH₂—CH₂—,
(xi) —CH₂—CF₂—CH₂—,
(xii) —CH₂—CH₂—CF₂—,
(xiii) —CHF—,
(xiv) —CHF—CH₂—,

### (xv) —CH₂—CHF—,

(xvi) —CHF—CH₂—CH₂—,
(xvii) —CH₂—CHF—CH₂—, and
(xviii) —CH₂—CH₂—CHF—.
-A₁- is selected from the group consisting of:
(v) —CD₂-CD₂-,
(vii) —CF₂—,
(viii) —CF₂—CH₂—,
(ix) —CH₂—CF₂—,
(xiv) —CHF—CH₂—, and

### (xv) —CH₂—CHF—.

-A₁- is selected from the group consisting of:

### (i) —CH₂—,

(ii) —CH₂—CH₂—,
(iii) —CH₂—CH₂—CH₂—,
(v) —CD₂-CD₂-,
(vii) —CF₂—,
(viii) —CF₂—CH₂—,
(ix) —CH₂—CF₂—,
(xiv) —CHF—CH₂—, and

### (xv) —CH₂—CHF—.

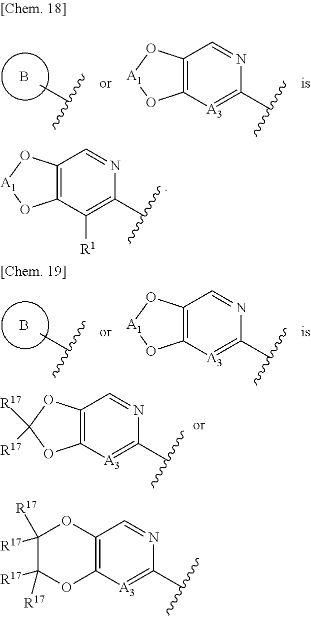

wherein R¹⁷ is each independently H, D, F or methyl.

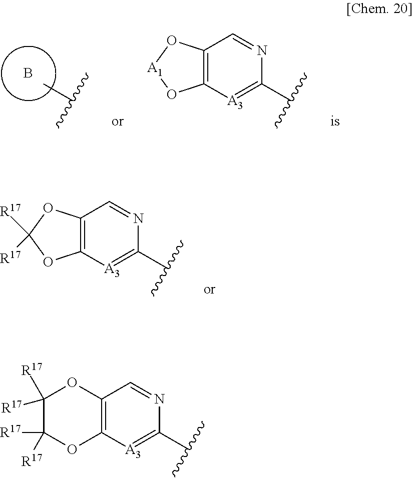

wherein R¹⁷ is each independently H, D, F.

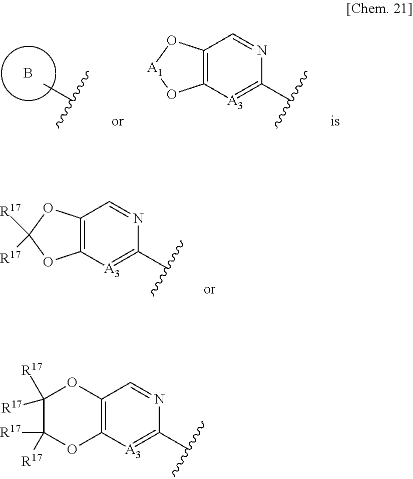

wherein R¹⁷ is each independently H, D, F; and at least one of R¹⁷ is D or F.

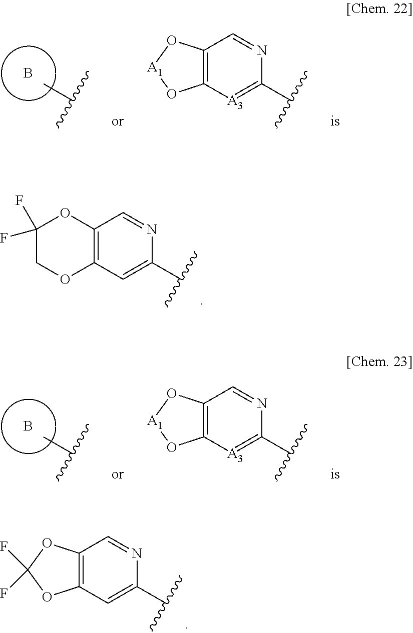

A₃ is N or CR¹.
A₃ is CR¹.
R¹ is a hydrogen atom, halogen, alkyl, haloalkyl or amino.
R¹ is a hydrogen atom, fluoro, chloro, methyl or amino.
R¹ is a hydrogen atom.
R² is substituted or unsubstituted alkyl.
R² is alkyl optionally substituted with one or more halogen.
R² is methyl optionally substituted with fluoro.
R² is methyl.
R² is fluoromethyl.
R³ and R⁴ are each independently a hydrogen atom, halogen, alkyl or haloalkyl.
R³ and R⁴ are each independently a hydrogen atom.
R⁵ is a hydrogen atom or halogen.
R⁵ is a hydrogen atom.
A₆ is CR¹⁸ or N and R¹⁸ is a hydrogen atom;
A₆ is CR¹⁸ and R¹⁸ is a hydrogen atom;
A₄ is N or CR⁶ wherein R⁶ is a hydrogen atom, halogen or substituted or unsubstituted alkyl.
A₄ is CR⁶ wherein R⁶ is halogen.
A₄ is CR⁶ wherein R⁶ is fluoro.

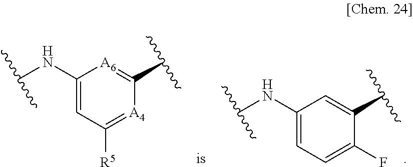

A₅ is NR⁷ or CR⁸R⁹.
A₅ is NR⁷.
A₅ is CR⁸R⁹.
R⁷ is substituted or unsubstituted alkyl.
R⁷ is C1-C3 alkyl.
R⁷ is methyl.
R⁸ and R⁹ are each independently a hydrogen atom, halogen, alkyl or haloalkyl.
R⁸ and R⁹ are each independently alkyl.
R⁸ and R⁹ are each independently C1-C3 alkyl.
R⁸ and R⁹ are each independently methyl.
R¹⁴ is each independently alkyl optionally substituted with one or more group(s) selected from halogen, cyano, alkyloxy, haloalkyloxy, and non-aromatic carbocyclyl; or heteroaryl optionally substituted with one or more alkyl;
two R¹⁴s attached to a same carbon atom may be taken together with the carbon atom to which they are attached to form a 3- to 5-membered non-aromatic carbocycle optionally substituted with one or more group(s) selected from halogen, alkyl and haloalkyl.
R¹⁴ is each independently C1-C3 alkyl optionally substituted with one or more group(s) selected from halogen.
t is an integer from 0 to 3.
t is an integer from 0 or 1.
t is 0.
R¹⁵ is alkyl optionally substituted with one or more group(s) selected from halogen.
R¹⁵ is C1-C3 alkyl optionally substituted with one or more group(s) selected from halogen.
R¹⁵ is alkyl.
R¹⁵ is C1-C3 alkyl.
R¹⁵ is methyl.
R¹⁶ is substituted or unsubstituted alkyl or non-aromatic carbocyclyl.
R¹⁶ is C1-C3alkyl, C1-C3haloalkyl or cyclopropyl.
R¹⁶ is methyl or ethyl.
R¹⁶ is methyl.

In one embodiment, in formula (IA-2) or (IA-2)′,

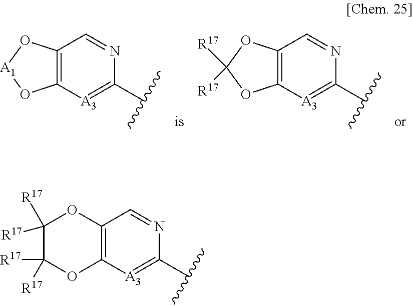

wherein R¹⁷ is each independently H, D, F or methyl;
A₃ is N or CR¹;
R¹ is a hydrogen atom;
R² is methyl optionally substituted with fluoro;
R³ and R⁴ are each independently a hydrogen atom;
R⁵ is a hydrogen atom or halogen;

### A₄ is CF;

A₅ is NR⁷ or CR⁸R⁹;
A₆ is CR¹⁸;
R¹⁸ is a hydrogen atom;

and

R⁷ is C1-C3 alkyl; and R⁸ and R⁹ are C1-C3 alkyl.

In one embodiment, in formula (IA-2) or (IA-2)′,

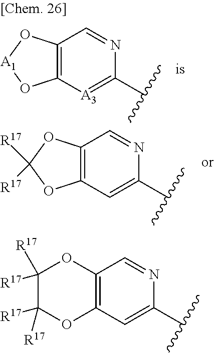

wherein R¹⁷ is each independently H, D, F or methyl, preferably at least one of R¹⁷ is D or F;
R¹ is a hydrogen atom;
R² is methyl;
R³ and R⁴ are each independently a hydrogen atom;
R⁵ is a hydrogen atom or halogen;

### A₄ is CF;

A₅ is NR⁷ or CR⁸R⁹;
A₆ is CR¹⁸;
R¹⁸ is a hydrogen atom;
and
R⁷ is methyl; and R⁸ and R⁹ are methyl.

In one embodiment, in formula (IC-2),

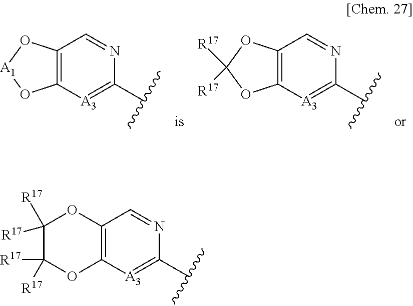

wherein R¹⁷ is each independently H, D, F or methyl;
A₃ is N or CR¹;
R¹ is a hydrogen atom or fluoro;
R² is methyl optionally substituted with fluoro;

### A₄ is CF;

A₆ is CR¹⁸;
R¹⁸ is a hydrogen atom;
R⁵ is a hydrogen atom or fluoro;
R¹⁵ is C1-C3 alkyl; and
R¹⁶ is C1-C3 alkyl.

The compound of formula (IA), (IB), or (IC) is not limited to a specific isomer, and includes all possible isomers such as keto-enol isomers, imine-enamine isomers, diastereoisomers, optical isomers and rotation isomers, racemate and the mixture thereof. For example, the compound of formula (IA), (IB), or (IC) includes the following tautomers.

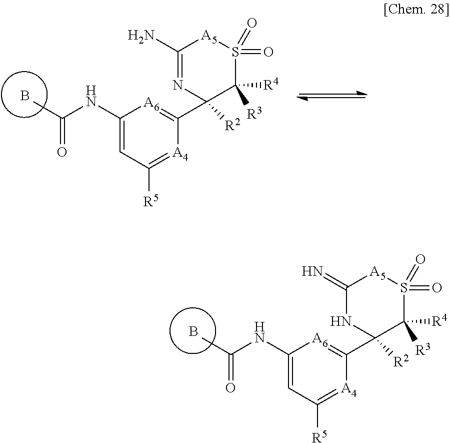

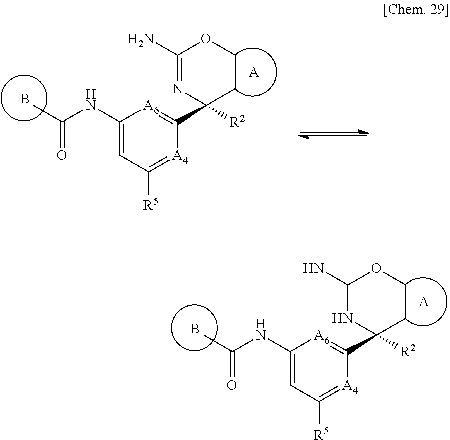

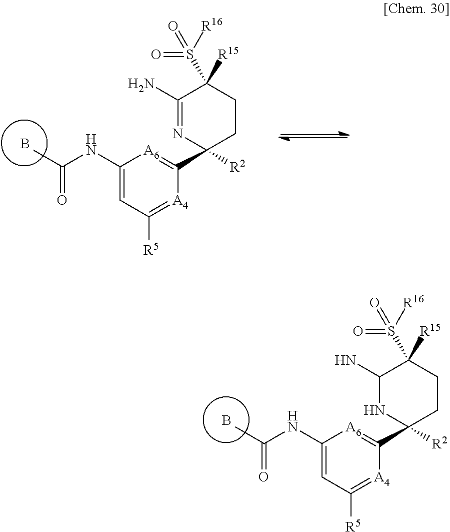

One or more hydrogen, carbon and/or other atoms of a compound of formula (IA), (IB), or (IC) can be replaced with an isotope of hydrogen, carbon and/or other atoms, respectively. Examples of isotopes include isotopes of hydrogen, carbon, nitrogen, oxygen, phosphorous, sulfur, fluorine, iodine and chlorine, such as ²H (D), ³H, ¹¹C, ¹³C, ¹⁴C, ¹⁵N, ¹⁸O, ¹⁷O, ³¹P, ³²P, ³⁵S, ¹⁸I, ¹²³I and ³⁶Cl, respectively. The compound of formula (IA), (IB), or (IC) also includes the compound replaced with such isotopes. The compound replaced with such isotopes is useful also as a medicament, and includes all the radiolabeled compounds of the compound of formula (IA), (IB), or (IC). The invention includes “radiolabelling method” for manufacturing the “radiolabeled compound” and the method is useful as a tool of metabolic pharmacokinetic research, the research in binding assay and/or diagnosis.

A radiolabeled compound of the compound of formula (IA), (IB), or (IC) can be prepared by methods known in the art. For example, tritiated compounds of formula (IA), (IB), or (IC) can be prepared by introducing tritium into the particular compound of formula (IA), (IB), or (IC) such as by catalytic dehalogenation with tritium. This method may include reacting a suitably halogenated precursor of a compound of formula (IA), (IB), or (IC) with tritium gas in the presence of a suitable catalyst such as Pd/C, in the presence or absence of a base. Other suitable methods for preparing tritiated compounds can be found in Isotopes in the Physical and Biomedical Sciences, Vol. 1, Labeled Compounds (Part A), Chapter 6 (1987). A ¹⁴C-labeled compound can be prepared by employing starting materials having ¹⁴C carbon.

As pharmaceutically acceptable salt of the compound of formula (IA), (IB), or (IC), examples include salts with alkaline metals (e.g. lithium, sodium and potassium), alkaline earth metals (e.g. calcium and barium), magnesium, transition metal (e.g. zinc and iron), ammonia, organic bases (e.g. trimethylamine, triethylamine, dicyclohexylamine, ethanolamine, diethanolamine, triethanolamine, meglumine, diethanolamine, ethylene diamine, pyridine, picoline, quinoline), and amino acids, and salts with inorganic acids (e.g. hydrochloric acid, sulfuric acid, nitric acid, carbonic acid, hydrobromic acid, phosphoric acid and hydroiodic acid) and organic acids (e.g. formic acid, acetic acid, propionic acid, trifluoroacetic acid, citric acid, lactic acid, tartaric acid, oxalic acid, maleic acid, fumaric acid, succinic acid, mandelic acid, glutaric acid, malic acid, benzoic acid, phthalic acid, ascorbic acid, benzenesulfonic acid, p-toluenesulfonic acid, methanesulfonic acid and ethanesulfonic acid). Specific Examples are salts with hydrochloric acid, sulfuric acid, phosphoric acid, tartaric acid, or methanesulfonic acid. These salts can be formed by the usual method.

The compounds of the present invention represented by formula (IA), (IB), or (IC) or pharmaceutically acceptable salts thereof may form solvates (e.g., hydrates etc.) and/or crystal polymorphs. The present invention encompasses those various solvates and crystal polymorphs. “Solvates” may be those wherein any number of solvent molecules (e.g., water molecules etc.) are coordinated with the compounds represented by formula (IA), (IB), or (IC). When the compounds represented by formula (IA), (IB), or (IC) or pharmaceutically acceptable salts are allowed to stand in the atmosphere, the compounds may absorb water, resulting in attachment of adsorbed water or formation of hydrates. Recrystallization of the compounds represented by formula (IA), (IB), or (IC) or pharmaceutically acceptable salts may produce crystal polymorphs.

Another Representative acids which may be used in the preparation of pharmaceutically acceptable salts include, but are not limited to, the following: acetic acid, 2,2-dichloroacetic acid, acylated amino acids, adipic acid, alginic acid, ascorbic acid,

L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The compounds of the present invention represented by formula (IA), (TB), or (IC) or pharmaceutically acceptable salts thereof may form prodrugs. The present invention also encompasses such various prodrugs. Prodrugs are derivatives of the compounds of the present invention that have chemically or metabolically degradable groups and are compounds that are converted to the pharmaceutically active compounds of the present invention through solvolysis or under physiological conditions in vivo. Prodrugs include compounds that are converted to the compounds represented by formula (IA), (IB), or (IC) through enzymatic oxidation, reduction, hydrolysis and the like under physiological conditions in vivo and compounds that are converted to the compounds represented by formula (IA), (IB), or (IC) through hydrolysis by gastric acid and the like. Methods for selecting and preparing suitable prodrug derivatives are described, for example, in the Design of Prodrugs, Elsevier, Amsterdam 1985. Prodrugs themselves may be active compounds.

When the compounds of formula (IA), (IB), or (IC) or pharmaceutically acceptable salts thereof have a hydroxy group, prodrugs include acyloxy derivatives and sulfonyloxy derivatives which can be prepared by reacting a compound having a hydroxy group with a suitable acid halide, suitable acid anhydride, suitable sulfonyl chloride, suitable sulfonylanhydride and mixed anhydride or with a condensing agent. Examples are CH₃COO—, C₂H₅COO—, t-BuCOO—, C₁₅H₃₁COO—, PhCOO—, (m-NaOOCPh)COO—, NaOOCCH₂CH₂COO—, CH₃CH(NH₂) COO—, CH₂N(CH₃)₂COO—, CH₃SO₃—, CH₃CH₂SO₃—, CF₃SO₃—, CH₂FSO₃—, CF₃CH₂SO₃—, p-CH₃—O-PhSO₃—, PhSO₃— and p-CH₃PhSO₃—.

The names of compounds were generated according to the nomenclature rules agreed upon by the Chemical Abstracts Service (CAS) or according to the nomenclature rules agreed upon by the International Union of Pure and Applied Chemistry (IUPAC).

The compounds of formula (IA), (IB), or (IC) may be prepared by the methods described below, together with synthetic methods known to a person skilled in the art.

The starting materials are commercially available or may be prepared in accordance with known methods.

During any of the following syntheses, it may be necessary or preferable to protect sensitive or reactive groups on any of molecules. In such case, these protections can be achieved by means of conventional protective groups such as those described in Greene's Protective Group in Organic Synthesis, John Wily & Sons, 2007.

It will be understood by a person skilled in the art that the compounds described below will be generated as a mixture of diastereomers and/or enantiomers, which may be separated at relevant stages of the following procedures using conventional techniques such as crystallization, silica gel chromatography, chiral or achiral high performance liquid chromatography (HPLC), and chiral supercritical fluid (SFC) chromatography to provide the single enantiomers of the invention.

During all the following steps, the order of the steps to be performed may be appropriately changed. In each step, an intermediate may be isolated and then used in the next step. All of reaction time, reaction temperature, solvents, reagents, protecting groups, etc. are mere exemplification and not limited as long as they do not cause an adverse effect on a reaction.

### General Procedure A

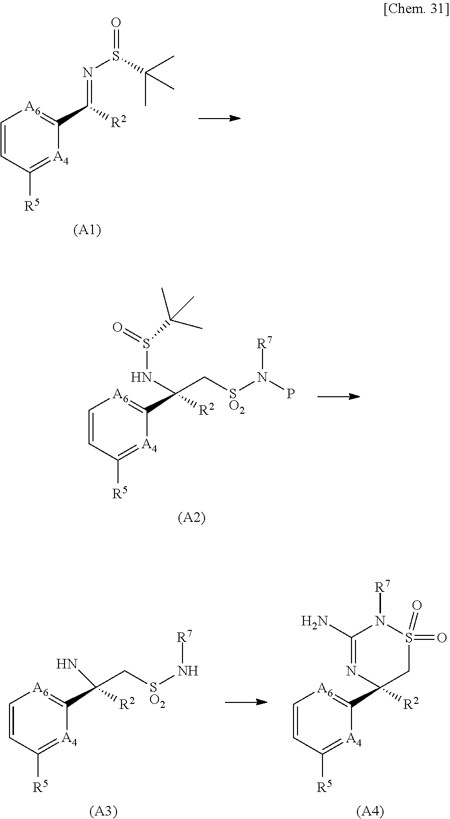

Wherein P is a protective group such as alkyl, benzoyl, benzyl, 4-methoxybenzyl or 2,4-dimethoxybenzyl, and the other symbols are the same as defined above (1).

General Procedure A is a method for preparing compounds of Compound A4 from Compounds A1 through multiple steps of Step 1 to Step 3. Those skilled in the art will be appreciate that protective groups P can be chosen depending on the reaction conditions used in later steps. The starting material of Compound A1 can be prepared in a manner similar to the conditions described in Chem. Rev. 2010, 110, 3600-3740.

### Step 1

Compound A2 can be prepared by means of the nucleophilic addition of an appropriate anion to Compound A1. This type of reactions can be conducted using the conditions described in J. Med. Chem. 2016, 59, 10435-10450. Preferably, the anions can be prepared from the corresponding methyl sulfonamides and an appropriate base, such as, for example, n-butyl lithium, which can be then reacted with Compound A1 to give Compound A2. The solvent used in this step is not particularly limited in so far as it does not interfere with the reaction. Examples of the solvent include tetrahydrofuran, 1,4-dioxane, 1,2-dimethoxyethane, diethyl ether, toluene, benzene, and mixed solvents thereof. The reaction temperature is preferably −78° C. to −30° C. The reaction time is not particularly limited and is usually 5 minutes to 24 hours, preferably 30 minutes to 24 hours.

### Step 2

Compound A3 can be prepared by deprotection of Compounds A2. This deprotection reaction is known to a person skilled in the art and can be performed under the conditions described in J. Med. Chem. 2016, 59, 10435-10450 and Org. Lett., 2016, 18 (22), 5780-5783. The reaction can be conducted under acidic conditions, preferably using hydrochloric acid or trifluoroacetic acid. The solvent used in this step is not particularly limited in so far as it does not interfere with the reaction. Examples of the solvent include dichloromethane, 1,4-dioxane, methanol, 1,3-dimethoxybenzene, toluene, and benzene and mixed solvents thereof. The reaction temperature is preferably room temperature to 60° C. The reaction time is not particularly limited and is usually 5 minutes to 24 hours, preferably 30 minutes to 24 hours.

### Step 3

Compound A4 can be prepared by cyclization of Compound A3. This cyclization reaction is known to a person skilled in the art and can be performed under the conditions described in J. Med. Chem. 2016, 59, 10435-10450 and Org. Lett., 2016, 18 (22), 5780-5783. The cyclization can be conducted using cyanogen bromide. The solvent used in this step is not particularly limited in so far as it does not interfere with the reaction. Examples of the solvent include acetonitrile, ethanol, 2-propanol, 1-butanol, mixed solvents thereof. The reaction temperature is usually 40° C. to 150° C. and is preferably 60° C. to 100° C. The reaction time is not particularly limited and is usually 5 minutes to 24 hours, preferably 30 minutes to 24 hours.

### General Procedure B:

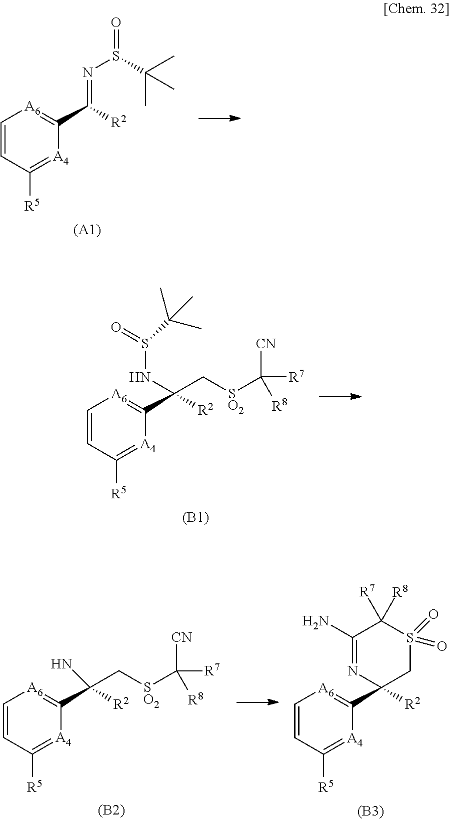

Wherein symbols are the same as defined above (1).

General Procedure B is a method for preparing Compound B3 from Compound A1 through multiple steps of Step 1 to Step 3.

### Step 1

Compound B1 can be prepared by means of the nucleophilic addition of an appropriate anion to Compound A1. Preferably, the anions can be prepared from the corresponding 2-(methylsulfonyl)acetonitriles, an appropriate base, such as, for example, n-butyl lithium, which can be then reacted with Compound A1 to give Compound B1. The solvent used in this step is not particularly limited in so far as it does not interfere with the reaction. Examples of the solvent include tetrahydrofuran, 1,4-dioxane, 1,2-dimethoxyethane, diethyl ether, toluene, benzene, and mixed solvents thereof. The reaction temperature is preferably −78° C. to −30° C. The reaction time is not particularly limited and is usually 5 minutes to 24 hours, preferably 30 minutes to 24 hours.

### Step 2

Compound B2 can be prepared by deprotection of Compound A2. The reaction can be conducted under acidic conditions, preferably using hydrochloric acid or trifluoroacetic acid. The solvent used in this step is not particularly limited in so far as it does not interfere with the reaction. Examples of the solvent include dichloromethane, 1,4-dioxane, methanol, 1.3-dimethoxybenzene, toluene, and benzene and mixed solvents thereof. The reaction temperature is preferably room temperature to 60° C. The reaction time is not particularly limited and is usually 5 minutes to 24 hours, preferably 30 minutes to 24 hours.

### Step 3

Compound B3 can be prepared by cyclization of Compound B2. The cyclization can be conducted under acidic conditions, preferably using hydrochloric acid. Alternatively, in the presence of a Lewis acid, such as, for example, trimethyl aluminium. The solvent used in this step is not particularly limited in so far as it does not interfere with the reaction. Examples of the solvent include dichloromethane, tetrahydrofuran, 1,4-dioxane, 1,2-dimethoxyethane, mixed solvents thereof. The reaction temperature is usually 40° C. to 150° C. and is preferably 60° C. to 110° C. The reaction time is not particularly limited and is usually 5 minutes to 24 hours, preferably 30 minutes to 24 hours.

### General Procedure C:

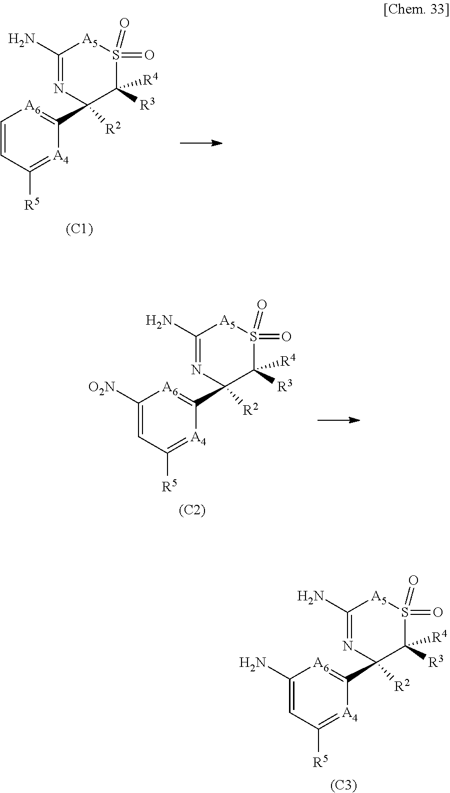

Wherein symbols are the same as defined above (1).

General Procedure C is a method for preparing Compound C3 from Compound C1 through multiple steps of Step 1 to Step 2.

### Step 1

Compound C2 can be prepared by nitration of Compound C1. A typical procedure involves the treatment of Compound C1 dissolved in sulfuric acid and trifluoroacetic acid, with a source of nitronium ion, such as, for example, potassium nitrate or nitric acid. The reaction temperature is preferably −20° C. to 0° C. The reaction time is not particularly limited and is usually 5 minutes to 5 hours, preferably 30 minutes to 2 hours.

### Step 2

Compound C3 can be prepared by reduction of Compound C2. The reduction can be conducted by a suitable catalyst, such as, for example, palladium on carbon, under hydrogen atmosphere, or the use of a reducing agent such as, for example, iron, zinc or tin(II) chloride. The solvent used in this step is not particularly limited in so far as it does not interfere with the reaction. Examples of the solvent include tetrahydrofuran, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### General Procedure D:

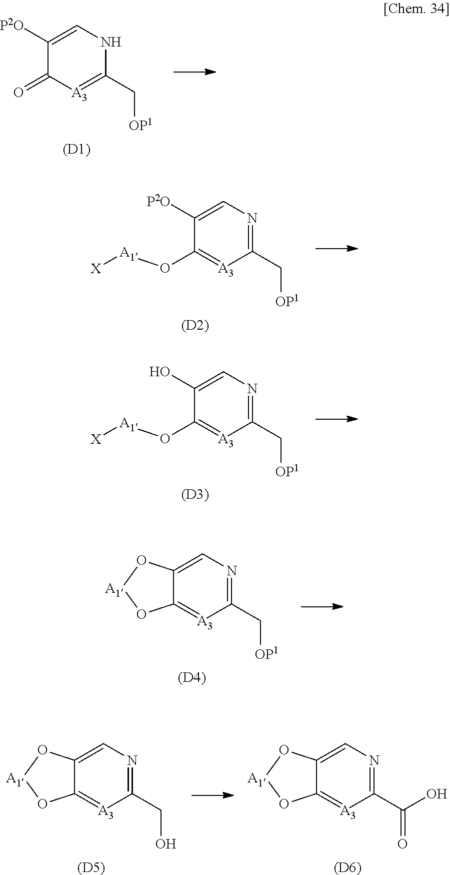

Wherein P¹ and P² are protective groups such as acetyl, benzoyl, benzyl, 4-methoxybenzyl, 2,4-dimethoxybenzyl, trityl or THP, and -A¹′- is alkylene optionally substituted with deuterium, and X is a leaving group such as OH, Cl, Br, and mesylate. Other symbols are the same as defined above (1).

### Step 1

General Procedure D is a method for preparing Compound D6 from Compound D1 through multiple steps of Step 1 to Step 5. Those skilled in the art will be appreciate that protective groups can be chosen depending on the reaction conditions used in later steps.

Compound D2 can be prepared by means of the alkylation of Compound D1. This type of reactions can be conducted with the corresponding alkylhalides using an appropriate base, such as, for example, sodium carbonate, potassium carbonate, and cesium carbonate. Alternatively, Compound D2 can be obtained by Mitsunobu reaction using the corresponding alcohol and reagents such as, for example, DEAD, DIAD or ADDP, and triphenylphosphine or tributylphosphine. The solvent used in this step is not particularly limited in so far as it does not interfere with the reaction. Examples of the solvent include tetrahydrofuran, 1,4-dioxane, DMF, DMA, NMP and mixed solvents thereof. The reaction temperature is usually room temperature to 150° C. and is preferably room temperature to 100° C. The reaction time is not particularly limited and is usually 5 minutes to 24 hours, preferably 30 minutes to 24 hours.

### Step 2

Compound D3 can be prepared by deprotection of Compound D2. This deprotection can be conducted under a suitable condition depending on the protecting group chosen. For example, when the protecting group is benzyl, Compound D3 can be prepared by a suitable catalyst, such as, for example, palladium on carbon, under hydrogen atmosphere. The solvent used in this step is not particularly limited in so far as it does not interfere with the reaction. Examples of the solvent include tetrahydrofuran 1,4-dioxane, methanol, ethanol, isopropanol, water and mixed solvents thereof. The reaction temperature is preferably room temperature to 60° C. The reaction time is not particularly limited and is usually 5 minutes to 24 hours, preferably 30 minutes to 24 hours.

### Step 3

Compound D4 can be prepared by means of the alkylation of Compound D3. For example, when X is a leaving group such as Cl, Br, and mesylate, this type of reactions can be conducted with an appropriate base, such as, for example, sodium carbonate, potassium carbonate, and cesium carbonate. Alternatively, when X is OH, Compound D3 can be obtained by Mitsunobu reaction using reagents such as, for example, DEAD, DIAD or ADDP, and triphenylphosphine or tributylphosphine. The solvent used in this step is not particularly limited in so far as it does not interfere with the reaction. Examples of the solvent include tetrahydrofuran, 1,4-dioxane, DMF, DMA, NMP and mixed solvents thereof. The reaction temperature is usually room temperature to 150° C. and is preferably room temperature to 100° C. The reaction time is not particularly limited and is usually 5 minutes to 24 hours, preferably 30 minutes to 24 hours.

### Step 4

Compound D5 can be prepared by deprotection of Compound D4. This deprotection can be conducted by a suitable condition for the chosen protecting group. For example, when the protecting group is trityl or THP, Compound D can be prepared under acidic conditions, such as p-toluenesulfonic acid or hydrochloric acid. The solvent used in this step is not particularly limited in so far as it does not interfere with the reaction. Examples of the solvent include tetrahydrofuran, 1,4-dioxane, methanol, ethanol, isopropanol, water and mixed solvents thereof. The reaction temperature is preferably room temperature to 100° C. The reaction time is not particularly limited and is usually 5 minutes to 24 hours, preferably 30 minutes to 24 hours.

Compound D6 can be prepared by oxidation of Compound D5. The oxidation can be conducted by a suitable condition, such as, for example, TEMPO oxidation using TEMPO, NaClO₂ and NaClO. The solvent used in this step is not particularly limited in so far as it does not interfere with the reaction. Examples of the solvent include dichloromethane, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### General Procedure E:

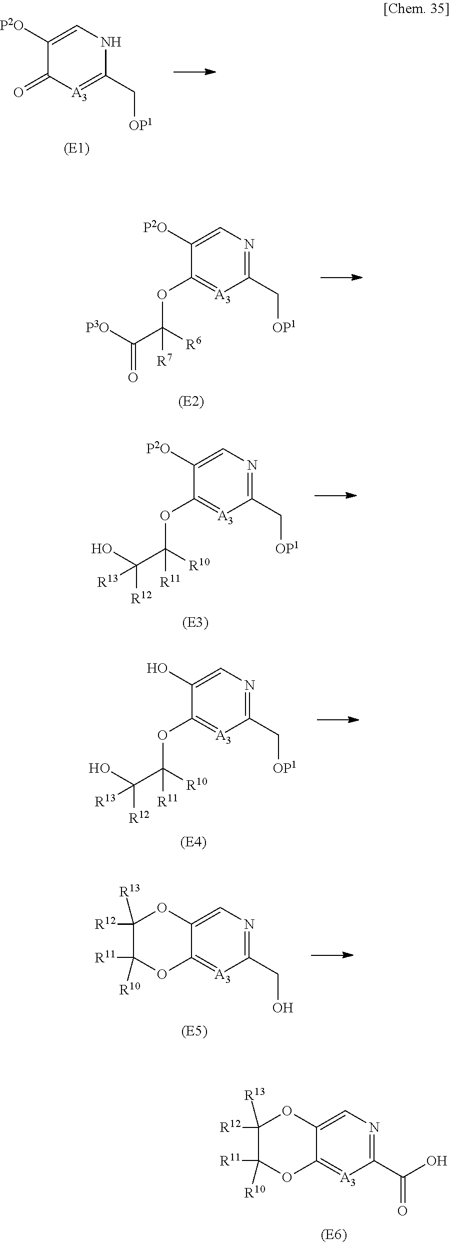

Wherein, P¹, P² and P² are protective groups such as alkyl, acetyl, benzoyl, benzyl, 4-methoxybenzyl, 2,4-dimethoxybenzyl, trityl or THP, and R¹⁰ and R¹¹ are each independently a hydrogen atom or fluoro, and R¹² and R¹³ are each independently a hydrogen atom, fluoro or alkyl. The other symbols are the same as defined above (1).

General Procedure E is a method for preparing Compound E6 from Compound E1 through multiple steps of Step 1 to Step 5. Those skilled in the art will be appreciate that protective groups can be chosen depending on the reaction conditions used in later steps.

### Step 1

Compound E2 can be prepared by means of the alkylation of Compound E1. This type of reactions can be conducted with the corresponding α-haloesters using an appropriate base, such as, for example, potassium carbonate, cesium carbonate and DBU. Alternatively, Compound E2 can be obtained by Mitsunobu reaction using the corresponding α-hydroxyesters and reagents such as, for example, DEAD, DIAD or ADDP, and triphenylphosphine or tributylphosphine. The solvent used in this step is not particularly limited in so far as it does not interfere with the reaction. Examples of the solvent include tetrahydrofuran, 1,4-dioxane, benzene, toluene, DMF, and mixed solvents thereof. The reaction temperature is usually room temperature to 150° C. and is preferably room temperature to 100° C. The reaction time is not particularly limited and is usually 5 minutes to 24 hours, preferably 30 minutes to 24 hours.

### Step 2

Compound E3 can be prepared by deprotection of Compound E2. This deprotection can be conducted by a suitable condition depending on the protecting group chosen. For example, when the protecting group is benzyl, Compound E3 can be prepared by a suitable catalyst, such as, for example, palladium on carbon, under hydrogen atmosphere. The solvent used in this step is not particularly limited in so far as it does not interfere with the reaction. Examples of the solvent include tetrahydrofuran, 1,4-dioxane, methanol, ethanol, isopropanol, water and mixed solvents thereof. The reaction temperature is preferably room temperature to 60° C. The reaction time is not particularly limited and is usually 5 minutes to 24 hours, preferably 30 minutes to 24 hours.

### Step 3

Compound E4 can be prepared by reduction of ester of Compound E3. The reduction can be conducted by a reducing reagent such as, for example, sodium borohydride, lithium borohydride and lithium aluminum hydride. The solvent used in this step is not particularly limited in so far as it does not interfere with the reaction. Examples of the solvent include tetrahydrofuran, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### Step 4

Compound E5 can be prepared by Mitsunobu reaction of Compound E4. This reaction can be conducted by suitable reagents such as for example, DEAD, DIAD or ADDP, and triphenylphosphine or tributylphosphine. The solvent used in this step is not particularly limited in so far as it does not interfere with the reaction. Examples of the solvent include tetrahydrofuran, 1,4-dioxane, DMF, DMA, NMP and mixed solvents thereof. The reaction temperature is usually room temperature to 150° C. and is preferably room temperature to 100° C. The reaction time is not particularly limited and is usually 5 minutes to 24 hours, preferably 30 minutes to 24 hours.

### Step 5

Compound E6 can be prepared by deprotection of Compound E5. This deprotection can be conducted by a suitable condition for the chosen protecting group. For example, when the protecting group is trityl or THP. Compound E6 can be prepared under acidic condition, preferably using p-toluenesulfonic acid or hydrochloric acid. The solvent used in this step is not particularly limited in so far as it does not interfere with the reaction. Examples of the solvent include, tetrahydrofuran 1,4-dioxane, methanol, ethanol, isopropanol, water and mixed solvents thereof. The reaction temperature is preferably room temperature to 100° C. The reaction time is not particularly limited and is usually 5 minutes to 24 hours, preferably 30 minutes to 24 hours.

### Step 6

Compound E7 can be prepared by oxidation of the alcohol of Compound E6. The oxidation can be conducted by a suitable condition, such as, for example, TEMPO oxidation using TEMPO, NaClO₂ and NaClO. The solvent used in this step is not particularly limited in so far as it does not interfere with the reaction. Examples of the solvent include dichloromethane, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### General Procedure F:

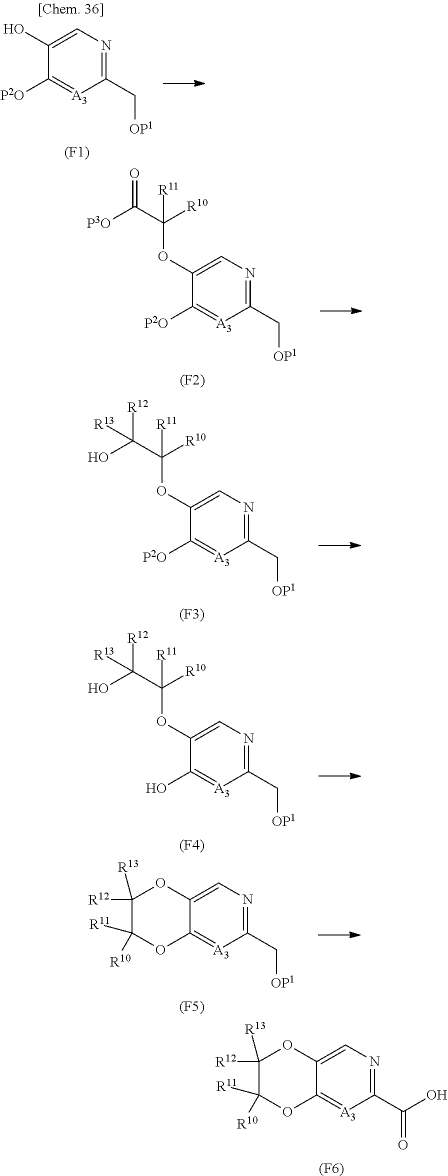

Wherein P¹, P² and P² are protective groups such as alkyl, acetyl, benzoyl, benzyl, 4-methoxybenzyl, 2,4-dimethoxybenzyl, trityl or THP, and R¹⁰ and R¹¹ are each independently a hydrogen atom or fluoro, and R¹² and R¹³ are each independently a hydrogen atom, fluoro or alkyl. The other symbols are the same as defined above (1).

General Procedure F is a method for preparing Compound F6 from Compound F1 through multiple steps of Step 1 to Step 5. Those skilled in the art will be appreciate that protective groups can be chosen depending on the reaction conditions used in later steps.

### Step 1

Compound F2 can be prepared by means of the alkylation of Compound F1. This type of reactions can be conducted with the corresponding α-haloesters using an appropriate base, such as, for example, potassium carbonate, cesium carbonate and DBU. Alternatively, Compound F2 can be obtained by Mitsunobu reaction using the corresponding α-hydroxyesters and reagents such as, for example, DEAD, DIAD or ADDP and triphenylphosphine or tributylphosphine. The solvent used in this step is not particularly limited in so far as it does not interfere with the reaction. Examples of the solvent include tetrahydrofuran, 1,4-dioxane, benzene, toluene, DMF, and mixed solvents thereof. The reaction temperature is usually room temperature to 150° C. and is preferably room temperature to 100° C. The reaction time is not particularly limited and is usually 5 minutes to 24 hours, preferably 30 minutes to 24 hours.

### Step 2

Compound F3 can be prepared by reduction of ester of Compound F2. The reduction can be conducted by a reducing reagent such as, for example, sodium borohydride, lithium borohydride and lithium aluminum hydride. The solvent used in this step is not particularly limited in so far as it does not interfere with the reaction. Examples of the solvent include tetrahydrofuran, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### Step 3

Compound F4 can be prepared by deprotection of Compound F3.

This deprotection can be conducted by a suitable condition for the chosen protecting group. For example, when the protecting group is benzyl, Compound F3 can be prepared by a suitable catalyst, such as, for example, palladium on carbon, under hydrogen atmosphere. The solvent used in this step is not particularly limited in so far as it does not interfere with the reaction. Examples of the solvent include, tetrahydrofuran, 1,4-dioxane, methanol, ethanol, isopropanol, water and mixed solvents thereof. The reaction temperature is preferably room temperature to 60° C. The reaction time is not particularly limited and is usually 5 minutes to 24 hours, preferably 30 minutes to 24 hours.

### Step 4

Compound F5 can be prepared by Mitsunobu reaction of Compound F4. This reaction can be conducted by suitable reagents such as for example, DEAD, DIAD or ADDP, and triphenylphosphine or tributylphosphine. The solvent used in this step is not particularly limited in so far as it does not interfere with the reaction. Examples of the solvent include tetrahydrofuran, 1,4-dioxane, DMF, DMA, NMP and mixed solvents thereof. The reaction temperature is usually room temperature to 150° C. and is preferably room temperature to 100° C. The reaction time is not particularly limited and is usually 5 minutes to 24 hours, preferably 30 minutes to 24 hours.

### Step 5

Compound F6 can be prepared by deprotection of Compound F5. This deprotection can be conducted by a suitable condition for the chosen protecting group. For example, when the protecting group is trityl or THP, Compound F6 can be prepared under acidic conditions, preferably using p-toluenesulfonic acid or hydrochloric acid. The solvent used in this step is not particularly limited in so far as it does not interfere with the reaction. Examples of the solvent include tetrahydrofuran, 1,4-dioxane, methanol, ethanol, isopropanol, water and mixed solvents thereof. The reaction temperature is preferably room temperature to 100° C. The reaction time is not particularly limited and is usually 5 minutes to 24 hours, preferably 30 minutes to 24 hours.

### Step 6

Compound F7 can be prepared by oxidation of the alcohol of Compound F6. The oxidation can be conducted by a suitable condition, such as, for example, TEMPO oxidation using TEMPO, NaClO₂ and NaClO. The solvent used in this step is not particularly limited in so far as it does not interfere with the reaction. Examples of the solvent include dichloromethane, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### General Procedure G

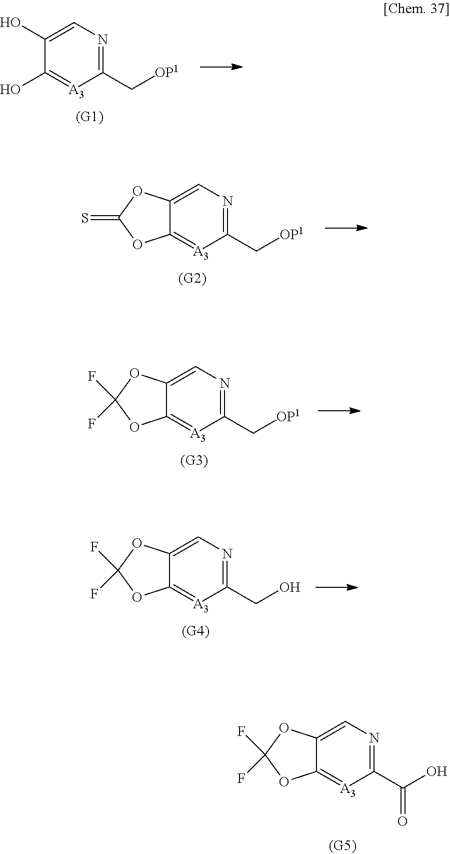

Wherein P¹ is protective groups such as alkyl, acetyl, benzoyl, benzyl, 4-methoxybenzyl, 2,4-dimethoxybenzyl, trityl or THP. Other symbols are the same as defined above.

General Procedure G is a method for preparing Compound F6 from Compound G1 through multiple steps of Step 1 to Step 4. Those skilled in the art will be appreciate that protective groups can be chosen depending on the reaction conditions used in the later steps.

### Step 1

Compound G2 can be prepared by the reaction with thiophosgene using an appropriate base such as for example, DMAP, pyridine or 2.5-lutidine. The solvent used in this step is not particularly limited in so far as it does not interfere with the reaction. Examples of the solvent include dichloromethane, 1,2-dichloroethane, tetrahydrofuran, 1,4-dioxane, and mixed solvents thereof. The reaction temperature is preferably −20° C. to room temperature. The reaction time is not particularly limited and is usually 5 minutes to 24 hours, preferably 30 minutes to 24 hours.

### Step 2

Compound G3 can be prepared by fluorination of Compound G2. The reaction can be conducted using an appropriate reagent such as for example, hydrogen fluoride pyridine. The solvent used in this step is not particularly limited in so far as it does not interfere with the reaction. Examples of the solvent include dichloromethane, or 1,2-dichloroethane. The reaction temperature is preferably Wherein P¹ is protective groups such as alkyl, acetyl, benzoyl, benzyl, 4-methoxybenzyl, 2,4-dimethoxybenzyl, trityl or THP. The other symbols are the same as defined above (1).

General Procedure G is a method for preparing Compound F6 from Compound G1 through multiple steps of Step 1 to Step 4. Those skilled in the art will be appreciate that protective groups can be chosen depending on the reaction conditions used in later steps.

### Step 1

Compound G2 can be prepared by the reaction with thiophosgene using an appropriate base such as for example, DMAP, pyridine or 2.5-lutidine. The solvent used in this step is not particularly limited in so far as it does not interfere with the reaction. Examples of the solvent include dichloromethane, 1,2-dichloroethane, tetrahydrofuran, 1,4-dioxane, and mixed solvents thereof. The reaction temperature is preferably −20° C. to room temperature. The reaction time is not particularly limited and is usually 5 minutes to 24 hours, preferably 30 minutes to 24 hours.

### Step 2

Compound G3 can be prepared by fluorination of Compound G2. The reaction can be conducted using an appropriate reagent such as for example, hydrogen fluoride pyridine. The solvent used in this step is not particularly limited in so far as it does not interfere with the reaction. Examples of the solvent include dichloromethane, or 1,2-dichloroethane. The reaction temperature is preferably −78° C. to room temperature, preferably −60° C. to 0° C. The reaction time is not particularly limited and is usually 5 minutes to 24 hours, preferably 30 minutes to 24 hours.

### Step 3

Compound G4 can be prepared by deprotection of Compound G3. This deprotection can be conducted by a suitable condition for the chosen protecting group. For example, when the protecting group is trityl or THP, Compound G4 can be prepared under acidic conditions, preferably using p-toluenesulfonic acid or hydrochloric acid. Alternatively, when the protecting group is acetyl or benzoyl, Compound G4 can be prepared under basic conditions, preferably using sodium carbonate, potassium carbonate, sodium hydroxide or potassium hydroxide. The solvent used in this step is not particularly limited in so far as it does not interfere with the reaction. Examples of the solvent include tetrahydrofuran, 1,4-dioxane, methanol, ethanol, isopropanol, water and mixed solvents thereof. The reaction temperature is preferably 0° C. to 100° C. The reaction time is not particularly limited and is usually 5 minutes to 24 hours, preferably 30 minutes to 24 hours.

### Step 4

Compound G5 can be prepared by oxidation of the alcohol of Compound G4. The oxidation can be conducted by a suitable condition, such as, for example, TEMPO oxidation using TEMPO, NaClO₂ and NaClO. The solvent used in this step is not particularly limited in so far as it does not interfere with the reaction. Examples of the solvent include dichloromethane, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### General Procedure H

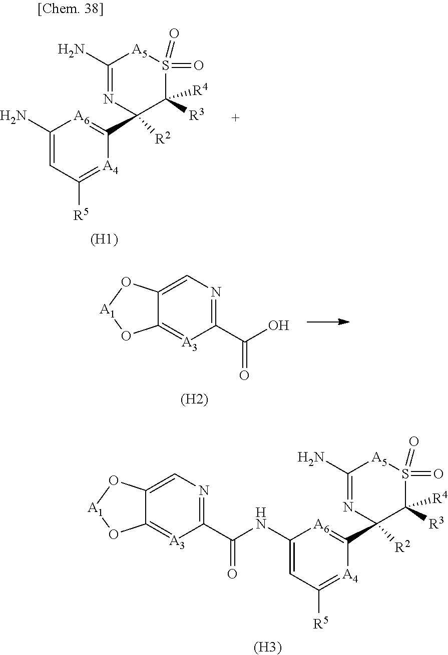

Wherein symbols are the same as defined above (1).

General Procedure H is a method for preparing Compound H3.

### Step 1

Compounds H3 can be prepared by amide coupling reaction of Compound H1 with Compound H2. This reaction can be conducted by a method known to a person skilled in the art, and suitable coupling conditions can be found in Chem. Rev. 2011, 111, 6557-6602, which includes: a) reactions using condensation reagents; b) reactions using acid chlorides or fluorides.

Reaction a) can be conducted by use of condensation reagents such as dicyclohexylcarbodiimide (DCC), diisopropylcarbodiimide (DIC), 1-ethyl-3-(3-dimethylaminopropyl) carbodiimide hydrochloride (EDC hydrochloride), O-(7-aza-1H-benzotriazol-1-yl)-N,N,N′,N′-tetramethyluronium hexafluorophosphate (HATU), and 1H-Benzotriazol-1-yloxy-tri(pyrrolidino) phosphonium hexafluorophosphate (PyBOP). When using uronium or phosphonium salts such as HATU and PyBOP, the reaction can be performed in the presence of bases such as triethylamine and diisopropylethylamine. The reaction may be accelerated by use of catalysts such as 1-hydroxy-benzotriazole (HOBt) and 1-hydroxy-7-aza-benzotriazole (HOAt). The solvent used in the reaction is not particularly limited in so far as it does not interfere with the reaction. Examples of the solvent include dichloromethane, N,N-dimethylformamide (DMF), N-methylpyrrolidone (NMP), and tetrahydrofuran. The reaction temperature is usually 0° C. to 50° C. and is preferably room temperature.

Reaction b) can be performed by use of commercially available acid chlorides or those synthesized by known methods to a person skilled in the art in solvents such as dichloromethane, tetrahydrofuran, and ethyl acetate in the presence of bases such as triethylamine, diisopropylethylamine, pyridine, and N,N-dimethyl-4-aminopyridine. The reaction temperature is usually 0° C. to 60° C. and is preferably 0° C. to room temperature. The reaction time is not particularly limited and is usually 5 minutes to 24 hours, preferably 20 minutes to 6 hours.

### General Procedure A′

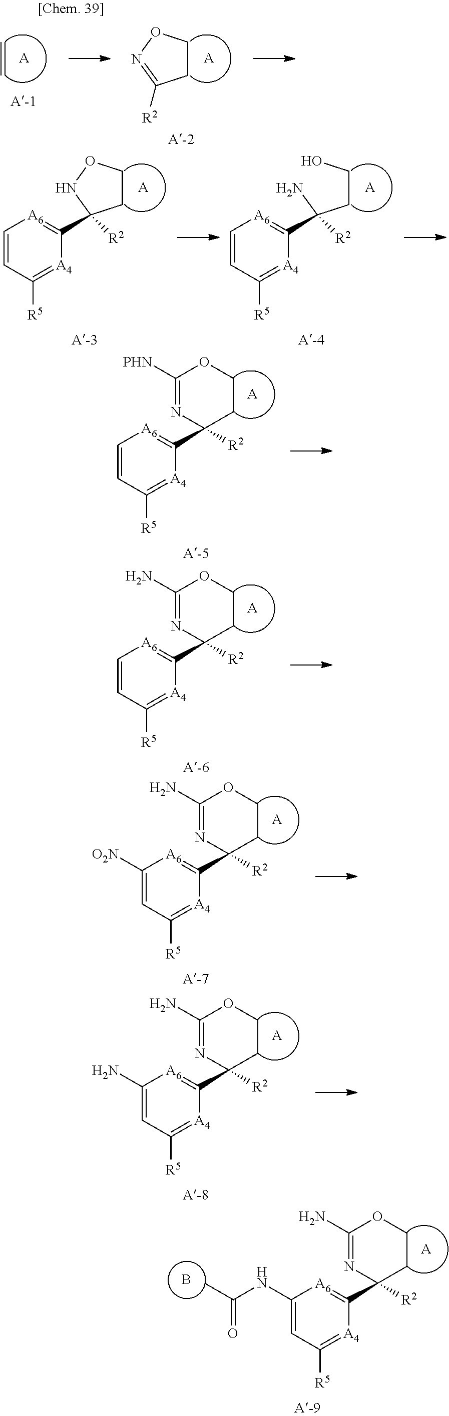

Wherein P is a protective group such as benzoyl or benzyl and the other symbols are the same as defined above (1).

General Procedure A′ is a method for preparing compounds of Compound A′-9 from Compounds A′-1 through multiple steps of Step 1 to Step 8. Those skilled in the art will be appreciate that protective groups P can be chosen depending on the reaction conditions used in later steps.

### Step 1

Compound A′-2 can be prepared by means of 1,3-dipolar cycloaddition. This type of reactions can be conducted using similar conditions described in J. Am. Chem. Soc., 1960, 82, 5339-5342 or J. Org. Chem. 1998, 63, 5272-5274. This 1,3-dipolar cycloadditions can be conducted with cyclic Compound A′-1 and the corresponding nitrile oxides generated in situ from the corresponding nitroalkanes using an appropriate dehydrating agents such as, for example, phenyl isocyanate, phenyl diisocyanate or (Boc)₂O, and an appropriate base such as, for example, triethylamine, dipropylethylamine or N-methylmorpholine. Alternatively, the nitrile oxides can be generated in situ from the corresponding hydroxamoyl chlorides with an appropriate base such as, for example, triethylamine, dipropylethylamine or N-methylmorpholine. The solvent used in this step is not particularly limited in so far as it does not interfere with the reaction. Examples of the solvent include tetrahydrofuran, 1,4-dioxane, 1, 2-dimethoxyethane, diethyl ether, toluene, benzene, and mixed solvents thereof. The reaction temperature is preferably room temperature to 120° C. The reaction time is not particularly limited and is usually 5 minutes to 24 hours, preferably 30 minutes to 24 hours.

### Step 2

Compound A′-3 can be prepared by means of the nucleophilic addition of an appropriate aryllithium reagents or Grignard reagents to Compound A′-2. This type of reactions can be conducted using similar conditions described in J. Am. Chem. Soc., 2005, 127, 5376-5384. Preferably, the aryllithium reagents or Grignard reagents can be prepared from the corresponding aromatic halides using an appropriate base, such as, for example, n-, sec- or tert-butyl lithium, isopropylmagnesium bromide or metallic magnesium, which can be then reacted to Compound A′-2 with Lewis acid such as, for example, BF₃—OEt₂ to give Compound A′-3. The solvent used in this step is not particularly limited in so far as it does not interfere with the reaction. Examples of the solvent include tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### Step 3

Compound A′-4 can be prepared by reductive cleavage reaction of the N—O bond of compound A′-3. This reductive cleavage can be conducted using zinc with an appropriate acid such as acetic acid, formic acid or hydrochloric acid. The solvent used in this step is not particularly limited in so far as it does not interfere with the reaction. Examples of the solvent include methanol, ethanol, tetrahydrofuran, water and mixed solvents thereof. The reaction temperature is preferably −20° C. to solvent reflux temperature. The reaction time is not particularly limited and is usually 5 minutes to 24 hours, preferably 30 minutes to 24 hours.

Alternatively, this reaction can be performed using a metal catalyst such as platinum oxide under hydrogens. The solvent used in this step is not particularly limited in so far as it does not interfere with the reaction. Examples of the solvent include methanol, ethanol, water and mixed solvents thereof. The reaction temperature is preferably room temperature to 50° C. The reaction time is not particularly limited and is usually 5 minutes to 24 hours, preferably 30 minutes to 24 hours.

Furthermore, this type of reaction can also be conducted using lithium aluminum hydride. The solvent used in this step is not particularly limited in so far as it does not interfere with the reaction. Examples of the solvent include tetrahydrofuran, 1,4-dioxane, 1,2-dimethoxyethane, diethyl ether and mixed solvents thereof. The reaction temperature is preferably −20° C. to room temperature. The reaction time is not particularly limited and is usually 5 minutes to 24 hours, preferably 30 minutes to 24 hours.

### Step 4

Compound A′-5 can be prepared by formation of the corresponding thioureas from Compound A′-4 in situ, followed by cyclization reaction. This type of reactions is known to a person skilled in the art and can be performed under the conditions described in WO2014/065434. The thiourea can be obtained in situ from Compound A-4 using an appropriate isothiocyanates such as, for example benzoyl isothiocyanate or benzyl isothiocyanate, then cyclization can be performed by adding reagents such as, for example m-CPBA, hydrogene peroxide, or carbodiimide reagents (e. g. DCC, DIC or EDC). Alternatively, this cyclization can be performed using alkylating reagents such as methyl iodide, and an appropriate base such as sodium hydride, sodium bicarbonate and potassium carbonate. The solvent used in this step is not particularly limited in so far as it does not interfere with the reaction. Examples of the solvent include chloroform, dichloromethane, dichloroethane, tetrahydrofuran, and mixed solvents thereof. The reaction temperature is usually 0° C. to 60° C. The reaction time is not particularly limited and is usually 5 minutes to 24 hours, preferably 30 minutes to 24 hours.

### Step 5

Compound A′-6 can be prepared by deprotection of Compound A-5. This deprotection reaction is known to a person skilled in the art and can be performed under the conditions described in Green's Protective Groups in Organic Synthesis, 4th ed. When the protecting group is benzoyl, the deprotecting reaction can be conducted under acidic conditions such as sulfuric acid or hydrochloric acid, or under basic condition such as hydrazine, DBU, or sodium hydroxide. The solvent used in this step is not particularly limited in so far as it does not interfere with the reaction. Examples of the solvent include dichloromethane, tetrahydrofuran, 1,4-dioxane, methanol, toluene, benzene and mixed solvents thereof. The reaction temperature is preferably room temperature to 100° C. The reaction time is not particularly limited and is usually 5 minutes to 24 hours, preferably 30 minutes to 24 hours.

### Step 6

Compound A′-7 can be prepared by nitration of Compound A′-6. A typical procedure involves the treatment of Compound A′-6 dissolved in sulfuric acid and trifluoroacetic acid, with a source of nitronium ion, such as, for example, potassium nitrate or nitric acid. The reaction temperature is preferably −20° C. to 0° C. The reaction time is not particularly limited and is usually 5 minutes to 5 hours, preferably 30 minutes to 2 hours.

### Step 7

Compound A′-8 can be prepared by reduction of Compound A′-7. The reduction can be conducted by a suitable catalyst, such as, for example, palladium on carbon under hydrogen atmosphere, or the use of a reducing agent such as, for example, iron, zinc or tin(II) chloride. The solvent used in this step is not particularly limited in so far as it does not interfere with the reaction. Examples of the solvent include tetrahydrofuran, methanol, ethanol, water, and mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

### Step 8

Compound A′-9 can be prepared by amide coupling reaction of Compound A′-8 with the corresponding carboxylic acids. This reaction can be conducted by a method known to a person skilled in the art, and suitable coupling conditions can be found in Chem. Rev. 2011, 111, 6557-6602, which includes: a) reactions using condensation reagents; b) reactions using acid chlorides or fluorides.

Reaction a) can be conducted by use of condensation reagents such as dicyclohexylcarbodiimide (DCC), diisopropylcarbodiimide (DIC), 1-ethyl-3-(3-dimethylaminopropyl) carbodiimide hydrochloride (EDC hydrochloride), O-(7-aza-1H-benzotriazol-1-yl)-N,N,N′,N′-tetramethyluronium hexafluorophosphate (HATU), and 1H-Benzotriazol-1-yloxy-tri(pyrrolidino) phosphonium hexafluorophosphate (PyBOP). When using uronium or phosphonium salts such as HATU and PyBOP, the reaction can be performed in the presence of bases such as triethylamine and diisopropylethylamine. The reaction may be accelerated by use of catalysts such as 1-hydroxy-benzotriazole (HOBt) and 1-hydroxy-7-aza-benzotriazole (HOAt). The solvent used in the reaction is not particularly limited in so far as it does not interfere with the reaction. Examples of the solvent include dichloromethane, N,N-dimethylformamide (DMF), N-methylpyrrolidone (NMP), and tetrahydrofuran. The reaction temperature is usually 0° C. to 50° C. and is preferably room temperature.

Reaction b) can be performed by use of commercially available acid chlorides or those synthesized by known methods to a person skilled in the art in solvents such as dichloromethane, tetrahydrofuran, and ethyl acetate in the presence of bases such as triethylamine, diisopropylethylamine, pyridine, and N,N-dimethyl-4-aminopyridine. The reaction temperature is usually 0° C. to 60° C. and is preferably 0° C. to room temperature. The reaction time is not particularly limited and is usually 5 minutes to 24 hours, preferably 20 minutes to 6 hours.

### General Procedure B′

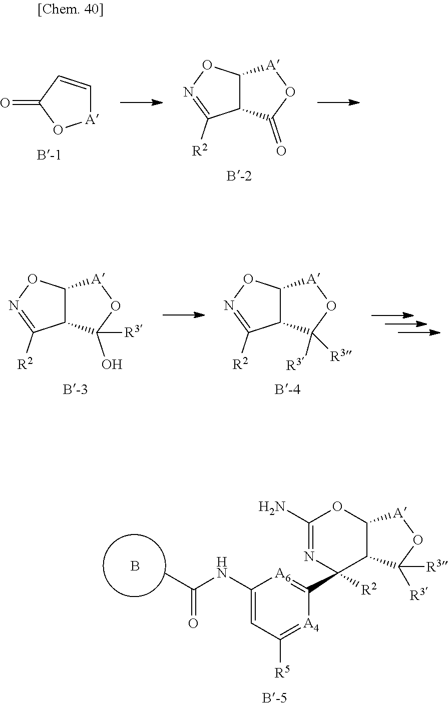

Wherein A′ is substituted or unsubstituted C1-2 alkylene, R³′ and R³″ are each independently selected from the group consisting of alkyl optionally substituted with halogen, cyano, alkyloxy, haloalkyloxy or non-aromatic carbocyclyl, and heteroaryl optionally substituted with alkyl, and other symbols are the same as defined above.

General Procedure B′ is a method for preparing Compound B′-5 from Compound B′-1 through multiple steps. Using Compound B′-4 and Compound B′-5 can be prepared according to the methods described in General procedure A′.

### Step 1

Compound B′-2 can be prepared by means of 1,3-dipolar cycloaddition. This type of reactions can be conducted using similar conditions described in J. Am. Chem. Soc. 1960, 82, 5339-5342 or J. Org. Chem. 1998, 63, 5272-5274. This 1,3-dipolar cycloadditions can be conducted with cyclic Compound B′-1 and the corresponding nitrile oxides generated in situ from the corresponding nitroalkanes using an appropriate dehydrating agents such as, for example, phenyl isocyanate, phenyl diisocyanate or (Boc)₂O, and an appropriate base such as, for example, triethylamine, diisopropylethylamine or N-methylmorpholine. Alternatively, the nitrile oxides can be generated in situ from the corresponding hydroxamoyl chlorides with an appropriate base such as, for example, triethylamine, diisopropylethylamine or N-methylmorpholine. The solvent used in this step is not particularly limited in so far as it does not interfere with the reaction. Examples of the solvent include tetrahydrofuran, 1,4-dioxane, 1,2-dimethoxyethane, diethyl ether, toluene, benzene, and mixed solvents thereof. The reaction temperature is preferably room temperature to 120° C. The reaction time is not particularly limited and is usually 5 minutes to 24 hours, preferably 30 minutes to 24 hours.

### Step 2

When R³′ is a hydrogen atom, Compound B′-3 can be prepared by carbonyl reduction of Compound B′-2. This type of reactions can be conducted using an appropriate metal hydrides such as, for example, DIBAL-H, lithium tri-tert-butoxyaluminum hydride or sodium bis(2-methoxyethoxy)aluminum, by means of the nucleophilic addition to Compound B′-2. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

When R³′ is other than a hydrogen atom, Compound B′-3 can be prepared by means of the nucleophilic addition to Compound B′-2. This type of reactions can be conducted using an appropriate nucleophiles such as, for example organic lithium, magnesium, zinc or silyl reagents, with or without Lewis acid such as, for example BF₃—OEt₂, AlCl₃ or TiCl₄. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### Step 3

When R³″ is a hydrogen atom, Compound B′-4 can be prepared by reduction of Compound B′-3. This type of reactions can be conducted using an appropriate reducing agents such as triethylsilane, sodium borohydride with or without Lewis acid such as BF₃—OEt₂. The solvent used in this step is not particularly limited in so far as it does not interfere with the reaction. Examples of the solvent include dichloromethane, acetonitrile, tetrahydrofuran, 1,4-dioxane, 1,2-dimethoxyethane, diethyl ether, toluene, benzene, and mixed solvents thereof. The reaction temperature is preferably −20° C. to room temperature. The reaction time is not particularly limited and is usually 5 minutes to 24 hours, preferably 30 minutes to 24 hours.

When R³″ is other than a hydrogen atom, Compound B′-4 can be prepared by means of the nucleophilic addition to Compound B′-2. This type of reactions can be conducted using an appropriate nucleophiles such as, for example organic lithium, magnesium, zinc or silyl reagents, with or without Lewis acid such as, for example BF₃—OEt₂, AlCl₃ or TiCl₄. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### General Procedure C′

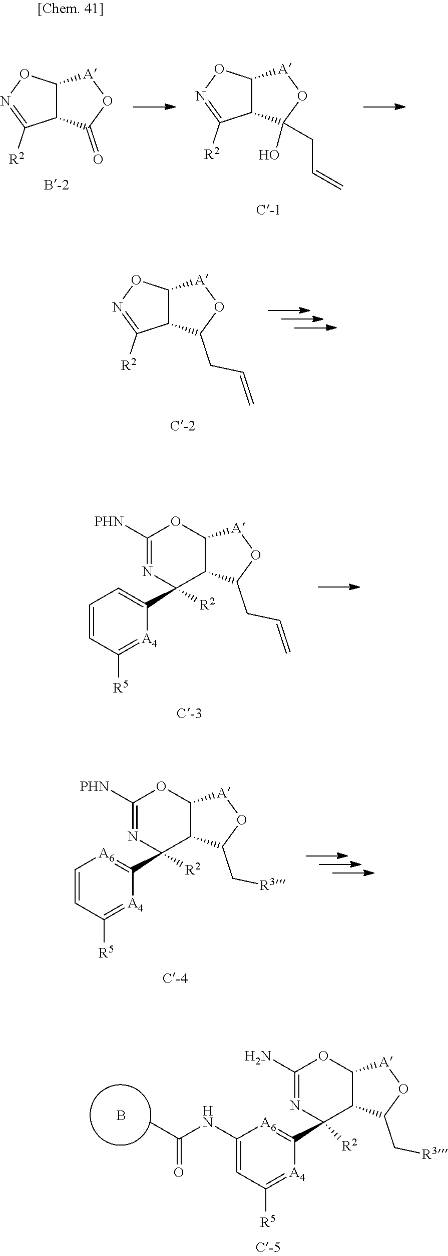

Wherein P is a protective group such as benzoyl or benzyl, R³′″ is ethyl or cyclopropyl, and the other symbols are the same as defined above (1).

General Procedure C′ is a method for preparing Compound C′-5 from Compound B-1 through multiple steps. Compound C′-3 and Compound C′-5 can be prepared from Compound C′-2 and C′-5 according to the methods described in General procedure A′.

### Step 1

Compound C′-1 can be prepared by means of the nucleophilic addition of allyl moiety to carbonyl group of Compound B′-2. This type of reactions can be conducted using an appropriate commercially available or in situ generated allyl reagents such as, for example allyl silane, lithium, magnesium, zinc reagents, with or without Lewis acid such as, for example BF₃—OEt₂, AlCl₃ or TiCl₄. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to room temperature. The reaction time is not particularly limited and is usually 5 minutes to 24 hours, preferably 30 minutes to 24 hours.

### Step 2

Compound C′-2 can be prepared by reduction of Compound C′-1. This type of reactions can be conducted using an appropriate reducing agents such as triethylsilane or sodium borohydride, with or without Lewis acid such as BF₃—OEt₂. The solvent used in this step is not particularly limited in so far as it does not interfere with the reaction. Examples of the solvent include dichloromethane, acetonitrile, tetrahydrofuran, 1,4-dioxane, 1,2-dimethoxyethane, diethyl ether, toluene, benzene, and mixed solvents thereof. The reaction temperature is preferably −20° C. to room temperature. The reaction time is not particularly limited and is usually 5 minutes to 24 hours, preferably 30 minutes to 24 hours.

### Step 3

When R³′″ is ethyl, Compound C′-5 can be obtained by hydrogenation of Compound C′-4. The hydrogenation can be performed using suitable catalyst such as, for example palladium on carbon under hydrogens atmosphere. The solvent used in this step is not particularly limited in so far as it does not interfere with the reaction. Examples of the solvent include tetrahydrofuran, methanol, ethanol, water, and mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

When R³′″ is cyclopropyl, Compound C′-5 can be obtained by means of cyclopropanation of Compound C′-4. This type of reaction can be performed using an appropriate reagent such as diazomethane with or without a suitable catalyst, or Simmons-Smith reaction condition such as, for example diiodomethane with diethylzinc. The solvent used in this step is not particularly limited in so far as it does not interfere with the reaction. Examples of the solvent include dichloromethane, diethylether, toluene, benzene, or mixed solvents thereof. The reaction temperature is usually −30° C. to room temperature. The reaction time is not particularly limited and is usually 5 minutes to 24 hours, preferably 30 minutes to 24 hours.

### General Procedure D′

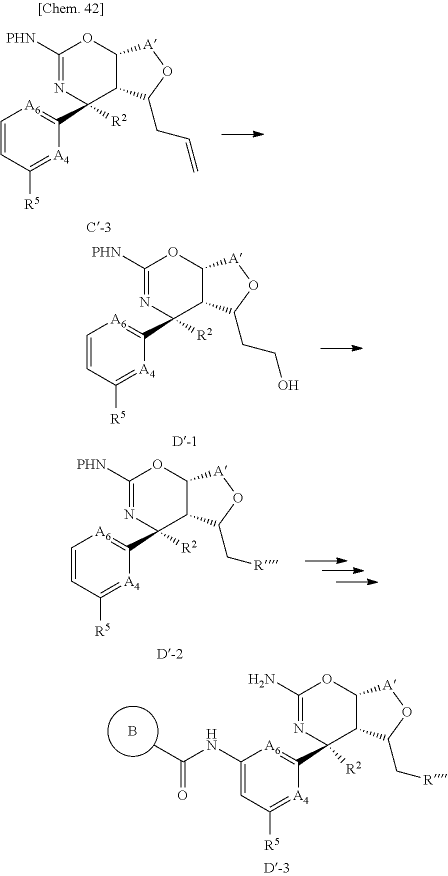

Wherein P is a protective group such as benzoyl or benzyl, R³″″ is alkyl substituted with fluorine or alkyloxy, and the other symbols are the same as defined above (1).

General Procedure D′ is a method for preparing compounds of Compound D′-3 from Compound C′-3 through multiple steps. Compound D′-3 can be prepared from Compound D′-2 according to the methods described in General procedure A′.

### Step 1

Compound D′-1 can be prepared by ozonolysis of Compound C′-3, followed by reduction of the resulting aldehyde. This reaction can be performed by a method known to a person skilled in the art. The ozonolysis can be performed under ozone atmosphere in suitable solvent such as dichloromethane, methanol, and mixed thereof, with an appropriate regents such as triphenylphosphine, pyridine, dimethylsulfide and trimethylamine under nitrogens atmosphere for reductive workup. The temperature for generation of ozonide is preferably −78° C., then the temperature can be allowed to warm to room temperature for reductive workup. The reaction time is not particularly limited and is usually 30 minutes to 5 hours, preferably 30 minutes to 2 hours. The reduction of the resulting aldehyde can be performed in one pot using an appropriate reducing agent such as sodium borohydride or lithium aluminum hydride. The reaction temperature is preferably 0° C. to room temperature. The reaction time is not particularly limited and is usually 30 minutes to 5 hours, preferably 30 minutes to 2 hours.

### Step 2

When R³″″ is CF₃, CHF₂ or CH₂F, Compound D′-2 can be obtained by two-step sequence; oxidation of Compound D-1 to the aldehyde or carboxylic acid followed by fluorination, or direct fluorination of Compound D′-1. This reaction can be performed by a method known to a person skilled in the art. For example, Compound D′-1 can be oxidized to the corresponding aldehyde under an appropriate oxidation condition such as, for example TEMPO, Dess-Martin or Swern oxidation. The corresponding carboxylic acid can be obtained by oxidation of the resulting aldehyde, or oxidizing Compound D′-1 directly using an appropriate condition such as for example, Pinnick, TEMPO or Jones oxidation. The solvent used in this step is not particularly limited in so far as it does not interfere with the reaction. The reaction temperature is usually −78° C. to room temperature. The reaction time is not particularly limited and is usually 5 minutes to 24 hours, preferably 30 minutes to 24 hours. The fluorination reaction can be performed using an appropriate reagent such as, for example DAST, Deoxofluor or sulfur tetrafluoride. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to 50° C. The reaction time is not particularly limited and is usually 5 minutes to 24 hours, preferably 30 minutes to 24 hours.

When R³′″ ‘is alkyloxy, Compound D′-2 can be obtained by means of alkylation of the terminal alcohol of Compound D′-1. This reaction can be performed using an appropriate base such as sodium hydride with the corresponding electrophiles such as alkyl halide, mesylate or triflate. The solvent used in this step is not particularly limited in so far as it does not interfere with the reaction. Examples of the solvent include acetone, acetonitrile, tetrahydrofuran, DMF, DMA, DMSO, toluene, and mixed solvents thereof. The reaction temperature is preferably 0° C. to 100° C. The reaction time is not particularly limited and is usually 5 minutes to 24 hours, preferably 30 minutes to 24 hours.

### General Procedure E′

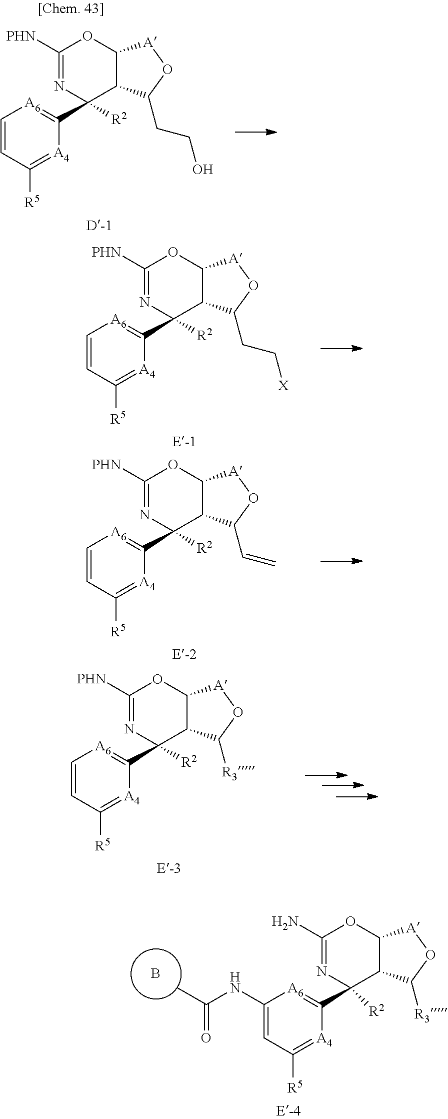

Wherein P is a protective group such as benzoyl or benzyl, R³′″″ is ethyl or cyclopropyl, X is leaving group such as halogene, mesylate or triflate, and other symbols are the same as defined above (1).

General Procedure E′ is a method for preparing compounds of Compound E′-4 from Compound D′-1 through multiple steps. Compound E′-4 can be prepared from Compound E′-2 according to the methods described in General procedure A′.

### Step 1

Compound E′-1 can be prepared by converting the terminal alcohol of Compound D′-3 to leaving group. This reaction can be performed by a method known to a person skilled in the art. Compound E′-1 can be obtained under suitable halogenation conditions such as, for example using SOX₂, POX₃ (X=Cl or Br), or Appel reaction conditions such as triphenylphosphine with CX₄ (X=Cl or Br) or iodine. The solvent used in this step is not particularly limited in so far as it does not interfere with the reaction. Examples of the solvent include dichloromethane, tetrahydrofuran, toluene, and mixed solvents thereof. The reaction temperature is preferably 0° C. to 100° C. The reaction time is not particularly limited and is usually 5 minutes to 24 hours, preferably 30 minutes to 24 hours.

### Step 2

Compound E′-2 can be prepared by converting the terminal alcohol of Compound D′-3 to a leaving group. This reaction can be performed by a method known to a person skilled in the art. Compound E-1 can be obtained under suitable halogenation conditions such as, for example using SOX₂, POX₃ (X=Cl or Br), or Appel reaction conditions such as triphenylphosphine with CX₄ (X=Cl or Br) or iodine. The solvent used in this step is not particularly limited in so far as it does not interfere with the reaction. Examples of the solvent include dichloromethane, tetrahydrofuran, toluene, and mixed solvents thereof. The reaction temperature is preferably 0° C. to 100° C. The reaction time is not particularly limited and is usually 5 minutes to 24 hours, preferably 30 minutes to 24 hours.

### Step 3

Compound E′-2 can be prepared by means of elimination reaction of compound E′-1. This reaction can be performed by a method known to a person skilled in the art. Compound E′-2 can be obtained using an appropriate base such as for example, sodium or potassium tert-butoxide, triethylamine, diisopropylethylamine, DBU or pyridine. The solvent used in this step is not particularly limited in so far as it does not interfere with the reaction. Examples of the solvent include dichloromethane, tetrahydrofuran, toluene, and mixed solvents thereof. The reaction temperature is preferably 0° C. to 60° C. The reaction time is not particularly limited and is usually 5 minutes to 24 hours, preferably 30 minutes to 24 hours.

### Step 4

When R³′″″ is ethyl, Compound E′-3 can be obtained by hydrogenation of Compound E′-2. The hydrogenation can be performed using suitable catalysts such as, for example palladium on carbon under hydrogene atmosphere. The solvent used in this step is not particularly limited in so far as it does not interfere with the reaction. Examples of the solvent include, tetrahydrofuran, methanol, ethanol, water, mixed solvents thereof. The reaction temperature is usually room temperature to 80° C. and is preferably room temperature to 60° C. The reaction time is not particularly limited and is usually 5 minutes to 24 hours, preferably 30 minutes to 24 hours.

When R³′″″ is cyclopropyl, Compound E′-3 can be obtained by means of cyclopropanation of Compound C′-4. This type of reaction can be performed using an appropriate reagent such as diazomethane with or without a suitable catalyst, or Simmons-Smith reaction conditions such as, for example diiodomethane with diethylzinc. The solvent used in this step is not particularly limited in so far as it does not interfere with the reaction. Examples of the solvent include dichloromethane, diethylether, toluene, benzene, or mixed solvents thereof. The reaction temperature is usually −30° C. to room temperature. The reaction time is not particularly limited and is usually 5 minutes to 24 hours, preferably 30 minutes to 24 hours.

### General Procedure F′

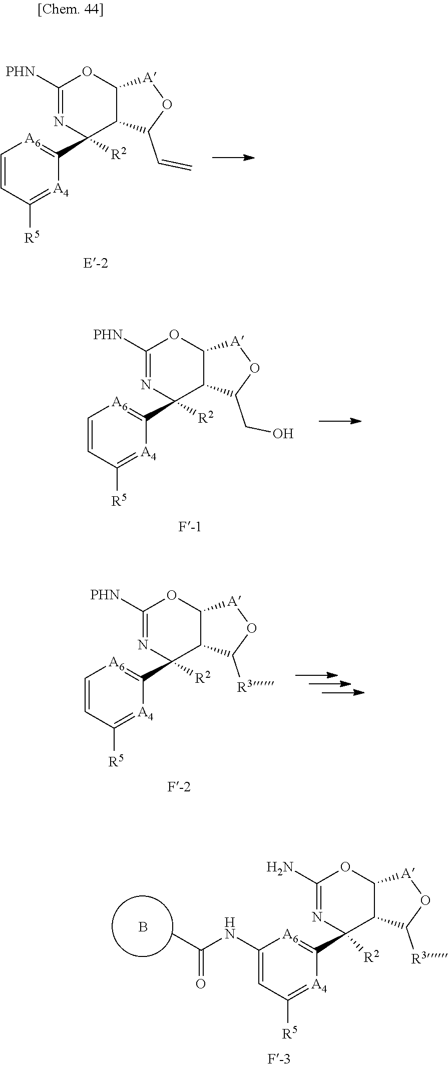

Wherein P is a protective group such as benzoyl or benzyl, R³″″ is alkyl substituted with fluorine or alkyloxy, and other symbols are the same as defined above.

General Procedure F′ is a method for preparing Compound F′-3 from Compounds E′-2 through multiple steps. Compounds F′-3 can be prepared from Compound F′-2 according to the methods described in General procedure A′.

### Step 1

Compound F′-1 can be prepared by ozonolysis of Compound F′-3, followed by reduction of the resulting aldehyde. This reaction can be performed by a method known to a person skilled in the art. The ozonolysis can be performed under ozone atmosphere in a suitable solvent such as dichloromethane, methanol, and mixed thereof, with an appropriate reagents such as triphenylphosphine, pyridine, dimethylsulfide and trimethylamine under nitrogen atmosphere for reductive workup. The temperature for generation of ozonide is preferably −78° C., then the temperature can be allowed to warm to room temperature for reductive workup. The reaction time is not particularly limited and is usually 30 minutes to 5 hours, preferably 30 minutes to 2 hours. The reduction of the resulting aldehyde can be performed in one pot using an appropriate reducing agent such as sodium borohydride or lithium aluminum hydride. The reaction temperature is preferably 0° C. to room temperature. The reaction time is not particularly limited and is usually 30 minutes to 5 hours, preferably 30 minutes to 2 hours.

### Step 2

When R³″″″ is CF₃, CHF₂ or CH₂F, Compound F′-2 can be obtained by two-step sequence; oxidation of Compound F-1 to the aldehyde or carboxylic acid followed by fluorination, or direct fluorination of Compound F′-1. This reaction can be performed by a method known to a person skilled in the art. For example, Compound F-1 can be oxidized to the corresponding aldehyde under an appropriate oxidation condition such as, for example TEMPO, Dess-Martin or Swern oxidation. The corresponding carboxylic acid can be obtained by oxidation of the resulting aldehyde, or oxidizing Compound F-1 directly using an appropriate condition such as for example, Pinnick, TEMPO or Jones oxidation. The solvent used in this step is not particularly limited in so far as it does not interfere with the reaction. The reaction temperature is usually −78° C. to room temperature. The reaction time is not particularly limited and is usually 5 minutes to 24 hours, preferably 30 minutes to 24 hours. The flurorination reaction can be performed using an appropriate reagent such as, for example DAST, Deoxofluor or sulfur tetrafluoride. The solvent used in this step is not particularly limited in so far as it does not interfere with the reaction. Examples of the solvent include dichloromethane, tetrahydrofuran, 1,4-dioxane, 1,2-dimethoxyethane, diethyl ether, toluene, benzene, and mixed solvents thereof. The reaction temperature is preferably −78° C. to 50° C. The reaction time is not particularly limited and is usually 5 minutes to 24 hours, preferably 30 minutes to 24 hours.

When R³″″″ is alkyloxy, Compound F′-2 can be obtained by means of alkylation of the terminal alcohol of Compound F′-1. This reaction can be performed using an appropriate base such as sodium hydride with the corresponding electrophiles such as alkyl halide, mesylate or triflate. The solvent used in this step is not particularly limited in so far as it does not interfere with the reaction. Examples of the solvent include acetone, acetonitrile, tetrahydrofuran, DMF, DMA, DMSO, toluene, and mixed solvents thereof. The reaction temperature is preferably 0° C. to 100° C. The reaction time is not particularly limited and is usually 5 minutes to 24 hours, preferably 30 minutes to 24 hours.

### General Procedure 1

The final compounds according to Formula (IC) can be prepared by reacting an intermediate compound of Formula (II-a) with a compound of Formula (XXIV) or the like according to following reaction scheme). The reaction is performed in a suitable reaction-inert solvent, such as, for example, dioxane, in the presence of a suitable base, such as, for example, potassium phosphate (K₃PO₄), a copper catalyst such as, for example, copper(I) iodide (CuI) and a diamine such as, for example, (1R,2R)-(−)-1,2-diaminocyclohexane or N, N-dimethylethylenediamine, under thermal conditions such as, for example, heating the reaction mixture at 100° C., for example for 16 hours. In reaction scheme (1) all variables are defined as in Formula (IC) and Z is chloro, bromo or iodo.

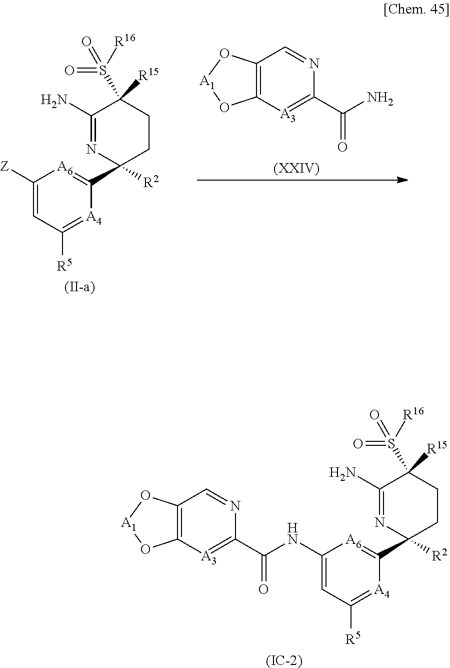

### General Procedure 2

Additionally, the final compounds according to Formula (IC) can be prepared by reacting an intermediate compound of Formula (II-b) with a compound of Formula (XXV) or the like according to following reaction scheme. The reaction is performed in a suitable reaction-inert solvent, such as, for example, methanol (MeOH), in the presence of an acid, such as, for example, hydrochloric acid (HCl), and of a carboxyl activating agent such as, for example, 1-ethyl-3-(3-dimethylaminopropyl)carbodiimide [EDCI, CAS 1892-57-5], under suitable conditions such as, for example, stirring the reaction mixture at 25° C., for example for 10 minutes. In reaction scheme (2) all variables are defined as in Formula (IC).

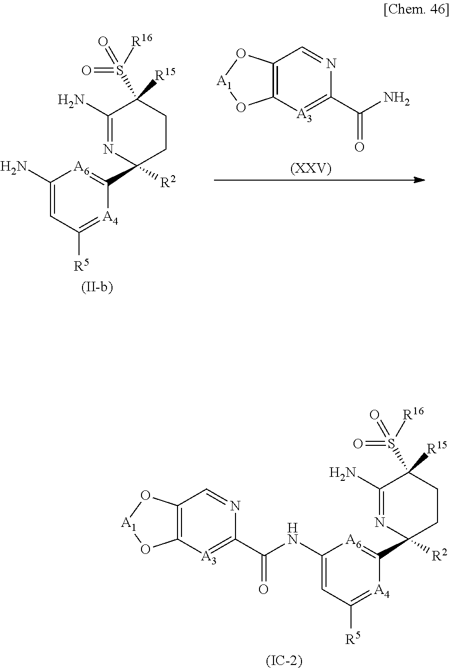

### General Procedure 3

Intermediate compounds according to Formula (II-b) can be prepared by subjecting an intermediate compound of Formula (III) to reducing conditions according to following reaction scheme. Typical examples are reduction by a suitable catalyst, such as, for example, palladium on carbon, under hydrogen atmosphere, or the use of a reducing agent such as, for example, tin(II) chloride. The reactions are typically performed in a suitable solvent, such as, for example, MeOH, or in a solvent mixture, such as tetrahydrofuran (THF)/ethanol (EtOH). Thermal conditions such as, for example, heating the mixture, may improve the reaction outcome. In reaction scheme (3) all variables are defined as in Formula (IC).

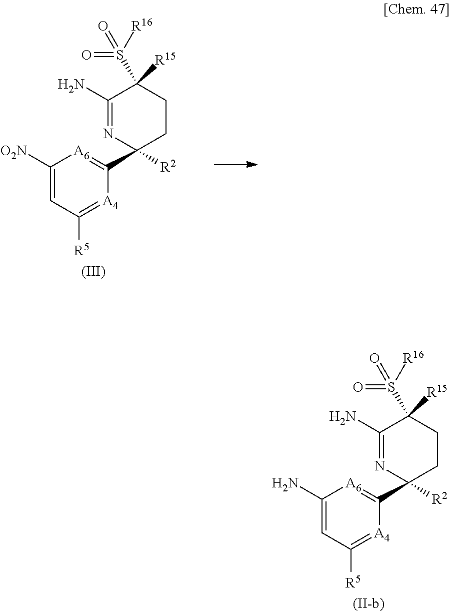

### General Procedure 4

Intermediate compound of Formula (II-b) can alternatively be prepared from intermediate of Formula (II-a) according to reaction scheme (4). In a typical procedure, a compound of Formula (II-a) in which Z is a halo, for example bromo, is reacted with sodium azide (NaN₃) to an intermediate compound of Formula (II-b). The reaction is performed in a suitable reaction-inert solvent, such as, for example acetonitrile, in the presence of a suitable base, such as, for example, sodium carbonate (Na₂CO₃), a copper catalyst such as, for example, copper(I) iodide (CuI) and a diamine such as, for example, N,N′-dimethylethylenediamine, under thermal conditions such as, for example, heating the reaction mixture at 100° C., for example for 16 hours. In reaction scheme (4) all variables are defined as in Formula (IC).

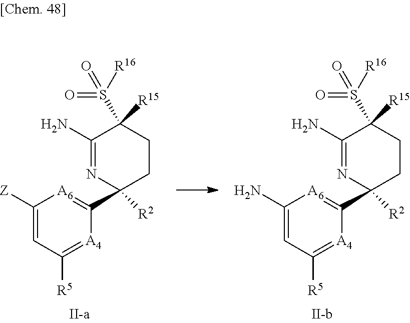

### General Procedure 5

Intermediate compounds according to Formula (III) can be prepared by nitration of an intermediate compound of Formula (II-c) according to the following reaction scheme. A typical procedure involves the treatment of intermediate (II), dissolved in sulphuric acid, with a source of nitronium ion, such as, for example, potassium nitrate, at low temperature, such as, for example, 0° C. In the following reaction scheme, R⁷ is hydrogen, and all other variables are defined as in Formula (IC).

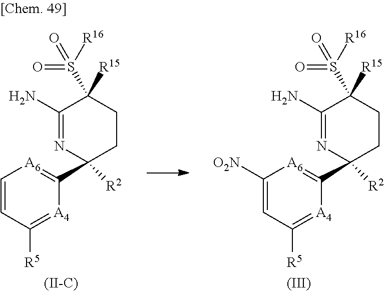

### General Procedure 6

Intermediate compounds according to Formulas (II-c) and (II-a) can be prepared by means of one-step or two-step procedures, according to the following reaction scheme, starting from a suitable compound of Formula (IV), where PG is a suitable protecting group, such as, for example, tert-butoxycarbonyl (BOC), trifluoroacetyl or tert-butylsulfinyl. In the two-step procedure the amino group in intermediate (IV) is first deprotected to give intermediate (V) by means of methods known to the person skilled in the art, such as, for example, by treating intermediate (IV) with an acid such as, for example, formic acid. Heating the reaction mixture, for example at 80° C. for about 4 hours, may improve the reaction outcome. Isolated intermediate (V) can then be dissolved in a suitable solvent, such as, for example, dichloromethane (DCM), and cyclised into the corresponding intermediate (II-c) or (II-a) in the presence of a Lewis acid, such as, for example, trimethyl aluminium. Alternatively, intermediate (IV) can be stirred in the presence of an acid, such as in-situ generated HCl in methanolic solution, pure formic acid, or trifluoroacetic acid in toluene under thermal conditions, such as, for example, heating the reaction mixture at about 120° C. for a period of time sufficient to drive the reaction to completion, to obtain corresponding intermediate (II-c) or (II-a) in one pot. In following reaction scheme, all variables are defined as in Formula (IC), Z is hydrogen or halo and PG is a protecting group.

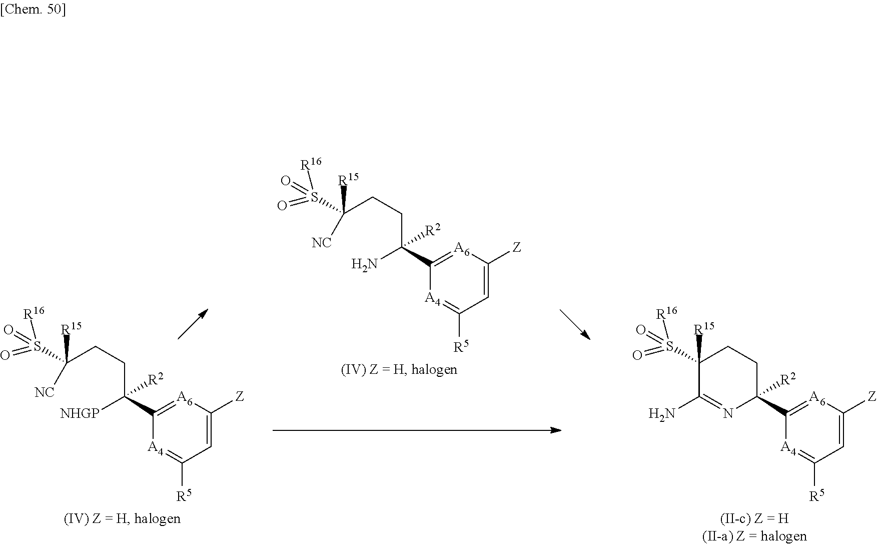

### General Procedure 7

Intermediate compounds according to Formula (IV) can be obtained by a two-step procedure starting from intermediate (VII) according to the following reaction scheme. Intermediate (VII) can be converted into intermediate (VI) by treatment with a reducing agent, such as, for example, sodium borohydride, in a suitable solvent, such as, for example, THF. Low temperature, such as, for example, 0° C., may improve the reaction outcome. Intermediate (VI) can then be converted into intermediate (IV) by means of standard alkylation reactions, such as, for example, by treating the compound, dissolved in a suitable solvent, such as, for example, THF, with a base, such as, for example, sodium hydride, and quenching the resulting anion with an alkylating agent, such as, for example, methyl iodide, at low temperature, such as, for example, at 0° C. Alternatively (VI) can be reacted with an aldehyde such as formaldehyde in presence of a suitable base such as triethylamine yielding a derivative (IV) in which R¹⁵ is a hydroxyalkyl, which can be further converted to a derivative in which R¹⁵ is a fluoroalkyl, for instance fluoromethyl, using an appropriate reagent such as, for example DAST, Deoxofluor or sulfur tetrafluoride. In the following reaction scheme, all variables are defined as in Formula (IC), Z is hydrogen or halo and PG is a protecting group for example, tert-butoxycarbonyl (BOC), benzyloxycarbonyl (CBZ), trifluoroacetyl or tert-butylsulfinyl.

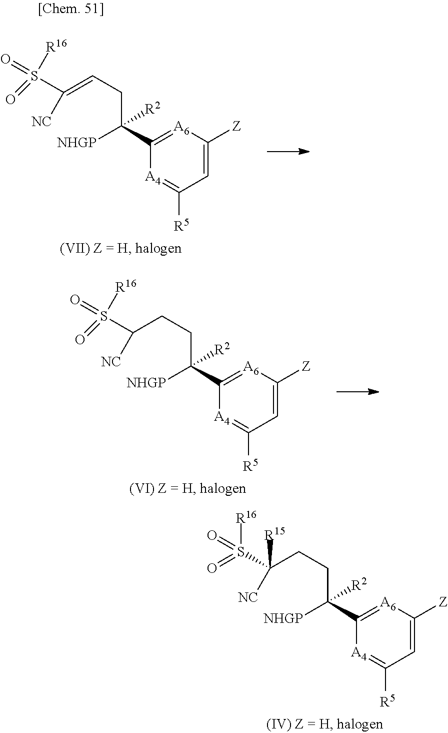

### General Procedure 8

Intermediate compounds according to Formula (VII) can be prepared in six steps starting from intermediate (XIII) according to the following reaction scheme. Intermediate (XIII) can be converted into intermediate (XII) by means of the nucleophilic addition of an appropriate anion. The anion can be generated by means of methods known to the person skilled in the art: Typical examples are treating the desired acetate, such as, for example, tert-butyl acetate, with an appropriate base, such as, for example, lithium diisopropylamide, in an inert solvent, such as, for example, THF, at a low temperature, such as, for example, at −78° C., or treating the corresponding alpha-bromoacetate with zinc in the presence of Cu(I) in an inert solvent, such as, for example, THF, at a temperature high enough to promote the insertion of the zinc into the carbon-bromine bond, such as, for example, at 40° C. The solution of the anion can then be reacted with a solution of intermediate (XIII) in an appropriate solvent, such as THF, at a temperature which allows smooth reaction, such as, for example, −78° C. or 0° C., to afford intermediate (XII). Using an intermediate (XIII) in which the tert-butylsulfinyl group has the R-configuration provides a mixture wherein there is an excess of that isomer wherein the aryl group is projected above the plane of the drawing (with the bond shown as a bold wedge). By choosing a suitable acid, such as, for example, hydrochloric acid, intermediate (XII) can then undergo hydrolysis of the ester and removal of the nitrogen protecting group in one pot to afford intermediate (XI). Stirring the reaction under thermal conditions, such as, for example, at 80° C. for 5 hours, may improve the reaction outcome. Intermediate (XI) can subsequently be reduced into the corresponding alcohol by treatment with a standard reducing agent, such as, for example, borane in THF, to afford intermediate (X). The amino group of intermediate (X) can be protected by means of methods known to the person skilled in the art, such as, for example, by treating intermediate (X), dissolved in a suitable solvent, such as, for example, DCM or THF, with an appropriate anhydride, such as, for example, trifluoroacetic anhydride or tert-butoxycarbonyl anhydride (BOC-anhydride), in the presence of a base, such as, for example, triethylamine or sodium hydrogenocarbonate. Protected intermediate (IX) can be subsequently oxidised to aldehyde (VIII) by means of standard oxidising agents, such as, for example, Dess-Martin periodinane in an inert solvent, such as, for example, DCM. Intermediate (VIII) can be finally converted into intermediate (VII) by means of a Knoevenagel condensation with a suitable active hydrogen component, such as, for example, 2-(alkylsulfonyl)acetonitrile, in the presence of a catalyst, such as, for example, magnesium oxide, in an inert solvent, such as, for example, MeOH. In the following reaction scheme, all variables are defined as in Formula (IC), Z is hydrogen or halo, PG is a protecting group and alkyl is a suitable alkyl group, e.g. ethyl.

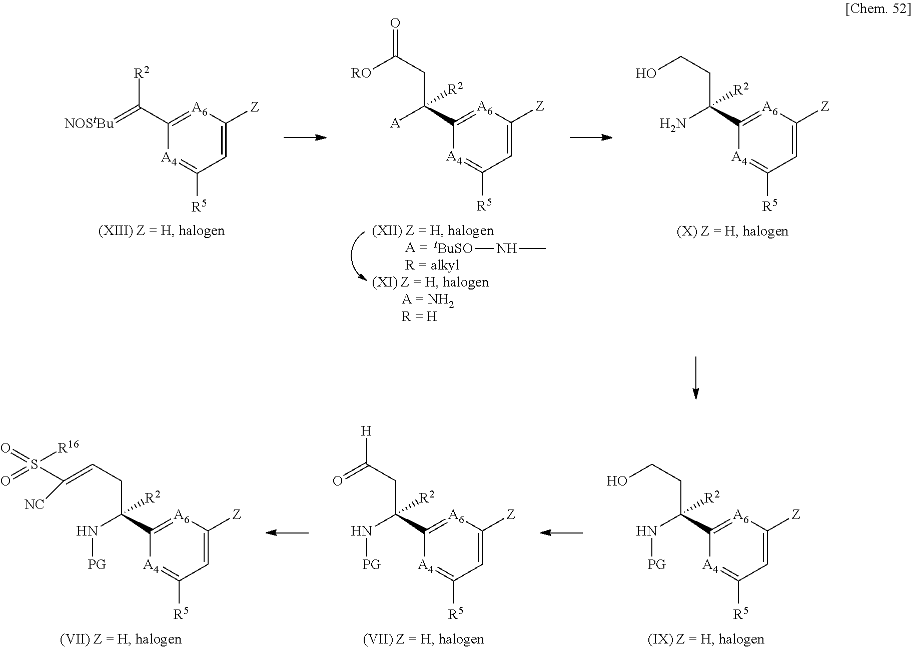

### General Procedure 8-a

Alternatively, intermediate compounds according to Formula (X) can be obtained in two steps starting from intermediate (XII-a), where Alk¹ is a suitable alkyl chain, such as, for example, ethyl, according to the following reaction scheme. Treatment of intermediate (XII-a) with an ester reducing agent, such as, for example, lithium borohydride, in an inert solvent, such as, for example, THF, at a temperature which allows smooth reaction, such as, for example, at 0° C., yields intermediate (XXII), which may be further deprotected into intermediate (X) by treatment with an appropriate acid, such as, for example, HCl, in an inert solvent, such as, for example, MeOH. In the following reaction scheme, all variables are defined as in Formula (IC), Z is hydrogen or halo and alkyl is a suitable alkyl chain, such as ethyl.

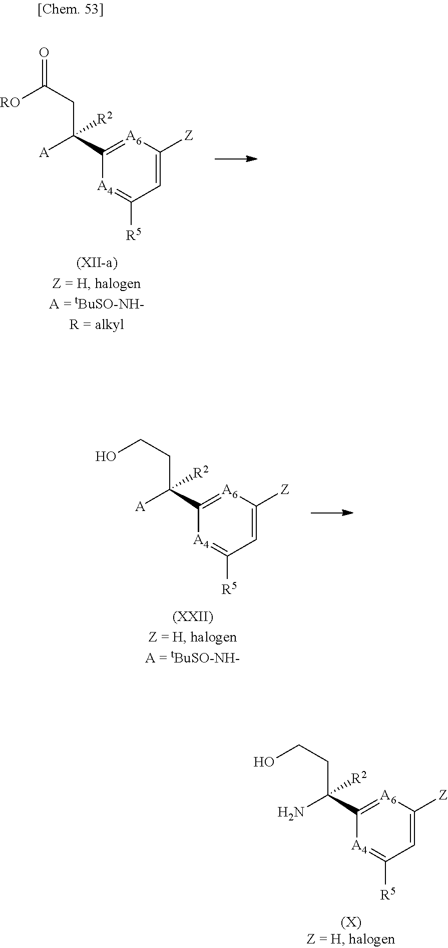

### General Procedure 9

Intermediate compounds according to Formula (VII-a) can be prepared in three steps starting from intermediate (XIII), according to the following reaction scheme. Intermediate (XIII), dissolved in a suitable solvent, such as, for example, DCM, can be reacted with a suitable nucleophile, such as, for example, allylmagnesium bromide, at low temperature, such as, for example, at −50° C., to give intermediate (XXI). Using an intermediate (XIII) in which the tert-butylsulfinyl group has the R-configuration provides a mixture wherein there is an excess of that isomer wherein the aryl group is projected above the plane of the drawing (with the bond shown as a bold wedge). Oxidative cleavage of the newly installed double bond by means of standard methods, such as, for example, ozonolysis at low temperature, such as, for example, at 0° C., affords intermediate (XX), which can be finally converted into intermediate (VII-a) by means of a Knoevenagel condensation with a suitable active hydrogen component, such as, for example, 2-(alkylsulfonyl)acetonitrile, in the presence of a catalyst, such as, for example, magnesium oxide, in an inert solvent, such as, for example, MeOH. In the following reaction scheme, all variables are defined as in Formula (IC) and Z is hydrogen or halo.

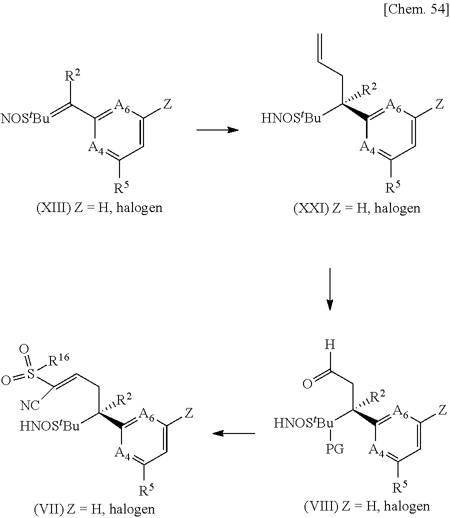

### General Procedure 10

Intermediate compounds according to Formula (VII-b) can be prepared in four steps starting from intermediate (XII-a), according to the following reaction scheme. Intermediate (XII-a) can be deprotected to give free-amino intermediate (XVIII) by means of standard deprotection techniques, such as by treating intermediate (XII-a), dissolved in a suitable solvent, such as MeOH, with an acid, such as, for example, HCl. Conversion to the mono-BOC derivative intermediate (XVII) can be achieved by submitting intermediate (XVIII) to conditions known to the person skilled in the art, such as, for example, by treating intermediate (XVIII), dissolved in an appropriate solvent, such as, for example, MeOH, with a BOC source, such as, for example, BOC-anhydride. Raising the temperature, for example to 60° C., for example for 7 hours, may improve the reaction outcome. Intermediate (XVII), dissolved in a suitable solvent, such as, for example, DCM or THF, can be reduced to the corresponding aldehyde (XVI) by means of selective reducing agents, such as, for example, diisobutylaluminium hydride, at low temperature, such as, for example, at −78° C., or lithium borohydride at low temperature, such as, for example, at 0° C. Possible overreduced alcohol side-products can be converted back into intermediate (XVI) by means of standard oxidation reagents, such as, for example, by using Dess-Martin periodinane in DCM. Intermediate (XVI) can be finally converted into intermediate (VII-b) by means of a Knoevenagel condensation with a suitable active hydrogen component, such as, for example, 2-(alkylsulfonyl)acetonitrile, in the presence of a catalyst, such as, for example, magnesium oxide, in a suitable solvent, such as, for example, MeOH. In the following reaction scheme, all variables are defined as in Formula (IC), Z is hydrogen or halo and R is a suitable alkyl group.

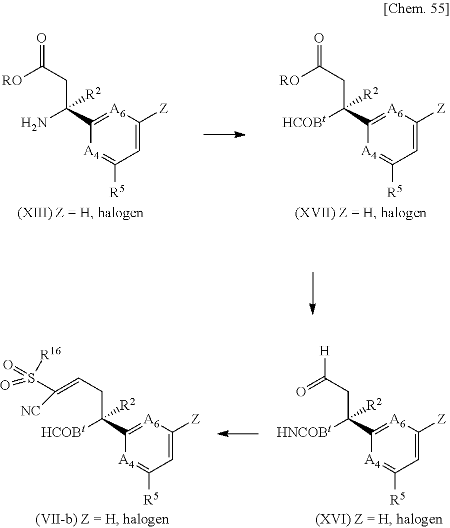

The bicyclic amides (XXIV) and acids (XXV) are conveniently available from various halogenated pyrones, pyridines, pyridones and pyrimidines.

The compounds of the present invention have BACE1 inhibitory activity and are effective in treatment and/or prevention, symptom improvement, and prevention of the progression of disease induced by the production, secretion or deposition of amyloid ß peptides, such as Alzheimer's disease, Alzheimer dementia, senile dementia of Alzheimer type, mild cognitive impairment (MCI), prodromal Alzheimer's disease (e.g., MCI due to Alzheimer's disease), Down's syndrome, memory impairment, prion disease (Creutzfeldt-Jakob disease), Dutch type of hereditary cerebral hemorrhage with amyloidosis, cerebral amyloid angiopathy, other type of degenerative dementia, mixed dementia such as coexist Alzheimer's disease with vascular type dementia, dementia with Parkinson's Disease, dementia with progressive supranuclear palsy, dementia with Cortico-basal degeneration, Alzheimer's disease with diffuse Lewy body disease, age-related macular degeneration, Parkinson's Disease, amyloid angiopathy or the like.

Furthermore, the compounds of the present invention are effective in preventing the progression in a patient asymptomatic at risk for Alzheimer dementia (preclinical Alzheimer's disease).

“A patient asymptomatic at risk for Alzheimer dementia” includes a subject who is cognitively and functionally normal but has potential very early signs of Alzheimer's disease or typical age related changes (e.g., mild white matter hyper intensity on MRI), and/or have evidence of amyloid deposition as demonstrated by low cerebrospinal fluid Aß₁₋₄₂ levels. For example, “a patient asymptomatic at risk for Alzheimer dementia” includes a subject whose score of the Clinical Dementia Rating (CDR) or Clinical Dementia Rating-Japanese version (CDR-J) is 0, and/or whose stage of the Functional Assessment Staging (FAST) is stage 1 or stage 2.

The compound of the present invention has not only BACE1 inhibitory activity but the beneficialness as a medicament. The compound has, preferably, any one or more of the following superior properties.

a) The compound has weak inhibitory activity for CYP enzymes such as CYP1A2, CYP2C9, CYP2C19, CYP2D6, CYP3A4.
b) The compound show excellent pharmacokinetics profiles such as high bioavailability or low clearance.
c) The compound has a high metabolic stability.
d) The compound does not show irreversible inhibitions to CYP enzymes such as CYP3A4 in the range of the concentrations of the measurement conditions described in this description.
e) The compound does not show a mutagenesis.
f) The compound is at a low risk for cardiovascular systems.
g) The compound shows a high solubility.
h) The compound shows a high brain distribution.
i) The compound has a high oral absorption.
j) The compound has a long half-life period.
k) The compound has a high protein unbinding ratio.
l) The compound is negative in the Ames test.
m) The compound has a high BACE1 selectivity over BACE2.
n) The compound has weak mechanism based inhibition against CYP enzymes. For example, the reactive metabolites of the compound have weak inhibition against CYP enzymes.
o) The compound generates little reactive metabolites.
p) The compound is a weak P-gp substrate.

Since the compound of the present invention has high inhibitory activity on BACE1 and/or high selectivity on other enzymes, for example, BACE2, it can be a medicament with reduced side effect. Further, since the compound has high effect of reducing amyloid ß production in a cell system, particularly, has high effect of reducing amyloid ß production in brain, it can be an excellent medicament. In addition, by converting the compound into an optically active compound having suitable stereochemistry, the compound can be a medicament having a wider safety margin on the side effect.

When a pharmaceutical composition of the present invention is administered, it can be administered orally or parenterally. The composition for oral administration can be administered in usual dosage forms such as oral solid formulations (e.g., tablets, powders, granules, capsules, pills, films or the like), oral liquid formulations (e.g., suspension, emulsion, elixir, syrup, lemonade, spirit, aromatic water, extract, decoction, tincture or the like) and the like may prepared according to the usual method and administered. The tablets can be sugar-coated tablets, film-coated tablets, enteric-coating tablets, sustained-release tablets, troche tablets, sublingual tablets, buccal tablets, chewable tablets or orally disintegrated tablets. Powders and granules can be dry syrups. Capsules can be soft capsules, micro capsules or sustained-release capsules.

The composition for parenteral administration can be administered suitably in usual parenteral dosage forms such as dermal, subcutaneous, intravenous, intraarterial, intramuscular, intraperitoneal, transmucosal, inhalation, transnasal, ophthalmic, inner ear or vaginal administration and the like. In case of parenteral administration, any forms, which are usually used, such as injections, drips, external preparations (e.g., ophthalmic drops, nasal drops, ear drops, aerosols, inhalations, lotion, infusion, liniment, mouthwash, enema, ointment, plaster, jelly, cream, patch, cataplasm, external powder, suppository or the like) and the like can be preferably administered. Injections can be emulsions whose type is O/W, W/O, O/W/O, W/O/W or the like.

The compounds of the present invention can be preferably administered in an oral dosage form because of their high oral absorbability.

A pharmaceutical composition can be formulated by mixing various additive agents for medicaments, if needed, such as excipients, binders, disintegrating agents, and lubricants which are suitable for the formulations with an effective amount of the compound of the present invention. Furthermore, the pharmaceutical composition can be for pediatric patients, geriatric patients, serious cases or operations by appropriately changing the effective amount of the compound of the present invention, formulation and/or various pharmaceutical additives. The pediatric pharmaceutical compositions are preferably administered to patients under 12 or 15 years old. In addition, the pediatric pharmaceutical compositions can be administered to patients who are under 27 days old after the birth, 28 days to 23 months old after the birth, 2 to 11 years old, 12 to 16 years old, or 18 years old. The geriatric pharmaceutical compositions are preferably administered to patients who are 65 years old or over.

The dosage of a pharmaceutical composition of the present invention should be determined in consideration of the patient's age and body weight, the type and degree of diseases, the administration route and the like. The usual oral dosage for adults is in the range of 0.05 to 100 mg/kg/day and preferable is 0.1 to 10 mg/kg/day. For parenteral administration, the dosage highly varies with administration routes and the usual dosage is in the range of 0.005 to 10 mg/kg/day and preferably 0.01 to 1 mg/kg/day. The dosage may be administered once or several times per day.

The compound of the present invention can be used in combination with other drugs for treating Alzheimer's disease, Alzheimer dementia or the like such as acetylcholinesterase inhibitor (hereinafter referred to as a concomitant medicament) for the purpose of enforcement of the activity of the compound or reduction of the amount of medication of the compound or the like. In this case, timing of administration of the compound of the present invention and the concomitant medicament is not limited and these may be administered to the subject simultaneously or at regular intervals. Furthermore, the compound of the present invention and concomitant medicament may be administered as two different compositions containing each active ingredient or as a single composition containing both active ingredient.

The dose of the concomitant medicament can be suitably selected on the basis of the dose used on clinical. Moreover, the mix ratio of the compound of the present invention and a concomitant medicament can be suitably selected in consideration of the subject of administration, administration route, target diseases, symptoms, combinations, etc. For example, when the subject of administration is human, the concomitant medicament can be used in the range of 0.01 to 100 parts by weight relative to 1 part by weight of the compounds of the present invention.

Examples of a concomitant medicament are Donepezil hydrochloride, Tacrine, Galanthamine, Rivastigmine, Zanapezil, Memantine and Vinpocetine.

### EXAMPLE

Following examples and test examples illustrate the present invention in more detail, but the present invention is not limited by these examples.

In examples, the meaning of each abbreviation is as follows:

### Ac: Acetyl

Et: ethyl
Bz: benzoyl
DCC: dicyclohexylcarbodiimide
DIC: diisopropylcarbodiimide
iPr: isopropyl
Me: methyl
Ph: phenyl
t-Bu: tert-butyl
TBS: tert-butyldimethylsilyl
AIBN: azobisisobutyronitrile
BOMCl benzyl chloromethyl etheroxymethyl chloride
DAST: N,N-diethylaminosulfur trifluoride
DBU: 1,8-Diazabicyclo[5.4.0]-7-undecene
DEAD: diethyl azodicarboxylate
DCM: dichloromethane
DIAD: diisopropyl azodicarboxylate

### ADDP: 1,1′-(Azodicarbonyl)dipiperidine

### DMA: N,N-dimethylacetamide

DMAP: 4-dimethylaminopyridine

### DMF: N,N-dimethylformamide

DMSO: dimethylsulfoxide
EDC: 1-ethyl-3-(3-dimethylaminopropyl)carbodiimide
HATU: O-(7-aza-1H-benzotriazol-1-yl)-N,N,N′,N′-tetramethyluronium hexafluorophosphate
HOAt: 1-hydroxy-7-aza-benzotriazole
HOBt: 1-hydroxy-benzotriazole
LDA: lithium diisopropylamide
LHMDS: lithium bis(trimethylsilyl)amide
mCPBA: m-chloroperoxybenzoic acid

### NCS: N-chlorosuccinimide

### NMP: N-methylpyrrolidone

PyBOP: 1H-Benzotriazol-1-yloxy-tri(pyrrolidino) phosphonium hexafluorophosphate
TEMPO: 2,2,6,6-tetramethylpiperidine 1-oxyl free radical
TFA: trifluoroacetic acid
THF: tetrahydrofuran
THP: 2-tetrahydropyranyl
DCC: dicyclohexylcarbodiimide
DIC: diisopropylcarbodiimide
TBS: tert-butyldimethylsilyl

### ADDP: 1,1′-(Azodicarbonyl)dipiperidine

(Boc)₂O: di-tert-butyl Dicarbonate
DIBAL: diisobutylaluminum Hydride

### DIPEA: N,N-diisopropylethylamine

PPTS: pyridinium p-toluenesulfonate

¹H NMR spectra were recorded on Bruker Advance 400 MHz spectrometer with chemical shift reported relative to tetramethylsilane or the residual solvent peak (CDCl₃=7.26 ppm, DMSO-d₆=2.50 ppm).

Analytical LC/MS (ESI positive or negative, retention time (RT)) data were recorded on Shimadzu UFLC or Waters UPLC system under the following conditions:

### Method A

Column: XBridge (Registered trademark) C18 (5 μm, i.d. 4.6×50 mm) (Waters)
Flow rate: 3 mL/min
UV detection wavelength: 254 nm
Mobile phases: [A] is 0.1% formic acid solution, and [B] is 0.1% formic acid in acetonitrile solvent.
Gradient: linear gradient of 10% to 100% solvent [B] for 3 minutes was performed, and 100% solvent FBI was maintained for 1 minute.

### Method B

Column: Shim-pack XR-ODS (2.2 μm, i.d. 50×3.0 mm) (Shimadzu)
Flow rate: 1.6 mL/min
Column oven: 50° C.
UV detection wavelength: 254 nm
Mobile phase: [A] 0.1% formic acid-containing aqueous solution; [B] 0.1% formic acid-containing acetonitrile solution
Gradient: linear gradient from 10% to 100% solvent [B] for 3 minutes and 100% solvent [B] for 1 minute

### Method C

Column: BEH C18 (1.7 μm, 2.1×50 mm) (Waters)
Flow rate: 0.8 mL/min
UV detection wavelength: 254 nm
Mobile phases: [A] is 10 mM CH₃COONH₄ in 95% H₂O+5% CH₃CN, and [B] is acetonitrile.
Gradient: linear gradient of 5% to 95% solvent [B] for 1.3 minutes was performed, and 95% solvent FBI was maintained for 0.7 minutes.

### Method D

Column: HSS T3 (1.8 μm, 2.1×100 mm) (Waters)
Flow rate: 0.7 mL/min
UV detection wavelength: 254 nm
Mobile phases: [A] is 10 mM CH₃COONH₄ in 95% H₂O+5% CH₃CN, and [B] is acetonitrile.
Gradient: linear gradient of 0% to 95% solvent [B] for 2.1 minutes was performed, and 95% solvent FBI was maintained for 0.5 minutes.

The following compounds are prepared in a manner similar to the above. In the tables, RT means LC/MS retention time (minute).

### Example 1

### Synthesis of Compound I-001

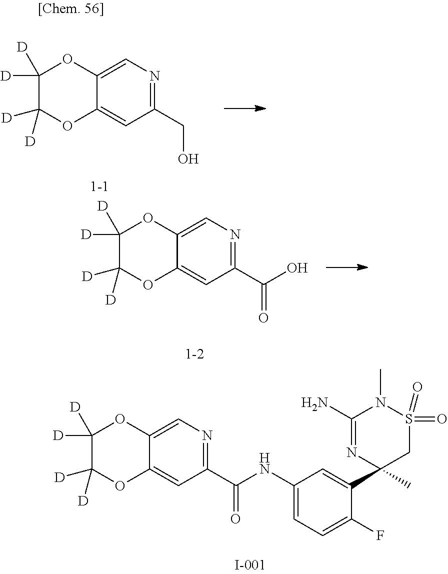 [SMILES: [2H]C1([2H])Oc2cnc(CO)cc2OC1([2H])[2H] ; [2H]C1([2H])Oc2cnc(C(=O)O)cc2OC1([2H])[2H] ; [2H]C1([2H])Oc2cnc(C(=O)Nc3ccc(F)c([C@]4(C)CS(=O)(=O)N(C)C(N)=N4)c3)cc2OC1([2H])[2H]]

### Step 1

TEMPO (0.82 g, 5.19 mmol) was added to a mixture of Compound I-1 (6.35 g, 37.1 mmol) and Phosphate buffer (pH=7, 101 mL) in acetonitrile (110 mL). The reaction was stirred at 35° C. NaClO₂ (16.8 g, 148 mmol) in H₂O (30 mL) and 15% NaClO (23 mL, 55.6 mmol) were added simultaneously in three slow additions each 30 min. The resulting reaction mixture was stirred at 35° C. for 16 hours. The reaction mixture was cooled to 5° C. and a solution of Na₂S₂O₃ (14.3 g, 59.4 mmol) in water 50 ml was added dropwise until the reaction mixture turned white. The reaction mixture was stirred for 30 min. Then 5M NaOH (7.5 mL, 37.1 mmol) was added. The reaction mixture was evaporated under reduced pressure. The mixture was extracted with EtOAc then aqueous phase as treated with HCl conc. at 5° C. until pH 3. The aqueous layer was concentrated under reduced pressure to dryness. The solid was washed with hot MeOH-DCM 1:1 twice and filtered. The filtrate was concentrated and purified by flash column chromatography (silica; MeOH in DCM 0/100 to 10/90). The desired fractions were collected and concentrated in vacuo to yield Compound I-2 (6.2 g, 3.34 mmol, 89%) as a pale yellow solid.

LC/MS: Method C, M+1=186, tR=0.22 min.

### Step 2

To a suspension of (R)-3-amino-5-(5-amino-2-fluorophenyl)-2,5-dimethyl-5,6-dihydro-2H-1,2,4-thiadiazine 1,1-dioxide (765 mg, 2.67 mmol) in MeOH (276 mL) was added HCl (6M in ⁱPrOH, 0.67 mL, 4.0 mmol) and the mixture was stirred for 5 minutes. Then, Compound I-2 (0.62 g, 3.34 mmol) and EDC hydrochloride (0.79 g, 4.0 mmol) were added and the reaction was stirred at room for 1 hour. The solvent was evaporated. The residue was taken up in DCM (50 mL) and a sat. Na₂CO₃ solution (30 mL). The organic layer was separated and the aqueous one was extracted with DCM (50 mL). The combined organic layers were dried over MgSO₄, filtered and concentrated under vacuum. The crude was purified by flash chromatography on silica gel (40 g, gradient: from DCM 100% up to DCM/MeOH(NH₃) 95/5). The desired fractions were collected and concentrated in vacuo to yield Compound I-001 (0.74 g, 1.62 mmol, 60%) as a white solid.

¹H NMR (400 MHz, CDCl₃) δ: 1.78 (3H, s), 3.23 (3H, s), 3.74 (1H, d, J=14.1 Hz), 3.81 (1H, d, J=14.1 Hz), 7.05 (1H, dd, J=11.7, 8.7 Hz), 7.76-7.81 (2H, m), 7.83 (1H, dd, J=7.2, 2.8 Hz), 8.11 (1H, s).

LC/MS: Method B, M+1=454, tR=0.75 min.

### Example 21

### Synthesis of Compound I-003

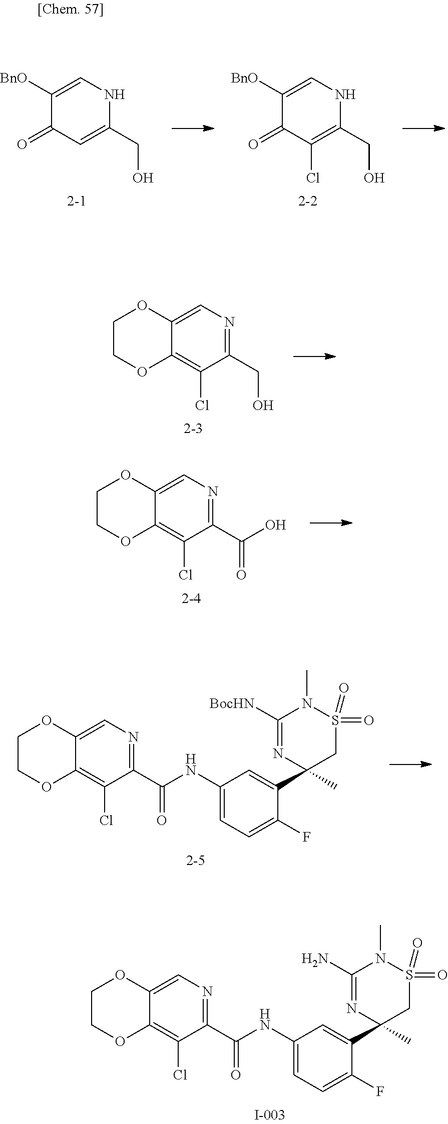 [SMILES: O=c1cc(CO)[nH]cc1OCc1ccccc1 ; O=c1c(OCc2ccccc2)c[nH]c(CO)c1Cl ; OCc1ncc2c(c1Cl)OCCO2 ; O=C(O)c1ncc2c(c1Cl)OCCO2 ; CN1C(NC(=O)OC(C)(C)C)=N[C@](C)(c2cc(NC(=O)c3ncc4c(c3Cl)OCCO4)ccc2F)CS1(=O)=O ; CN1C(N)=N[C@](C)(c2cc(NC(=O)c3ncc4c(c3Cl)OCCO4)ccc2F)CS1(=O)=O]

### Step 1

To a suspension of Compound 2-1 (4.77 g, 20.6 mmol) in chloroform (47.7 mL) was added NCS (3.31 g, 24.8 mmol). After being stirred for 2 hours at 70° C., the reaction mixture was cooled to room temperature and chloroform (48 mL) was added to the reaction mixture. The suspension was filtered to give Compound 2-2 (5.97 g, 20.6 mmol, 100%) as a white solid.

¹H NMR (400 MHz, CDCl₃) δ: 3.15-3.70 (1H, br), 4.34 (1H, s), 5.03 (1H, 7.22-7.50 (6H, m).

### Step 2

To a suspension of Compound 2-2 (5.97 g, 20.6 mmol) in THF (59.7 mL) were added an aqueous 2 mol/L sodium hydroxide (12.4 mL, 24.8 mmol) and 10 w/w % palladium on carbon (3 g). After being stirred for 3 hours at room temperature under 1 atm hydrogen. The reaction mixture was filtered through Celite (Registered trademark) pad. The filtrate was evaporated. To a suspension of the residue in DMF (59.7 mL) were added potassium carbonate (8.55 g, 61.9 mmol) and 1, 2-dibromoethane (2.67 mL, 30.9 mmol). The reaction mixture was stirred for 1 hour at 70° C. and for 3 hours at 90° C. To the reaction mixture was added toluene (100 mL), and the suspension was filtered. The filtrate was evaporated. The crude product was added to a silica gel column and eluted with hexane/EtOAc 50% to 100%. Collected fractions were evaporated to afford Compound 2-3 (2.65 g, 13.1 mmol, 64%) as a mixture of yellow oil and solid.

¹H NMR (400 MHz, CDCl₃) δ: 4.14-4.18 (1H, br), 4.30-4.34 (2H, m), 4.45-4.48 (2H, m), 4.68-4.71 (2H, m), 8.09 (1H, s).

### Step 3

To a suspension of Compound 2-3 (2.65 g, 13.1 mmol) in DCM (26.5 mL) was added manganese dioxide (15.0 g, 173 mmol). After being stirred for 1 hour at room temperature, the reaction mixture was filtered through Celite (Registered trademark) pad. The filtrate was evaporated. The crude product was added to a silica gel column and eluted with hexane/EtOAc 10% to 50%. Collected fractions were evaporated to afford Compound 2-4 (1.03 g, 5.15 mmol, 39%) as a white solid. ¹H NMR (400 MHz, CDCl₃) δ: 4.39-4.43 (2H, m), 4.49-4.52 (2H, m), 8.39 (1H, s), 10.2 (1H, s).

### Step 4

To a solution of Compound 2-4 (1.03 g, 5.15 mmol) in acetone (30.8 mL) and water (10.3 mL) were added Sodium dihydrogen phosphate (927 mg, 7.73 mL), 2-methyl-2-butene (5.46 mL, 51.5 mmol) and sodium chlorite (1.75 g, 15.5 mmol) at 0° C. After being stirred for 1 hour at room temperature, aqueous 2 mol/L hydrochloric acid (7 mL) was added to the reaction mixture. The mixture was evaporated and cooled to 0° C. The suspension was filtered to give aldehyde (433 mg, 2.01 mmol, 39%) as a white solid.

¹H NMR (400 MHz, DMSO-d6) δ: 4.37-4.41 (2H, m), 4.48-4.52 (2H, m), 8.13 (1H, s). To a solution of the aldehyde (1.03 g, 5.15 mmol) in acetone (30.8 mL) and water (10.3 mL) were added sodium dihydrogen phosphate (927 mg, 7.73 mL), 2-methyl-2-butene (5.46 mL, 51.5 mmol) and sodium chlorite (1.75 g, 15.5 mmol) at 0° C. After being stirred for 1 hour at room temperature, aqueous 2 mol/L hydrochloric acid (7 mL) was added to the reaction mixture. The mixture was evaporated and cooled to 0° C. The suspension was filtered to give Compound 2-5 (433 mg, 2.01 mmol, 39%) as a white solid.

¹H NMR (400 MHz, d6-DMSO) δ: 4.37-4.41 (2H, m), 4.48-4.52 (2H, m), 8.13 (1H, s).

### Step 5

To a suspension of tert-butyl (R)-(5-(5-amino-2-fluorophenyl)-2,5-dimethyl-1, 1-dioxido-5,6-dihydro-2H-1,2,4-thiadiazin-3-yl)carbamate (58.0 mg, 0.15 mmol), Compound 2-5 (38.8 mg, 0.18 mmol), HOBt (24.3 mg, 0.180 mmol) and DMAP (3.7 mg, 0.030 mol) in DMF (1.16 mL) was added EDC hydrochloride (34.5 mg, 0.180 mmol). After being stirred for 90 minutes at room temperature, the reaction mixture was diluted with water and extracted with ethyl acetate. The combined organic layers were washed with water and brine, dried over sodium sulfate, and filtered. The solvent was evaporated. The crude product was added to a silica gel column and eluted with hexane/EtOAc 10% to 70%. Collected fractions were evaporated to afford Compound 2-6 (67.0 mg, 0.115 mmol, 77%) as a white foam.

LC/MS: Method A, M+1=584, 586, tR=2.14 min.

### Step 6

To a solution of Compound 2-6 (67.0 mg, 0.115 mmol) in dichloromethane (1 mL) was added TFA (1 mL, 13.0 mmol). After being stirred for 60 minutes at room temperature, the reaction mixture was evaporated. The residue was quenched with aqueous potassium carbonate and extracted with ethyl acetate. The combined organic layers were washed with brine, dried over sodium sulfate, and filtered. The solvent was evaporated. The crude product was triturated with n-hexane/ethyl acetate to give Compound I-003 (42.1 mg, 0.087 mmol, 76%) as a white solid.

¹H NMR (400 MHz, CDCl₃) δ: 1.79 (3H, s), 3.24 (3H, s), 3.74 (1H, d, J=13.9 Hz), 3.82 (1H, d, J=13.9 Hz), 4.36-4.40 (2H, m), 4.49-4.52 (2H, m), 7.05 (1H, dd, J=11.4, 8.9 Hz), 7.71 (1H, dd, J=7.0, 2.6 Hz), 7.85-7.94 (1H, m), 8.07 (1H, s), 9.84 (1H, s).

### Example 31

### Synthesis of Compound I-004

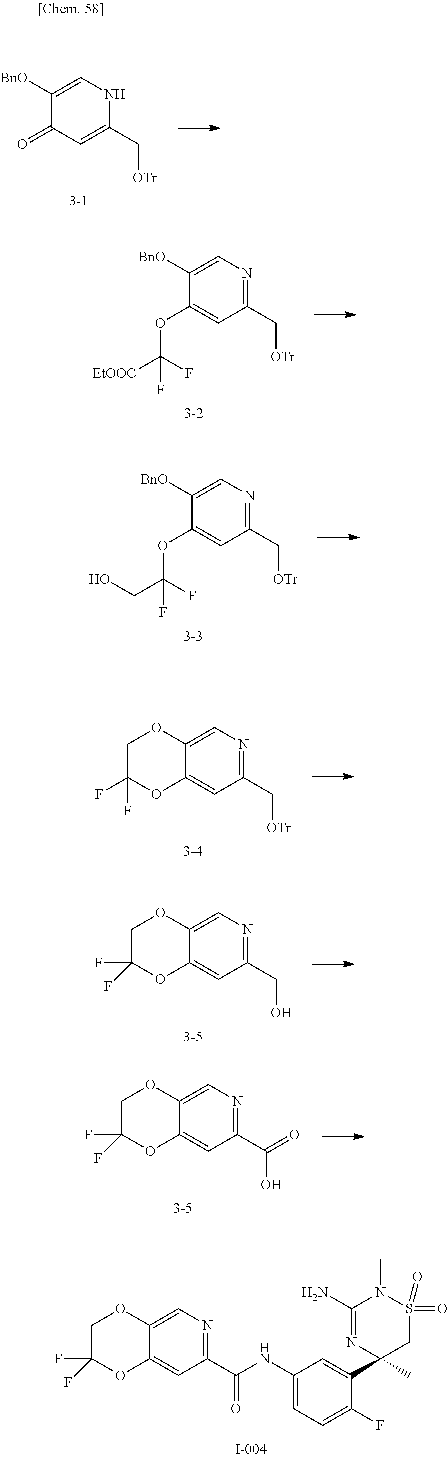

### Step 1

To a suspension of Compound 3-1 (1.89 g, 4 mmol) in toluene (18.9 mL) were added ethyl 2-bromo-2,2-difluoroacetate (1.54 mL, 12.0 mmol) and DBU (1.21 mL, 8.00 mmol). The reaction mixture was stirred for 5 hours at 80° C. The reaction mixture was diluted with aqueous citric acid and extracted with ethyl acetate. The combined organic layers were washed with water and brine, dried over sodium sulfate, and filtered. The solvent was evaporated. The crude product was added to a silica gel column and eluted with hexane/EtOAc 20% to 50%. Collected fractions were evaporated to afford Compound 3-2 (1.63 g, 2.74 mmol, 69%) as a white solid.

¹H NMR (400 MHz, CDCl₃) δ: 1.34 (3H, s), 4.27 (2H, s), 4.38 (2H, q, J=7.1H), 5.18 (2H, s), 7.21-7.51 (20H, m), 7.61 (1H, s), 8.22 (1H, s).

### Step 2

To a solution of Compound 3-2 (1.63 g, 2.74 mmol) in THF (16.3 mL), methanol (8.16 mL) and water (1.63 mL) was added sodium borohydride (207 mg, 5.48 mmol) at 0° C. The reaction mixture was stirred for 2.5 hours at room temperature. The reaction mixture was quenched with saturated aqueous ammonium chloride and extracted with ethyl acetate. The combined organic layers were washed with water-brine (4:1) and brine, dried over sodium sulfate, and filtered. The solvent was evaporated. The crude product was added to a silica gel column and eluted with hexane/EtOAc 10% to 50%. Collected fractions were evaporated to afford Compound 3-3 (1.02 g, 1.85 mmol, 62%) as a white solid.

¹H NMR (400 MHz, CDCl₃) δ: 2.43 (1H, s, J=7.5 Hz), 4.01 (2H, q, J=8.2 Hz), 4.22 (2H, s), 5.16 (2H, s), 7.21-7.51 (20H, m), 7.66 (1H, s), 8.23 (1H, s).

### Step 3

To a solution of Compound 3-3 (1.02 g, 1.85 mmol) in THF (20.4 mL) was added 10 wt. % palladium on carbon (500 mg). After being stirred for 2 hours at room temperature under hydrogen (1 atm), the reaction mixture was filtered through Celite (Registered trademark) pad. The filtrate was evaporated. To a solution of the residue and triphenylphosphine (968 mg, 3.69 mmol) in THF (20.4 mL) was added 1.9 mol/L of DIAD in toluene (1.94 mL, 3.69 mmol) at 0° C. The reaction mixture was stirred for 1 hour at 0° C. and for 1 hour at room temperature. The reaction mixture was evaporated. The crude product was added to a silica gel column and eluted with hexane/EtOAc 10% to 50%. Collected fractions were evaporated to afford the product. For further purification, it was added to an amino silica gel column and eluted with hexane/EtOAc 10% to 50%. Collected fractions were evaporated to afford Compound 3-4 (433 mg, 2.01 mmol, 39%) as a yellow solid.

¹H NMR (400 MHz, CDCl₃) δ: 4.28 (2H, s), 4.31 (2H, t, J=6.0 Hz), 7.22-7.34 (9H, m), 7.42 (1H, s), 7.48 (6H, d, J=7.5 Hz), 8.22 (1H, s).

### Step 4

To a solution of Compound 3-4 (858 mg, 1.93 mmol) in methanol (17.2 mL) was added p-toluenesulfonic acid hydrate (550 mg, 2.89 mmol). After being stirred for 2 hours at 70° C., the reaction mixture was cooled to room temperature and quenched with triethylamine (0.801 mL, 5.78 mmol). The mixture was evaporated. The crude product was added to a silica gel column and eluted with hexane/EtOAc 50% to 100%. Collected fractions were evaporated to afford Compound 3-5 (334 mg, 1.65 mmol, 85%) as a white solid.

¹H NMR (400 MHz, CDCl₃) δ: 3.12-3.27 (1H, br), 4.31 (2H, t, J=6.0 Hz), 4.70 (2H, s), 6.99 (1H, s), 8.31 (1H, s).

### Step 5

The Compound 3-6 was prepared in a manner similar to the above protocols. (Example 2)(yield; 65%)

¹H NMR (400 MHz, DMSO-d6) δ: 4.81 (2H, t, J=6.5 Hz), 7.84 (1H, s), 8.53 (1H, s).

### Step 6

The Compound I-004 was prepared in a manner similar to the above protocols. (Example 2) (yield; 73%)

¹H NMR (400 MHz, CDCl₃) δ: 1.82 (3H, s), 3.25 (3H, s), 3.74 (1H, d, J=14.2 Hz), 3.92 (1H, d, J=14.2 Hz), 4.39 (2H, t, J=5.9 Hz), 7.97 (1H, s), 7.08 (1H, dd, J=11.5, 8.7 Hz), 7.78-7.85 (2H, m), 7.97 (1H, s), 8.32 (1H, s), 9.79 (1H, s). LC/MS: Method A, M+1=586, tR=2.35 min.

### Example 41

### Synthesis of Compound I-011

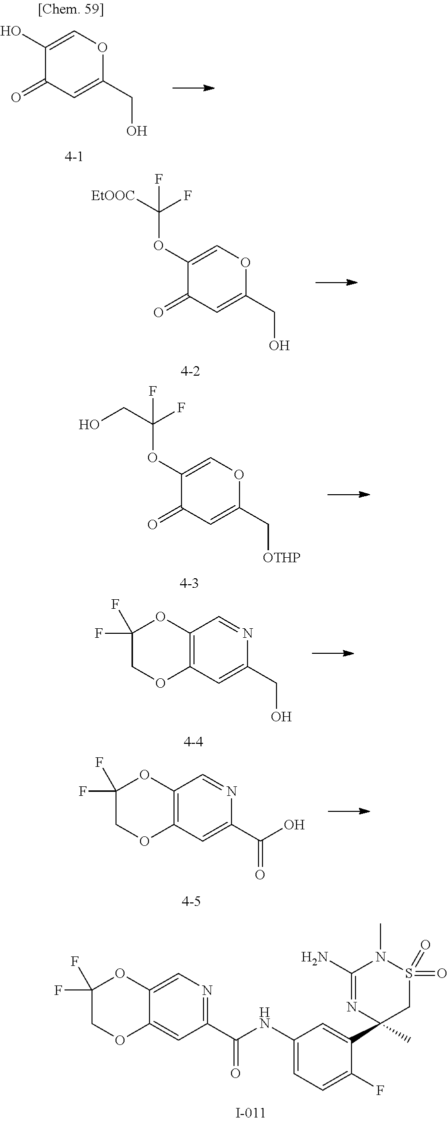

### Step 1

To a solution of Compound 4-1 (120 g, 844 mmol) in THF (480 mL) were added 3,4-dihydro-2H-pyran (81 mL, 887 mmol) and p-toluenesulfonic acid mono hydrate (642 mg, 3.38 mmol). After being stirred for 27.5 hours at room temperature, to the reaction mixture were added DBU (129 mL, 853 mmol) and ethyl bromodifluoroacetate (162 mL, 1.27 mol) at 5° C. After being stirred for 1 hour at 5° C. and then for 4 hours at room temperature, the reaction mixture was diluted with sodium dihydrogen phosphate solution (1.50 L, 2.25 mol, 1.5 mol/L in water), extracted with ethyl acetate, and washed with water. The combined organic layers were added to activated carbon (90 g), filtered through Celite, and evaporated to give Compound 4-2 (257.3 g, 680 mmol, 81%) as a brown oil. It was used for the next reaction without further purification.

¹H NMR (400 MHz, CDCl₃) δ: 1.40 (3H, t, J=7.2 Hz), 1.50-1.92 (6H, m), 3.53-3.59 (1H, m), 3.78-3.86 (1H, m), 4.35 (1H, d, J=14.6 Hz), 4.41 (2H, q, J=7.2 Hz), 4.55 (1H, d, J=14.6 Hz), 4.72-4.75 (1H, m), 6.57 (1H, s), 7.99 (1H, s).

### Step 2

To a solution of Compound 4-2 (207 g, 594 mmol) in THF (1000 mL) and water (1000 mL) was added sodium borohydride (22.5 g, 594 mmol) portionwise over 30 minutes at 0° C. After being stirred for 2 hours at 0° C., the reaction mixture was quenched with a saturated solution of ammonium chloride, extracted with ethyl acetate, and washed with water and brine. The combined organic layers were dried over sodium sulfate and evaporated to give the crude product. To a solution of the crude product in ethanol (500 mL) was added ammonium hydroxide (207 mL, 2.70 mol). After being stirred for 4 hours at 60° C., the reaction mixture was evaporated. The residue was triturated with ethyl acetate to give Compound 4-3 (43.0 g, 141 mmol, 24%) as a white solid.

¹H NMR (400 MHz, CDCl₃) δ:1.54-1.92 (6H, m), 3.56-3.64 (1H, m), 3.69-3.76 (1H, m), 3.98-4.06 (1H, m), 4.61-4.65 (1H, m), 4.66 (2H, s), 6.34-6.47 (1H, br), 6.48 (1H, s), 7.71 (1H, s), 10.3 (1H, s)

### Step 3

To a solution of 4-3 (5.32 g, 17.4 mmol) and triphenylphosphine (5.94 g, 22.7 mmol) in THF (26.6 mL) was added DIAD (11.9 mL, 22.7 mmol, 1.9 mol/L in toluene) at 0° C. After being stirred for 2.5 hours at room temperature, to the reaction mixture were added triphenylphosphine (1.37 g, 5.23 mmol) and DIAD (2.75 mL, 5.23 mmol, 1.9 mol/L in toluene). After being stirred for 30 minutes at room temperature, the reaction mixture was evaporated. The residue was diluted with a mixture of DMF/water (2:1), extracted with a mixture of n-heptane/toluene (2:1), and washed with water. The combined organic layers were evaporated to the crude product. To a solution of the crude product in methanol (13.3 mL) was added hydrogen chloride (13.7 mL, 26.1 mol, 2 mol/L in water). After being stirred for 1 hour at room temperature, the reaction mixture was extracted water, washed with ethyl acetate. The combined aqueous layers were basified with a solution of sodium hydroxide, extracted with ethyl acetate, and washed with water. The combined organic layers were evaporated to give Compound 4-4 (2.52 g, 12.4 mmol, 71%). It was used for the next reaction without further purification.

¹H NMR (400 MHz, CDCl₃) δ: 3.18-3.25 (1H, br), 4.35 (2H, t, J=9.0 Hz), 4.69 (2H, s), 6.95 (1H, s), 8.30 (1H, s).

### Steps 4

To a solution of Compound 4-4 (2.52 g, 12.4 mmol), sodium dihydrogen phosphate (4.54 g, 37.9 mmol), disodium hydrogen phosphate (1.79 g, 12.6 mmol), and sodium chlorite (4.21 g, 37.2 mmol) in water (25.2 mL) and acetonitrile (25.2 mL) were added TEMPO (194 mg, 1.24 mmol) and a solution of sodium hypochlorite (0.076 mL, 0.062 mmol, 5 wt. % in water) at 35° C. After being stirred for 15 minutes at 40° C., to the reaction mixture was added an additional solution of sodium hypochlorite (0.076 mL, 0.062 mmol, 5 wt. % in water). After being stirred for 30 minutes at 40° C., the reaction mixture was diluted with hydrogen chloride (2 mol/L in water), extracted with a mixture of ethyl acetate/THF (1:1) and washed with a solution of sodium hydrogen sulfite and brine. The combined organic layers were dried over sodium sulfate and evaporated to give a crude product. The residue was triturated with ethyl acetate and methanol to give Compound 4-5 (2.46 g, 11.3 mmol, 91%) as a white solid.

¹H NMR (400 MHz, DMSO-d6) δ: 4.82 (2H, t, J=6.4 Hz), 7.76 (1H, s), 8.55 (1H, s).

### Step 5

The Compound I-011 was prepared in a manner similar to the above protocols. (Example 2)(yield; 55%)

¹H NMR (400 MHz, CDCl₃) δ: 1.79 (3H, s), 3.24 (3H, s), 3.72 (1H, d, J=13.8 Hz), 3.85 (1H, d, J=13.8 Hz), 4.41 (2H, t, J=5.8 Hz), 7.07 (1H, dd, J=11.8, 8.9 Hz), 7.77-7.82 (1H, m), 7.84 (1H, dd, J=7.2, 2.6 Hz), 7.94 (1H, s), 8.30 (1H, s), 9.80 (1H, s).

### Example 51

### Synthesis of Compound I-012

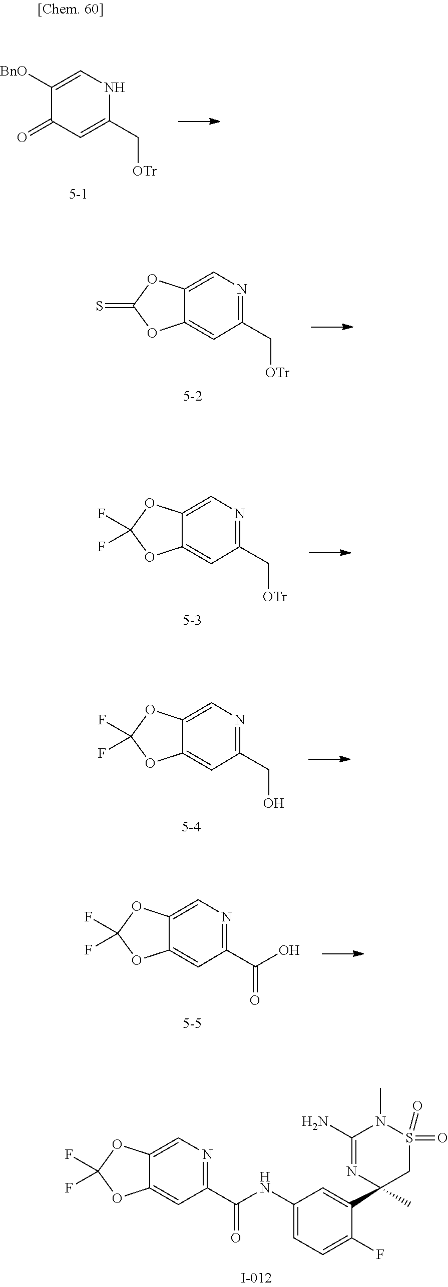

### Step 1

To a solution Compound 5-1 (4.74 g, 10.0 mmol) in THF (95 mL), water (9.47 mL) and 2 mol/L aqueous sodium hydroxide (5.50 mL, 11.00 mmol) was added 10 wt. % palladium on carbon (2.37 g). After being stirred for 4.5 hours at room temperature under 1 atm hydrogen, the reaction mixture was filtered through Celite (Registered trademark) pad. The filtrate was evaporated. The residue was dehydrated by azeotropic distillation with acetonitrile. To a suspension of the residue in dichloromethane (95 mL) were added DMAP (2.44 g, 20.0 mmol) and thiophosgene (1.15 mL, 15.0 mmol). After being for 4 hours at room temperature, the reaction mixture was quenched with water and extracted with chloroform. The combined organic layers were washed with brine, dried over sodium sulfate, and filtered. The solvent was evaporated. The crude product was added to a silica gel column and eluted with hexane/EtOAc 10% to 100%. Collected fractions were evaporated to afford Compound 5-2 (3.61 g, 8.48 mmol, 62%) as a yellow solid. LC/MS: Method A, M+23(Na)=448, tR=2.89 min.

### Step 2

To a solution of Compound 5-2 (2.34 g, 5.50 mL) in dichloromethane (70.2 mL) were added hydrogen fluoride pyridine (23 mL) and 1,3-dibromo-5,5-dimethylimidazolidine-2,4-dione (4.72 g, 16.5 mmol) keeping the temperature below −60° C. The temperature was allowed to rise to 0° C. for 20 minutes. After being stirred for 2 hours at this temperature, the reaction mixture was quenched with 2 mol/L aqueous sodium hydroxide. The mixture was filtered through Celite (Registered trademark) pad. The filtrate was extracted with dichloromethane. The combined organic layers were washed with brine, dried over sodium sulfate, and filtered. The solvent was evaporated. The crude product was added to a silica gel column and eluted with hexane/EtOAc 10% to 90%. Collected fractions were evaporated to afford Compound 5-3 (1.24 g, 3.35 mmol, 61%) as a yellow oil.

¹H NMR (400 MHz, CDCl₃) δ: 3.31 (1H, t, J=5.1 Hz), 4.75 (2H, d, J=5.3 Hz), 7.09 (1H, s), 8.31 (1H, s).

### Step 3

The Compound 5-4 was prepared in a manner similar to the above protocols. (Example 3)(yield; crude)

LC/MS: Method A, M+19=206, tR=1.43 min.

### Step 4

The Compound 5-5 was prepared in a manner similar to the above protocols. (Example 2)(yield; 63%)

¹H NMR (400 MHz, DMSO-d6) δ: 8.14 (1H, s), 8.77 (1H, s).

### Step 5

The Compound I-006 was prepared in a manner similar to the above protocols. (Example 2)(yield; 27%)

¹H NMR (400 MHz, CDCl₃) δ: 1.80 (3H, s), 3.24 (3H, s), 3.71 (1H, d, J=13.8 Hz), 3.92 (1H, d, J=13.8 Hz), 7.08 (1H, dd, J=11.4, 8.9 Hz), 7.73-7.88 (2H, m), 8.05 (1H, s), 8.32 (1H, s), 9.75 (1H, s).

### Example 61

### Synthesis of Compound I-029

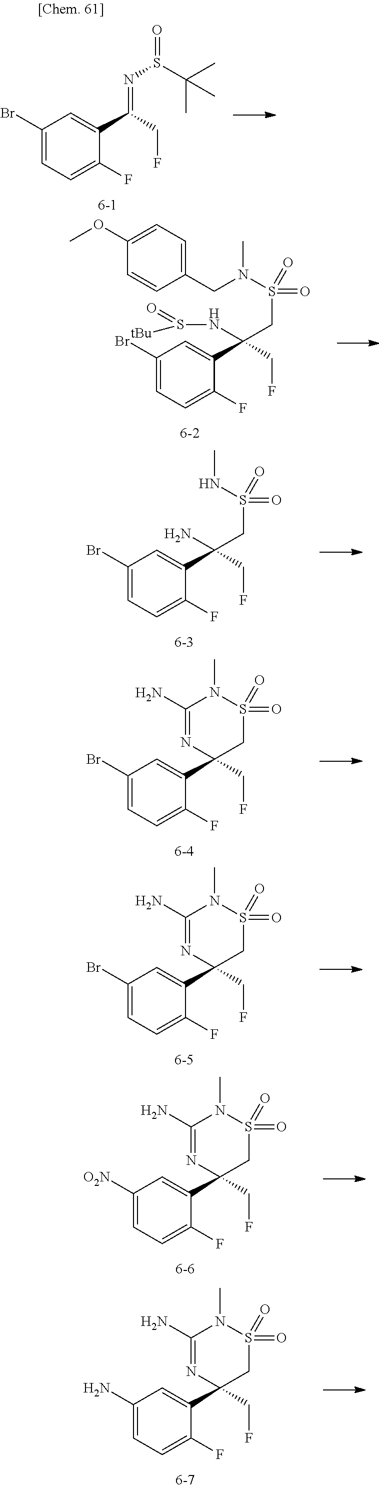

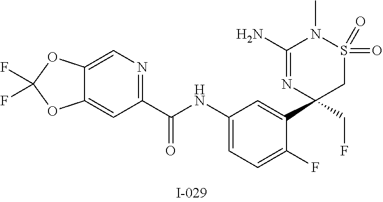 [SMILES: CN1C(N)=N[C@](CF)(c2cc(NC(=O)c3cc4c(cn3)OC(F)(F)O4)ccc2F)CS1(=O)=O]

### Step 1

To a solution of N-(4-methoxybenzyl)-N-methylmethanesulfonamide (11 g, 48.0 mmol) in anhydrous THF (200 mL) was slowly added nBuLi (1.6 M in hexane, 30 mL, 48.0 mmol) at −78° C. under N2. The reaction solution was stirred at −78° C. for 30 minutes. After that time, a precooled (−78° C.) solution of (R,Z)—N-(1-(5-bromo-2-fluorophenyl)-2-fluoroethylidene)-2-methylpropane-2-sulfinamide (8.5 g, 25.1 mmol) in anhydrous THF (80 mL, then 20 mL to rinse) was added to the reaction mixture via cannula. The reaction mixture was allowed to stir at −78° C. for 30 minutes. After that time, water and EtOAc were added to the reaction mixture. The mixture was allowed to warm to room temperature. The aqueous layer was then separated and extracted with EtOAc. The combined organic layers were washed with brine, dried, filtered and concentrated. The crude product was purified via flash chromatography (220 g silica gel) using a gradient from 1:0 to 6:4 heptane:EtOAc. Product fractions were collected and evaporated, to afford Compound 6-2 (12.6 g, 22.2 mmol, 88%) as an orange oil.

### Step 2

To a solution of Compound 6-2 (12.6 g, 22.2 mmol) in DCM (150 mL) and MeOH (50 mL) was added HCl (6M in ⁱPrOH, 15 mL, 90.0 mmol). The reaction solution was stirred at room temperature for 45 minutes. The solution was then concentrated. The residue was dissolved in DCM. To this solution were added TFA (12 mL, 157 mmol) and 3-methoxybenzene (9.0 mL, 68.7 mmol). The reaction solution was stirred at room temperature for 72 hours, and then at 45° C. for 3 hours. The reaction mixture was cooled to room temperature. The reaction solution was then concentrated and then partitioned between 1 M HCl (aq.) and Et₂O. The layers were separated and the aqueous one adjusted to approximately pH 10 with the slow addition of solid Na₂CO₃, and then extracted with DCM. The organic layers were combined, dried, filtered and concentrated to afford Compound 6-3 (5.3 g, 15.3 mmol, 70%) as a yellow oil.

### Step 3

To a solution of Compound 6-3 in n-butanol (120 mL) was added cyanogen bromide (1.4 g, 13.2 mmol) in the small hastelloy reactor. N2 was bubbled for 5 minutes, and then the reaction mixture was heated to 110° C. for 6 hours. The mixture was partitioned between EtOAc and sat. Na₂CO₃ (aq.). The aqueous layer was extracted with EtOAc. The combined organic layers were washed with brine, dried over Na₂SO₄, filtered and concentrated. The residue was purified via flash column chromatography (80 g silica) using an eluent DCM:NH₃ 7N in methanol from 100:0 to 98:2. Product fractions were collected and evaporated to give Compound 6-4 (2.4 g) as a yellow oil.

### Step 4

To a solution of Compound 6-4 in EtOAc (150 mL) and Et₃N (2.4 mL, 17.2 mmol) was added Pd/C (10%, 0.5 g, 0.47 mmol). The flask was evacuated and backfilled with H2 three times, then stirred at room temperature for 90 minutes. H2 was removed and the reaction mixture was filtered over a path of Dicalite. It was rinsed several times. The filtrate was evaporated to dryness and the residue was purified via flash column chromatography (40 g silica) using an eluent from 100% heptane to 100% EtOAc. Product fractions were collected and evaporated to give Compound 6-5 (1.65 g, 5.7 mmol, 43% for 2 steps) as a colorless oil.

### Step 5

Compound 6-5 was dissolved in TFA (25 mL) and then cooled to 0° C. Sulfuric acid (1.3 mL, 24.4 mmol) was added at 0° C., followed by a portionwise addition of potassium nitrate (630 mg, 6.23 mmol). After 30 minutes of stirring at 0° C., 0.5 eq more potassium nitrate and 1 mL of sulfuric acid were added. After 30 minutes, the reaction was complete. The reaction mixture was slowly poured to a mixture of ice, DCM and NH₃ 25%. The mixture was stirred for a few minutes, then Na₂CO₃ sat. was added to the mixture and the layers were separated. The aqueous layer was extracted with DCM several times. The combined organic layers were dried, filtered and evaporated to dryness. The residue was recrystallized from isopropanol and filtered to afford Compound 6-6 (450 mg, 1.34 mmol, 23%) as a beige powder. The filtrate was evaporated to dryness and then purified by flash column chromatography (12 g silica) using an eluent DCM:NH₃ 7N in methanol from 1:0 to 98:2. Product fractions were collected and evaporated, to afford Compound 6-6 (855 mg, 2.55 mmol, 44%).

### Step 6

Compound 6-6 was dissolved in MeOH (16 mL), H₂O (8 mL) and THF (16 mL), then Fe (2.9 g, 35.8 mmol) and NH₄Cl (2.0 g, 37.4 mmol) were added. The reaction mixture was stirred at 63° C. for 1 hour, then cooled to room temperature. Dicalite, Na₂CO₃ and DCM were added and the mixture was filtered. The layers were separated and the aqueous layer was extracted with DCM. The combined organic layers were dried, filtered and evaporated to dryness, to afford Compound 6-7 (820 mg, 2.69 mmol, 75%) as a white solid foam.

### Step 7

The compound I-029 was prepared in a manner similar to the above protocols. (yield; 77%)

### Example 71

### Synthesis of Compound I-023

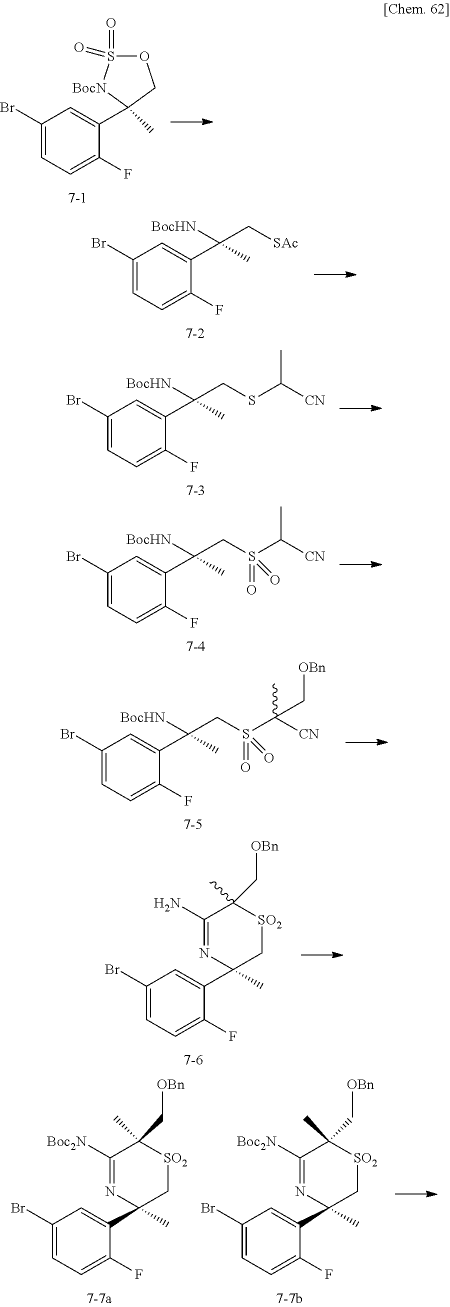

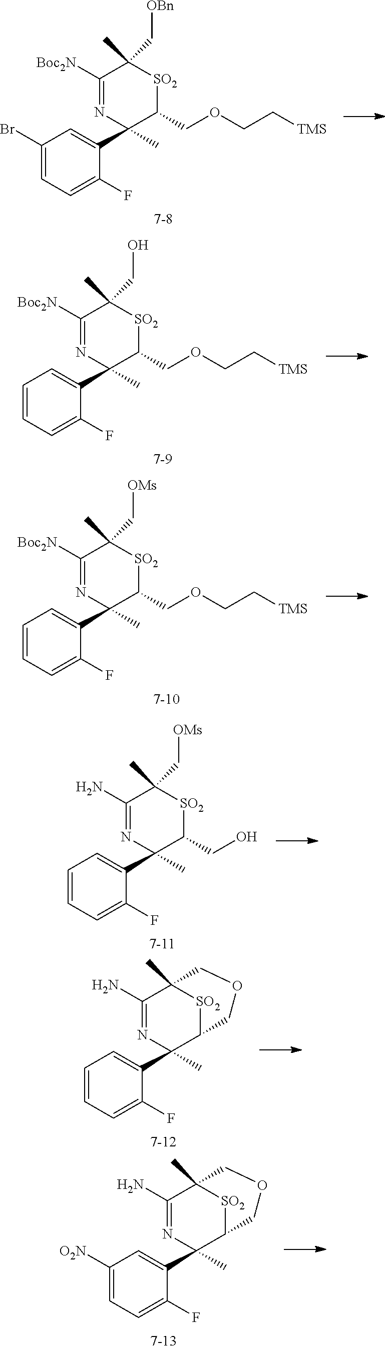

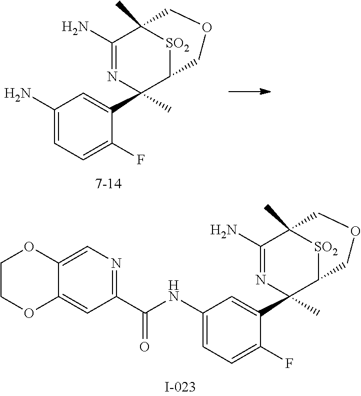 [SMILES: C[C@]12COC[C@H]([C@@](C)(c3cc(N)ccc3F)N=C1N)S2(=O)=O ; C[C@]12COC[C@H]([C@@](C)(c3cc(NC(=O)c4cc5c(cn4)OCCO5)ccc3F)N=C1N)S2(=O)=O]

### Step 1: Synthesis of Compound 7-2

To a solution of 7-1 (3.01 g, 3.79 mmol) in DMF (30 mL) was added KSAc (0.92 g, 8.07 mmol) at room temperature. After being stirred at 50° C. for 1 hour, the mixture was diluted with saturated H₂O and EtOAc. The aqueous layer was separated and extracted with EtOAc. The combined organic layers were dried over MgSO₄, filtered, and evaporated. The residue was purified by column chromatography (silica gel; hexane/EtOAc, gradient: 0-30% EtOAc) to give Compound 7-2 (2.94 g, 99% yield) as a colorless oil.

¹H NMR (400 MHz, CDCl₃) δ 1.41 (9H, br s), 1.71 (3H, s), 2.38 (3H, s), 3.52 (1H, d, J=14.0 Hz), 3.65 (1H, d, J=13.6 Hz), 6.92 (1H, dd, J=11.6, 8.8 Hz), 7.34-7.37 (1H, m), 7.43 (1H, d, J=6.4 Hz). MS-ESI (m/z): 406 [M+H]+.

### Step 2: Synthesis of Compound 7-3

To a solution of Compound 7-2 (1.21 g, 2.97 mmol) in EtOH (18 mL) was added NaOMe (1 N in MeOH; 2.97 mL, 2.97 mmol) at room temperature. After being stirred for 10 minutes, 2-bromopropionitrile (0.31 mL, 3.56 mmol) was added. After being stirred for 1 hour, the mixture was diluted with H₂O and EtOAc. The aqueous layer was separated and extracted with EtOAc. The combined organic extracts were washed with water and brine, dried over MgSO₄, filtered, and evaporated. The residue was purified by column chromatography (silica gel; hexane/EtOAc, gradient: 0-30% EtOAc) to give Compound 7-3 (1.33 g, 100% yield) as a colorless oil.

¹H NMR (400 MHz, CDCl₃) (mixture of diastereomers) δ 1.38 (9H, br s), 1.56 (3H, s), 1.76 (3H, d, J=3.6 Hz), 3.30-3.35 (1H, m), 3.48-3.56 (1H, m), 3.73-3.81 (1H, m), 5.09 (1H, br s), 6.94 (1H, dd, J=11.6, 8.4 Hz), 7.35-7.44 (2H, m). MS-ESI (m/z): 417 [M+H]+.

### Step 3: Synthesis of Compound 7-4

To a solution of Compound 7-3 (12.2 g, 29.1 mmol) in DCM (182 mL) was added mCPBA (21.5 g, 87 mmol) at room temperature. After being stirred for 2 hours, the mixture was diluted with saturated NaHCO₃ and EtOAc. The aqueous layer was separated and extracted with EtOAc. The combined organic extracts were washed with water and brine, dried over MgSO₄, filtered, and evaporated. The residue was purified by column chromatography (silica gel; hexane/EtOAc, gradient: 0-30% EtOAc) to give Compound 7-4 (10 g, 76% yield) as a colorless oil.

¹H NMR (400 MHz, CDCl₃) (mixture of diastereomers) δ 1.44-1.48 (9H, m), 1.73-1.76 (3H, m), 1.93-1.95 (3H, m), 3.78-3.87 (1H, m), 3.93-4.07 (1H, m), 4.60-4.74 (1H, m), 5.32-5.40 (1H, m), 6.94-7.01 (1H, m), 7.39-7.45 (1H, m). MS-ESI (m/z): 449 [M+H]+.

### Step 4: Synthesis of Compound 7-5

To a suspension of Compound 7-4 (8.0 g, 17.8 mmol) and K₂CO₃ (3.2 g, 23.2 mmol) in DMF (80 mL) was added BOMCl (3.62 g, 23.2 mmol) at 0° C. After being stirred for 20 hours at room temperature, the mixture was diluted with saturated NaHCO₃ and EtOAc. The aqueous layer was separated and extracted with EtOAc. The combined organic extracts were washed with water and brine, dried over MgSO₄, filtered, and evaporated. The residue was purified by column chromatography (silica gel; hexane/EtOAc, gradient: 0-30% EtOAc) to give Compound 7-5 (6 g, 59% yield) as a colorless oil.

¹H NMR (400 MHz, CDCl₃) (mixture of diastereomers) δ 1.40-1.50 (9H, m), 1.60-1.62 (3H, m), 1.90-1.96 (3H, m), 3.68-3.76 (1H, m), 3.84-3.96 (1H, m), 4.09-4.24 (1H, m), 4.53-4.72 (3H, m), 5.50-5.55 (1H, m), 6.85-6.95 (1H, m), 7.30-7.44 (7H, m). MS-ESI (m/z): 569 [M+H]+.

### Step 5: Synthesis of Compound 7-6

A solution of Compound 7-5 (5.94 g, 10.4 mmol) in formic acid (12.0 mL, 313 mmol) was stirred at room temperature for 20 h. After consumption of the starting material, the reaction mixture was concentrated under reduced pressure. The residue was diluted with CH₃CN (80 mL). After being stirred at 60° C. for 4 hours, the mixture was diluted with saturated NaHCO₃ and EtOAc. The aqueous layer was separated and extracted with EtOAc. The combined organic layers were dried over MgSO₄, filtered, and evaporated to afford Compound 7-6 (3.38 g, 69% yield) as a white amorphous.

¹H NMR (400 MHz, CDCl₃) (mixture of diastereomers) δ 1.55-1.75 (6H, m), 3.57-3.68 (2H, m), 3.73-4.07 (2H, m), 4.42-4.54 (1H, m), 4.59-5.10 (3H, m), 6.85-6.95 (1H, m), 7.12-7.16 (1H, m), 7.24-7.40 (5H, m), 7.55-7.72 (1H, m). MS-ESI (m/z): 469 [M+H]+.

### Step 6: Synthesis of Compound 7-7 b

To a solution of Compound 7-6 (3.38 g, 7.20 mmol) and Boc₂O (5.02 mL, 21.6 mmol) in THF (50 mL) was added DMAP (0.26 g, 2.16 mmol) at room temperature. After being stirred for 3 hours, the reaction mixture was concentrated under reduced pressure. The residue was purified by column chromatography (amino silica gel; hexane/EtOAc, gradient: 0-30% EtOAc) to give Compound 7-7a (1.66 g, 34% yield) as a white solid and Compound 7-7 b (1.60 g, 33% yield) as a white amorphous.

¹H NMR (400 MHz, CDCl₃) (7a) δ 1.57 (3H, s), 1.58 (9H, s), 1.86 (3H, s), 3.71-3.77 (3H, m), 3.88 (1H, d, J=14.4 Hz), 4.30 (1H, d, J=11.2 Hz), 4.40 (1H, d, J=10.8 Hz), 6.82 (1H, dd, J=11.6, 8.8 Hz), 6.98-7.03 (2H, m), 7.08-7.13 (3H, m), 7.26-7.31 (1H, m), 7.63 (1H, dd, J=6.8, 2.8 Hz). MS-ESI (m/z): 669 [M+H]+.

¹H NMR (400 MHz, CDCl₃) (7b) δ 1.55 (9H, s), 1.57 (3H, s), 1.62 (3H, s), 3.70 (1H, d, J=14.8 Hz), 3.88 (2H, s), 3.96 (1H, d, J=14.8 Hz), 4.54 (1H, d, J=11.2 Hz), 4.59 (1H, d, J=11.2 Hz), 6.94 (1H, dd, J=11.6, 8.8 Hz), 7.28-7.42 (5H, m), 7.60 (1H, dd, J=6.8, 2.8 Hz). MS-ESI (m/z): 669 [M+H]+.

### Step 7: Synthesis of Compound 7-8

To a solution of Compound 7-7b (444 mg, 0.66 mmol) in THF (8 mL) was added LHMDS (1.66 mL, 1.66 mmol) at −78° C. After being stirred for 30 minutes, 2-(chloromethoxy)ethyltrimethylsilane (0.31 mL, 1.66 mmol) was added at −78° C. After being stirred at −78° C. for 1 hour, the mixture was diluted with saturated NH₄Cl and EtOAc. The aqueous layer was separated and extracted with EtOAc. The combined organic extracts were washed with water and brine, dried over MgSO₄, filtered, and evaporated. The residue was purified by column chromatography (silica gel; hexane/EtOAc, gradient: 0-30% EtOAc) to give Compound 7-8 (520 mg, 59% yield) as a white amorphous.

¹H NMR (400 MHz, CDCl₃) δ −0.08 (9H, s), 0.60-0.75 (2H, m), 1.44 (18H, s), 1.90 (3H, s), 1.95 (3H, s), 3.26-3.33 (2H, m), 3.82 (1H, dd, J=10.8, 6.4 Hz), 3.80-4.00 (2H, m), 4.07-4.11 (2H, m), 4.57 (1H, d, J=12.0 Hz), 4.65 (1H, d, J=12.0 Hz), 6.96 (1H, dd, J=11.6, 8.8 Hz), 7.26-7.43 (6H, m), 7.56-7.58 (1H, m). MS-ESI (m/z): 799 [M+H]+.

### Step 8: Synthesis of Compound 7-10

A suspension of Compound 7-8 (105 mg, 0.13 mmol) and 10% Pd—C (20 mg) in MeOH (3 mL) was stirred at room temperature under H2 for 1 hour. After consumption of the starting material, the reaction mixture was filtered through Celite, and concentrated under reduced pressure to afford Compound 7-9. To a solution of Compound 7-9 (70 mg, 0.11 mmol), Et₃N (0.046 mL, 0.33 mmol) and trimethylamine hydrochloride (2.12 mg, 0.02 mmol) was added MsCl (0.017 mL, 0.22 mmol) at 0° C. After being stirred for 3 hours at room temperature, the mixture was diluted with saturated NH₄Cl and EtOAc. The aqueous layer was separated and extracted with EtOAc. The combined organic extracts were washed with water and brine, dried over MgSO₄, filtered, and evaporated. The residue was purified by column chromatography (silica gel; hexane/EtOAc, gradient: 0-30% EtOAc) to give Compound 7-10 (33 mg, 42% yield) as a colorless oil.

¹H NMR (400 MHz, CDCl₃) δ −0.09 (9H, s), 0.62-0.75 (2H, m), 1.53 (18H, s), 1.91 (3H, s), 1.98 (3H, s), 3.13 (3H, s), 3.26-3.31 (2H, m), 3.74-3.78 (1H, m), 4.00-4.07 (2H, m), 4.62 (1H, d, J=10.4 Hz), 5.00 (1H, d, J=10.4 Hz), 7.09 (1H, dd, J=12.0, 8.0 Hz), 7.20 (1H, t, J=7.6 Hz), 7.32-7.34 (1H, m), 7.45 (1H, t, J=8.0 Hz). MS-ESI (m/z): 709 [M+H]+.

### Step 9: Synthesis of Compound 7-11

To a solution of Compound 7-10 (248 mg, 0.35 mmol) in DCM (3.5 mL) was added BF₃-OEt₂ (0.13 mL, 1.05 mmol) at 0° C. After being stirred for 1 hour at room temperature, the mixture was diluted with saturated NaHCO₃ and EtOAc. The aqueous layer was separated and extracted with EtOAc. The combined organic extracts were washed with water and brine, dried over MgSO₄, filtered, and evaporated. The residue was purified by column chromatography (amino silica gel; hexane/EtOAc, gradient: 30-90% EtOAc) to give Compound 7-11 (137 mg, 96% yield) as a white amorphous.

¹H NMR (400 MHz, CDCl₃) δ 1.67 (3H, s), 1.91 (3H, s), 3.16 (3H, s), 3.62-3.67 (1H, m), 3.92 (1H, d, J=8.0 Hz), 4.39 (1H, dd, J=12.4, 8.4 Hz), 4.72 (2H, s), 7.09 (1H, dd, J=12.0, 8.0 Hz), 7.17 (1H, t, J=7.6 Hz), 7.30-7.37 (2H, m). MS-ESI (m/z): 409 [M+H]+.

### Step 10: Synthesis of Compound 7-12

To a solution of Compound 7-11 (23 mg, 0.056 mmol) in DMF (0.5 mL) was added t-BuOK (1 N in THF; 0.73 mL, 0.073 mmol) at 0° C. After being stirred for 30 minutes, the mixture was diluted with H₂O and EtOAc. The aqueous layer was separated and extracted with EtOAc. The combined organic extracts were washed with water and brine, dried over MgSO₄, filtered, and evaporated. The residue was purified by column chromatography (silica gel; hexane/EtOAc, gradient: 30-50% EtOAc) to give Compound 7-12 (9 mg, 51% yield) as a white amorphous.

¹H NMR (400 MHz, CDCl₃) δ 1.48 (3H, s), 1.83 (3H, s), 3.70-3.73 (1H, m), 3.94-4.00 (2H, m), 4.38-4.41 (1H, m), 4.62-4.67 (1H, m), 6.98-7.06 (1H, m), 7.09-7.13 (1H, m), 7.21-7.25 (1H, m), 7.45-7.50 (1H, m). MS-ESI (m/z): 313 [M+H]+.

### Step 11: Synthesis of Compound 7-13

To a solution of Compound 7-12 (11 mg, 0.036 mmol) in TFA (0.1 ml) was added sulfuric acid (0.016 mL, 0.29 mmol) at −20° C. After stirring for 5 minutes at 0° C., the reaction mixture was added HNO₃ (0.004 mL, 0.06 mmol) at −20° C. After stirring for 1 hour at 0° C., the reaction mixture was treated with aqueous K₂CO₃. The aqueous layer was separated and extracted with EtOAc. The combined organic extracts were washed with water and brine, dried over MgSO₄, filtered, and evaporated. The residue was purified by column chromatography (amino silica gel; hexane/EtOAc, gradient: 30-50% EtOAc) to give Compound 7-13 (8 mg, 61% yield) as a yellow amorphous.

¹H NMR (400 MHz, CDCl₃) δ 1.50 (3H, s), 1.81 (3H, s), 3.63 (1H, s), 3.95-4.01 (2H, m), 4.38-4.42 (1H, m), 4.62-4.67 (1H, m), 7.16 (1H, dd, J=11.2, 9.2 Hz), 8.13-8.17 (1H, m), 8.40 (1H, dd, J=7.2, 2.8 Hz), MS-ESI (m/z): 358 [M+H]+.

### Step 12: Synthesis of Compound I-023

A suspension of Compound 7-13 (8 mg, 0.022 mmol) and 10% Pd—C (5 mg) in MeOH (1 mL) was stirred at room temperature under H2 for 1 hour. After consumption of the starting material, the reaction mixture was filtered through Celite, and concentrated under reduced pressure. The residue was used for next reaction without further purification. To a solution of Compound 7-14 (6 mg, 0.018 mmol), 2N HCl aq. (0.009 mL, 0.018 mmol) and 2,3-dihydro-[1,4]dioxino[2,3-c]pyridine-7-carboxylic acid (3.3 mg, 0.018 mmol) was added WSCD-HCl (3.5 mg, 0.018 mmol) at room temperature. After being stirred for 2 hours, the mixture was diluted with saturated NaHCO₃ and EtOAc. The aqueous layer was separated and extracted with EtOAc. The combined organic extracts were washed with water and brine, dried over MgSO₄, filtered, and evaporated. The residue was purified by column chromatography (amino silica gel; hexane/EtOAc, gradient: 20-50% EtOAc) to give Compound I-023 (3.6 mg, 33% yield) as a colorless oil.

¹H NMR (400 MHz, CDCl₃) δ 1.49 (3H, s), 1.82 (3H, s), 3.74 (1H, s), 3.94-4.00 (2H, m), 4.34-4.42 (5H, m), 4.62-4.66 (1H, m), 7.04 (1H, dd, J=11.2, 9.2 Hz), 7.51 (1H, dd, J=7.2, 2.4 Hz), 7.77 (1H, s), 7.93-7.96 (1H, m), 8.11 (1H, s), 9.83 (1H, s). MS-ESI (m/z): 491 [M+H]+.

### Example 81

### Synthesis of Compound III-001

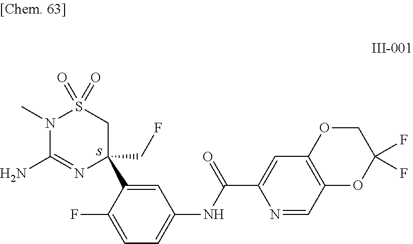

The Compound III-001 was prepared in a manner similar to the above protocols. (Example 6) and further purified by chiral SFC to remove unwanted enantiomer (Stationary phase: Chiralpak Daicel IC 20×250 mm, Mobile phase: CO₂, EtOH+0.4 iPrNH₂).

General SFC protocol: The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

¹H NMR spectra were recorded on a Bruker DPX-400 spectrometer operating at 400 MHz. ¹H NMR (400 MHz, CHLOROFORM-d) δ ppm 3.20-3.32 (m, 3H) 3.78-3.87 (m, 1H) 4.02-4.13 (m, 1H) 4.36-4.44 (m, 2H) 4.46-4.62 (m, 1H) 4.66-4.84 (m, 1H) 7.09 (dd, J=11.7, 8.8 Hz, 1H) 7.09-7.09 (m, 1H) 7.81-7.90 (m, 2H) 7.86 (s, 1H) 7.91 (s, 1H) 8.29 (s, 1H) 9.82 (s, 1H)

LC-MS: M+H: 504, tR: 1.67 (min), method E.

TABLE 1

| Method | Instrument | column | mobile phase | gradient | $\frac{Flow}{Col T}$ | Run time (min) |
|---|---|---|---|---|---|---|
| E | Waters: Acquity ® | Waters: BEH C18 | A: 10 mM CH₃COONH₄ | From 100% A to 5% A in 2.10 min, | $\frac{0.6}{55}$ | 3.5 |
| | UPLC ®- | (1.8 μm, | in 95% H2O + | to 0% A in 0.9 min, | | |
| | DAD and | 2.1*100 mm) | 5% CH₃CN | to 5% A in 0.5 min | | |
| | SQD | | B: CH3CN | | | |

### Example 91

### Synthesis of Compound III-002 and III-003

The Compound III-002 was prepared in a manner similar to the above protocols.

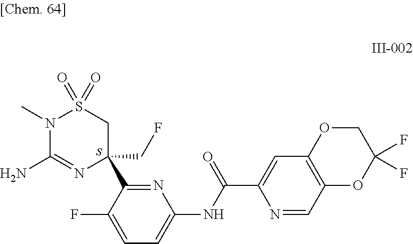

¹H NMR spectra were recorded on a Bruker DPX-400 spectrometer operating at 400 MHz. ¹H NMR (400 MHz, CHLOROFORM-d) δ ppm 3.26 (s, 3H), 3.83 (d, J=14.0 Hz, 1H), 4.31 (d, J=14.0 Hz, 1H), 4.42 (t, J=5.9 Hz, 2H), 4.73-5.03 (m, 2H), 7.50 (dd, J=10.7, 8.9 Hz, 1H), 7.95 (s, 1H), 8.35-8.40 (m, 2H), 10.27 (s, 1H).

LC-MS: M+H: 505, Rt (min): 1.73, method F:

The Compound III-003 was prepared in a manner similar to the above protocols.

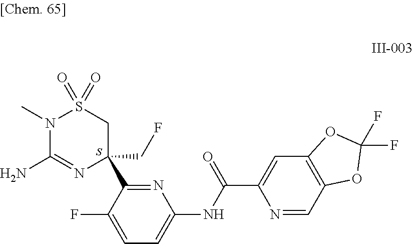

¹H NMR spectra were recorded on a Bruker DPX-400 spectrometer operating at 400 MHz. ¹H NMR (400 MHz, CHLOROFORM-d) δ ppm 3.25 (s, 3H), 3.83 (d, J=14.0 Hz, 1H), 4.32 (d, J=14.0 Hz, 1H), 4.71-5.03 (m, 2H), 7.52 (dd, J=10.6, 8.9 Hz, 1H), 8.08 (s, 1H), 8.37 (dd, J=8.9, 3.0 Hz, 1H), 8.43 (s, 1H), 10.24 (s, 1H). LC-MS: M+H: 491, Rt (min): 1.83, method F:

TABLE 2

| | | | | | | Run |
|---|---|---|---|---|---|---|
| Method code | Instrument | Column | Mobile phase | Gradient | $\frac{Flow}{Col T}$ | time (min) |
| F | Waters: Acquity ® | Waters: BEH | A: 10 mM CH3COONH4 in | From 100% A to 5% A in 2.10 min, | $\frac{0.6}{55}$ | 3.5 |
| | UPLC ®- | (1.8 μm, | 95% H2O + 5% | to 0% A in | | |
| | DAD and | 2.1*100 mm) | CH3CN | 0.90 min, to 5% A | | |
| | SQD | | B: CH3CN | in 0.5 min | | |

### Reference Example 1

### Synthesis of Compound 1′-18

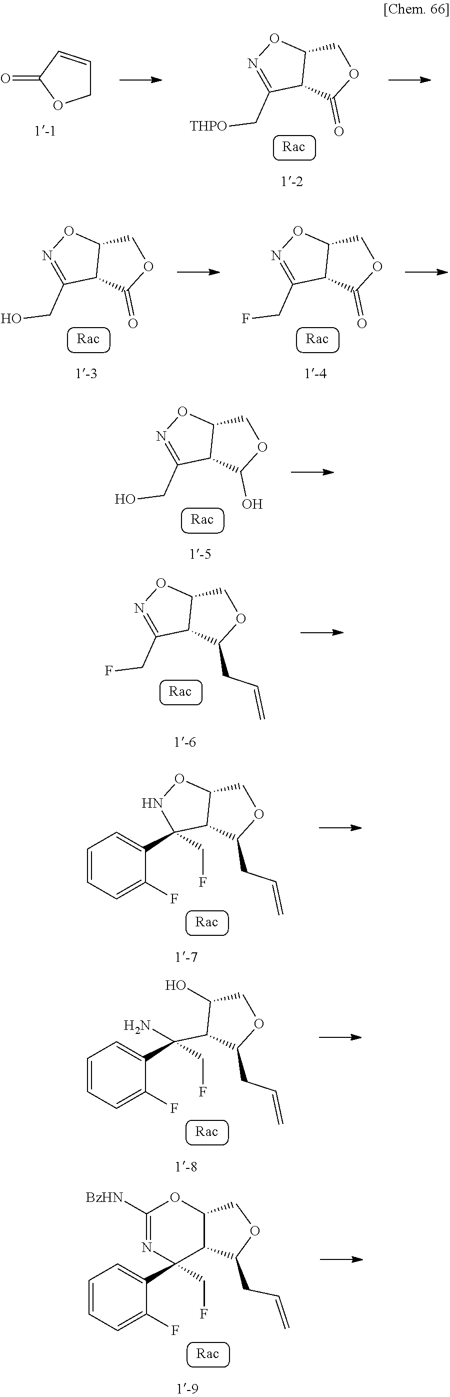

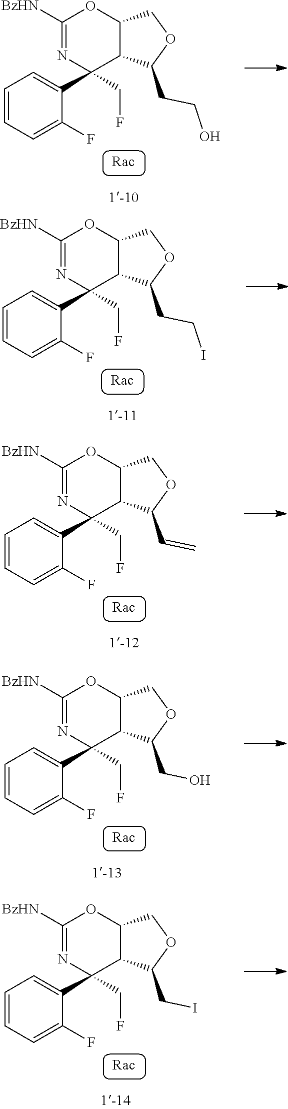

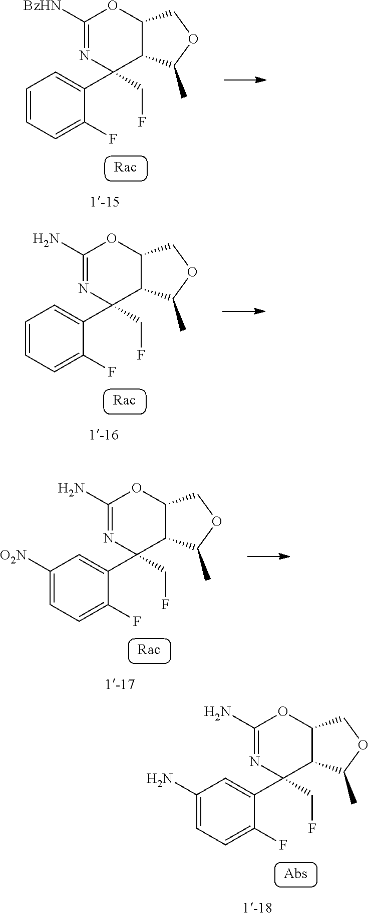

### Step 1: Synthesis of Compound 1′-2

To a solution of gamma-Crotonolactone 1′-1 (4.80 g, 57.1 mmol) and phenyl isocyanate (12.4 ml, 114 mmol) in toluene (72 ml) were added 2-(2-nitroethoxy)tetrahydro-2H-pyran (15.0 g, 86.0 mmol) and DIPEA (0.499 ml, 2.85 mmol) in toluene (24 ml) at 110° C. After stirring for 3 hours at reflux temperature, the reaction mixture was added to DIPEA (0.499 ml, 2.85 mmol). After stirring for 1 h at reflux temperature, the reaction mixture was cooled to room temperature. The mixture was filtered through Celite (Registered trademark) pad and the filtrate was concentrated in vacuo. The crude product was added to a silica gel column and eluted with hexane/EtOAc 20% to 50%. Collected fractions were evaporated to afford Compound I-2 (6.50 g, 26.9 mmol, 47%) as a brown oil.

¹H NMR (CDCl₃) δ: 1.57-1.90 (6H, m), 3.51-3.62 (1H, m), 3.90 (1H, dt, J=38.1, 9.9 Hz), 4.33-4.48 (2H, m), 4.54-4.79 (4H, m), 5.56-5.49 (1H, m).

### Step 2: Synthesis of Compound 1′-3

To a solution of Compound 1′-2 (6.50 g, 26.9 mmol) in MeOH (65 ml) was added TsOH—H₂O (0.513 g, 2.69 mmol) at room temperature. After stirring for 2 hours at the same temperature, Et₃N (0.373 ml, 2.69 mmol) was added to the reaction mixture, and then concentrated in vacuo. The crude product was added to a silica gel column and eluted with hexane/EtOAc 30% to 100%. Collected fractions were evaporated to afford compound 1′-3 (2.78 g, 17.7 mmol, 66%) as a brown oil.

¹H NMR (CDCl₃) δ: 2.38 (1H, br s), 4.44 (1H, d, J=9.7 Hz), 4.74-4.58 (4H, m), 5.57-5.52 (1H, m).

### Step 3: Synthesis of Compound 1′-4

To a solution of compound 1′-3 (2.78 g, 17.7 mmol) in CH₂Cl₂ (28 ml) was added 90% DAST (3.12 ml, 21.2 mmol) at −78° C. The reaction mixture was stirred for 5 hours at room temperature and was treated with aqueous potassium carbonate. The mixture was extracted with EtOAc, and the organic layer was washed with water. The organic layer was concentrated in vacuo. The crude product was added to a silica gel column and eluted with hexane/EtOAc 30%. Collected fractions were evaporated to afford compound 1′-4 (1.58 g, 9.93 mmol, 56%) as a brown oil.

¹H NMR (CDCl₃) δ: 4.41 (1H, d, J=9.7 Hz), 4.62 (1H, d, J=11.3 Hz), 4.69 (1H, dd, J=11.3, 5.3 Hz), 5.20-5.41 (2H, m), 5.63-5.57 (1H, m).

### Step 4: Synthesis of Compound 1′-5

To a solution of Compound 1′-4 (1.58 g, 9.93 mmol) in CH₂Cl₂ (12 ml) and toluene (24 ml) was added to DIBAL (1.02 M in hexane, 10.7 ml, 10.9 mmol) at −78° C. After stirring for 30 min at the same temperature, the reaction mixture was added to MeOH (1.33 ml, 32.8 mmol), THF (24 ml) and H₂O (0.885 ml, 49.2 mmol). After stirring for 30 min at room temperature, the mixture was filtered through Celite (Registered trademark) pad and the filtrate was concentrated in vacuo. The crude product was added to a silica gel column and eluted with hexane/EtOAc 30%. Collected fractions were evaporated to afford Compound 1′-5 (829 mg, 5.14 mmol, 52%) as a yellow solid.

¹H NMR (CDCl₃) δ: 2.60 (1H, d, J=2.3 Hz), 3.95 (1H, d, J=8.9 Hz), 4.25-4.31 (2H, m), 5.15 (1H, dd, J=18.4, 11.7 Hz), 5.26 (1H, dd, J=18.4, 11.7 Hz), 5.40-5.36 (1H, m), 5.72 (1H, d, J=2.1 Hz).

### Step 5: Synthesis of Compound 1′-6

To a solution of Compound 1′-5 (770 mg, 4.78 mmol) and allyltrimethylsilane (3.80 ml, 23.9 mmol) in DCM (15 ml) and MeCN (15 ml) was added BF₃—OEt₂ (3.03 ml, 23.9 mmol) at 0° C. After being stirred for 1 hour at room temperature, the reaction was quenched with aqueous sodium carbonate solution. The mixture was extracted with ethyl acetate and the combined organic layers were washed with water. The organic layer was concentrated in vacuo. The crude product was added to a silica gel column and eluted with hexane/EtOAc 30%. Collected fractions were evaporated to afford Compound 1′-6 (711 mg, 3.84 mmol, 80%) as a colorless oil.

¹H NMR (CDCl₃) δ: 2.25-2.44 (2H, m), 3.74 (1H, d, J=9.5 Hz), 4.03-4.14 (2H, m), 4.24-4.29 (1H, m), 5.08-5.26 (4H, m), 5.35-5.29 (1H, m), 5.86-5.75 (1H, m).

### Step 6: Synthesis of Compound I-7

To a solution of 1-bromo-2-fluorobenzene (1.68 g, 9.60 mmol) in toluene (28 mL) and THF (7 mL) was added n-BuLi (1.64 M in n-hexane, 5.85 mL, 9.60 mmol) at −78° C. and stirred for 10 minutes at the same temperature. To the reaction mixture were added BF₃—OEt₂ (0.487 ml, 3.84 mmol) and a solution of Compound I-6 in toluene (7 mL) at −78° C. and stirred for 1 hour at the same temperature. To the reaction mixture was added aqueous NH₄Cl solution and the aqueous layer was extracted with EtOAc. The organic layer was washed with water and concentrated in vacuo. The crude product was added to a silica gel column and eluted with hexane/EtOAc 30%. Collected fractions were evaporated to afford Compound 1′-7 (883 mg, 3.14 mmol, 82%) as a colorless oil.

¹H NMR (CDCl₃) δ: 2.34 (2H, t, J=6.7 Hz), 3.23-3.29 (1H, m), 3.96 (2H, d, J=3.3 Hz), 4.52-4.76 (3H, m), 4.95 (1H, dd, J=10.4, 47.1 Hz), 5.14 (1H, s), 5.18 (1H, d, J=5.1 Hz), 5.80-5.93 (1H, m), 6.18 (1H, brs), 7.03-7.12 (1H, m), 7.15-7.20 (1H, m), 7.28-7.34 (1H, m), 7.69 (1H, brs).

### Step 7: Synthesis of Compound 1′-8

To a solution of Compound 1′-7 (883 mg, 3.14 mmol) in AcOH (8.8 ml) was added Zn (2.05 g, 31.4 mmol) at room temperature. After stirring for 1 hour at 60° C., the reaction mixture was cooled to room temperature, and aqueous potassium carbonate was added to this mixture. The mixture was filtered through Celite (Registered trademark) pad and the filtrate was extracted with EtOAc. The organic layer was washed with water and concentrated in vacuo. The crude product was added to a silica gel column and eluted with Hexane/EtOAc 30% to 100%. Collected fractions were evaporated to afford Compound 1′-8 (783 mg, 2.76 mmol, 88%) as a colorless oil.

¹H NMR (CDCl₃) δ: 2.21-2.31 (1H, m), 2.50-2.58 (1H, m), 2.65 (1H, dd, J=8.5, 4.6 Hz), 3.63-3.73 (2H, m), 3.96 (1H, t, J=3.3 Hz), 4.34-4.40 (1H, m), 4.50 (1H, dd, J=47.8, 9.3 Hz), 4.90 (1H, ddd, J=47.8, 9.3, 2.8 Hz), 5.12 (1H, s), 5.15 (1H, s), 5.84-5.96 (1H, m), 7.09 (1H, dd, J=12.7, 8.2 Hz), 7.15-7.20 (1H, m), 7.21-7.28 (1H, m), 7.32-7.38 (1H, m), 7.62-7.68 (1H, m).

### Step 8: Synthesis of Compound 1′-9

To a solution of Compound 1′-8 (783 mg, 2.76 mmol) in CH₂Cl₂ (7.8 ml) was added benzoyl isothiocyanate (0.417 ml, 3.04 mmol) at room temperature. After stirring for 1 day at the same temperature, the reaction mixture was added to EDC-HCl (1.06 g, 5.53 mmol). After stirring for 1 day at the same temperature, the reaction mixture was concentrated in vacuo. The crude product was added to a silica gel column and eluted with Hexane/EtOAc 10% to 50%. Collected fractions were evaporated to afford Compound 1′-9 (864 mg, 2.10 mmol, 76%) as a white amorphous.

¹H NMR (CDCl₃) δ: 2.31-2.40 (1H, m), 2.65-2.73 (1H, m), 3.15 (1H, dd, J=8.8, 4.2 Hz), 3.86 (1H, dd, J=10.7, 2.5 Hz), 4.19 (1H, d, J=10.7 Hz), 4.41-4.47 (1H, m), 4.57-4.61 (1H, m), 4.85 (2H, dt, J=9.5, 46.6 Hz), 5.22-5.29 (2H, m), 5.87-5.98 (1H, m), 7.14-7.25 (2H, m), 7.33-7.55 (5H, m), 8.27 (2H, d, J=7.4 Hz), 12.16 (1H, brs).

### Step 9: Synthesis of Compound 1′-10

A solution of Compound 1′-9 (864 mg, 2.10 mmol) in CH₂Cl₂ (17 ml) was stirred under ozone atmosphere at −78° C. After stirring for 20 minutes at the same temperature, to the reaction mixture was added PPh₃ (1.26 g, 4.82 mmol) under N₂ atmosphere. After stirring for 1.5 hours at room temperature, the reaction mixture was added MeOH (8.6 ml) and NaBH₄ (238 mg, 6.28 mmol). After stirring for 2 h at the same temperature, to the reaction mixture was added aqueous NH₄Cl solution, and the aqueous layer was extracted with EtOAc. The organic layer was washed with water and was concentrated in vacuo. The crude product was added to a silica gel column and eluted with hexane/EtOAc 30% to 100%. Collected fractions were evaporated to afford Compound 1′-10 (872 mg, 2.10 mmol, 100%) as a white amorphous.

¹H NMR (CDCl₃) δ: 1.87-1.97 (1H, m), 1.99-2.08 (1H, m), 2.29-2.34 (1H, m), 3.11 (1H, dd, J=9.1, 4.2 Hz), 3.83-3.97 (3H, m), 4.21 (1H, d, J=10.5 Hz), 4.51 (1H, t, J=8.8 Hz), 4.58-4.62 (1H, m), 4.86 (2H, ddd, J=46.7, 20.7, 9.3 Hz), 7.14-7.25 (2H, m), 7.35-7.51 (5H, m), 8.27 (2H, d, J=7.3 Hz), 12.18 (1H, brs).

### Step 10: Synthesis of Compound 1′-11

To a solution of Compound 1′-10 (872 mg, 2.10 mmol), PPh₃ (1.10 g, 4.19 mmol) and imidazole (285 mg, 4.19 mmol) in THF (17 ml) was added 12 (1.06 g, 4.19 mmol) at 0° C. After stirring for 1.5 hours at the same temperature, to the reaction mixture was added aqueous NaHSO₃ solution, and the aqueous layer was extracted with EtOAc. The organic layer was washed with water and was concentrated in vacuo. The crude product was added to a silica gel column and eluted with hexane/EtOAc 10% to 50%. Collected fractions were evaporated to afford Compound I-11 (911 mg, 1.73 mmol, 83%) as a white amorphous.

¹H NMR (CDCl₃) δ: 2.12-2.29 (2H, m), 3.04 (1H, dd, J=8.7, 4.4 Hz), 3.29 (1H, q, J=8.7 Hz), 3.38-3.44 (1H, m), 3.86 (1H, dd, J=10.8, 2.2 Hz), 4.20 (1H, d, J=10.8 Hz), 4.37 (1H, t, J=8.7 Hz), 4.59-4.63 (1H, m), 4.85 (2H, ddd, J=46.7, 22.6, 9.5 Hz), 7.13-7.26 (2H, m), 7.33-7.56 (5H, m), 8.28 (2H, d, J=7.5 Hz), 12.18 (1H, brs).

### Step 11: Synthesis of Compound 1′-12

To a solution of KO^{t}Bu (1.0 M in THF, 6.92 ml, 6.92 mmol) in THF (9 ml) was added Compound I-11 (911 mg, 1.73 mmol) in THF (9 ml) at 0° C. After stirring for 30 min at the same temperature, the reaction mixture was treated with aqueous NH₄Cl solution, and the aqueous layer was extracted with AcOEt. The combined organic layers were washed with H₂O and brine, dried over Na₂SO₄ and filtered. The filtrate was concentrated under vacuum to give Compound I-12 (677 mg, 1.70 mmol, 98%) as a white solid that was used for the next step without purification.

¹H NMR (CDCl₃) δ: 3.07 (1H, dd, J=9.6, 4.1 Hz), 4.02 (1H, dd, J=10.7, 2.9 Hz), 4.24 (1H, d, J=10.7 Hz), 4.61-5.00 (4H, m), 5.42 (1H, d, J=10.2 Hz), 5.54 (1H, d, J=16.9 Hz), 5.91-6.01 (1H, m), 7.13-7.25 (2H, m), 7.34-7.56 (5H, m), 8.29 (2H, d, J=7.5 Hz), 12.23 (1H, brs).

### Step 12: Synthesis of Compound 1′-13

A solution of Compound 1′-12 (452 mg, 1.14 mmol) in CH₂Cl₂ (23 ml) was stirred under ozone atmosphere at −78° C. After stirring for 20 minutes at the same temperature, to the reaction mixture was added PPh₃ (684 mg, 2.61 mmol) under N₂ atmosphere. After stirring for 1.5 hours at room temperature, to the reaction mixture were added MeOH (11 ml) and NaBH₄ (129 mg, 3.40 mmol). After stirring for 1.5 hours at the same temperature, to the reaction mixture was added aqueous NH₄Cl solution, and the aqueous layer was extracted with EtOAc. The organic layer was washed with water and was concentrated in vacuo. The crude product was added to a silica gel column and eluted with hexane/EtOAc 30% to 100%. Collected fractions were evaporated to afford Compound 1′-13 (457 mg, 1.14 mmol, 100%) as a white amorphous.

¹H NMR (CDCl₃) δ: 1.98 (1H, dd, J=5.4, 7.8 Hz), 3.50 (1H, dd, J=8.9, 4.3 Hz), 3.70-3.78 (1H, m), 3.92 (1H, dd, J=10.5, 2.4 Hz), 3.99-4.05 (1H, m), 4.24 (1H, d, J=10.5 Hz), 4.46-4.40 (1H, m), 4.65 (1H, t, J=3.2 Hz), 4.86 (2H, ddd, J=10.2, 12.5, 47.2 Hz), 7.14-7.25 (2H, m), 7.34-7.52 (5H, m), 8.27 (2H, d, J=7.4 Hz), 12.17 (1H, brs).

### Step 13: Synthesis of Compound 1′-14

To a solution of Compound 1′-13 (457 mg, 1.14 mmol), PPh₃ (596 mg, 2.27 mmol) and imidazole (155 mg, 2.27 mmol) in THF (9 ml) was added 12 (576 mg, 2.27 mmol) at 0° C. After stirring for 1.5 hours at room temperature, to the reaction mixture was added aqueous NaHSO₃ and the aqueous layer was extracted with EtOAc. The organic layer was washed with water and concentrated in vacuo. The crude product was added to a silica gel column and eluted with hexane/EtOAc 10% to 50%. Collected fractions were evaporated to afford Compound 1′-14 (418 mg, 0.816 mmol, 72%) as a white amorphous.

¹H NMR (CDCl₃) δ: 3.42 (1H, dd, J=8.6, 4.2 Hz), 3.51 (1H, dd, J=11.5, 2.8 Hz), 3.78 (1H, dd, J=11.5, 2.8 Hz), 4.06-4.16 (2H, m), 4.21 (1H, d, J=10.7 Hz), 4.62-5.01 (3H, m), 7.17-7.27 (2H, m), 7.35-7.57 (5H, m), 8.27 (2H, d, J=6.8 Hz), 12.19 (1H, brs).

### Step 14: Synthesis of Compound 1′-15

To a solution of Compound 1′-14 (418 mg, 0.816 mmol) in toluene (4 ml) were added Bu₃SnH (0.263 ml, 0.979 mmol) and AIBN (6.70 mg, 0.0410 mmol) at room temperature. After stirring for 1 hour at 80° C., the reaction mixture was concentrated. The resulting residue was added to an amino silica gel column and eluted with Hexane/EtOAc 10% to 50%. Collected fractions were evaporated to afford Compound 1′-15 (280 mg, 0.725 mmol, 89%) as a white amorphous. ¹H NMR (CDCl₃) δ: 1.48 (3H, d, J=5.9 Hz), 2.89 (1H, dd, J=9.2, 4.1 Hz), 3.98 (1H, dd, J=10.8, 2.9 Hz), 4.17 (1H, d, J=10.8 Hz), 4.35-4.44 (1H, m), 4.61 (1H, t, J=3.5 Hz), 4.79 (1H, dd, J=10.0, 46.5 Hz), 4.94 (1H, dd, J=10.0, 46.5 Hz), 7.13-7.26 (2H, m), 7.34-7.55 (5H, m), 8.28 (2H, d, J=7.4 Hz), 12.18 (1H, brs).

### Step 15: Synthesis of Compound 1′-16

To a solution of Compound 1′-15 (280 mg, 0.725 mmol) in EtOH (3 ml) and THF (3 ml) was added hydrazine hydrate (0.352 ml, 7.25 mmol) at room temperature. After stirring for 14 hours at the same temperature, the reaction mixture was concentrated. The resulting residue was added to an amino silica gel column and eluted with Hexane/EtOAc 40% to 60%. Collected fractions were evaporated to afford Compound I-16 (205 mg, 0.725 mmol, 100%) as a white amorphous.

¹H NMR (CDCl₃) δ: 1.44 (3H, d, J=5.9 Hz), 2.77 (1H, dd, J=8.9, 4.4 Hz), 3.81-3.88 (2H, m), 4.24-4.37 (3H, m), 4.54-4.76 (2H, m), 7.06 (1H, dd, J=12.5, 8.2 Hz), 7.20-7.15 (1H, m), 7.27-7.33 (1H, m), 7.42-7.47 (1H, m).

### Step 16: Synthesis of Compound 1′-18

To a solution of Compound 1′-16 (205 mg, 0.725 mmol) in TFA (3 ml) was added sulfuric acid (0.774 ml, 14.5 mmol) at −8° C. After stirring for 5 minutes at the same temperature, to the reaction mixture was added HNO₃ (0.0490 ml, 1.09 mmol). After stirring for 10 minutes at the same temperature, the reaction mixture was treated with aqueous K₂CO₃ solution. The aqueous layer was extracted with AcOEt, and the organic layer was dried over Na₂SO₄ and filtered. The filtrate was concentrated under vacuum to give Compound 1′-17 as a white amorphous that was used for the next step without purification.

A solution of Compound 1′-17 and 10% Pd—C (245 mg, 0.109 mmol) in MeOH (2 ml) was stirred under H2 atmosphere at room temperature. After stirring for 2 hours at the same temperature, the mixture was filtered through Celite (Registered trademark) pad. The filtrate was concentrated under vacuum. The resulting residue was purified by supercritical fluid chromatography (SFC) (Chiralpak (Registered trademark) IC; 40% isopropylalcohol with 0.1% diethylamine) to give Compound 1′-18 (78.0 mg, 0.262 mmol, 36%).

¹H NMR (CDCl₃) δ: 1.42 (3H, d, J=6.0 Hz), 2.75 (1H, dd, J=9.0, 4.2 Hz), 3.61 (1H, brs), 3.84 (2H, dd, J=14.2, 10.4 Hz), 4.25-4.34 (1H, m), 4.34-4.37 (1H, m), 4.50-4.74 (2H, m), 6.59-6.53 (1H, m), 6.72 (1H, dd, J=6.7, 2.9 Hz), 6.85 (1H, dd, J=11.9, 8.6 Hz).

### Reference Example 21

### Synthesis of Compound 2′-5

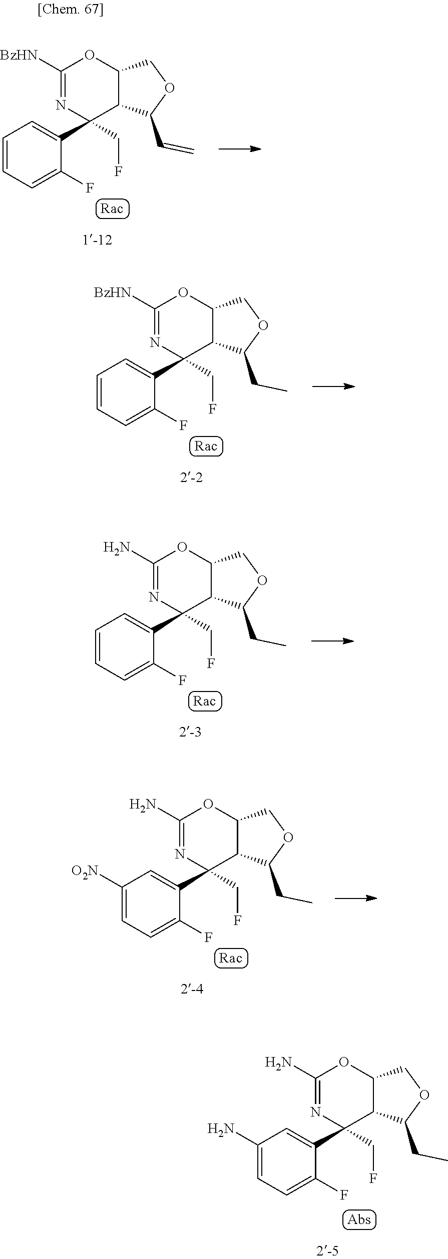

### Step 1: Synthesis of Compound 2′-3

A solution of Compound 1′-12 (200 mg, 0.502 mmol) and 10% Pd—C (203 mg, 0.0900 mmol) in THF (4 ml) was stirred under H2 atmosphere at room temperature. After stirring for 3 hours at the same temperature, the reaction mixture was filtered through Celite (Registered trademark) pad. The filtrate was concentrated under vacuum to give Compound 2′-2 as a white amorphous that was used for the next step without purification.

To a solution of Compound 2′-2 in EtOH (4 ml) was added hydrazine hydrate (0.244 ml, 5.02 mmol) at room temperature. After stirring for 30 minutes at 50° C., the reaction mixture was concentrated. The resulting residue was added to an amino silica gel column and eluted with Hexane/EtOAc 50% to 60%. Collected fractions were evaporated to afford Compound 2′-3 (124 mg, 0.418 mmol, 83%) as a white amorphous.

¹H NMR (CDCl₃) δ: 1.07 (3H, t, J=7.3 Hz), 1.52-1.63 (1H, m), 1.78-1.90 (1H, m), 2.89 (1H, dd, J=8.7, 4.4 Hz), 3.73 (1H, dd, J=10.3, 2.0 Hz), 3.87 (1H, d, J=10.3 Hz), 4.14-4.20 (1H, m), 4.27-4.30 (1H, m), 4.33 (1H, brs), 4.57 (1H, dd, J=16.3, 8.9 Hz), 4.69 (1H, dd, J=16.3, 8.9 Hz), 7.06 (1H, dd, J=12.5, 7.9 Hz), 7.17 (1H, t, J=7.6 Hz), 7.27-7.33 (1H, m), 7.44 (1H, t, J=7.9 Hz).

### Step 2: Synthesis of Compound 2′-5

To a solution of Compound 2′-3 (124 mg, 0.418 mmol) in TFA (1.8 ml) was added sulfuric acid (0.446 ml, 8.37 mmol) at −8° C. After stirring for 5 minutes at the same temperature, to the reaction mixture was added HNO₃ (0.0280 ml, 0.628 mmol). After stirring for 10 minutes at the same temperature, the reaction mixture was treated with aqueous K₂CO₃ solution. The aqueous layer was extracted with AcOEt and the organic layer was dried over Na₂SO₄ and filtered. The filtrate was concentrated under vacuum to give Compound 2′-4 as a white amorphous that was used for the next step without purification.

A solution of Compound 2′-4 and 10% Pd—C (141 mg, 0.0630 mmol) in MeOH (6 ml) was stirred under H2 atmosphere at room temperature. After stirring for 2 hours at the same temperature, the reaction mixture was filtered through Celite (Registered trademark) pad. The filtrate was concentrated under vacuum. The resulting residue was purified by supercritical fluid chromatography (SFC) (Chiralpak (Registered trademark) IC; 40% isopropylalcohol with 0.1% diethylamine) to give Compound 2′-5 (52.0 mg, 0.167 mmol, 40%).

¹H NMR (CDCl₃) δ: 1.06 (3H, t, J=7.3 Hz), 1.51-1.63 (1H, m), 1.77-1.89 (1H, m), 2.87 (1H, dd, J=8.7, 4.3 Hz), 3.62 (1H, brs), 3.74 (1H, dd, J=10.2, 2.2 Hz), 3.87 (1H, d, J=10.2 Hz), 4.11-4.18 (1H, m), 4.33-4.37 (1H, m), 4.54 (1H, dd, J=25.9, 8.7 Hz), 4.66 (1H, dd, J=25.9, 8.7 Hz), 6.59-6.54 (1H, m), 6.72 (1H, dd, J=6.7, 2.9 Hz), 6.84 (1H, dd, J=11.9, 8.5 Hz).

### Reference Example 31

### Synthesis of Compound 3′-12

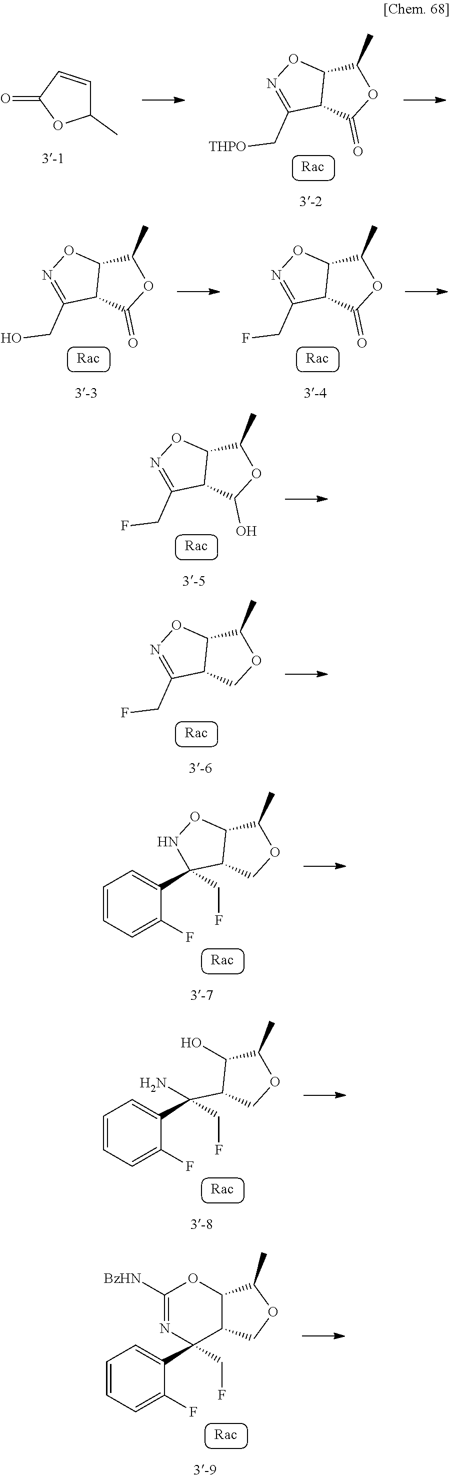

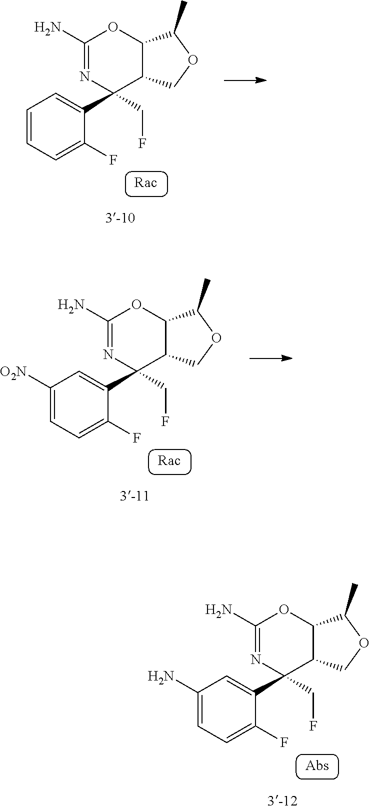 [SMILES: C[C@H]1OC[C@H]2[C@@H]1OC(N)=N[C@]2(CF)c1ccccc1F ; C[C@H]1OC[C@H]2[C@@H]1OC(N)=N[C@]2(CF)c1cc([N+](=O)[O-])ccc1F ; C[C@H]1OC[C@H]2[C@@H]1OC(N)=N[C@]2(CF)c1cc(N)ccc1F]

### Step 1: Synthesis of Compound 3′-2

To a solution of 5-Methylfuran-2(5H)-one 3′-1 (racemate) (10.0 g, 102 mmol) and phenyl isocyanate (22.2 ml, 204 mmol) in toluene (150 ml) were added 2-(2-nitroethoxy)tetrahydro-2H-pyran (26.8 g, 153 mmol) and DIPEA (0.890 ml, 5.10 mmol) in toluene (50 ml) at 110° C. After stirring for 3 hours at reflux temperature, the reaction mixture was cooled to room temperature. The mixture was filtered through Celite (Registered trademark) pad and the filtrate was concentrated in vacuo. The crude product was added to a silica gel column and eluted with hexane/EtOAc 20% to 50%. Collected fractions were evaporated to afford Compound 3-2(a mixture of four diasteromers) (14.4 g, 56.4 mmol, 55%) containing impurities as a brown oil.

### Step 2: Synthesis of Compound 3-′3

To a solution of Compound 3′-2 (14.4 g, 56.4 mmol) in EtOH (43 ml) was added PPTS (2.84 g, 11.3 mmol) at room temperature. After stirring for 3.5 hours at 60° C., the reaction mixture was concentrated in vacuo. The crude product was added to a silica gel column and eluted with hexane/EtOAc 50% to 100%. Collected fractions were evaporated to afford Compound 3-3(enantiomer mixture) (3.69 g, 21.6 mmol, 38%) as a brown oil.

¹H NMR (CDCl₃) δ: 1.51 (3H, d, J=6.8 Hz), 2.52-2.58 (1H, m), 4.50 (1H, d, J=9.5 Hz), 4.60-4.64 (2H, m), 4.82 (1H, dq, J=2.0, 6.8 Hz), 5.09 (1H, dd, J=9.5, 2.0 Hz).

### Step 3: Synthesis of Compound 3′-4

To a solution of Compound 3′-3 (3.69 g, 21.6 mmol) in CH₂Cl₂ (37 ml) was added 90% DAST (3.80 ml, 25.9 mmol) at −78° C. The reaction mixture was stirred for 2 hours at room temperature and was treated with aqueous potassium carbonate solution. The mixture was extracted with EtOAc, and the organic layer was washed with water. The organic layer was concentrated in vacuo. The crude product was added to a silica gel column and eluted with hexane/EtOAc 20% to 50%. Collected fractions were evaporated to afford Compound 3′-4 (3.31 g, 19.1 mmol, 89%) as a yellow oil.

¹H NMR (CDCl₃) δ: 1.51 (3H, d, J=6.8 Hz), 4.46 (1H, dd, J=9.5, 1.6 Hz), 4.80-4.86 (1H, m), 5.16 (1H, d, J=9.5 Hz), 5.24 (1H, dd, J=19.3, 11.5 Hz), 5.36 (1H, dd, J=19.3, 11.5 Hz).

### Step 4: Synthesis of Compound 3′-5

To a solution of Compound 3′-4 (3.31 g, 19.1 mmol) in CH₂Cl₂ (66 ml) was added DIBAL (1.02 M in hexane, 22.5 ml, 22.9 mmol) at −78° C. After stirring for 20 minutes at the same temperature, to the reaction mixture was added aqueous Rochelle's salt. After stirring for 3 hours at room temperature, to the mixture was added 2 mol/L HCl (pH=4). The mixture was extracted with EtOAc, and the organic layer was washed with water. The organic layer was concentrated in vacuo. The crude product was added to a silica gel column and eluted with hexane/EtOAc 20% to 80%. Collected fractions were evaporated to afford Compound 3′-5 (2.19 g, 12.5 mmol, 65%) containing diastereomer as a yellow solid.

### Step 5: Synthesis of Compound 3′-7

To a solution of Compound 3′-5 (1.96 g, 11.2 mmol) and triethylsilane (8.94 ml, 55.9 mmol) in DCM (14 ml) and MeCN (14 ml) was added BF₃-OEt₂ (7.09 ml, 55.9 mmol) at 0° C. After stirring for 15 minutes at the same temperature, the reaction mixture was treated with aqueous sodium carbonate. The aqueous layer was extracted with AcOEt and the organic layer was dried over Na₂SO₄ and filtered. The filtrate was concentrated under vacuum to give Compound 3′-6 as an yellow oil that was used for the next step without purification.

To a solution of 1-bromo-2-fluorobenzene (4.90 g, 28.0 mmol) in toluene (80 mL) and THF (10 mL) was added n-BuLi (1.62 M in n-hexane, 17.1 mL, 28.0 mmol) at −78° C. and the reaction mixture was stirred for 10 minutes at the same temperature. To the reaction mixture were added BF₃—OEt₂ (1.42 ml, 11.2 mmol) and a solution of Compound 3-6 in THF (10 mL) and toluene (18 ml) at −78° C. and the reaction mixture was stirred for 30 minutes at the same temperature. To the reaction mixture was added aqueous NH₄Cl solution, and the aqueous layer was extracted with EtOAc. The organic layer was washed with water and was concentrated in vacuo. The crude product was added to a silica gel column and eluted with hexane/EtOAc 0% to 20%. Collected fractions were evaporated to afford Compound 3′-7 (1.31 g, 5.13 mmol, 46%) as a yellow oil.

¹H NMR (CDCl₃) δ: 1.18 (3H, d, J=6.8 Hz), 3.52-3.59 (1H, m), 4.03 (1H, dd, J=10.6, 7.3 Hz), 4.21-4.38 (3H, m), 4.68 (1H, dd, J=47.4, 9.8 Hz), 4.85-5.03 (1H, m), 6.16 (1H, s), 7.07 (1H, dd, J=11.9, 8.2 Hz), 7.18 (1H, t, J=7.2 Hz), 7.28-7.35 (1H, m), 7.67 (1H, brs).

### Step 7: Synthesis of Compound 3′-8

To a solution of Compound 3′-7 (1.31 g, 5.13 mmol) in AcOH (13 ml) was added Zn (2.01 g, 30.8 mmol) at room temperature. After stirring for 2 hours at 60° C., the reaction mixture was cooled to room temperature, and to this mixture was added aqueous potassium carbonate solution. The mixture was filtered through Celite (Registered trademark) pad and the filtrate was extracted with EtOAc. The organic layer was washed with water and concentrated in vacuo. The crude product was added to a silica gel column and eluted with Hexane/EtOAc 30% to 100%. Collected fractions were evaporated to afford Compound 3′-8 (1.08 g, 4.20 mmol, 82%) as a colorless oil.

¹H NMR (CDCl₃) δ: 1.06 (3H, d, J=6.8 Hz), 2.84-2.92 (1H, m), 3.55 (1H, d, J=4.3 Hz), 3.95 (1H, q, J=6.8 Hz), 4.00-4.06 (1H, m), 4.09-4.19 (1H, m), 4.34 (1H, dd, J=48.2, 9.1 Hz), 4.90 (1H, ddd, J=48.2, 9.1, 2.5 Hz), 7.08-7.15 (1H, m), 7.23-7.27 (1H, m), 7.33-7.40 (1H, m), 7.70-7.64 (1H, m).

### Step 8: Synthesis of Compound 3′-9

To a solution of Compound 3′-8 (1.08 g, 4.20 mmol) in CH₂Cl₂ (11 ml) was added benzoyl isothiocyanate (0.633 ml, 4.62 mmol) at room temperature. After stirring for 3 hours at the same temperature, to the reaction mixture was added EDC-HCl (1.61 g, 8.40 mmol). After stirring for 14 hours at the same temperature, the reaction mixture was concentrated in vacuo. The crude product was added to a silica gel column and eluted with Hexane/EtOAc 10% to 50%. Collected fractions were evaporated to afford Compound 3′-9 (1.04 g, 2.69 mmol, 64%) as a white solid.

¹H NMR (CDCl₃) δ: 1.18 (3H, d, J=6.8 Hz), 3.27-3.35 (1H, m), 3.98 (1H, t, J=10.0 Hz), 4.24-4.31 (2H, m), 4.42 (1H, q, J=6.8 Hz), 4.71 (1H, dd, J=46.4, 9.4 Hz), 4.85 (1H, dd, J=46.4, 9.4 Hz), 7.15-7.26 (2H, m), 7.56-7.35 (5H, m), 8.27 (2H, d, J=7.4 Hz), 12.14 (1H, s).

### Step 8: Synthesis of Compound 3′-10

To a solution of Compound 3′-9 (1.04 g, 2.69 mmol) in MeOH (10 ml) and THF (10 ml) was added hydrazine hydrate (1.31 ml, 26.9 mmol) at room temperature. After stirring for 1 hour at 50° C., the reaction mixture was concentrated. The resulting residue was added to an amino silica gel column and eluted with Hexane/EtOAc 50% to 80%. Collected fractions were evaporated to afford Compound 3′-10 (734 mg, 2.60 mmol, 97%) as a white solid.

¹H NMR (CDCl₃) δ: 1.11 (3H, d, J=6.8 Hz), 3.16-3.24 (1H, m), 3.89 (1H, t, J=9.9 Hz), 3.95 (1H, d, J=4.4 Hz), 4.17-4.30 (3H, m), 4.51 (1H, dd, J=9.2, 47.1 Hz), 4.66 (1H, ddd, J=47.1, 9.0, 1.5 Hz), 7.07 (1H, dd, J=12.3, 7.7 Hz), 7.18 (1H, t, J=7.2 Hz), 7.27-7.35 (1H, m), 7.51-7.44 (1H, m).

### Step 9: Synthesis of Compound 3′-11

To a solution of Compound 3′-10 (734 mg, 2.60 mmol) in TFA (5.6 ml) was added sulfuric acid (1.39 ml, 26.0 mmol) at −8° C. After stirring for 5 minutes at the same temperature, to the reaction mixture was added HNO₃ (0.174 ml, 3.90 mmol). After stirring for 10 minutes at the same temperature, the reaction mixture was treated with aqueous K₂CO₃ solution, and the aqueous layer was extracted with EtOAc. The organic layer was washed with water and was concentrated in vacuo. The crude product was added to an amino silica gel column and eluted with hexane/EtOAc 60%. Collected fractions were evaporated to afford Compound 3′-11 (820 mg, 2.51 mmol, 96%) as a white amorphous.

¹H NMR (CDCl₃) δ: 1.14 (3H, d, J=6.7 Hz), 3.13-3.20 (1H, m), 3.88 (1H, t, J=9.8 Hz), 3.97 (1H, d, J=4.8 Hz), 4.09-4.23 (2H, m), 4.34 (2H, s), 4.48 (1H, dd, J=20.6, 8.2 Hz), 4.60 (1H, dd, J=20.6, 8.2 Hz), 7.23-7.28 (1H, m), 8.26-8.20 (1H, m), 8.45 (1H, dd, J=6.8, 2.8 Hz).

### Step 10: Synthesis of Compound 3′-12

A solution of Compound 3′-11 (820 mg, 2.51 mmol) and 10% Pd—C (169 mg, 0.0750 mmol) in MeOH (16 ml) was stirred under H2 atmosphere at room temperature. After stirring for 2 hours at the same temperature, the mixture was filtered through Celite (Registered trademark) pad. The filtrate was concentrated under vacuum. The resulting residue was purified by supercritical fluid chromatography (SFC) (Chiralpak (Registered trademark) ID; 20% isopropylalcohol with 0.1% diethylamine) to give Compound 3′-12 (300 mg, 1.01 mmol, 40%).

¹H NMR (CDCl₃) δ: 1.11 (3H, d, J=6.8 Hz), 3.14-3.21 (1H, m), 3.61 (2H, s), 3.86 (1H, t, J=9.9 Hz), 4.01 (1H, d, J=4.3 Hz), 4.08-4.20 (2H, m), 4.47 (1H, dd, J=46.9, 8.8 Hz), 4.63 (1H, ddd, J=46.9, 8.8, 1.6 Hz), 6.60-6.55 (1H, m), 6.75 (1H, dd, J=6.7, 2.9 Hz), 6.86 (1H, dd, J=11.9, 8.7 Hz).

### Reference Example 41

### Synthesis of Compound 7′-10

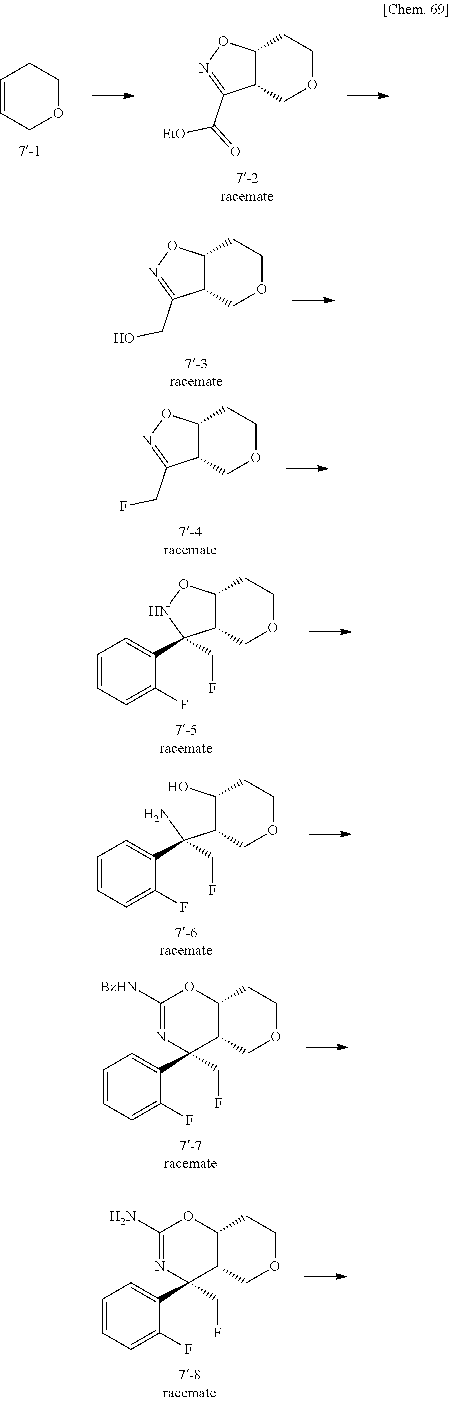

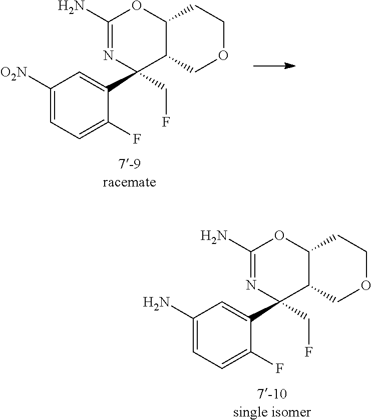 [SMILES: NC1=N[C@](CF)(c2cc([N+](=O)[O-])ccc2F)[C@H]2COCC[C@H]2O1 ; NC1=N[C@](CF)(c2cc(N)ccc2F)[C@H]2COCC[C@H]2O1]

### Step 1: Synthesis of Compound 7′-2

To a solution of 3,6-dihydro-2H-pyran 7-1 (6.20 g, 73.7 mmol) and Et₃N (10.2 ml, 73.7 mmol) in toluene (60 ml) was added ethyl (Z)-2-chloro 2-(hydroxyimino)acetate (22.3 g, 147 mmol) in toluene (120 ml) at 100° C. After stirring for 4 hours at reflux temperature, to the reaction mixture was added Et₃N (10.2 ml, 73.7 mmol). After stirring for 6 hours at reflux temperature, to the reaction mixture were added ethyl (Z)-2-chloro 2-(hydroxyimino)acetate (11.2 g, 73.7 mmol) and Et₃N (10.2 ml, 73.7 mmol). After stirring for 5 hours at reflux temperature, to the reaction mixture were added ethyl (Z)-2-chloro 2-(hydroxyimino)acetate (5.58 g, 36.9 mmol) and Et₃N (5.1 ml, 36.9 mmol). After stirring for 1 hour at reflux temperature, the reaction mixture was cooled to room temperature. To the mixture was added H₂O, and the aqueous layer was extracted with EtOAc. The organic layer was washed with water, dried over Na₂SO₄ and filtered. The filtrate was concentrated in vacuo. The crude product was added to a silica gel column and eluted with hexane/EtOAc 0% to 40%. Collected fractions were evaporated to afford Compound 7′-2 (5.10 g, 25.6 mmol, 35%) as an orange oil.

¹H NMR (CDCl₃) δ: 1.38 (3H, t, J=7.2 Hz), 2.07-2.12 (2H, m), 3.43 (1H, dd, J=14.6, 7.9 Hz), 3.55 (1H, dd, J=11.8, 7.9 Hz), 3.66 (1H, dt, J=15.7, 5.3 Hz), 3.77-3.82 (1H, m), 4.04 (1H, dd, J=11.9, 6.3 Hz), 4.32-4.38 (2H, m), 4.78-4.83 (1H, m).

### Step 2: Synthesis of Compound 7′-3

To a solution of NaBH₄ (3.26 g, 86.0 mmol) in EtOH (140 ml) was added a solution of Compound 7′-2 (14.3 g, 71.9 mmol) in EtOH (140 mL) at 0° C. The reaction mixture was stirred for 3 hours at 40° C. and was treated with AcOH at 0° C. The reaction mixture was concentrated in vacuo. The crude product was added to a silica gel column and eluted with hexane/EtOAc 50% to 70%. Collected fractions were evaporated to afford Compound 7′-3 (7.24 g, 46.1 mmol, 64%) as a colorless oil.

¹H NMR (CDCl₃) δ: 1.88-2.08 (2H, m), 3.25 (1H, q, J=6.9 Hz), 3.64-3.76 (3H, m), 3.94 (1H, dd, J=12.0, 6.0 Hz), 4.44-4.49 (2H, m), 4.67-4.70 (1H, m).

### Step 3: Synthesis of Compound 7′-4

To a solution of Compound 7′-3 (7.24 g, 46.1 mmol) in CH₂Cl₂ (109 ml) was added 90% DAST (13.5 ml, 92.0 mmol) at −78° C. The reaction mixture was stirred for 2.5 hours at room temperature and was treated with aqueous potassium carbonate at 0° C. The mixture was extracted with CHCl₃ and dried over Na₂SO₄, filtered and concentrated. The crude product was added to a silica gel column and eluted with hexane/EtOAc 20% to 40%. Collected fractions were evaporated to afford Compound 7′-4 (4.39 g, 27.6 mmol, 60%) as a yellow oil.

¹H NMR (CDCl₃) δ: 1.91-2.02 (1H, m), 2.02-2.12 (1H, m), 3.29-3.31 (1H, m), 3.62 (1H, dd, J=12.2, 7.0 Hz), 3.71 (2H, dd, J=7.0, 4.5 Hz), 3.95 (1H, dd, J=12.2, 6.0 Hz), 4.70-4.74 (1H, m), 5.12-5.23 (2H, m).

### Step 4: Synthesis of Compound 7′-5

To a solution of 1-bromo-2-fluorobenzene (4.39 g, 27.6 mmol) in toluene (176 mL) and THF (44 mL) was added n-BuLi (1.64 M in n-hexane, 50.5 mL, 83.0 mmol) at −78° C. and the reaction mixture was stirred for 5 minutes at the same temperature. To the reaction mixture were added BF₃—OEt₂ (4.2 ml, 33.1 mmol) and a solution of Compound 7′-4 (4.39 g, 27.6 mmol) in toluene (97 mL) at −78° C. and the reaction mixture was stirred for 10 minutes at the same temperature. To the reaction mixture was added aqueous NH₄Cl solution, and the aqueous layer was extracted with EtOAc. The organic layer was washed with water and was concentrated in vacuo. The crude product was added to a silica gel column and eluted with hexane/EtOAc 10% to 20%. Collected fractions were evaporated to afford Compound 7′-5 (4.91 g, 19.2 mmol, 70%) as a yellow oil.

¹H NMR (CDCl₃) δ: 1.91-1.85 (2H, m), 2.89-2.95 (1H, m), 3.60-3.66 (1H, m), 3.69-3.74 (1H, m), 3.76-3.82 (1H, m), 4.01-4.04 (1H, m), 4.08-4.13 (2H, m), 4.54 (1H, dd, J=47.9, 10.2 Hz), 5.04 (1H, dd, J=47.2, 10.2 Hz), 6.46 (1H, br s), 7.03-7.09 (1H, m), 7.19-7.22 (1H, m), 7.28-7.34 (1H, m), 7.92-7.96 (1H, m).

### Step 5: Synthesis of Compound 7′-6

To a solution of Compound 7′-5 (4.91 g, 19.2 mmol) in AcOH (49 ml) was added Zn (12.6 g, 192 mmol) at room temperature. After stirring for 1 hour at 60° C., the reaction mixture was cooled to room temperature and was filtered through Celite (Registered trademark) pad. To the filtrate was added aqueous potassium carbonate solution. The mixture was filtered through Celite (Registered trademark) pad, and the filtrate was extracted with EtOAc. The organic layer was washed with water and concentrated in vacuo. The crude product was added to a silica gel column and eluted with Hexane/EtOAc 50%. Collected fractions were evaporated to afford Compound 7′-6 (3.80 g, 14.8 mmol, 77%) as a colorless oil.

¹H NMR (CDCl₃) δ: 1.51-1.55 (1H, m), 1.61-1.69 (1H, m), 2.38-2.43 (1H, m), 3.57 (1H, br s), 3.67 (1H, dd, J=11.0, 5.1 Hz), 3.81-3.87 (1H, m), 3.94 (1H, t, J=11.2 Hz), 4.02-4.07 (1H, m), 4.45 (1H, dd, J=47.7, 9.3 Hz), 4.97 (1H, ddd, J=48.1, 9.3, 3.7 Hz), 7.08 (1H, dd, J=12.4, 8.2 Hz), 7.22-7.24 (1H, m), 7.33-7.37 (1H, m), 7.62-7.63 (1H, m).

### Step 6: Synthesis of Compound 7′-7

To a solution of Compound 7′-6 (3.80 g, 14.8 mmol) in CH₂Cl₂ (38 ml) was added benzoyl isothiocyanate (2.18 ml, 16.2 mmol) at 0° C. After stirring for 19 hours at room temperature, to the reaction mixture was added EDC-HCl (5.66 g, 29.5 mmol) at the same temperature. After stirring for 3 hours at 40° C., the reaction mixture was concentrated in vacuo. The crude product was added to a silica gel column and eluted with Hexane/EtOAc 10% to 40%. Collected fractions were evaporated to afford Compound 7′-7 (4.22 g, 10.9 mmol, 74%) as a white solid.

¹H NMR (CDCl₃) δ: 1.85-1.93 (1H, m), 2.04-2.08 (1H, m), 2.87-2.92 (1H, m), 3.74 (1H, t, J=11.7 Hz), 3.80-3.84 (2H, m), 4.04-4.09 (1H, m), 4.37 (1H, br s), 4.70-4.98 (2H, m), 7.13-7.25 (2H, m), 7.38-7.46 (4H, m), 7.51-7.54 (1H, m), 8.28 (2H, d, J=7.5 Hz), 12.14 (1H, s).

### Step 7: Synthesis of Compound 7′-8

To a solution of Compound 7′-7 (4.22 g, 10.9 mmol) in MeOH (42 ml) was added DBU (1.81 ml, 12.0 mmol) at room temperature. After stirring for 7 hours at 60° C., to the reaction mixture were added 2 mol/L HCl and Et₂O. The organic layer was back-extracted with H₂O. The aqueous layer was alkalinized with K₂CO₃ (pH=8) and extracted with AcOEt. The organic layer was washed with water and concentrated in vacuo. The crude product was triturated with CHCl₃ to give Compound 7′-8 (2.39 g, 8.47 mmol, 78%) as a yellow solid.

¹H NMR (CDCl₃) δ: 1.71-1.85 (2H, m), 2.69-2.74 (1H, m), 3.58-3.65 (2H, m), 3.75 (1H, dd, J=11.5, 5.0 Hz), 4.00-4.04 (2H, m), 4.25 (2H, s), 4.54-4.74 (2H, m), 7.04 (1H, dd, J=12.3, 8.2 Hz), 7.14-7.17 (1H, m), 7.27-7.31 (1H, m), 7.43-7.47 (1H, m).

### Step 8: Synthesis of Compound 7′-9

To a solution of Compound 7′-8 (3.00 g, 10.6 mmol) in TFA (17.2 ml) was added sulfuric acid (4.25 ml, 80 mmol) at −15° C. After stirring for 10 minutes at the same temperature, to the reaction mixture was added HNO₃ (0.712 ml, 15.9 mmol). After stirring for 15 minutes at the same temperature, the reaction mixture was treated with aqueous K₂CO₃ solution. The aqueous layer was extracted with AcOEt. The organic layer was washed with brine, dried over Na₂SO₄ and filtered. The filtrate was concentrated under vacuum to give Compound 7′-9 as a pale yellow solid that was used for the next step without purification.

### Step 9: Synthesis of Compound 7′-10

A solution of Compound 7′-9 and 10% Pd—C (674 mg, 3.00 mmol) in MeOH (101 ml) was stirred under H2 atmosphere at room temperature. After stirring for 2 hours at the same temperature, the mixture was filtered through Celite (Registered trademark) pad. The filtrate was concentrated under vacuum. The crude product was purified by supercritical fluid chromatography (SFC) (Chiralpak (Registered trademark) IC; 40% ethanol with 0.1% diethylamine) SFC to afford Compound 7′-10 (1.35 g, 4.56 mmol, 44%) as a yellow solid.

¹H NMR (CDCl₃) δ: 1.71-1.85 (2H, m), 2.68-2.73 (1H, m), 3.55-3.64 (4H, m), 3.75 (1H, dd, J=11.5, 5.1 Hz), 3.98-4.03 (1H, m), 4.07 (1H, br s), 4.25 (2H, br s), 4.49-4.72 (2H, m), 6.53-6.56 (1H, m), 6.74 (1H, dd, J=6.7, 3.0 Hz), 6.83 (1H, dd, J=11.7, 8.6 Hz).

### Reference Example 51

### Synthesis of Compound 8′-12

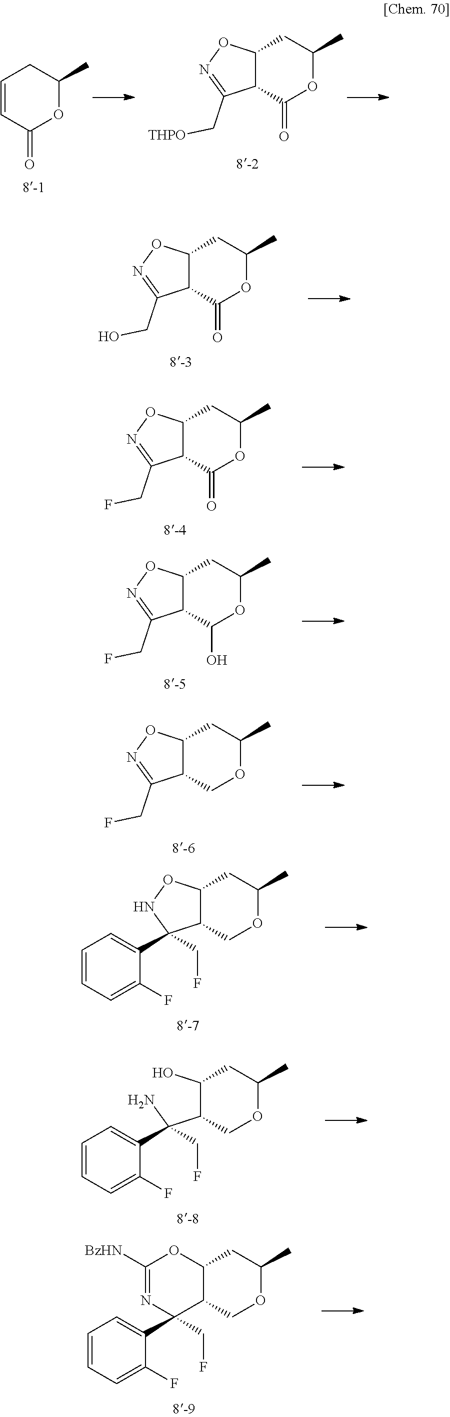

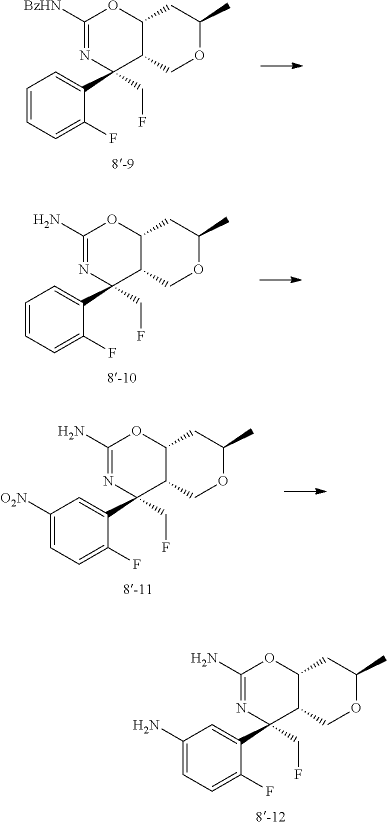

### Step 1: Synthesis of Compound 8′-2

To a solution of Compound 8′-1 (4.10 g, 36.6 mmol), which was prepared according to a known procedure, and phenyl isocyanate (12.0 ml, 110 mmol) in toluene (80 ml) were added 2-(2-nitroethoxy)tetrahydro-2H-pyran (9.62 g, 54.9 mmol) and DIPEA (0.320 ml, 1.83 mmol) in toluene (30 ml) at 110° C. After stirring for 2 hours at reflux temperature, to the reaction mixture were added DIPEA (0.639 ml, 3.66 mmol) and phenyl isocyanate (12.0 ml, 110 mmol). After stirring for 4 hours at reflux temperature, the reaction mixture was cooled to room temperature. The mixture was filtered, and the filtrate was concentrated in vacuo. The crude product was added to a silica gel column and eluted with hexane/EtOAc 10% to 30%. Collected fractions were evaporated to afford Compound 8′-2 (8.05 g, 29.9 mmol, 82%) as an orange oil.

¹H NMR (CDCl₃) δ: 1.41 (3H, dd, J=6.3, 1.5 Hz), 1.60-1.74 (5H, m), 1.78-1.86 (2H, m), 2.17-2.21 (1H, m), 3.53-3.57 (1H, m), 3.82-3.94 (1H, m), 4.25 (1H, dd, J=12.5, 6.9 Hz), 4.37-4.41 (1H, m), 4.50-4.59 (2H, m), 4.70-4.76 (1H, m), 5.07-5.11 (1H, m).

### Step 2: Synthesis of Compound 8′-3

To a solution of Compound 8′-2 (8.05 g, 29.9 mmol) in EtOH (81 ml) was added PPTS (1.50 g, 5.98 mmol) at room temperature. After stirring for 2 hours at 60° C., the reaction mixture was concentrated in vacuo. The crude product was added to a silica gel column and eluted with hexane/EtOAc 10% to 80%. Collected fractions were evaporated to afford Compound 8′-3 (4.46 g, 29.9 mmol, 81%) as a brown oil.

¹H NMR (CDCl₃) δ: 1.44 (3H, d, J=6.3 Hz), 1.83-1.91 (1H, m), 2.21-2.25 (1H, m), 2.47-2.50 (1H, m), 4.41 (1H, d, J=10.9 Hz), 4.51 (2H, dd, J=13.9, 6.7 Hz), 4.55-4.63 (1H, m), 5.06-5.10 (1H, m).

### Step 3: Synthesis of Compound 8′-4

To a solution of Compound 8′-3 (4.93 g, 26.6 mmol) in CH₂Cl₂ (49 ml) was added 90% DAST (5.86 ml, 39.9 mmol) at −78° C. The reaction mixture was stirred for 2 hours at room temperature and was treated with aqueous potassium carbonate solution. The mixture was extracted with CH₂C12. The organic layer was dried over Na₂SO₄ and concentrated in vacuo. The crude product was added to a silica gel column and eluted with hexane/EtOAc 10% to 30%. Collected fractions were evaporated to afford Compound 8′-4 (4.50 g, 24.1 mmol, 90%) as a yellow oil.

¹H NMR (CDCl₃) δ: 1.43 (3H, d, J=6.4 Hz), 1.85-1.92 (1H, m), 2.25 (1H, d, J=15.3 Hz), 4.39 (1H, dd, J=11.0, 3.0 Hz), 4.54-4.58 (1H, m), 5.16-5.27 (3H, m).

### Step 4: Synthesis of Compound 8′-5

To a solution of Compound 8′-4 (4.50 g, 24.1 mmol) in CH₂Cl₂ (45 ml) was added DIBAL (1.03 M in hexane, 24.5 ml, 10.9 mmol) at −78° C. After stirring for 20 minutes at the same temperature, to the reaction mixture was added Rochelle's salt. After stirring for 3 hours at room temperature, to the mixture was added 2 mol/L HCl (pH=4). To the mixture was added NaCl, which was then extracted with CH₂Cl₂ and AcOEt. The combined organic layers were dried over Na₂SO₄ and filtered. The filtrate was concentrated under vacuum. The crude product was added to a silica gel column and eluted with hexane/EtOAc 30% to 50%. Collected fractions were evaporated to afford Compound 8′-5 (2.65 g, 14.0 mmol, 58%) as a white solid as diastereomer mixture.

¹H NMR (CDCl₃) δ: 1.30 (3H, d, J=6.3 Hz), 1.76-1.84 (1H, m), 2.11-2.15 (1H, m), 3.06 (1H, t, J=7.7 Hz), 3.14 (1H, d, J=4.5 Hz), 3.83-3.91 (1H, m), 4.71-4.74 (1H, m), 4.77 (1H, dd, J=7.1, 4.7 Hz), 5.16-5.26 (2H, m).

### Step 5: Synthesis of Compound 8′-6

To a solution of Compound 8′-5 (2.55 g, 13.5 mmol) and triethylsilane (10.8 ml, 67.5 mmol) in DCM (41 ml) and MeCN (41 ml) was added BF₃—OEt₂ (8.56 ml, 67.5 mmol) at 0° C. After stirring for 40 minutes at the same temperature, the reaction mixture was treated with aqueous sodium carbonate solution. The aqueous layer was extracted with CH₂C12, and the organic layer was dried over Na₂SO₄ and filtered. The filtrate was concentrated under vacuum to give Compound 8-6 as an yellow oil that was used for the next step without purification.

### Step 6: Synthesis of Compound 8′-7

To a solution of 1-bromo-2-fluorobenzene (6.11 g, 34.9 mmol) in toluene (128 mL) and THF (16 mL) was added n-BuLi (1.64 M in n-hexane, 21.3 mL, 34.9 mmol) at −78° C., and the reaction mixture was stirred for 5 minutes at the same temperature. To the reaction mixture was added BF₃—OEt₂ (1.77 ml, 14.0 mmol). After stirring for 10 minutes at the same temperature, to the mixture was added a solution of crude Compound 8-6 in THF (16 mL) and toluene (32 ml) at −78° C., and the reaction mixture was stirred for 15 minutes at the same temperature. To the reaction mixture was added aqueous NH₄Cl solution, and the aqueous layer was extracted with EtOAc. The organic layer was washed with water and concentrated in vacuo. The crude product was added to a silica gel column and eluted with hexane/EtOAc 0% to 20%. Collected fractions were evaporated to afford Compound 8′-7 (3.19 g, 11.8 mmol, 85%) as a yellow oil.

¹H NMR (CDCl₃) δ: 1.18 (3H, d, J=6.1 Hz), 1.45-1.54 (1H, m), 1.91-1.94 (1H, m), 2.89-2.95 (1H, m), 3.58-3.66 (1H, m), 3.68-3.76 (1H, m), 3.92-3.93 (1H, m), 4.15-4.21 (1H, m), 4.49 (1H, dd, J=48.2, 10.4 Hz), 5.03 (1H, dd, J=46.9, 10.4 Hz), 6.50 (1H, br s), 7.04-7.10 (1H, m), 7.19-7.23 (1H, m), 7.29-7.34 (1H, m), 7.90-7.94 (1H, m).

### Step 7: Synthesis of Compound 8′-8

To a solution of Compound 8′-7 (3.19 g, 11.9 mmol) in AcOH (31.9 ml) was added Zn (7.74 g, 118 mmol) at room temperature. After stirring for 2 hours at 60° C., the reaction mixture was cooled to room temperature and was filtered through Celite (Registered trademark) pad. The filtrate was treated with aqueous potassium carbonate solution, and the mixture was extracted with EtOAc. The organic layer was washed with water and concentrated in vacuo to afford Compound 8′-8 (3.07 g), which was used for the next reaction without further purification.

### Step 8: Synthesis of Compound 8′-9

To a solution of crude Compound 8′-8 (11.3 g) in CH₂Cl₂ (30.7 ml) was added benzoyl isothiocyanate (1.67 ml, 12.5 mmol) at 0° C. After stirring for 14 hours at room temperature, to the reaction mixture was added EDC-HCl (4.34 g, 22.7 mmol). After stirring for 5 h at 40° C., the reaction mixture was concentrated in vacuo. The crude product was added to a silica gel column and eluted with CHCl₃/AcOEt 20%. Collected fractions were evaporated to afford Compound 8′-9 (3.73 g, 9.32 mmol, 82%) as a yellow solid.

¹H NMR (CDCl₃) δ: 1.20 (3H, d, J=6.3 Hz), 1.50 (1H, td, J=10.0, 4.8 Hz), 2.10-2.14 (1H, m), 2.83-2.86 (1H, m), 3.80 (1H, t, J=11.7 Hz), 3.86-3.94 (1H, m), 4.07 (1H, dd, J=12.7, 6.1 Hz), 4.36 (1H, br s), 4.70-4.82 (1H, m), 4.92 (1H, dd, J=46.2, 9.5 Hz), 7.16 (1H, dd, J=12.3, 8.3 Hz), 7.20-7.24 (1H, m), 7.38-7.46 (4H, m), 7.50-7.54 (1H, m), 8.27-8.29 (2H, m), 12.13 (1H, br s).

### Step 9: Synthesis of Compound 8′-10

To a solution of Compound 8′-9 (3.73 g, 9.32 mmol) in MeOH (37 ml) and THF (37 ml) was added hydrazine hydrate (4.53 ml, 93.0 mmol) at room temperature. After stirring for 14 hours at the same temperature, the reaction mixture was concentrated. The resulting residue was added to an amino silica gel column and eluted with Hexane/EtOAc 40%. Collected fractions were evaporated to afford Compound 8-10 (2.30 g, 7.77 mmol, 83%) as a white solid.

¹H NMR (CDCl₃) δ: 1.16 (3H, d, J=6.3 Hz), 1.37-1.44 (1H, m), 1.82-1.77 (1H, m), 2.64-2.68 (1H, m), 3.65-3.73 (2H, m), 4.00-4.04 (2H, m), 4.25 (2H, br s), 4.59-4.71 (2H, m), 7.04 (1H, dd, J=12.3, 8.0 Hz), 7.14-7.17 (1H, m), 7.29-7.31 (1H, m), 7.43-7.47 (1H, m).

### Step 10: Synthesis of Compound 8′-11

To a solution of Compound 8′-10 (2.30 g, 7.76 mmol) in TFA (12.6 ml) was added sulfuric acid (3.10 ml, 58.2 mmol) at −17° C. After stirring for 10 minutes at the same temperature, to the reaction mixture was added HNO₃ (0.520 ml, 11.6 mmol). After stirring for 20 minutes at the same temperature, the reaction mixture was treated with aqueous K₂CO₃ solution, and the aqueous layer was extracted with EtOAc. The organic layer was washed with water and concentrated in vacuo to afford Compound 8-11 (2.83 g), which was used for the next reaction without further purification.

### Step 11: Synthesis of Compound 8′-12

A solution of crude Compound 8′-11 (2.83 g, 7.76 mmol) and 10% Pd—C (566 mg) in MeOH (85 ml) was stirred under H2 atmosphere at room temperature. After stirring for 2 hours at the same temperature, the mixture was filtered through Celite (Registered trademark) pad. The filtrate was concentrated under vacuum. The residue was triturated with AcOEt, then the resulting solid was collected, washed with AcOEt and dried under reduced pressure to afford Compound 8-12 (1.50 g, 4.83 mmol, 62%) as a white solid. The filtrate was concentrated, and then the residue was purified by column chromatography (silica-gel AcOEt/MeOH=10/1) to afford Compound 8′-12 (374 mg, 1.20 mmol, 16%) as a white solid.

¹H NMR (CDCl₃) δ: 1.16 (3H, d, J=6.3 Hz), 1.38-1.45 (1H, m), 1.78-1.83 (1H, m), 2.62-2.67 (1H, m), 3.58 (2H, br s), 3.62-3.73 (2H, m), 3.99-4.03 (1H, m), 4.07 (1H, br s), 4.23 (2H, br s), 4.51-4.71 (2H, m), 6.52-6.56 (1H, m), 6.74 (1H, dd, J=6.7, 2.9 Hz), 6.83 (1H, dd, J=11.8, 8.5 Hz).

### Reference Example 61

### Synthesis of Compound 12′-9

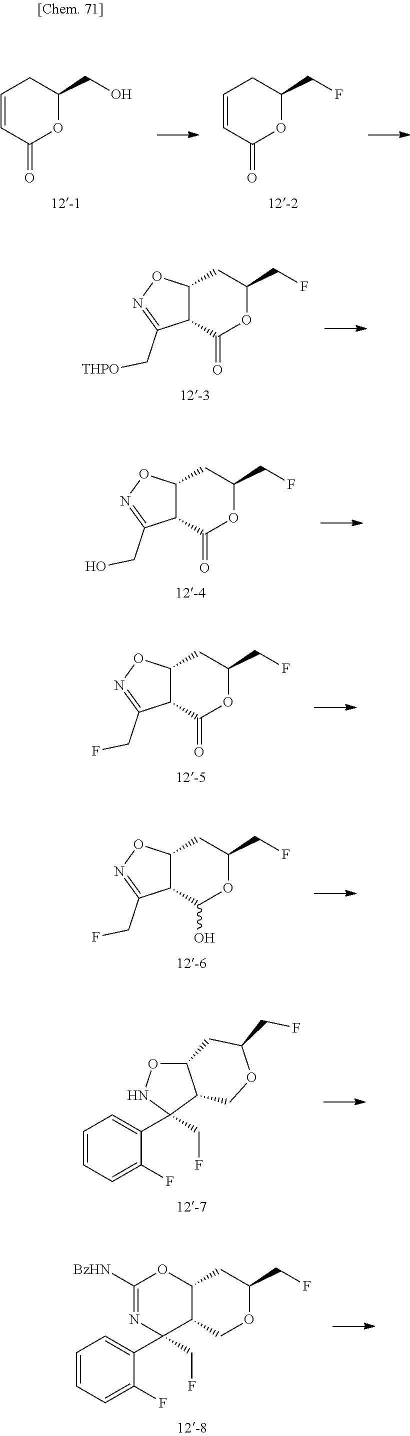

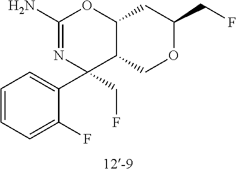 [SMILES: NC1=N[C@](CF)(c2ccccc2F)[C@H]2CO[C@H](CF)C[C@H]2O1]

### Step 1

DAST (4.73 mL, 35.8 mmol) was added dropwise to a solution of Compound 12′-1 (3.06 g, 23.9 mmol) in DCM (60 mL) at −78° C. After being stirred for 5 hours, the reaction was quenched with aq. K₂CO₃ solution. The mixture was extracted with DCM, and the combined organic layers were washed with H₂O and filtered. The solvent was evaporated, and the crude product was purified by silica gel chromatography eluted with Hexane/EtOAc 33% to 50%. Collected fractions were evaporated to afford Compound 12-2 (730 mg, 12%, purity 50%) as a tan oil. ¹H NMR (400 MHz, CDCl₃) δ: 1.91-2.13 (2H, m), 4.36-4.82 (3H, m), 6.06-6.09 (1H, m), 6.92-7.00 (1H, m).

### Step 2

2-(2-nitroethoxy)tetrahydro-2H-pyran (1.23 g, 7.07 mmol) and Hunig's base (0.041 mL, 0.236 mmol) in toluene (4 mL) was added dropwise to a solution of Compound 12-2 (1.04 g, 4.72 mmol) and phenyl isocyanate (1.55 mL, 14.2 mmol) in toluene (10 mL) at reflux. After being stirred for 4.5 hours, Hunig's base (0.082 mL, 0.472 mmol) and phenyl isocyanate (1.55 mL, 14.2 mmol) were added to the mixture. The resulting mixture was stirred at reflux for 5 hours. After being allowed to cool to room temperature, the mixture was filtered and evaporated. The crude product was purified by silica gel chromatography eluted with Hexane/EtOAc 33% to 40%. Collected fractions were evaporated to afford Compound 12′-3 (983 mg, 73%) as a brown solid.

¹H NMR (400 MHz, CDCl₃) δ: 1.52-1.85 (5H, m), 2.07-2.16 (1H, m), 2.21-2.26 (1H, m), 3.52-3.59 (1H, m), 3.82-3.93 (1H, m), 4.24-4.29 (1H, m), 4.34-4.76 (7H, m), 5.14-5.19 (1H, m).

### Step 3

A solution of Compound 12′-3 (983 mg, 3.42 mmol) and PPTS (172 mg, 0.684 mmol) in EtOH (10 mL) was stirred at 60° C. for 4 hours. After being allowed to cool to room temperature, the mixture was evaporated. The crude product was purified by silica gel chromatography eluted with Hexane/EtOAc 50% to 100%. Collected fractions were evaporated to afford Compound 12′-4 (430 mg, 62%) as a tan solid.

¹H NMR (400 MHz, CDCl₃) δ: 2.15-2.45 (2H, m), 2.44-2.47 (1H, m), 4.32-4.77 (6H, m), 5.16 (1H, dt, J=10.7, 2.9 Hz).

### Step 4

DAST (0.419 mL, 3.17 mmol) was added dropwise to a solution Compound 12′-4 (430 mg, 2.12 mmol) in DCM (7 mL) at −78° C. After being stirred at 0° C. for 2 hours, the reaction was quenched with aq K₂CO₃ solution. The mixture was extracted with EtOAc, and the combined organic layers were washed with brine, dried over Na₂SO₄ and filtered. The solvent was evaporated, and the crude product was purified by silica gel chromatography eluted with Hexane/EtOAc 33% to 50%. Collected fractions were evaporated to afford Compound 12′-5 (287 mg, 66%) as a tan oil.

¹H NMR (400 MHz, CDCl₃) δ: 2.17-2.32 (1H, m), 4.41-4.76 (4H, m), 5.20 (2H, d, J=46.4 Hz), 5.23 (1H, dt, J=10.8, 3.0 Hz).

### Step 5

DIBAL (1.02 M; 1.44 mL, 1.47 mmol) was added dropwise to a solution of Compound 12′-5 (287 mg, 1.40 mmol) in DCM (6 mL) at −78° C. After being stirred at the same temperature for 1 hour, the reaction was quenched with aq Rochelle's salt solution. The mixture was extracted with DCM, and the combined organic layers were washed with brine, dried over Na₂SO₄ and filtered. The solvent was evaporated, and the crude product was purified by silica gel chromatography eluted with Hexane/EtOAc 40% to 50%. Collected fractions were evaporated to afford Compound 12′-6 (191 mg, 66%) as a white solid.

¹H NMR (400 MHz, CDCl₃, major isomer) δ: 1.97-2.26 (2H, m), 3.11 (1H, d, J=3.8 Hz), 3.32-3.45 (1H, m), 4.01-4.11 (1H, m), 4.31-4.61 (2H, m), 4.92-4.94 (1H, m), 5.06-5.32 (2H, m).

### Step 6

BF₃—OEt₂ (0.584 mL, 4.61 mmol) was added to a solution of Compound 12′-6 (191 mg, 0.922 mmol) and triethylsilane (0.736 mL, 4.61 mmol) in DCM/CH₃CN (1:1, 2.8 mL) at 0° C. After being stirred at room temperature for 2 hours, the reaction was quenched with saturated aq NaHCO₃ solution. The mixture was extracted with EtOAc, and the combined organic layers were washed with brine, dried over Na₂SO₄ and filtered. The solvent was evaporated to afford the crude product as a tan oil, which was used for the next reaction without further purification.

n-BuLi (1.63 M; 1.41 mL, 2.30 mmol) was added dropwise to a solution of 2-bromofluorobenzene (0.249 mL, 2.30 mmol) at −78° C. After being stirred for 5 minutes, BF₃—OEt₂ (0.117 mL, 0.921 mmol) followed by the crude product were added to the mixture at the same temperature. The reaction was quenched with saturated aqueous NH₄Cl solution, and the reaction mixture was diluted with EtOAc. The mixture was extracted with EtOAc, and the combined organic layers were washed with brine, dried over Na₂SO₄ and filtered. The solvent was evaporated, and the crude product was purified by silica gel chromatography eluted with Hexane/EtOAc 20% to 25%. Collected fractions were evaporated to afford Compound 12′-7 (193 mg, 73%) as a tan oil. ¹H NMR (400 MHz, CDCl₃) δ: 1.68-1.76 (1H, m), 1.85-1.90 (1H, m), 2.95-3.00 (1H, m), 3.74-3.87 (1H, m), 4.03 (1H, brs), 4.23-4.57 (3H, m), 5.05 (1H, dd, J=46.9, 10.3 Hz), 6.51 (1H, brs), 7.05-7.11 (1H, m), 7.19-7.24 (1H, m), 7.29-7.40 (1H, m), 7.91 (1H, t, J=7.3 Hz).

### Step 7

A suspension of Compound 12′-7 (193 mg, 0.672 mmol) and zinc (439 mg, 6.72 mmol) in AcOH (2 mL) was stirred at 60° C. for 3 hours. After being allowed to cool to room temperature, the reaction mixture was filtered and evaporated. The residue was taken up to EtOAc and aq K₂CO₃ solution and stirred at room temperature for 15 minutes. The mixture was extracted with EtOAc, and the combined organic layers were washed with brine, dried over Na₂SO₄ and filtered. The solvent was evaporated to afford the crude product as a colorless oil, which was used for the next reaction without further purification. A solution of the crude product and benzoyl isothiocyanate (0.095 mL, 0.707 mmol) in DCM (2 mL) was stirred at room temperature for 5 hours. EDC-HCl (247 mg, 1.28 mmol) was added to the mixture, and the resulting mixture was stirred at 40° C. for 1.5 hours. After being allowed to cool to room temperature, the mixture was evaporated. The crude product was purified by silica gel chromatography eluted with Hexane/EtOAc 25% to 40%. Collected fractions were evaporated to afford Compound 12′-8 (124 mg, 46%) as a white solid.

¹H NMR (400 MHz, CDCl₃) δ: 1.76-1.84 (1H, m), 2.05-2.11 (1H, m), 2.88-2.93 (1H, m), 3.86 (1H, t, J=11.7 Hz), 3.99-4.08 (1H, m), 4.17 (1H, dd, J=11.7, 5.0 Hz), 4.29-4.58 (3H, m), 4.72-4.96 (2H, m), 7.14-7.24 (2H, m), 7.39-7.55 (5H, m), 8.26-8.28 (2H, m), 12.1 (1H, brs).

### Step 8

A solution of Compound 12′-8 (124 mg, 0.296 mmol) and hydrazine monohydrate (0.144 mmol, 2.96 mmol) in MeOH/THF (1:1, 2 mL) was stirred at 50° C. for 1 hour. After being allowed to cool to room temperature, the mixture was evaporated. The crude product was purified by amino silica gel chromatography eluted with Hexane/EtOAc 50% to 100%. Collected fractions were evaporated to afford Compound 12′-9 (82.0 mg, 88% over 2 steps) as a colorless amorphous.

¹H NMR (400 MHz, CDCl₃) δ: 1.62-1.67 (1H, m), 1.78 (1H, dt, J=14.1, 2.8 Hz), 2.70-2.74 (1H, m), 3.73 (1H, t, J=11.7 Hz), 3.80-3.90 (1H, m), 4.08-4.16 (2H, m), 4.27-4.77 (2H, m), 4.72-4.96 (2H, m), 7.05 (1H, ddd, J=12.4, 8.2, 1.1 Hz), 7.16 (1H, td, J=7.6, 1.2 Hz), 7.28-7.32 (2H, m), 7.46 (1H, td, J=8.0, 1.8 Hz).

### Reference Example 71

### Synthesis of Compound 14′-12

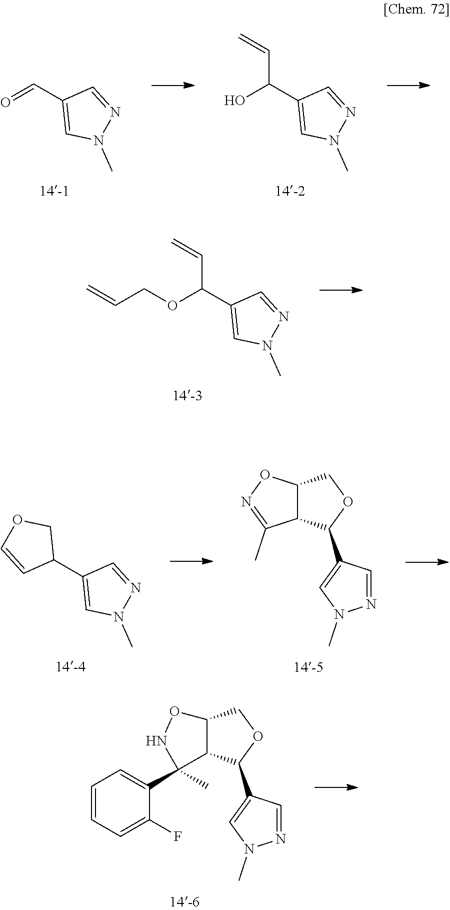

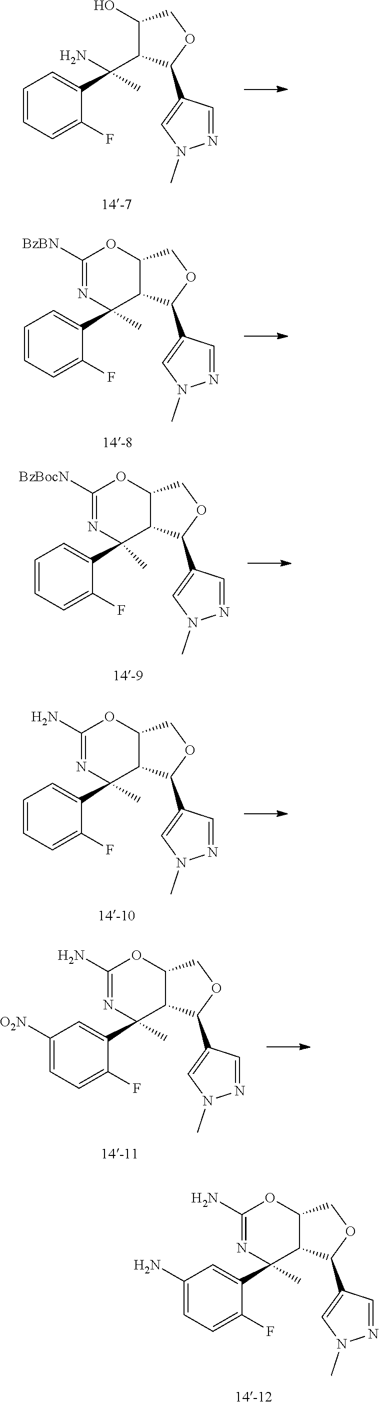

### Step 1

To a solution of Compound 14′-1 (10.9 g, 99 mmol) in THF (219 mL) was added 1.45 mol/L of vinylmagnesium chloride in THF (82 mL, 119 mmol) at −78° C. After being stirred for 3 hours at −78° C., the reaction mixture was quenched with aqueous ammonium chloride, basified with potassium carbonate and extracted with ethyl acetate. The combined organic layers were washed with water. The solvent was evaporated and the residue was added to a amino silica gel column and eluted with hexane/EtOAc 20% to 100%. Collected fractions were evaporated to afford Compound 14′-2 (9.97 g, 72.2 mmol, 73%) as a colorless oil.

¹H NMR (400 MHz, CDCl₃) δ: 1.76 (1H, d, J=6.4 Hz), 3.88 (3H, s), 5.18-5.23 (2H, m), 5.36 (1H, d, J=17.3 Hz), 6.09 (1H, ddd, J=17.1, 10.4, 6.0 Hz), 7.33 (1H, s), 7.45 (1H, s).

### Step 2

To a solution of Compound 14′-2 (9.97 g, 72.2 mmol) in DMF (150 mL) was added 60 wt. % sodium hydride (4.33 g, 108 mmol). After being stirred for 10 minutes at room temperature, to the reaction mixture was added allyl bromide (12.5 mL, 144 mmol). After being stirred for 1 hour at room temperature, the reaction mixture was quenched with cold water and extracted with ethyl acetate. The combined organic layers were washed with water and brine, dried over sodium sulfate, and filtered. The solvent was evaporated. The crude product was added to a silica gel column and eluted with hexane/EtOAc 30% to 80%. Collected fractions were evaporated to afford Compound 14′-3 (12.0 g, 67.3 mmol, 93%) as a white foam.

¹H NMR (400 MHz, CDCl₃) δ: 3.88 (3H, s), 3.94-4.05 (2H, m), 4.81 (1H, d, J=7.0 Hz), 5.15-5.33 (4H, m), 5.87-6.02 (2H, m), 7.31 (1H, s), 7.42 (1H, s).

### Step 3

To a solution of Compound 14′-3 (12.0 g, 67.3 mmol) in dichloromethane (60 mL) was added [1,3-bis(2,4,6-trimethylphenyl)imidazolidin-2-ylidene](chloro)(phenylmethylidene)ruthenium; tricyclohexylphosphane (1.14 g, 1.35 mmol). After being stirred for 3 hours at 40° C., the solvent was evaporated. The crude product was added to a silica gel column and eluted with hexane/EtOAc 20% to 50%. Collected fractions were evaporated to afford Compound 14′-4 (4.05 g, 27.0 mmol, 40%) as a white amorphous.

¹H NMR (400 MHz, CDCl₃) δ: 3.87 (3H, s), 4.64-4.70 (1H, m), 4.73-4.80 (1H, m), 5.78-5.82 (1H, m), 5.86-5.90 (1H, m), 6.02-6.05 (1H, m), 7.42 (1H, s), 7.45 (1H, s).

### Step 4

To a solution of Compound 14′-4 (4.05 g, 27.0 mmol) in toluene (81 mL) were added nitroethane (5.81 mL, 81 mmol), isocyanatobenzene (11.7 mL, 108 mmol) and diisopropylethylamine (1.18 mL, 6.74 mmol). After being stirred for 10 hours at 130° C., the reaction mixture was cooled to room temperature and filtered. The filtrate was evaporate and added to a silica gel column and eluted with chloroform/methanol 0% to 20%. Collected fractions were evaporated to afford a crude product. The crude product was added to a silica gel column and eluted with hexane/EtOAc 30% to 70%. Collected fractions were evaporated to afford Compound 14′-5 (2.75 g, 13.3 mmol, 49% as a mixture of isomers) as an orange solid.

¹H NMR (400 MHz, CDCl₃) δ: 1.98-2.08 (2H, m), 3.77-4.12 (6H, m), 5.10-5.30 (2H, m), 7.29-7.48 (2H, m).

### Step 5

To a solution of 1-bromo-2-fluorobenzene (11.6 g, 66.5 mmol) in toluene (110 mL) and THF (27.5 mL) was added 1.6 mol/L of n-butyl lithium in n-hexane (41.5 mL, 66.5 mmol) at −78° C. followed by boron trifluoride diethyl etherate (5.05 mL, 39.9 mmol) and a solution of Compound 14-5 (2.75 g, 13.3 mmol) in toluene (110 mL). After being stirred for 1 hour at −78° C., the reaction mixture was quenched with aqueous ammonium chloride solution and extracted with ethyl acetate. The combined organic layers were washed with brine, dried over sodium sulfate, and filtered. The solvent was evaporated. The crude product was added to a silica gel column and eluted with hexane/EtOAc 40% to 100%. Collected fractions were evaporated to afford Compound 14′-6 (2.31 g, 7.62 mmol, 57% as a mixture of isomers) as a yellow amorphous. LC/MS: Method A, M+1=304, tR=1.37, 1.47 min.

### Step 6

To a solution of Compound 14′-6 (2.31 g, 7.62 mmol) in acetic acid (23.1 mL) was added zinc (4.98 g, 76 mmol). After being stirred for 1 hour at 90° C., to the reaction mixture was added additional zinc (4.98 g, 76 mmol). After being stirred for 1 hour at 90° C., the reaction was quenched with 2 mol/L of aqueous sodium hydroxide solution (200 mL), and the mixture was diluted with ethyl acetate. The mixture was filtered through Celite (Registered trademark) pad. The filtrate was extracted with ethyl acetate. The organic layer was washed with brine, dried over sodium sulfate, and filtered to afford Compound 14′-7 (2.02 g, 6.61 mmol, 87%) as a white amorphous. The product was used for the next reaction without further purification. LC/MS: Method A, M+1=306, tR=0.36 min.

### Step 7

To a solution of Compound 14′-7 (2.02 g, 6.61 mmol) in dichloromethane (10.1 mL) was added benzoyl isothiocyanate (0.978 mL, 7.27 mL). After being stirred for 30 minutes at room temperature, to the reaction mixture was added EDC hydrochloride (1.52 g, 7.93 mmol). After being stirred 1 hour for 40° C., to the reaction mixture was added additional EDC hydrochloride (0.380 g, 1.98 mmol). After being stirred additional 1 hour for 40° C., the reaction mixture was evaporated. The residue was added to a silica gel column and eluted with hexane/EtOAc 10% to 100%. Collected fractions were evaporated to afford Compound 14′-8 (1.08 g, 2.48 mmol, 38%) as a yellow amorphous.

¹H NMR (400 MHz, CDCl₃) δ: 3.47-3.53 (1H, m), 3.94 (3H, s), 4.05-4.15 (1H, m), 4.22 (1H, d, J=11.0 Hz), 4.64-4.68 (1H, m), 5.16 (1H, d, J=9.0 Hz), 7.09-7.56 (10H, m), 7.64 (1H, s), 8.28 (2H, d, J=7.3 Hz), 11.66-11.75 (1H, br).

### Step 8

To a solution of Compound 14′-8 (1.07 g, 2.46 mmol) in THF (10.7 mL) were added di-tert-butyl dicarbonate (0.686 mL, 2.95 mmol) and DMAP (60.1 mg, 0.492 mmol). After being stirred for 3 hours at room temperature, the reaction mixture was evaporated. The residue was added to a silica gel column and eluted with hexane/EtOAc 50% to 100%. Collected fractions were evaporated to afford Compound 14′-9 (839 mg, 1.57 mmol, 64%) as a white foam.

¹H NMR (400 MHz, CDCl₃) δ: 1.29 (3H, s), 1.46 (9H, s), 3.24 (1H, dd, J=10.3, 3.8 Hz), 3.92 (3H, s), 4.00-4.05 (2H, m), 4.51-4.53 (1H, m), 4.95 (1H, d, J=10.2 Hz), 7.05 (1H, ddd, J=12.7, 7.9, 1.1 Hz), 7.16 (1H, td, J=7.5, 1.2 Hz), 7.23-7.30 (1H, m), 7.43-7.52 (3H, m), 7.55-7.64 (3H, m), 7.81 (2H, d, J=6.9 Hz).

(2.21 m, 535)

### Step 9

To a suspension of Compound 14′-9 (839 mg, 1.57 mmol) in methanol (8.30 mL) was added potassium carbonate (325 mg, 2.35 mmol). After being stirred for 3 hours at room temperature, the reaction mixture was diluted with water and extracted with ethyl acetate. The combined organic layers were washed with brine, dried over sodium sulfate, and filtered. The solvent was evaporated. To a solution of the residue in dichloromethane (4.2 mL) was added TFA (4.2 mL, 54.5 mmol). After being stirred for 1 hour at room temperature, the reaction mixture was quenched with aqueous potassium carbonate solution and extracted with ethyl acetate. The combined organic layers were washed with brine, dried over sodium sulfate, and filtered. The residue was added to an amino silica gel column and eluted with chloroform/methanol 0% to 10%. Collected fractions were evaporated to afford Compound 14′-10 (565 mg, 1.51 mmol, 96%) as a white foam.

¹H NMR (400 MHz, CDCl₃) δ: 1.39 (3H, s), 3.32 (1H, dd, J=9.3, 4.3 Hz), 3.89-3.97 (5H, m), 4.32-4.35 (1H, m), 5.07 (1H, d, J=9.4 Hz), 7.05 (1H, dd, J=12.5, 8.0 Hz), 7.13 (1H, t, J=7.5 Hz), 7.22-7.29 (1H, m), 7.38 (1H, td, J=8.0, 1.3 Hz), 7.46 (1H, s), 7.62 (1H, s).

### Step 10

To a solution of Compound 14′-10 (565 mg, 1.51 mmol) in TFA (2.44 mL, 31.7 mmol) was added concentrated sulfuric acid followed by 70 wt. % nitric acid (0.116 mL, 1.81 mmol), and the mixture was stirred for 1 hour at −10° C. After being stirred for 1 hour at −10° C., the reaction mixture was quenched with aqueous potassium carbonate solution and extracted with ethyl acetate. The combined organic layers were washed with brine, dried over sodium sulfate, and filtered. The crude product was added to an amino silica gel column and eluted with chloroform/methanol 0% to 10%. Collected fractions were evaporated to afford Compound 14′-11 (566 mg, 1.51 mmol, 100%) as a yellow solid.

¹H NMR (400 MHz, CDCl₃) δ: 1.39 (3H, s), 3.27 (1H, dd, J=9.3, 4.4 Hz), 3.94 (3H, s), 3.94-4.01 (2H, m), 4.32-4.36 (1H, m), 5.09 (1H, d, J=5.1 Hz), 7.21 (1H, dd, J=11.0, 9.0 Hz), 7.45 (1H, s), 7.61 (1H, s), 8.18 (1H, ddd, J=8.9, 4.0, 3.0 Hz), 8.35 (1H, dd, J=7.0, 2.9 Hz).

### Step 11

To a suspension of Compound 14′-11 (566 mg, 1.51 mmol) in methanol (11.3 mL) was added concentrated hydrochloric acid (1.51 mL, 18.1 mmol) followed by zinc (690 mg, 10.6 mmol) at 0° C. After being stirred for 1.5 hours at 0° C., the reaction mixture was diluted with water and ethyl acetate, and basified with 2 mol/L of aqueous sodium hydroxide solution. The combined organic layers were washed with brine, dried over sodium sulfate, and filtered to afford Compound 14′-12 (372 mg, 1.08 mmol, 71%) as a white amorphous. The product was used for the next reaction without further purification.

¹H NMR (400 MHz, CDCl₃) δ: 1.35 (3H, s), 3.30-3.35 (1H, m), 3.51-3.69 (2H, m), 3.89-3.98 (5H, m), 4.39-4.44 (1H, m), 5.03 (1H, d, J=9.3 Hz), 6.49-6.54 (1H, m), 6.66-6.71 (1H, m), 6.84 (1H, J=9.3 Hz), 7.45 (1H, s), 7.60 (1H, s).

(0.38 min, 346)

### Example 11

### Synthesis of Compound I-035

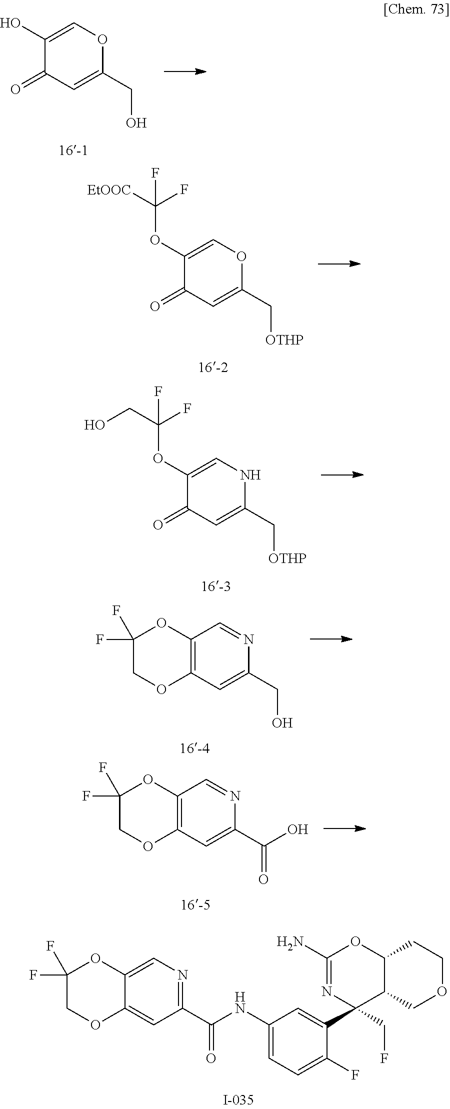

### Step 1

To a solution of Compound 16′-1 (120 g, 844 mmol) in THF (480 mL) were added 3,4-dihydro-2H-pyran (81 mL, 887 mmol) and p-toluenesulfonic acid mono hydrate (642 mg, 3.38 mmol). After being stirred for 27.5 hours at room temperature, to the reaction mixture were added DBU (129 mL, 853 mmol) and ethyl bromodifluoroacetate (162 mL, 1.27 mol) at 5° C. After being stirred for 1 hour at 5° C. and then for 4 hours at room temperature, the reaction mixture was diluted with sodium dihydrogen phosphate (1.50 L, 2.25 mol, 1.5 mol/L in water), extracted with ethyl acetate, and washed with water. The combined organic layers were added to activated carbon (90 g), filtered through Celite, and evaporated to give Compound 16′-2 (257.3 g, 680 mmol, 81%) as a brown oil. It was used for the next reaction without further purification.

¹H NMR (400 MHz, CDCl₃) δ: 1.40 (3H, t, J=7.2 Hz), 1.50-1.92 (6H, m), 3.53-3.59 (1H, m), 3.78-3.86 (1H, m), 4.35 (1H, d, J=14.6 Hz), 4.41 (2H, q, J=7.2 Hz), 4.55 (1H, d, J=14.6 Hz), 4.72-4.75 (1H, m), 6.57 (1H, s), 7.99 (1H, s).

### Step 2

To a solution of Compound 16′-2 (207 g, 594 mmol) in THF (1000 mL) and water (1000 mL) was added sodium borohydride (22.5 g, 594 mmol) portionwise over 30 minutes at 0° C. After being stirred for 2 hours at 0° C., the reaction mixture was quenched with a saturated solution of ammonium chloride solution, extracted with ethyl acetate, and washed with water and brine. The combined organic layers were dried over sodium sulfate and evaporated to give the crude product. To a solution of the crude product in ethanol (500 mL) was added ammonium hydroxide (207 mL, 2.70 mol). After being stirred for 4 hours at 60° C., the reaction mixture was evaporated. The residue was triturated with ethyl acetate to give Compound 16′-3 (43.0 g, 141 mmol, 24%) as a white solid.

¹H NMR (400 MHz, CDCl₃) δ:1.54-1.92 (6H, m), 3.56-3.64 (1H, m), 3.69-3.76 (1H, m), 3.98-4.06 (1H, m), 4.61-4.65 (1H, m), 4.66 (2H, s), 6.34-6.47 (1H, br), 6.48 (1H, s), 7.71 (1H, s), 10.3 (1H, s).

### Step 3

To a solution of Compound 16′-3 (5.32 g, 17.4 mmol) and triphenylphosphine (5.94 g, 22.7 mmol) in THF (26.6 mL) was added DIAD (11.9 mL, 22.7 mmol, 1.9 mol/L in toluene) at 0° C. After being stirred for 2.5 hours at room temperature, to the reaction mixture were added triphenylphosphine (1.37 g, 5.23 mmol) and DIAD (2.75 mL, 5.23 mmol, 1.9 mol/L in toluene). After being stirred for 30 minutes at room temperature, the reaction mixture was evaporated. The residue was diluted with a mixture of DMF/water (2:1), extracted with a mixture of n-heptane/toluene (2:1), and washed with water. The combined organic layers were evaporated to afford the crude product. To a solution of the crude product in methanol (13.3 mL) was added hydrogen chloride (13.7 mL, 26.1 mol, 2 mol/L in water). After being stirred for 1 hour at room temperature, the reaction mixture was extracted with water and washed with ethyl acetate. The combined aqueous layers were basified with aqueous sodium hydroxide solution, extracted with ethyl acetate, washed with water. The combined organic layers were evaporated to give Compound 16′-4 (2.52 g, 12.4 mmol, 71%). It was used for the next reaction without further purification.

¹H NMR (400 MHz, CDCl₃) δ: 3.18-3.25 (1H, br), 4.35 (2H, t, J=9.0 Hz), 4.69 (2H, s), 6.95 (1H, s), 8.30 (1H, s).

### Steps 4

To a solution of Compound 16′-4 (2.52 g, 12.4 mmol), sodium dihydrogen phosphate (4.54 g, 37.9 mmol), disodium hydrogen phosphate (1.79 g, 12.6 mmol), and sodium chlorite (4.21 g, 37.2 mmol) in water (25.2 mL) and acetonitrile (25.2 mL) were added TEMPO (194 mg, 1.24 mmol) and a solution of sodium hypochlorite (0.076 mL, 0.062 mmol, 5 wt. % in water) at 35° C. After being stirred for 15 minutes at 40° C., to the reaction mixture was added additional solution of sodium hypochlorite (0.076 mL, 0.062 mmol, 5 wt. % in water). After being stirred for 30 minutes at 40° C., to the reaction mixture was diluted with hydrogen chloride (2 mol/L in water). The mixture was extracted with a mixture of ethylacetate/THF (1:1) and washed with a solution of sodium hydrogen sulfite and brine. The combined organic layers were dried over sodium sulfate and evaporated to give the residue. The residue was triturated with ethyl acetate and methanol to give Compound 16′-5 (2.46 g, 11.3 mmol, 91%) as a white solid.

¹H NMR (400 MHz, DMSO-d6) δ: 4.82 (2H, t, J=6.4 Hz), 7.76 (1H, s), 8.55 (1H, s).

### Step 5

The Compound I-035 was prepared in a manner similar to the above protocols. (yield: 86%)

¹H NMR (CDCl₃) δ: 1.84-1.73 (2H, m), 2.73-2.77 (1H, m), 3.59-3.66 (2H, m), 3.76 (1H, dd, J=11.4, 4.9 Hz), 4.00-4.05 (1H, m), 4.08 (1H, br s), 4.35-4.45 (4H, m), 4.55-4.72 (2H, m), 7.09 (1H, dd, J=11.4, 8.9 Hz), 7.51 (1H, dd, J=6.8, 2.6 Hz), 7.94 (1H, s), 7.98-8.02 (1H, m), 8.31 (1H, s), 9.82 (1H, s).

### Example 121

### Synthesis of Compound I-056

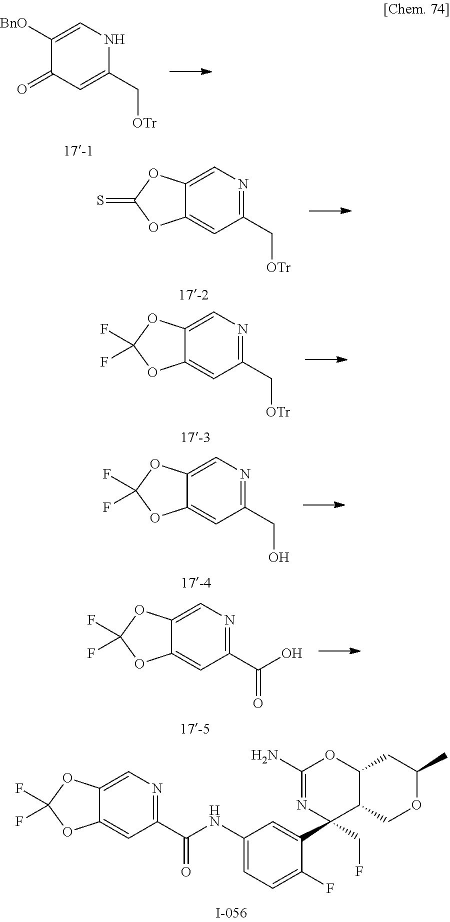

### Step 1

To a solution Compound 17′-1 (4.74 g, 10.0 mmol) in THF (95 mL), water (9.47 mL) and 2 mol/L aqueous sodium hydroxide (5.50 mL, 11.00 mmol) was added 10 wt. % palladium on carbon (2.37 g). After being stirred for 4.5 hours at room temperature under 1 atm hydrogen, the reaction mixture was filtered through Celite (Registered trademark) pad. The filtrate was evaporated. The residue was dehydrated by azeotropic distillation with acetonitrile. To a suspension of the residue in dichloromethane (95 mL) were added DMAP (2.44 g, 20.0 mmol) and thiophosgene (1.15 mL, 15.0 mmol). After being for 4 hours at room temperature, the reaction mixture was quenched with water and extracted with chloroform. The combined organic layers were washed with brine, dried over sodium sulfate, and filtered. The solvent was evaporated. The crude product was added to a silica gel column and eluted with hexane/EtOAc 10% to 100%. Collected fractions were evaporated to afford Compound 17′-2 (3.61 g, 8.48 mmol, 62%) as a yellow solid. LC/MS: Method A, M+23(Na)=448, tR=2.89 min.

### Step 2

To a solution of Compound 17′-2 (2.34 g, 5.50 mL) in dichloromethane (70.2 mL) were added hydrogen fluoride pyridine (23 mL) and 1,3-dibromo-5,5-dimethylimidazolidine-2,4-dione (4.72 g, 16.5 mmol) at less than −60° C. The bath was removed. The temperature was allowed to rise to 0° C. for 20 minutes. After being stirred for 2 hours at this temperature, the reaction mixture was quenched with 2 mol/L aqueous sodium hydroxide. The mixture was filtered through Celite (Registered trademark) pad. The filtrate was extracted with dichloromethane. The combined organic layers were washed with brine, dried over sodium sulfate, and filtered. The solvent was evaporated. The crude product was added to a silica gel column and eluted with hexane/EtOAc 10% to 90%. Collected fractions were evaporated to afford Compound 17′-3 (1.24 g, 3.35 mmol, 61%) as a yellow oil.

¹H NMR (400 MHz, CDCl₃) δ: 3.31 (1H, t, J=5.1 Hz), 4.75 (2H, d, J=5.3 Hz), 7.09 (1H, s), 8.31 (1H, s).

### Step 3

The Compound 17′-4 was prepared in a manner similar to the above protocols. (Example 3) The yield was not determined because the product was used in the next step without purification.

LC/MS: Method A, M+19=206, tR=1.43 min.

### Step 4

The Compound 17′-5 was prepared in a manner similar to the above protocols. (Example 2)(yield: 63%)

¹H NMR (400 MHz, DMSO-d6) δ: 8.14 (1H, s), 8.77 (1H, s).

### Step 5

The Compound I-056 was prepared in a manner similar to the above protocols. (yield: 56%)

¹H NMR (CDCl₃) δ: 1.17 (3H, d, J=6.3 Hz), 1.40-1.47 (1H, m), 1.79-1.85 (1H, m), 2.67-2.72 (1H, m), 3.66-3.73 (2H, m), 4.01-4.07 (2H, m), 4.34 (2H, br s), 4.41 (2H, t, J=5.9 Hz), 4.64 (2H, d, J=47.2 Hz), 7.08 (1H, dd, J=11.5, 9.0 Hz), 7.49 (1H, dd, J=6.8, 2.8 Hz), 7.95 (1H, s), 7.99-8.02 (1H, m), 8.32 (1H, s), 9.82 (1H, s).

The following compounds are prepared in a manner similar to the above protocols. In the tables, tR means LC/MS retention time (minute).

TABLE 3

| | | | M + H | tR | LC/MS |
|---|---|---|---|---|---|
| No. | Structure | ¹H NMR | observed | (min) | method |
| I-002 | 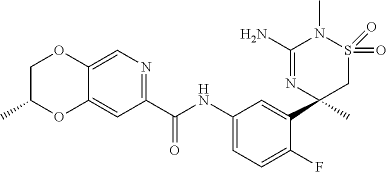 [SMILES: C[C@@H]1COc2cnc(C(=O)Nc3ccc(F)c([C@]4(C)CS(=O)(=O)N(C)C(N)=N4)c3)cc2O1] | ¹H NMR (400 MHz, CDCl₃) δ: 1.43 (3H, d, J = 6.4 Hz), 1.79 (3H, s), 3.24 (3H, s), 3.74 (1H, d, J = 14.0 Hz), 3.84 (1H, d, J = 14.0 Hz), 3.91 (1H, dd, J = 11.3, 7.9 Hz), 4.31-4.43 (2H, m), 7.05 (1H, dd, J = 11.7, 8.7 Hz), 7.77 (1H, s), 7.77-7.86 (2H, m), 8.12 (1H, s), 9.86 (1H, s). | 464 | 1.26 | B |
| I-005 | 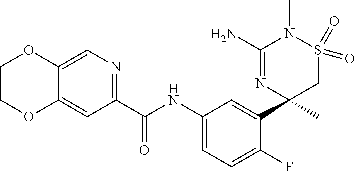 [SMILES: CN1C(N)=N[C@](C)(c2cc(NC(=O)c3cc4c(cn3)OCCO4)ccc2F)CS1(=O)=O] | ¹H NMR (400 MHz, CDCl₃) δ: 1.79 (3H, s), 3.23 (3H, s), 3.74 (1H, d, J = 13.9 Hz), 3.80 (1H, d, J = 13.9 Hz), 4.34-4.41 (4H, m), 7.05 (1H, t, J = 9.9 Hz), 7.77-7.85 (3H, m), 8.12 (1H, s), 9.84 (1H, s). | 450 | 1.2 | B |

TABLE 4

| | | | | | |
|---|---|---|---|---|---|
| I- 006 | 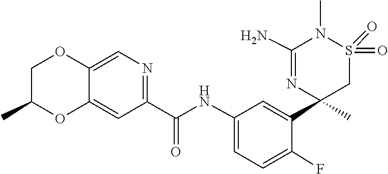 [SMILES: C[C@H]1COc2cnc(C(=O)Nc3ccc(F)c([C@]4(C)CS(=O)(=O)N(C)C(N)=N4)c3)cc2O1] | ¹H NMR (400 MHz, CDCl₃) δ: 1.43 (3H, d, J = 6.4 Hz), 1.79 (3H, s), 3.24 (3H, s), 3.74 (1H, d, J = 14.0 Hz), 3.82 (1H, d, J = 14.0 Hz), 3.91 (1H, dd, J = 11.5, 7.9 Hz), 4.32-4.44 (2H, m), 7.05 (1H, dd, J = 11.2, 9.4 Hz), 7.77 | 464 | 1.28 | B |
| | | (1H, s), 7.77-7.84 | | | |
| | | (2H, m), 8.12 (1H, | | | |
| | | s), 9.87 (1H, s). | | | |
| I- 007 | 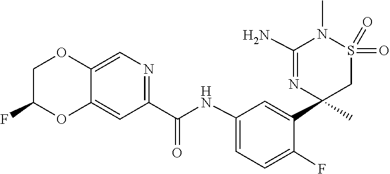 [SMILES: CN1C(N)=N[C@](C)(c2cc(NC(=O)c3cc4c(cn3)OC[C@H](F)O4)ccc2F)CS1(=O)=O] | ¹H NMR (400 MHz, CDCl₃) δ: 1.79 (3H, s), 3.23 (3H, s), 3.73 (1H, d, J = 13.9 Hz), 3.84 (1H, d, J = 13.9 Hz), 4.15 (1H, dd, J = 27.7, 12.3 Hz), 4.52-4.58 (1H, m), 6.13 (1H, d, J = 52.2 Hz), 7.06 (1H, dd, J = | 468 | 1.22 | B |
| | | 11.7, 8.7 Hz), 7.75- | | | |
| | | 7.81 (1H, m), 7.84 | | | |
| | | (1H, dd, J = 7.3, 2.6 | | | |
| | | Hz), 7.90 (H, s), | | | |
| | | 8.21 (1H, s), 9.80 | | | |
| | | (1H, s). | | | |
| I- 008 | 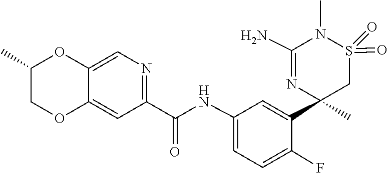 [SMILES: C[C@H]1COc2cc(C(=O)Nc3ccc(F)c([C@]4(C)CS(=O)(=O)N(C)C(N)=N4)c3)ncc2O1] | ¹H NMR (400 MHz, CDCl₃) δ: 1.44 (3H, d, J = 6.4 Hz), 1.80 (3H, s), 3.24 (3H, s), 3.74 (1H, d, J = 13.9 Hz), 3.85 (1H, d, J = 13.9 Hz), 3.94 (1H, dd, J = 11.8, 8.5 Hz), 4.30-4.38 (2H, m), 7.06 (1H, dd, J = | 464 | 1.26 | B |
| | | 11.5, 8.8 Hz), 7.76- | | | |
| | | 7.86 (H, m), 7.78 | | | |
| | | (1H, s), 8.11 (1H, s), | | | |
| | | 9.85 (1H, s). | | | |

TABLE 5

| | | | | | |
|---|---|---|---|---|---|
| I- 009 | 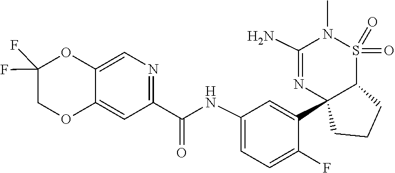 [SMILES: CN1C(N)=N[C@@]2(c3cc(NC(=O)c4cc5c(cn4)OC(F)(F)CO5)ccc3F)CCC[C@H]2S1(=O)=O] | ¹H NMR (400 MHz, CDCl₃) 8: 1.88-1.97 (1H, m), 2.02-2.10 (1H, m), 2.16-2.27 (1H, m), 2.13-2.44 (1H, m), 2.50-2.60 (1H, m), 4.23 (1H, dd, J = 9.4, 6.0 Hz), 4.41 (1H, t, J = 5.8 Hz), 7.07 (1H, dd, J = 11.8, | 512 | 1.41 | B |
| | | 8.7 Hz), 7.78-7.84 | | | |
| | | (2H, m), 7.94 (1H, s), | | | |
| | | 8.30 (1H, m), 9.80 | | | |
| | | (1H, m). | | | |
| I- 010 | 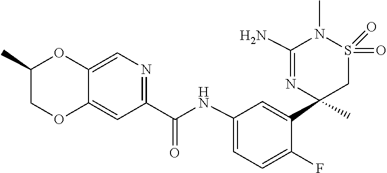 [SMILES: C[C@@H]1COc2cc(C(=O)Nc3ccc(F)c([C@]4(C)CS(=O)(=O)N(C)C(N)=N4)c3)ncc2O1] | ¹H NMR (400 MHz, CDCl₃) 8: 1.44 (3H, d, J = 6.3 Hz), 1.80 (3H, s), 3.24 (3H, s), 3.74 (1H, d, J = 13.9 Hz), 3.87 (1H, d, J = 13.9 Hz), 3.94 (1H, dd, J = 11.7, 8.3 Hz), 4.30- 4.39 (2H, m), 7.06 (1H, dd, J = 11.3, 8.9 | 464 | 1.24 | B |
| | | Hz), 7.77-7.84 (3H, | | | |
| | | m), 8.11 (1H, s), 9.86 | | | |
| | | (1H, s). | | | |
| I- 013 | 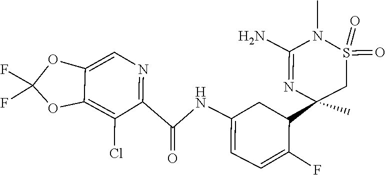 [SMILES: CN1C(N)=N[C@](C)(C2CCC(NC(=O)C3=NC=C4OC(F)(F)OC4=C(Cl)C3)=CC=C2F)CS1(=O)=O] | ¹H NMR (400 MHz, CDCl₃) 8: 1.78 (3H, s), 3.74 (3H, s), 3.72 (1H, d, J = 14.1 Hz), 3.82 (1H, d, J = 14.1 Hz), 7.08 (1H, dd, J = 11.7, 8.8 Hz), 7.74 (1H, dd, J = 7.0, 2.8 Hz), 7.83- 7.89 (1H, m), 8.28 (1H, s), 9.68 (1H, s). | 506 | 1.43 | B |
| I- 014 | 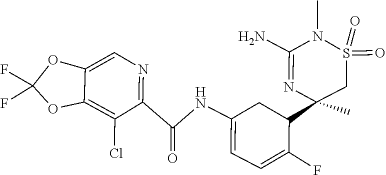 [SMILES: CN1C(N)=N[C@](C)(C2CCC(NC(=O)C3=NC=C4OC(F)(F)OC4=C(Cl)C3)=CC=C2F)CS1(=O)=O] | ¹H NMR (400 MHz, CDCl₃) 8: 1.78 (3H, s), 2.76 (3H, s), 3.24 (3H, s), 3.74 (1H, d, J = 14.1 Hz), 3.81 (1H, d, J = 14.1 Hz), 7.07 (1H, dd, J = 11.7, 8.8 Hz), 7.73 (1H, dd, J = 7.2, 2.6 Hz), 7.80-7.87 (1H, m), 8.22 (1H, s), 9.93 (1H, s). | 486 | 1.52 | B |

TABLE 6

| | | | | | |
|---|---|---|---|---|---|
| I- 015 | 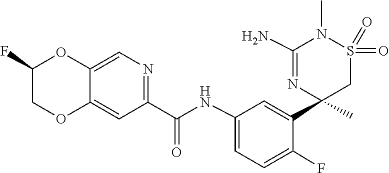 [SMILES: CN1C(N)=N[C@](C)(c2cc(NC(=O)c3cc4c(cn3)O[C@H](F)CO4)ccc2F)CS1(=O)=O] | ¹H NMR (400 MHz, CDCl₃) δ: 1.79 (3H, s), 3.23 (3H, s), 3.73 (1H, d, J = 13.9 Hz), 3.82 (1H, d, J = 13.9 Hz), 4.18 (1H, dd, J = 28.5, 12.5 Hz), 4.53-4.58 (1H, m), 6.14 (1H, d, J = 51.7 Hz), 7.06 (1H, d, J = 11.7, 8.9 | 468 | 1.21 | B |
| | | Hz), 7.74-7.88 (3H, | | | |
| | | m), 8.23 (1H, s), 9.83 | | | |
| | | (1H, s). | | | |
| I- 016 | 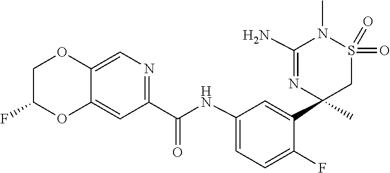 [SMILES: CN1C(N)=N[C@](C)(c2cc(NC(=O)c3cc4c(cn3)OC[C@@H](F)O4)ccc2F)CS1(=O)=O] | ¹H NMR (400 MHz, CDCl₃) δ: 1.79 (3H, s), 3.23 (3H, s), 3.73 (1H, d, J = 14.0 Hz), 3.83 (1H, d, J = 14.0 Hz), 4.14 (1H, dd, J = 28.0, 13.3 Hz), 4.54 (1H, ddd, J = 12.3, 4.0, 1.0 Hz), 6.13 (111, d, J = 52.0 Hz), 7.06 (1H, | 468 | 1.21 | B |
| | | dd, J = 11.6, 8.8 Hz), | | | |
| | | 7.76-7.81 (1H, m), | | | |
| | | 7.85 (1H, dd, J = 7.2, | | | |
| | | 2.6 Hz), 7.90 (1H, s), | | | |
| | | 8.21 (1H, 0, 9.81 (1H, | | | |
| | | s). | | | |
| I- 017 | 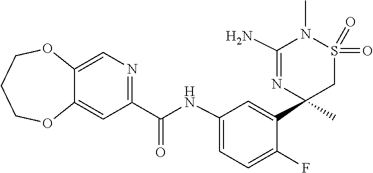 [SMILES: CN1C(N)=N[C@](C)(c2cc(NC(=O)c3cc4c(cn3)OCCCO4)ccc2F)CS1(=O)=O] | ¹H NMR (360 MHz, CDCl₃) δ : 1.78 (3H, s), 2.31 (2H, quin, J = 5.9 Hz), 3.23 (3H, s), 3.68-3.88 (2H, m), 4.38 (4H, dt, J = 18.1, 5.8 Hz), 7.05 (1H, dd, J = 11.7, 8.8 Hz), 7.75-7.80 (2H, m), 7.83 (1H, dd, | 464 | 0.81 | C |
| | | J = 7.1, 2.7 Hz), 8.16 | | | |
| | | (1H, s), 9.83 (1H, s) | | | |

TABLE 7

| | | | | | |
|---|---|---|---|---|---|
| I- 018 | 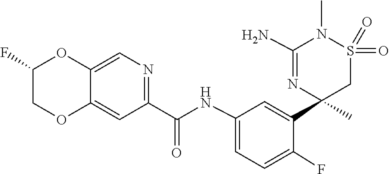 [SMILES: CN1C(N)=N[C@](C)(c2cc(NC(=O)c3cc4c(cn3)O[C@@H](F)CO4)ccc2F)CS1(=O)=O] | ¹H NMR (400 MHz, CDCl₃) δ: 1.79 (3H, s), 3.23 (3H, s), 3.73 (1H, d, J = 13.9 Hz), 3.82 (1H, d, J = 13.9 Hz), 4.18 (1H, dd, J = 28.5, 12.5 Hz), 4.53-4.58 (1H, m), 6.14 (1H, d, J = 51.7 Hz), 7.06 (1H, d, J = 11.7, 8.9 | 468 | 1.2 | B |
| | | Hz), 7.74-7.88 (3H, | | | |
| | | m), 8.23 (1H, s), 9.83 | | | |
| | | (1H, s). | | | |
| I- 019 | 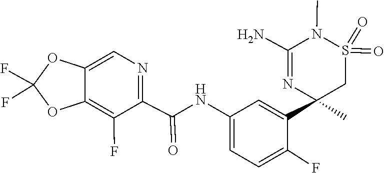 [SMILES: CN1C(N)=N[C@](C)(c2cc(NC(=O)c3ncc4c(c3F)OC(F)(F)O4)ccc2F)CS1(=O)=O] | ¹H NMR (400 MHz, CDCl₃) δ: 1.78 (3H, s), 3.24 (3H, s), 3.72 (1H, d, J = 14.1 Hz), 3.82 (1H, d, J = 14.1 Hz), 7.07 (1H, dd, J = 11.3, 9.8 Hz), 7.78-7.84 (2H, m), 8.24 (1H, s), 9.58 (1H, s). | 490 | 1.49 | B |
| I- 020 | 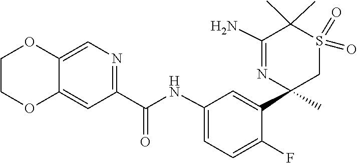 [SMILES: CC1(C)C(N)=N[C@](C)(c2cc(NC(=O)c3cc4c(cn3)OCCO4)ccc2F)CS1(=O)=O] | ¹H NMR (400 MHz, CDCl₃) δ: 1.63 (3H, s), 1.70 (3H, s), 1.80 (3H, s), 3.56 (1H, d, J = 14.9 Hz), 3.63 (1H, d, J = 14.9 Hz), 4.34- 4.41 (4H, m), 7.06 (1H, dd, J = 11.6, 8.8 Hz), 7.66-7.70 (1H, m), 7.80 (1H, s), 7.83- | 463 | 1.22 | B |
| | | 7.88 (1H, m), 8.12 | | | |
| | | (1H, s), 9.83 (1H, s). | | | |
| I- 021 | 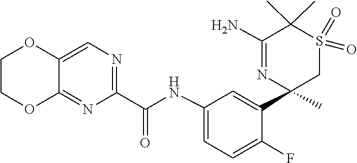 [SMILES: CC1(C)C(N)=N[C@](C)(c2cc(NC(=O)c3ncc4c(n3)OCCO4)ccc2F)CS1(=O)=O] | ¹H NMR (400 MHz, CDCl₃) δ: 1.63 (3H, s), 1.70 (3H, s), 1.81 (3H, s), 3.55 (1H, d, J = 14.7 Hz), 3.63 (1H, d, J = 14.7 Hz), 4.39- 4.43 (2H, m), 4.58- 4.63 (2H, m), 7.07 (1H, dd, J = 11.6, 8.8 Hz), 7.74-7.78 (1H, | 464 | 0.93 | B |
| | | m), 7.85-7.80 (1H, m), | | | |
| | | 8.37 (1H, s), 9.62 (1H, | | | |
| | | s). | | | |

TABLE 8

| | | | | | |
|---|---|---|---|---|---|
| I- 022 | 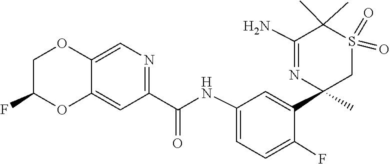 [SMILES: CC1(C)C(N)=N[C@](C)(c2cc(NC(=O)c3cc4c(cn3)OC[C@H](F)O4)ccc2F)CS1(=O)=O] | ¹H NMR (400 MHz, CDCl₃) δ: 1.63 (3H, s), 1.70 (3H, s), 1.81 (3H, s), 3.56 (1H, d, J = 15.2 Hz), 3.64 (1H, d, J = 15.2 Hz), 4.15 (1H, dd, J = 12.5, 26.7 Hz), 4.55 (1H, dd, J = 12.5, 3.8 Hz), 6.14 (1H, d, J = 52.1 Hz), | 481 | 1.25 | B |
| | | 7.08 (1H, dd, J = 11.7, | | | |
| | | 8.8 Hz), 7.71-7.66 | | | |
| | | (1H, m), 7.88-7.83 | | | |
| | | (1H, m), 7.91 (1H, s), | | | |
| | | 8.22 (1H, s), 9.80 (1H, | | | |
| | | s). | | | |
| I- 024 | 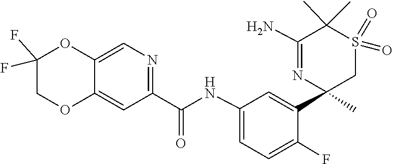 [SMILES: CC1(C)C(N)=N[C@](C)(c2cc(NC(=O)c3cc4c(cn3)OC(F)(F)CO4)ccc2F)CS1(=O)=O] | ¹H NMR (400 MHz, CDCl₃) δ: 1.63 (3H, s), 1.71 (3H, s), 1.80 (3H, s), 3.57 (1H, d, J = 15.2 Hz), 3.63 (1H, d, J = 15.2 Hz), 4.41 (2H, t, J = 5.8 Hz), 7.05-7.12 (1H, m), 7.65-7.70 (1H, m), 7.89-7.83 (1H, m), | 499 | 1.38 | B |
| | | 7.96 (1H, s), 8.32 (1H, | | | |
| | | s), 9.79 (1H, s). | | | |
| I- 025 | 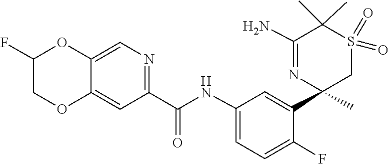 [SMILES: CC1(C)C(N)=N[C@](C)(c2cc(NC(=O)c3cc4c(cn3)OC(F)CO4)ccc2F)CS1(=O)=O] | ¹H NMR (400 MHz, CDCl₃) δ: 1.63 (3H, s), 1.70 (3H, s), 1.80 (3H, s), 3.56 (1H, d, J = 15.2 Hz), 3.63 (1H, d, J = 15.2 Hz), 4.19 (1H, dd, J = 28.2, 12.0 Hz), 4.52-4.59 (1H, m), 6.15 (1H, d, J = 51.8 Hz), 7.04-7.11 | 481 | 1.21 | B |
| | | (1H, m), 7.65-7.70 | | | |
| | | (1H, m), 7.89-7.84 | | | |
| | | (1H, m), 7.87 (1H, s), | | | |
| | | 8.23 (1H, s), 9.81 (1H, | | | |
| | | s). | | | |

TABLE 9

| | | | | | |
|---|---|---|---|---|---|
| I- 026 | 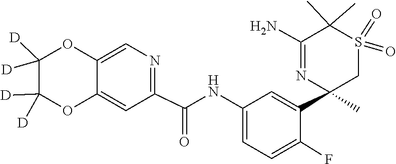 [SMILES: [2H]C1([2H])Oc2cnc(C(=O)Nc3ccc(F)c([C@]4(C)CS(=O)(=O)C(C)(C)C(N)=N4)c3)cc2OC1([2H])[2H]] | ¹H NMR (400 MHz, DMSO-d6) δ: 1.47 (3H, s), 1.53-1.60 (3H, m), 1.62 (3H, s), 3.44-3.74 (2H, m), 6.03 (2H, brs), 7.04- 7.21 (1H, m), 7.58 (1H, s), 7.65-8.03 (2H, m), 8.24 (1H, s), 10.32 (1H, brs) | 467 | 0.79 | C |
| I- 027 | 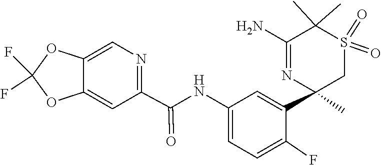 [SMILES: CC1(C)C(N)=N[C@](C)(c2cc(NC(=O)c3cc4c(cn3)OC(F)(F)O4)ccc2F)CS1(=O)=O] | ¹H NMR (400 MHz, CDCl₃) δ: 1.63 (3H, s), 1.71 (3H, s), 1.80 (3H, s), 3.58 (1H, d, J = 15.2 Hz), 3.63 (1H, d, J = 15.2 Hz), 7.06- 7.13 (1H, m), 7.66- 7.70 (1H, m), 7.88- 7.83 (1H, m), 8.09 (1H, s), 8.36 (1H, s), | 485 | 1.44 | B |
| | | 9.76 (1H, s). | | | |
| I- 028 | 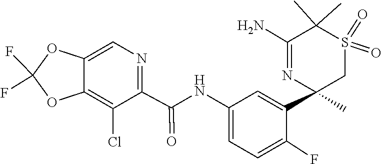 [SMILES: CC1(C)C(N)=N[C@](C)(c2cc(NC(=O)c3ncc4c(c3Cl)OC(F)(F)O4)ccc2F)CS1(=O)=O] | ¹H NMR (400 MHz, CDCl₃) δ: 1.63 (3H, s), 1.71 (3H, s), 1.80 (3H, s), 3.58 (1H, d, J = 14.8 Hz), 3.63 (1H, d, J = 14.8 Hz), 7.06- 7.12 (1H, m), 7.54- 7.59 (1H, m), 7.95- 7.89 (1H, m), 8.29 (1H, s), 9.66 (1H, s). | 519 | 1.47 | B |

TABLE 10

| | | | | | |
|---|---|---|---|---|---|
| I- 064 | 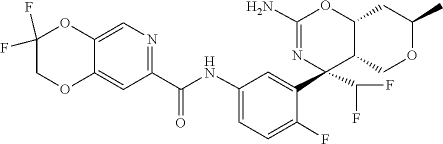 [SMILES: C[C@@H]1C[C@H]2OC(N)=N[C@@](c3cc(NC(=O)c4cc5c(cn4)OC(F)(F)CO5)ccc3F)(C(F)F)[C@H]2CO1] | ¹H NMR (400 MHz, CDCl₃) δ: 1.39 (3H, s), 3.31-3.37 (1H, m), 3.91-3.99 (2H, m), 3.93 (3H, s), 4.32- 4.45 (5H, m), 5.07 (1H, d, J = 9.3 Hz), 7.07 (1H, t, J = 10.1 Hz), 7.40-7.45 (1H, | 529 | 1.29 | B |
| | | 7.46 (1H, s), 7.61 | | | |
| | | (1H, s), 7.80 (1H, s), | | | |
| | | 7.93-7.99 (1H, m), | | | |
| | | 8.14 (1H, s), 9.84 | | | |
| | | (1H, s). | | | |
| I- 066 | 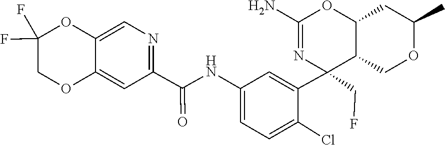 [SMILES: C[C@@H]1C[C@H]2OC(N)=N[C@](CF)(c3cc(NC(=O)c4cc5c(cn4)OC(F)(F)CO5)ccc3Cl)[C@H]2CO1] | ¹H NMR (400 MHz, CDCl₃) δ: 1.17 (3H, d, J = 6.3 Hz), 1.40- 1.47 (m, 1H), 1.78- 1.84 (m, 1H), 3.14- 3.19 (1H, m), 3.66- 3.77 (2H, m), 3.98- 4.05 (2H, m), 4.42 (1H, t, J = 5.9 Hz), | 527 | 1.55 | B |
| | | 4.77-5.02 (2H, m), | | | |
| | | 5.07 (1H, d, J = 9.3 | | | |
| | | Hz), 7.39 (1H, d, J = | | | |
| | | 8.7 Hz), 7.63 (1H, d, | | | |
| | | J = 2.6 Hz), 7.95 | | | |
| | | (1H, s), 8.06 (1H, dd, | | | |
| | | J = 8.6, 2.7 Hz), 8.32 | | | |
| | | (1H, s), 9.86 (1H, | | | |
| | | brs). | | | |
| I- 071 | 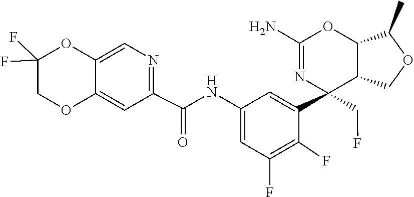 [SMILES: C[C@H]1OC[C@H]2[C@@H]1OC(N)=N[C@]2(CF)c1cc(NC(=O)c2cc3c(cn2)OC(F)(F)CO3)cc(F)c1F] | ¹H NMR (400 MHz, CDCl₃) δ: 1.39 (3H, s), 3.31-3.37 (1H, m), 3.91-3.99 (2H, m), 3.93 (3H, s), 4.32- 4.45 (5H, m), 5.07 (1H, d, J = 9.3 Hz), 7.07 (1H, t, J = 10.1 Hz), 7.40-7.45 (1H, m), 7.46 (1H, s), 7.61 (1H, s), 7.80 (1H, s), 7.93-7.99 (1H, m), | 515 | 1.43 | B |
| | | 8.14 (1H, s), 9.84 | | | |
| | | (1H, s). | | | |

TABLE 11

| | | | | | |
|---|---|---|---|---|---|
| I- 032 | 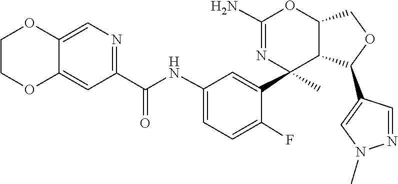 [SMILES: Cn1cc([C@@H]2OC[C@H]3OC(N)=N[C@](C)(c4cc(NC(=O)c5cc6c(cn5)OCCO6)ccc4F)[C@H]32)cn1] | ¹H NMR (400 MHz, CDCl₃) δ: 1.39 (3H, s), 3.31-3.37 (1H, m), 3.91-3.99 (2H, m), 3.93 (3H, s), 4.32- 4.45 (5H, m), 5.07 (1H, d, J =9.3 Hz), 7.07 (1H, t, J =10.1 Hz) 7.40-7.45 (1H, 7.46 (1H, s), 7.61 (1H, s), 7.80 (1H, s), | 509 | 1.11 | B |
| | | 7.93-7.99 (1H, m), | | | |
| | | 8.14 (1H, s), 9.84 | | | |
| | | (1H, s). | | | |
| I- 033 | 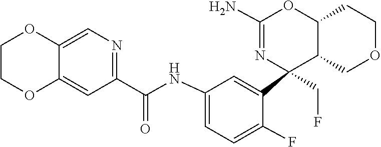 [SMILES: NC1=N[C@](CF)(c2cc(NC(=O)c3cc4c(cn3)OCCO4)ccc2F)[C@H]2COCC[C@H]2O1] | ¹H NMR (CDCl₃) δ: 1.73-1.87 (2H, m), 2.72-2.77 (1H, m), 3.59-3.66 (2H, m), 3.76 (1H, dd, J = 11.6, 5.0 Hz), 4.00- 4.05 (1H, m), 4.08 (1H, br s), 4.35-4.41 (6H, m), 4.53-4.73 | 461 | 1.12 | B |
| | | (2H, m), 7.07 (1H, | | | |
| | | dd, J = 11.6, 8.8 Hz), | | | |
| | | 7.47 (1H, dd, J = 6.9, | | | |
| | | 2.8 Hz), 7.79 (1H, s), | | | |
| | | 8.00-8.04 (1H, m), | | | |
| | | 8.13 (1H, s), 9.86 | | | |
| | | (1H, s). | | | |
| I- 034 | 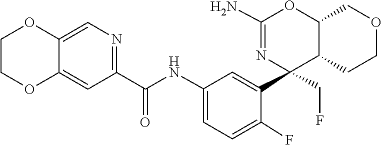 [SMILES: NC1=N[C@](CF)(c2cc(NC(=O)c3cc4c(cn3)OCCO4)ccc2F)[C@H]2CCOC[C@H]2O1] | ¹H NMR (400 MHz, CDCl₃) δ: 1.62-1.66 (1H, m), 1.85-1.95 (1H, m), 2.59-2.64 (1H, m), 3.41-3.48 (2H, m), 3.74 (1H, br s), 3.98 (1H, d, J = 13.0 Hz), 4.05 (1H, dd, J = 12.1, 4.5 Hz), | 461 | 1.28 | B |
| | | 4.36-4.40 (8H, m), | | | |
| | | 4.60 (1H, ddd, J = | | | |
| | | 47.4, 9.3, 1.5 Hz), | | | |
| | | 4.78 (1H, dd, J = | | | |
| | | 46.9, 9.3 Hz), 7.07 | | | |
| | | (1H, dd, J = 11.7, 8.9 | | | |
| | | Hz), 7.45 (1H, dd, J = | | | |
| | | 7.0, 2.6 Hz), 7.79 | | | |
| | | (1H, s), 8.03-8.07 | | | |
| | | (1H, m), 8.13 (1H, s), | | | |
| | | 9.86 (1H, s). | | | |
| I- 036 | 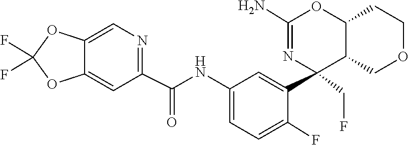 [SMILES: NC1=N[C@](CF)(c2cc(NC(=O)c3cc4c(cn3)OC(F)(F)O4)ccc2F)[C@H]2COCC[C@H]2O1] | ¹H NMR (400 MHz, CDCl₃) δ: 1.84-1.73 (2H, m), 2.74-2.78 (1H, m), 3.58-3.66 (2H, m), 3.77 (1H, dd, J = 11.2, 5.0 Hz), 4.00-4.05 (1H, m), 4.09 (1H, s) 4.37 (2H, br s), 4.57-4.69 | 483 | 1.33 | B |
| | | (2H, m), 7.10 (1H, | | | |
| | | dd, J = 11.4, 8.8 Hz), | | | |
| | | 7.49-7.52 (1H, m) | | | |
| | | 7.99-8.03 (1H, m), | | | |
| | | 8.09 (1H, s), 8.37 | | | |
| | | (1H, s), 9.79 (1H, s). | | | |

TABLE 12

| | | | | | |
|---|---|---|---|---|---|
| I- 037 | 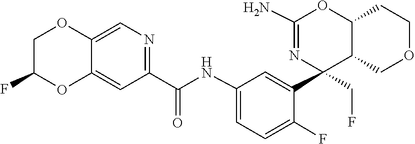 [SMILES: NC1=N[C@](CF)(c2cc(NC(=O)c3cc4c(cn3)OC[C@H](F)O4)ccc2F)[C@H]2COCC[C@H]2O1] | ¹H NMR (400 MHz, CDCl₃) δ: 1.73-1.84 (2H, m), 2.73-2.78 (1H, m), 3.58-3.66 (2H, m), 3.76 (1H, dd, J = 11.3, 4.9 Hz), 4.01-4.05 (1H, m), 4.07-4.20 (2H, m), 4.39 (2H, br s), 4.53- | 479 | 1.15 | B |
| | | 4.73 (3H, m), 6.14 | | | |
| | | (1H, d, J = 52.2 Hz), | | | |
| | | 7.08 (1H, dd, J = | | | |
| | | 12.0, 9.1 Hz), 7.45- | | | |
| | | 7.47 (1H, m), 7.91 | | | |
| | | (1H, s), 8.02-8.06 | | | |
| | | (1H, m), 8.23 (1H, s), | | | |
| | | 9.83 (1H, s). | | | |
| I- 038 | 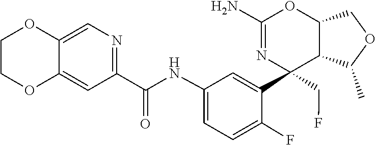 [SMILES: C[C@H]1OC[C@H]2OC(N)=N[C@](CF)(c3cc(NC(=O)c4cc5c(cn4)OCCO5)ccc3F)[C@@H]12] | ¹H NMR (400 MHz, CDCl₃) δ: 1.37 (3H, d, J = 6.7 Hz), 3.03 (1H, dd, J = 7.1, 5.0 Hz), 3.95-4.00 (2H, m), 4.35-4.40 (7H, m), 4.48-4.55 (1H, m), 4.65-4.93 (2H, m), 7.08 (1H, dd, J = | 461 | 1 | B |
| | | 11.7, 8.8 Hz), 7.51 | | | |
| | | (1H, dd, J = 7.2, 3.5 | | | |
| | | Hz), 7.78 (1H, s), | | | |
| | | 7.98 (1H, dt, J = 8.8, | | | |
| | | 3.5 Hz), 8.11 (1H, s), | | | |
| | | 9.86 (1H, s). | | | |
| I- 039 | 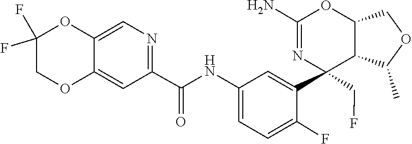 [SMILES: C[C@H]1OC[C@H]2OC(N)=N[C@](CF)(c3cc(NC(=O)c4cc5c(cn4)OC(F)(F)CO5)ccc3F)[C@@H]12] | ¹H NMR (400 MHz, CDCl₃) δ: 1.37 (3H, d, J = 6.5 Hz), 3.03 (1H, dd, J = 7.0, 5.1 Hz), 3.95-4.01 (2H, m), 4.36-4.55 (6H, m), 4.71-4.87 (2H, 11.7, 8.8 Hz), 7.53 | 497 | 1.11 | B |
| | | (1H, dd, J = 7.0, 2.6 | | | |
| | | Hz), 7.94-7.97 (2H, | | | |
| | | m), 8.32 (1H, s), 9.82 | | | |
| | | (1H, s). | | | |
| I- 040 | 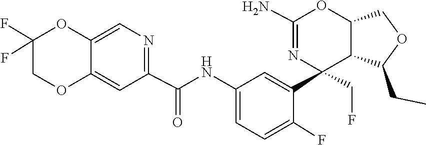 [SMILES: CC[C@@H]1OC[C@H]2OC(N)=N[C@](CF)(c3cc(NC(=O)c4cc5c(cn4)OC(F)(F)CO5)ccc3F)[C@@H]12] | ¹H NMR (400 MHz, CDCl₃) δ: 1.08 (3H, t, J = 7.3 Hz), 1.78- 1.90 (1H, m), 2.90- 2.95 (1H, m), 3.76 (1H, dd, 1 = 10.3, 2.4 Hz), 3.89 (1H, d, J = 10.3 Hz), 4.20-4.14 (1H, m), 4.35-4.38 | 511 | 1.27 | B |
| | | (1H, m) 4.41 (2H, t , | | | |
| | | J = 5.8 Hz), 4.62 | | | |
| | | (2H, d, J = 46.9 Hz), | | | |
| | | 7.10 (1H, dd, J = | | | |
| | | 11.4, 9.0 Hz), 7.48- | | | |
| | | 7.53 (1H, m), 7.96 | | | |
| | | (1H, s), 7.97-8.03 | | | |
| | | (1H, m), 8.33 (1H, s), | | | |
| | | 9.82 (1H, s). | | | |

TABLE 13

| | | | | | |
|---|---|---|---|---|---|
| I- 041 | 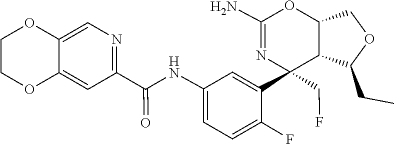 [SMILES: CC[C@@H]1OC[C@H]2OC(N)=N[C@](CF)(c3cc(NC(=O)c4cc5c(cn4)OCCO5)ccc3F)[C@@H]12] | ¹H NMR (400 MHz, CDCl₃) δ: 1.08 (3H, t, J = 7.3 Hz), 1.79- 1.89 (1H, m), 2.89- 2.94 (1H, m), 3.76 (1H, dd, J = 10.2, 2.1 Hz), 3.89 (1H, d, J = 10.2 Hz), 4.14-4.20 (1H, m), 4.34-4.42 | 475 | 1.25 | B |
| | | (5H, m), 4.56 (1H, | | | |
| | | dd, J = 18.8 8.8 Hz), | | | |
| | | 4.68 (1H, dd, J = | | | |
| | | 18.8, 8.8 Hz), 7.08 | | | |
| | | (1H, dd, J = 11.7, 8.8 | | | |
| | | Hz), 7.46-7.50 (1H, | | | |
| | | m), 7.79 (1H, s), | | | |
| | | 8.00-8.04 (1H, m), | | | |
| | | 8.13 (1H, s), 9.87 | | | |
| | | (1H, s). | | | |
| I- 042 | 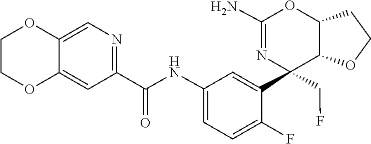 [SMILES: NC1=N[C@](CF)(c2cc(NC(=O)c3cc4c(cn3)OCCO4)ccc2F)[C@H]2OCC[C@H]2O1] | ¹H NMR (400 MHz, CDCl₃) δ: 2.10-2.13 (2H, m), 3.97-4.03 (1H, m), 4.03-4.11 (1H, m), 4.27 (2H, br s), 4.37-4.40 (6H, m), 4.64 (1H, dd, J = 48.1, 8.9 Hz), 5.08 (1H, dd, J = 46.8 9.2 | 447 | 1.15 | B |
| | | Hz), 7.07 (1H, dd, J = | | | |
| | | 11.2, 9.2 Hz), 7.46- | | | |
| | | 7.48 (1H, m), 7.79 | | | |
| | | (1H, s), 8.02-8.06 | | | |
| | | (1H, m), 8.12 (1H, s), | | | |
| | | 9.86 (1H, s). | | | |
| I- 043 | 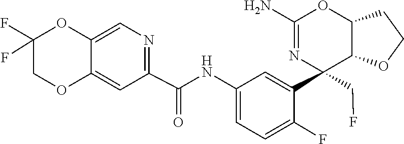 [SMILES: NC1=N[C@](CF)(c2cc(NC(=O)c3cc4c(cn3)OC(F)(F)CO4)ccc2F)[C@H]2OCC[C@H]2O1] | ¹H NMR (400 MHz, CDCl₃) δ: 2.09-2.14 (2H, m), 3.98-4.03 (1H, m), 4.08 (1H, q, J = 8.1 Hz), 4.27 (2H, br s), 4.40-4.42 (4H, m), 4.64 (1H, dd, J = 48.7, 8.8 Hz), 5.09 (1H, dd, J = | 483 | 1.3 | B |
| | | 47.3, 8.8 Hz), 7.09 | | | |
| | | (1H, dd, J = 9.2, 11.5 | | | |
| | | Hz), 7.50 (1H, dd, J = | | | |
| | | 6.6, 2.3 Hz), 7.95 | | | |
| | | (1H, s), 8.01-8.05 | | | |
| | | (1H, m), 8.32 (1H, s), | | | |
| | | 9.82 (1H, s). | | | |
| I- 044 | 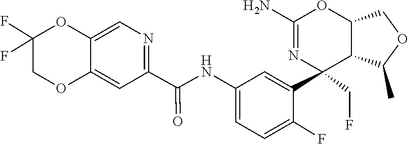 [SMILES: C[C@@H]1OC[C@H]2OC(N)=N[C@](CF)(c3cc(NC(=O)c4cc5c(cn4)OC(F)(F)CO5)ccc3F)[C@@H]12] | ¹H NMR (400 MHz, CDCl₃) δ: 1.45 (3H, d, J = 6.0 Hz), 2.79 (1H, dd, J = 9.0, 4.2 Hz), 3.86 (2H, t, J = 11.5 Hz), 4.28-4.45 (4H, m), 4.59 (1H, dd, J = 18.4, 8.7 Hz), 4.71 (1H, dd, J = | 497 | 1.3 | B |
| | | 18.4, 8.7 Hz) 7.10 | | | |
| | | (1H, dd, J = 11.7, 8.9 | | | |
| | | Hz), 7.50-7.54 (1H, | | | |
| | | m), 7.96 (1H, s), | | | |
| | | 7.97-8.02 (1H, m), | | | |
| | | 8.33 (1H, s), 9.83 | | | |
| | | (1H, s). | | | |

TABLE 14

| | | | | | |
|---|---|---|---|---|---|
| I- 045 | 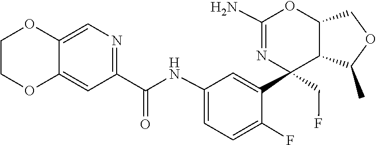 [SMILES: C[C@@H]1OC[C@H]2OC(N)=N[C@](CF)(c3cc(NC(=O)c4cc5c(cn4)OCCO5)ccc3F)[C@@H]12] | ¹H NMR (400 MHz, CDCl₃) δ: 1.45 (3H, d, J = 5.8 Hz), 2.79 (1H, dd, J = 9.0, 4.3 Hz), 3.86 (2H, s, 4.28-4.43 (6H, m), 4.53-4.76 (2H, m), 7.08 (1H, dd, J = 11.6, 8.8 Hz), 7.47- | 461 | 1.13 | B |
| | | 7.52 (1H, m), 7.79 | | | |
| | | (1H, s), 8.04-7.98 | | | |
| | | (1H, m), 8.13 (1H, s), | | | |
| | | 9.87 (1H, s). | | | |
| I- 046 | 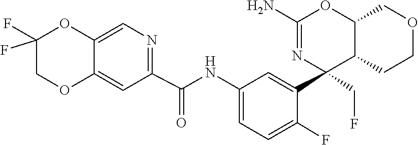 [SMILES: NC1=N[C@](CF)(c2cc(NC(=O)c3cc4c(cn3)OC(F)(F)CO4)ccc2F)[C@H]2CCOC[C@H]2O1] | ¹H NMR (400 MHz, CDCl₃) δ: 1.66-1.61 (2H, m), 1.85-1.96 (1H, m), 2.60-2.65 (1H, m), 3.41-3.48 (2H, m), 8.74 (1H, br s), 8.99 (1H, d, J = 12.8 Hz), 4.05-4.06 (1H, m), 4.29-4.43 | 497 | 2.17 | B |
| | | (4H, m), 4.54-4.65 | | | |
| | | (1H, m), 4.79 (1H, | | | |
| | | dd, J = 46.7, 8.7 Hz), | | | |
| | | 7.09 (1H, dd, J = | | | |
| | | 11.5, 9.0 Hz), 7.47- | | | |
| | | 7.49 (1H, m), 7.95 | | | |
| | | (1H, s), 8.02-8.06 | | | |
| | | (1H, m), 8.32 (1H, s), | | | |
| | | 9.82 (1H, s). | | | |
| I- 048 | 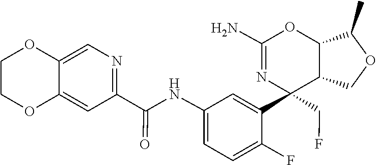 [SMILES: C[C@H]1OC[C@H]2[C@@H]1OC(N)=N[C@]2(CF)c1cc(NC(=O)c2cc3c(cn2)OCCO3)ccc1F] | ¹H NMR (400 MHz, CDCl₃) δ: 1.12 (3H, d, J = 6.8 Hz), 3.17- 3.24 (1H, m), 3.86- 3.93 (1H, m), 4.02 (1H, d, J = 4.5 Hz), 4.18 (1H, q, J = 6.8 Hz), 4.17-4.23 (1H, m), 4.30 (1H, br s), 4.85-4.42 (4H, m), | 461 | 1.11 | B |
| | | 4.44-4.74 (2H, m), | | | |
| | | 7.10 (1H, dd, J = | | | |
| | | 11.8, 8.8 Hz), 7.50- | | | |
| | | 7.55 (1H, m), 7.79 | | | |
| | | (1H, s), 8.04-7.99 | | | |
| | | (1H, m), 8.13 (1H, s), | | | |
| | | 9.87 (1H, s). | | | |
| I- 049 | 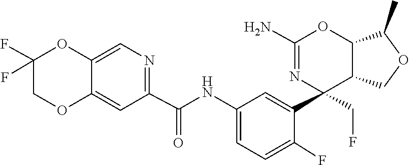 [SMILES: C[C@H]1OC[C@H]2[C@@H]1OC(N)=N[C@]2(CF)c1cc(NC(=O)c2cc3c(cn2)OC(F)(F)CO3)ccc1F] | ¹H NMR (400 MHz, CDCl₃) δ: 1.13 (3H, d, J = 6.8 Hz), 3.18- 3.25 (1H, m), 8.86- 3.98 (1H, m), 4.03 (1H, d, J = 4.8 Hz), 4.18 (1H, q, J = 6.8 Hz), 4.17-4.23 (1H, m), 4.29 (1H, br s), 4.42 (2H, t, J = 5.9 | 497 | 1.31 | B |
| | | Hz), 4.45-4.72 (2H, | | | |
| | | m), 7.11 (1H, dd, J = | | | |
| | | 11.7, 8.9 Hz), 7.52- | | | |
| | | 7.56 (1H, m), 7.96 | | | |
| | | (1H, s), 8.03-7.97 | | | |
| | | (1H, m), 8.33 (1H, s), | | | |
| | | 9.83 (1H, s). | | | |

TABLE 15

| | | | | | |
|---|---|---|---|---|---|
| I- 050 | 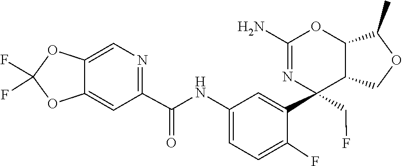 [SMILES: C[C@H]1OC[C@H]2[C@@H]1OC(N)=N[C@]2(CF)c1cc(NC(=O)c2cc3c(cn2)OC(F)(F)O3)ccc1F] | ¹H NMR (400 MHz, CDCl₃) δ: 1.13 (3H, d, J = 6.7 Hz), 3.17- 3.25 (1H, m), 3.89 (1H, t, J = 9.8 Hz), 4.03 (1H, d, J = 4.4 Hz), 4.10-4.16 (1H, m), 4.21 (1H, t, J = 8.1 Hz), 4.32 (11-1, br s), 4.45-4.72 (2H, m), | 483 | 1.43 | B |
| | | 7.12 (1H, dd, J = | | | |
| | | 11.4, 8.8 Hz), 7.53- | | | |
| | | 7.57 (1H, m), 8.02- | | | |
| | | 7.97 (1H, m), 8.09 | | | |
| | | (1H, s), 8.37 (1H, s), | | | |
| | | 9.80 (1H, s). | | | |
| I- 052 | 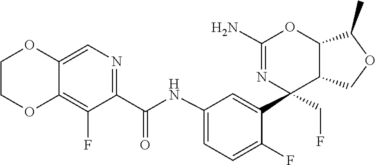 [SMILES: C[C@H]1OC[C@H]2[C@@H]1OC(N)=N[C@]2(CF)c1cc(NC(=O)c2ncc3c(c2F)OCCO3)ccc1F] | ¹H NMR (400 MHz, CDCl₃) δ: 1.12 (3H, d, J = 6.8 Hz), 3.18- 3.25 (1H, m), 3.89 (1H, t, J = 9.8 Hz), 4.02 (1H, d, J = 4.1 Hz), 4.12 (1H, q, J = 6.8 Hz), 4.17-4.23 (1H, m), 4.31 (1H, br s), 4.38-4.73 (6H, m), | 479 | 1.14 | B |
| | | 7.10 (1H, dd, J = | | | |
| | | 11.7, 8.8 Hz), 7.43 | | | |
| | | (1H, dd, J = 7.0, 2.7 | | | |
| | | Hz), 8.02 (1H, s), | | | |
| | | 8.11-8.06 (1H, m), | | | |
| | | 9.70 (1H, s). | | | |
| I- 053 | 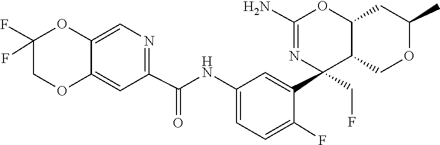 [SMILES: C[C@@H]1C[C@H]2OC(N)=N[C@](CF)(c3cc(NC(=O)c4cc5c(cn4)OC(F)(F)CO5)ccc3F)[C@H]2CO1] | ¹H NMR (400 MHz, CDCl₃) δ: 1.17 (3H, d, J = 6.3 Hz), 1.40- 1.47 (1H, m), 1.79- 1.85 (1H, m), 2.67- 2.72 (1H, m), 3.66- 3.73 (2H, m), 4.01- 4.07 (2H, m), 4.34 (2H, br s), 4.41 (2H, | 5.11 | 1.47 | B |
| | | t, J = 5.9 Hz), 4.64 | | | |
| | | (2H, d, J = 47.2 Hz), | | | |
| | | 7.08 (1H, dd, J = | | | |
| | | 11.5, 9.0 Hz), 7.49 | | | |
| | | (1H, dd, J = 6.8, 2.8 | | | |
| | | Hz), 7.95 (1H, s), | | | |
| | | 7.99-8.02 (1H, m), | | | |
| | | 8.32 (1H, s), 9.82 | | | |
| | | (1H, s). | | | |
| I- 054 | 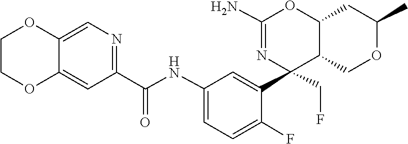 [SMILES: C[C@@H]1C[C@H]2OC(N)=N[C@](CF)(c3cc(NC(=O)c4cc5c(cn4)OCCO5)ccc3F)[C@H]2CO1] | ¹H NMR (400 MHz, CDCl₃) δ: 1.17 (3H, d, J = 6.3 Hz), 1.39- 1.46 (1H, m), 1.79- 1.84 (1H, m), 2.67- 2.72 (1H, m), 3.65- 3.73 (2H, m) 4.01- 4.08 (2H, m), 4.41- 4.36 (4H, m), 4.55- | 475 | 1.28 | B |
| | | 4.74 (2H , m) , 7.07 | | | |
| | | (1H, dd, J = 11.5, 8.8 | | | |
| | | Hz), 7.46 (1H, dd, J = | | | |
| | | 6.9, 2.6 Hz), 7.79 | | | |
| | | (1H, s), 8.00-8.04 | | | |
| | | (1H, m), 8.13 (1H, s), | | | |
| | | 9.86 (1H, s). | | | |

TABLE 16

| | | | | | |
|---|---|---|---|---|---|
| I- 057 | 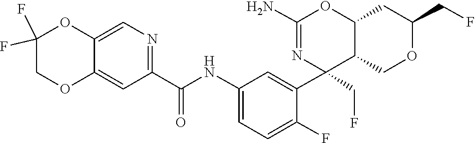 [SMILES: NC1=N[C@](CF)(c2cc(NC(=O)c3cc4c(cn3)OC(F)(F)CO4)ccc2F)[C@H]2CO[C@H](CF)C[C@H]2O1] | ¹H NMR (400 MHz, CDCl3) δ: 1.65-1.81 (2H, m), 2.74-2.77 (1H, m), 3.73 (1H, t, J = 11.8 z), 3.81- 3.91 (1H, m), 4.10- 4.17 (2H, m), 4.28- 4.74 (6H, m), 7.09 | 529 | 1.4 | B |
| | | (1H, dd, J = 11.5, 8.8 | | | |
| | | Hz), 7.53 (1H, dd, J = | | | |
| | | 6.9, 2.6 Hz), 7.95 | | | |
| | | (1H, s), 7.96-8.00 | | | |
| | | (1H, m), 8.25 (1H, 5), | | | |
| | | 8.32 (1H, s), 9.82 | | | |
| | | (1H, brs). | | | |
| I- 058 | 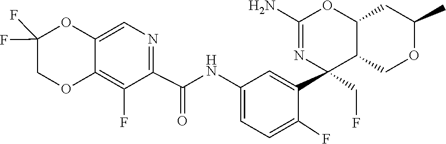 [SMILES: C[C@@H]1C[C@H]2OC(N)=N[C@](CF)(c3cc(NC(=O)c4ncc5c(c4F)OCC(F)(F)O5)ccc3F)[C@H]2CO1] | ¹H NMR (400 MHz, CDCl₃) δ: 1.17 (3H, d, J = 6.1 Hz), 1.47- 1.40 (1H, m), 1.79- 1.84 (1H, m), 2.68- 2.73 (1H, m), 3.65- 3.74 (2H, m), 4.00- 4.09 (2H, m), 4.36 (2H, hr s), 4.47-4.50 | 529 | 1.39 | B |
| | | (2H, m), 4.63 (2H, d, | | | |
| | | J = 47.1 Hz), 7.08 | | | |
| | | (1H, dd, J = 11.5, 8.8 | | | |
| | | Hz), 7.39 (1H, dd, J = | | | |
| | | 6.8, 2.8 Hz), 8.04- | | | |
| | | 8.08 (1H, m , 8.20 | | | |
| | | (1H, s), 9.64 1H, s). | | | |
| I- 060 | 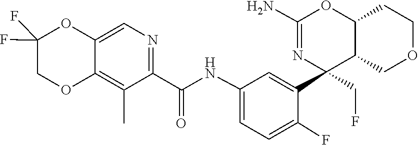 [SMILES: Cc1c(C(=O)Nc2ccc(F)c([C@@]3(CF)N=C(N)O[C@@H]4CCOC[C@@H]43)c2)ncc2c1OCC(F)(F)O2] | ¹H NMR (400 MHz, CDCl₃) δ: 1.67-1.90 (2H, m), 2.75(3H s), 2.76 (11-1, ddd, J = 11.5, 5.0, 2.3 Hz), 3.58-3.66 (2H, m) 3.77 (1H, dd, J = 11.0, 4.5 Hz), 3.99- 4.10 (2H, m), 4.42 | 511 | 1.43 | B |
| | | (2H, dd, J = 6.2, 5.7 | | | |
| | | Hz), 4.53-4.74 (2H, | | | |
| | | m), 7.07 (1H, dd, J = | | | |
| | | 11.5, 8.8 Hz), 7.37 | | | |
| | | (1H, dd, J = 6.8, 2.8 | | | |
| | | Hz), 8.04-8.09 (1H, | | | |
| | | m), 8.18 (1H, s), | | | |
| | | 10.00 (1H, s). | | | |
| I- 062 | 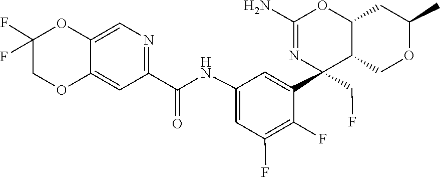 [SMILES: C[C@@H]1C[C@H]2OC(N)=N[C@](CF)(c3cc(NC(=O)c4cc5c(cn4)OC(F)(F)CO5)cc(F)c3F)[C@H]2CO1] | ¹H NMR (400 MHz, CDCl₃) δ: 1.39 (3H, s), 3.31-3.37 (1H, m), 3.91-3.99 (2H, m), 3.93 (3H, s), 4.32- 4.45 (5H, m), 5.07 (1H, d, J = 9.3 Hz), 7.07 (1H, t, J = 10.1 Hz), 7.40-7.45 (1H, m), 7.46 (1H, s), 7.61 | 529 | 1.55 | B |
| | | (1H, s), 7.80 (1H, s), | | | |
| | | 7.93-7.99 (1H, m), | | | |
| | | 8.14 (1H, s), 9.84 | | | |
| | | (1H, s). | | | |

TABLE 17

| | | M + H | tR | LC/MS |
|---|---|---|---|---|
| No. | Structure | observed | (min) | method |
| III-004 | 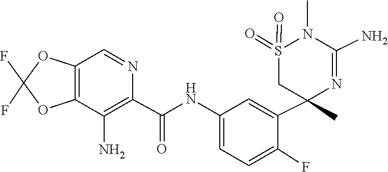 [SMILES: CN1C(N)=N[C@](C)(c2cc(NC(=O)c3ncc4c(c3N)OC(F)(F)O4)ccc2F)CS1(=O)=O] | 487.1 | 1.36 | B |
| III-005 | 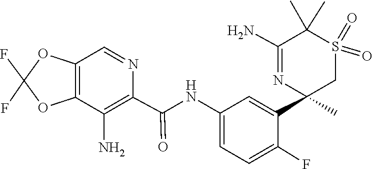 [SMILES: CC1(C)C(N)=N[C@](C)(c2cc(NC(=O)c3ncc4c(c3N)OC(F)(F)O4)ccc2F)CS1(=O)=O] | 500.1 | 1.35 | B |

TABLE 18

| No. | NMR |
|---|---|
| III-004 | 1H-NMR (CDCl3) δ: 1.78 (3H, s), 3.23 (3H, s), |
| | 3.69-3.86 (2H, m), 6.03 (2H, br), |
| | 7.64 (1H, dd, J = 8.8, 11.6 Hz), |
| | 7.68-7.72 (1H, m), 7.74 (1H, s), 7.78-7.81 (1H, |
| | m), 9.80 (1H, br) |
| III-005 | 1H-NMR (CDCl3) δ: 1.63 (3H, s), 1.71 (3H, s), |
| | 1.80 (3H, s), |
| | 3.50-3.65 (2H, m), 6.06 (2H, br), 7.08 (1H, dd, J = |
| | 8.4, 11.6 Hz), |
| | 7.63 (1H, br), 7.77 (2H, m), 9.81 (1H, s) |

Hereinafter, the term “m.p.” means melting point, “min” means minutes, “aq.” means aqueous, “r.m.” or “RM” means reaction mixture, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “RP” means reversed phase, “UPLC” means ultra-performance liquid chromatography, “DAD” “means Diode Array Detector, “DSC” means differential scanning calorimetry, “SQD” means Single Quadrupole Detector, “NaH” means sodium hydride, “BEH” means bridged ethylsiloxane/silica hybrid, “CSH” means charged surface hybrid “Rₜ” means retention time (in minutes), “[M+H]⁺” means the protonated mass of the free base of the compound, “wt” means weight, “NaHCO₃” means sodium bicarbonate, “Na₂CO₃” means sodium carbonate, “K₂CO₃” means potassium carbonate, “DMAP” means N,N-dimethylpyridin-4-amine, “M” means molar, “THF” means tetrahydrofuran, “EtOAc” means ethyl acetate, “MeCN” means acetonitrile, “BuLi” means butyl lithium, “h” means hours, “Et₂O” means diethyl ether, “DCM” means dichloromethane, “DMF” means N,N-dimethylformamide, “KF” means potassium fluoride, “KNO₃” means potassium nitrate, “H₂SO₄” means sulfuric acid, “BH₃-THF” means borane-tetrahydrofuran complex, “MeOH” means methanol, “Et₃N” means triethylamine, “org.” means organic, “OL” means organic layer, “N” means normal, “MeI” means iodomethane, “AcCl” means acetyl chloride, “sol.” means solution, “BOC” means tert-butoxycarbonyl, “TLC” means thin layer chromatography, “EtOH” means ethanol, “iPrNH₂” means isopropylamine, “DIPE” means diisopropyl ether, “NH₄Ac” means ammonium acetate, “iPrOH” means isopropanol, and “EDCI” means 1-ethyl-3-(3-dimethylaminopropyl)carbodiimide, “TFA” means trifluoroacetic acid, “ACN” means acetonitrile, “NP” means normal phase, “TPP” means triphenylphosphine, “DPPP” means 1,3-bis(diphenylphosphino)propane, “TMP” means 2,2,6,6-tetramethylpiperidine, “DIAD” means diisopropyl azodicarboxylate, “DMEAD” means di-2-methoxyethyl azodicarboxylate, “TEMPO” means (2,2,6,6-tetramethylpiperidin-1-yl)-N-oxide, “DBU” means 1,8-diazabicyclo[5.4.0]undec-7-ene, “NCS” means N-chlorosuccinimide, “Celite (Registered trademark)” and “Dicalite (Registered trademark)” are diatomaceous earths, “Teflon (Registered trademark)” means polytetrafluoroethylene, and Hastelloy (Registered trademark) metals are corrosion resistant nickel alloys.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds. The enantiomeric excess of compounds reported herein was determined by analysis of the racemic mixture by supercritical fluid chromatography (SFC) followed by SFC comparison of the separated enantiomer(s).

The absolute configuration of chiral centres (indicated as R and/or S) can be rationalized. The synthesis of all final compounds started from intermediates of known absolute configuration in agreement with literature precedent (e.g. intermediate 20) or obtained from appropriate synthetic procedures (e.g. the formation of Ellman's sulfonamide in intermediate 3). The assignment of the absolute configuration of additional stereocentres could then be assigned by standard NMR methods.

### Preparation of the Acid Intermediates of Formula (XXV)

### Preparation of Intermediate A5″

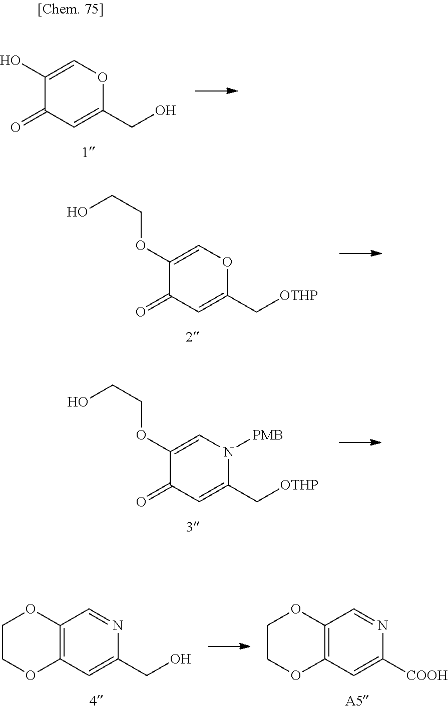

### Step 1

To a solution of 1″ (42.6 g, 300 mmol) in THF (171 mL) were added 3,4-dihydro-2H-pyran (30.2 mL, 330 mL) and p-toluenesulfonic acid monohydrate (228 mg, 1.20 mmol). After being stirred for 3 days at room temperature, the reaction mixture was quenched with a solution of sodium hydroxide (0.900 mL, 1.80 mmol, 2 mol/L in water). The mixture was evaporated to dryness. To a solution of the resulting residue in ethanol (85 mL) was added a solution of sodium hydroxide (42 mL, 360 mmol, 8.57 mol/L in water) and washed with ethanol (85 mL). After being stirred for 15 min at room temperature, to the reaction mixture was added 2-chloroethanol (81 mL, 1.20 mol). After being stirred for 7 h at 100° C., to the reaction mixture was added an additional solution of sodium hydroxide (30 mL, 240 mmol) and stirred for an additional 2.5 h at 100° C. The reaction mixture was evaporated to remove the ethanol. The residue was diluted with water, extracted with toluene, and washed with brine. The combined organic layers were dried over sodium sulfate and evaporated. To a solution of the residue in toluene (200 mL) was added activated carbon (40 g), filtered through diatomaceous earth, and evaporated to give 2″ (88.6 g) as a brown oil. It was used for the next reaction without further purification. ¹H NMR (400 MHz, CDCl₃) δ: 1.53-1.91 (m, 6H), 3.53-3.58 (m, 1H), 3.79-3.93 (m, 3H), 3.98-4.03 (m, 2H), 4.35 (d, J=14.4 Hz, 1H), 4.54 (d, J=14.4 Hz, 1H), 4.72-4.75 (m, 1H), 6.55 (s, 1H), 7.71 (s, 1H).

### Step 2

To a solution of crude 2″ (44.2 g) in ethanol (88 mL) was added p-methoxybenzylamine (39.2 mL, 300 mmol). After being stirred for 19 h at 100° C., the reaction mixture was diluted with ethyl acetate. The mixture was evaporated to dryness. The crude product was triturated with ethyl acetate to give 3″ (29.7 g, 76% over 2 steps) as a white solid. ¹H NMR (400 MHz, DMSO-d₆) δ: 1.39-1.67 (m, 6H), 3.41-3.47 (m, 1H), 3.59-3.64 (m, 2H), 3.67-3.74 (m, 1H), 3.73 (s, 3H), 3.85-3.89 (m, 2H), 4.32 (d, J=12.9 Hz, 1H), 4.51 (d, J=12.9 Hz, 1H), 4.62-4.66 (m, 1H), 6.30 (s, 1H), 6.94 (d, J=8.5 Hz, 2H), 7.10 (d, J=8.5 Hz, 2H), 7.65 (s, 1H).

### Step 3

To a suspension of 3″ (29.6 g, 76 mmol) in dichloromethane (296 mL) were added triethylamine (31.6 mL, 228 mmol) and methanesulfonylchloride (8.89 mL, 114 mmol) at 0° C. After being stirred for 2 h at room temperature, the reaction mixture was evaporated to dryness. To a solution of the residue in trifluoroacetic acid (266 mL, 3.45 mol) was added triethylsilane (29.6 mL, 185 mmol). After being stirred for 45 min at 120° C., the reaction mixture was evaporated to distill off c.a. 60 mL of a solvent. After being stirred for additional 45 min at 140° C., the reaction mixture was evaporated to distill off additional c.a. 60 mL of a solvent. After being stirred for additional 60 min at 140° C., the reaction mixture was evaporated. The residue was basified with a solution of sodium hydroxide and extracted with dichloromethane, chloroform, and a mixture of methanol-chloroform (1:9). The combined organic layers were evaporated. The residue was diluted with toluene and extracted with a solution of hydrochloric acid (2 mol/L in water) and water, washed with toluene. The combined aqueous layers were basified with a solution of sodium hydroxide, extracted with chloroform and a mixture of methanol-chloroform (1:4), washed with brine. The combined organic layers were dried over sodium sulfate, evaporated to give 4″ (10.4 g, 62.0 mmol, 82%) as a white solid. ¹H NMR (400 MHz, CDCl₃) δ: 4.27-4.31 (m, 2H), 4.33-4.36 (m, 2H), 4.62 (s, 2H), 6.77 (s, 1H), 8.12 (s, 1H).

### Step 4

To a solution of 4″ (5.01 g, 30 mmol), sodium dihydrogen phosphate (11.0 g, 92 mmol), disodium hydrogen phosphate (4.33 g, 30.5 mmol), and sodium chlorite (10.2 g, 90 mmol) in water (50.1 mL) and acetonitrile (50.1 mL) were added TEMPO (469 mg, 3.0 mmol) and a solution of sodium hypochlorite (0.185 mL, 0.150 mmol, 5 w/w % in water) at 35° C. After being stirred for 1.5 h at 40° C., the reaction mixture was quenched with a solution of sodium hydrogen sulfite, neutralized with a solution of sodium hydroxide, and evaporated to distill off the acetonitrile. The suspension was filtered to give a crude product. The crude product was triturated with water and filtered to give A5″ (4.28 g, 23.6 mmol, 79%) as a white solid. ¹H NMR (400 MHz, CDCl₃) δ 4.30-4.42 (m, 4H), 7.52 (s, 1H), 8.21 (s, 1H).

### Preparation of Intermediate A9″

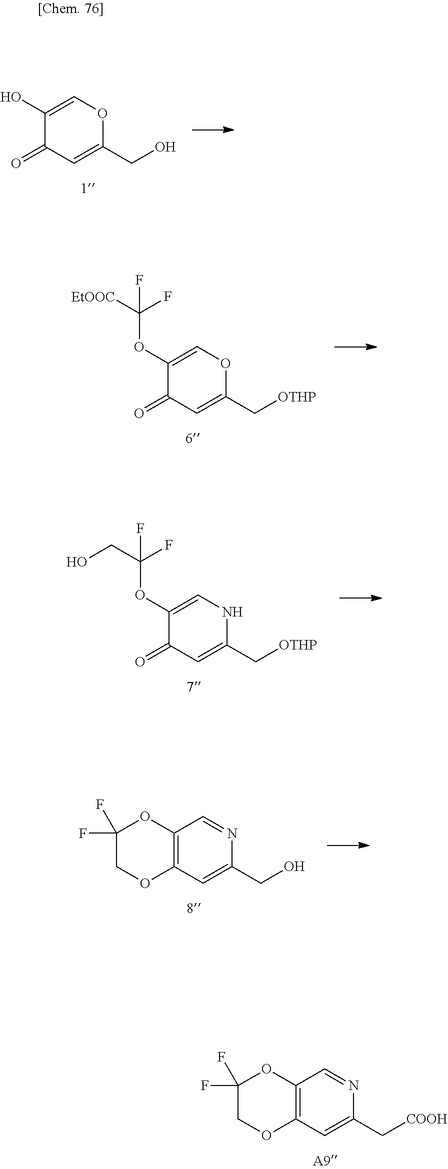

### Step 1

To a solution of 1″ (120 g, 844 mmol) in THF (480 mL) were added 3,4-dihydro-2H-pyran (81 mL, 887 mmol) and p-toluenesulfonic acid monohydrate (642 mg, 3.38 mmol). After being stirred for 27.5 h at room temperature, to the reaction mixture were added DBU (129 mL, 853 mmol) and ethyl bromodifluoroacetate (162 mL, 1.27 mol) at 5° C. After being stirred for 1 h at 5° C. room temperature and then for 4 h at room temperature, the reaction mixture was diluted with sodium dihydrogen phosphate (1.50 L, 2.25 mol, 1.5 mol/L in water), extracted with ethyl acetate, and washed with water. The combined organic layers were added to activated carbon (90 g), filtered through diatomaceous earth, and evaporated to give 6″ (257.3 g, 680 mmol, 81%) as a brown oil. It was used for the next reaction without further purification. ¹H NMR (400 MHz, CDCl₃) δ: 1.40 (t, J=7.2 Hz, 3H), 1.50-1.92 (m, 6H), 3.53-3.59 (m, 1H), 3.78-3.86 (m, 1H), 4.35 (d, J=14.6 Hz, 1H), 4.41 (q, J=7.2 Hz, 2H), 4.55 (d, J=14.6 Hz, 1H), 4.72-4.75 (m, 1H), 6.57 (s, 1H), 7.99 (s, 1H).

### Step 2

To a solution of 6″ (207 g, 594 mmol) in THF (1000 mL) and water (1000 mL) was added sodium borohydride (22.5 g, 594 mmol) portionwise over 30 min at 0° C. After being stirred for 2 h at 0° C., the reaction mixture was quenched with a saturated solution of ammonium chloride, extracted with ethyl acetate, and washed with water and brine. The combined organic layers were dried over sodium sulfate and evaporated to give the crude product. To a solution of the crude product in ethanol (500 mL) was added ammonium hydroxide (207 mL, 2.70 mol). After being stirred for 4 h at 60° C., the reaction mixture was evaporated. The residue was triturated with ethyl acetate to give 7″ (43.0 g, 141 mmol, 24%) as a white solid. ¹H NMR (400 MHz, CDCl₃) δ:1.54-1.92 (m, 6H), 3.56-3.64 (m, 1H), 3.69-3.76 (m, 1H), 3.98-4.06 (m, 1H), 4.61-4.65 (m, 1H), 4.66 (s, 2H), 6.34-6.47 (br, 1H), 6.48 (s, 1H), 7.71 (s, 1H), 10.3 (s, 1H)

### Step 3

To a solution of 7″ (5.32 g, 17.4 mmol) and triphenylphosphine (5.94 g, 22.7 mmol) in THF (26.6 mL) was added DIAD (11.9 mL, 22.7 mmol, 1.9 mol/L in toluene) at 0° C. After being stirred for 2.5 h at room temperature, to the reaction mixture were added triphenylphosphine (1.37 g, 5.23 mmol) and DIAD (2.75 mL, 5.23 mmol, 1.9 mol/L in toluene). After being stirred for 30 min at room temperature, the reaction mixture was evaporated. The residue was diluted with a mixture of DMF/water (2:1), extracted with a mixture of n-heptane/toluene (2:1), and washed with water. The combined organic layers were evaporated to the crude product. To a solution of the crude product in methanol (13.3 mL) was added hydrogen chloride (13.7 mL, 26.1 mol, 2 mol/L in water). After being stirred for 1 h at room temperature, the reaction mixture was extracted with water, washed with ethyl acetate. The combined aqueous layers were basified with a solution of sodium hydroxide, extracted with ethyl acetate, washed with water. The combined organic layers were evaporated to give 8″ (2.52 g, 12.4 mmol, 71%). It was used for the next reaction without further purification. ¹H NMR (400 MHz, CDCl₃) δ: 3.18-3.25 (br, 1H), 4.35 (t, J=9.0 Hz, 2H), 4.69 (s, 2H), 6.95 (s, 1H), 8.30 (s, 1H).

### Step 4

To a solution of 8″ (2.52 g, 12.4 mmol), sodium dihydrogen phosphate (4.54 g, 37.9 mmol), disodium hydrogen phosphate (1.79 g, 12.6 mmol), and sodium chlorite (4.21 g, 37.2 mmol) in water (25.2 mL) and acetonitrile (25.2 mL) were added TEMPO (194 mg, 1.24 mmol) and a solution of sodium hypochlorite (0.076 mL, 0.062 mmol, 5 w/w % in water) at 35° C. After being stirred for 15 min at 40° C., to the reaction mixture was added additional solution of sodium hypochlorite (0.076 mL, 0.062 mmol, 5 w/w % in water). After being stirred for 30 min at 40° C., to the reaction mixture was diluted with hydrogen chloride (2 mol/L in water), extracted with a mixture of ethylacetate/THF (1:1) and washed with a solution of sodium hydrogen sulfite and brine. The combined organic layers were dried over sodium sulfate, evaporated to give a crude product. The residue was triturated with ethyl acetate and methanol to give A9″ (2.46 g, 11.3 mmol, 91%) as a white solid. ¹H NMR (400 MHz, DMSO-d₆) δ: 4.82 (t, J=6.4 Hz, 2H), 7.76 (s, 1H), 8.55 (s, 1H).

### Preparation of Intermediate A18″

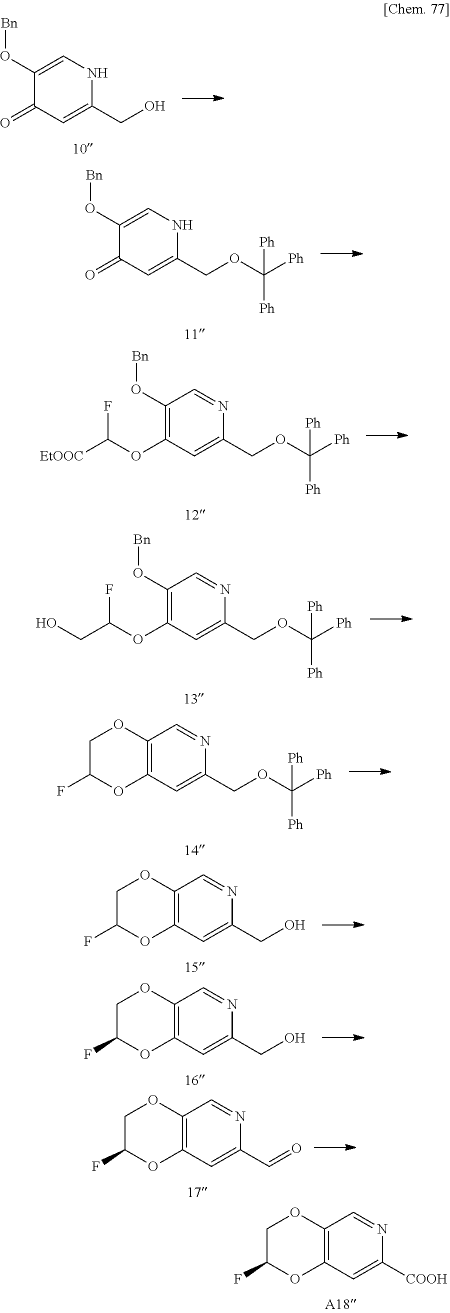

### Step 1

A suspension of 10″ (Australian Journal of Chemistry 1977, 30, 649-55) (20.81 g, 90.0 mmol), chlorotriphenylmethane (27.6 g, 99.0 mmol) and DMAP (12.1 g, 99.0 mmol) in DMF (125 mL) was stirred for 5 h at 95° C. After being stirred at 15° C., to the mixture was added cooled water (500 mL). After being stirred at room temperature for several minutes, the resulting solid was collected, which was then triturated with water and methanol to give 11″ (30.5 g, 64.4 mmol, 72%) as a white solid. ¹H NMR (400 MHz, DMSO-d₆) δ: 3.96 (s, 2H), 5.05 (s, 2H), 7.22-7.63 (m, 22H), 10.2-11.6 (br, 1H).

### Step 2

To a suspension of 11″ (15.3 g, 32.4 mmol) in DMF (153 mL) were added ethyl 2-bromo-2-fluoroacetate (7.59 mL, 64.8 mmol) and cesium carbonate (21.11 g, 64.8 mmol). After being stirred for 1 h at 80° C., the mixture was quenched with a saturated solution of ammonium chloride and extracted with ethyl acetate. The combined organic layers were washed with water, a saturated solution of sodium bicarbonate, and brine. The mixture was dried over magnesium sulfate and evaporated, and the crude product was purified by flash column chromatography (silica gel, 100:0-3:2 hexane/ethyl acetate) to give 12″ (13.8 g, 23.8 mmol, 74%) as a white solid. ¹H NMR (400 MHz, DMSO-d6) δ: 1.34 (t, J=7.2 Hz, 3H), 4.28 (s, 2H), 4.36 (q, J=7.2 Hz, 2H), 5.17 (s, 2H), 6.13 (d, J=58 Hz, 1H), 7.18-7.54 (m, 21H), 8.18 (s, 1H).

### Step 3

To a suspension of 12″ (13.8 g, 23.8 mmol) in THF (138 mL), ethanol (69 mL) and water (14 mL) was added sodium borohydride (1.80 g, 47.6 mmol) at 0° C. After being stirred for 15 min at 0° C. and then for 1 h at room temperature, the mixture was quenched with a saturated solution of ammonium chloride and extracted with ethyl acetate. The combined organic layers were washed with water and brine. The mixture was dried over magnesium sulfate and evaporated, and the crude product was purified by flash column chromatography (silica gel, 100:0-3:2 hexane/ethyl acetate) to give 13″ (11.6 g, 21.6 mmol, 91% yield) as a white solid. ¹H NMR (400 MHz, DMSO-d₆) δ: 3.78 (ddd, J=16, 6.1, 4.4 Hz, 2H), 4.06 (d, J=15 Hz, 1H), 4.10 (d, J=15 Hz, 1H), 5.23 (s, 2H), 5.47 (t, J=6.1 Hz, 1H), 6.18 (dt, J=61, 4.4 Hz, 1H), 7.26-7.44 (m, 21H), 8.26 (s, 1H).

### Step 4

To a solution of 13″ (11.57 g, 21.60 mmol) in THF (110 mL) was added palladium on carbon (0.861 g, 0.383 mmol, 10 w/w %). After being stirred for 2 h at room temperature under hydrogen (1 atm), The reaction mixture was filtered and evaporated. To a solution of the residue in THF (96 mL) were added triphenylphosphine (8.48 g, 32.3 mmol) and DMEAD (7.57 g, 32.3 mmol) at 0° C. After stirred for 10 min at 0° C. and for 2 h at room temperature, the mixture was quenched with cooled water and extracted with ethyl acetate. The combined organic layers were washed with water and brine. The mixture was dried over magnesium sulfate and evaporated, and the crude product was purified by flash column chromatography (silica gel, 100:0-1:1 hexane/ethyl acetate) to give 14″ (6.60 g, 15.4 mmol, 72% yield) as a white foam. ¹H NMR (400 MHz, CDCl₃) δ: 4.07 (dd, J=28, 12 Hz, 1H), 4.22 (d, J=17 Hz, 1H), 4.26 (d, J=17 Hz, 1H), 4.46 (dd, J=12, 1.4 Hz, 1H), 6.10 (d, J=53 Hz, 1H), 7.22-7.51 (m, 16H), 8.13 (s, 1H).

### Step 5

To a solution of 14″ (6.60 g, 15.4 mmol) in methanol (66 mL) was added p-toluenesulfonic acid mono hydrate (4.41 g, 23.2 mmol). After being stirred for 2 h at 70° C., the mixture was quenched with triethylamine (6.42 mL, 46.3 mmol) and evaporated. The crude product was purified by flash column chromatography (silica gel, 100:0-46:4 chloroform:methanol) to give 15″ (2.64 g, 14.3 mmol, 92% yield) as a white solid. ¹H NMR (400 MHz, DMSO-d6) δ:4.16 (dd, J=30, 13 Hz, 1H), 4.44 (d, J=15 Hz, 1H), 4.46 (d, J=15 Hz, 1H), 4.54 (dd, J=13, 4.3 Hz, 1H), 5.39 (t, J=6.0 Hz, 1H), 6.42 (d, J=52 Hz, 1H), 7.67 (s, 1H), 8.14 (s, 1H).

### Step 6

15″ (3.75 g, 20.3 g) was purified by SFC to give 16″ (1.75 g, 9.45 mmol, 47% yield) as a white solid. ¹H NMR (400 MHz, DMSO-d6) δ: 4.16 (dd, J=30, 13 Hz, 1H), 4.44 (d, J=15 Hz, 1H), 4.46 (d, J=15 Hz, 1H), 4.54 (dd, J=13, 4.3 Hz, 1H), 5.39 (t, J=6.0 Hz, 1H), 6.42 (d, J=52 Hz, 1H), 7.67 (s, 1H), 8.14 (s, 1H).

### Step 7

To a solution of 16″ (1.75 g, 9.45 mmol) in dichloromethane (44 mL) was added manganese dioxide (9.86 g, 113 mmol). After stirred for 3 h at room temperature. the reaction mixture was filtered and evaporated. The crude product was triturated with diisopropylether to give 17″ (1.40 g, 7.64 mmol, 81% yield) as a white solid. ¹H NMR (400 MHz, DMSO-d₆) δ: 4.34 (dd, J=30, 13 Hz, 1H), 4.69 (dd, J=13. 1.0 Hz, 1H), 6.53 (d, J=52 Hz, 1H), 7.61 (s, 1H), 8.49 (s, 1H), 9.85 (s, 1H).

### Step 8

To a solution of 17″ (1.40 g, 7.64 mmol) in acetone (42 mL) and water (14 mL) were added 2-methylbut-2-ene (1.45 mL, 13.7 mmol), sodium dihydrogen phosphate (246 mg, 2.048 mmol) and sodium chlorite (494 mg, 4.10 mmol). After being stirred for 30 min at 0° C. and for 1.5 h at room temperature, to the mixture was added a solution of hydrogen chloride (2 mol/L in water) and evaporated to remove acetone. The aqueous mixture was extracted with chloroform/methanol (1:4). The combined organic layers were dried over magnesium sulfate and evaporated, and the obtained solid was triturated with chloroform to give a crude product. The crude product was added to chloroform/methanol (3:7). The mixture was filtered to remove an insoluble material. The filtrate was evaporated and triturated with chloroform to give A18″ (992 mg, 4.98 mmol, 65.2% yield) as a white solid. ¹H NMR (400 MHz, DMSO-d6) δ: 4.30 (dd, J=30, 13 Hz, 1H), 4.64 (dd, J=13, 4.0 Hz, 1H), 6.50 (d, J=52 Hz, 1H), 7.68 (s, 1H), 8.35 (s, 1H).

### Preparation of Intermediate A24″

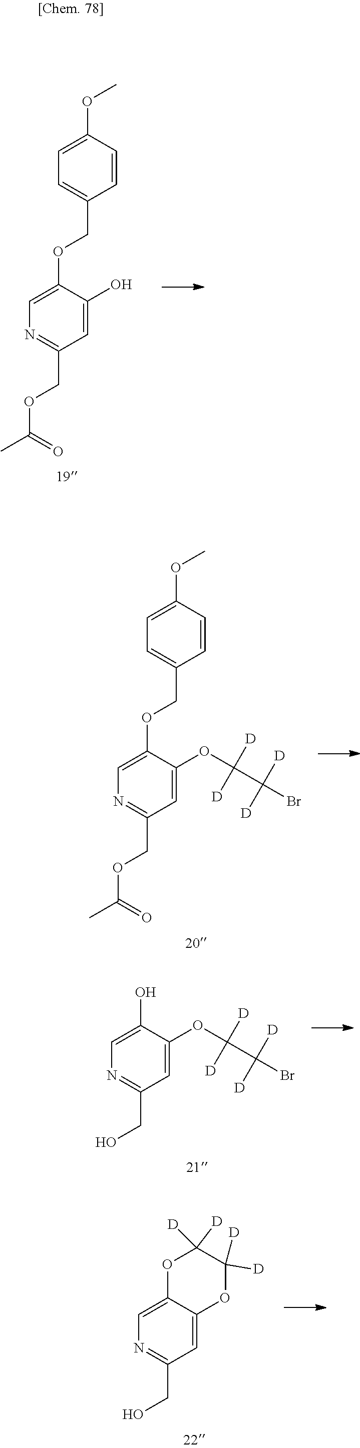 [SMILES: COc1ccc(COc2cnc(COC(C)=O)cc2O)cc1 ; [2H]C([2H])(Br)C([2H])([2H])Oc1cc(COC(C)=O)ncc1OCc1ccc(OC)cc1 ; [2H]C([2H])(Br)C([2H])([2H])Oc1cc(CO)ncc1O ; [2H]C1([2H])Oc2cnc(CO)cc2OC1([2H])[2H]]

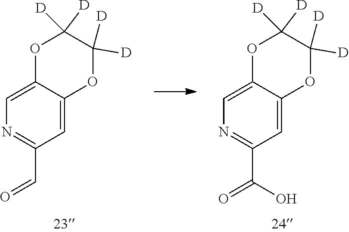 [SMILES: [2H]C1([2H])Oc2cnc(C=O)cc2OC1([2H])[2H] ; [2H]C1([2H])Oc2cnc(C(=O)O)cc2OC1([2H])[2H]]

### Step 1

A mixture of intermediate 19″ (Journal of Medicinal Chemistry 2013, 56, 5541-5552; WO 2004058144) (5.00 g, 16.5 mmol), 1,2-dibromoethane-d4 (7.91 g, 41.2 mmol, CAS 22581-63-1), K₂CO₃ (4.56 g, 33.0 mmol) in DMF (40 mL) was stirred at 75° C. for 2 hours. The mixture was then evaporated under vacuum. Water (50 mL) was added to the residue. The mixture was extracted 3× with ethyl acetate (60 mL). The organic layer was dried (MgSO₄), filtered and concentrated under vacuum. The residue was purified by column chromatography over silica gel (80 g) using a gradient (heptane/EtOAc, 1:0 to 3:7). The pure fractions were collected and the solvent was evaporated under vacuum to give the product 20″ as a white solid (3.58 g, 52%).

### Step 2

Concentrated HCl (37% in water, 10 mL, 119.7 mmol) was added to a mixture of intermediate 20″ (3.58 g, 8.65 mmol) and acetic acid (20 mL, 349.4 mmol) at 0° C. The mixture was then stirred at 60° C. overnight. The mixture was concentrated under vacuum to afford the crude product 21″ as a yellow solid (3.67 g, 59% pure) which was used as such in the next step.

### Step 3

A mixture of intermediate 21″ (3.67 g, 8.6 mmol, 59% pure), K₂CO₃ (2.37 g, 17.2 mmol) in DMF (40 mL) was stirred at 75° C. for 3 hours. Next, the mixture was evaporated under vacuum. To the residue was added water (25 mL) and the aq. Layer was extracted 3 times with ethyl acetate (30 mL). The combined organic layers were dried with MgSO₄, filtered and concentrated under vacuum to afford the crude product as a brown oil. To deprotect remaining acetylated product from the previous step, 1 N NaOH in water (4.29 mL, 4.29 mmol) was added and the mixture stirred 10 minutes after which it was extracted 3 times with EtOAc (30 mL). The combined organic layers were collected, dried (MgSO₄), filtered and evaporated to dryness to afford a brown oil. This oil was purified by flash column chromatography (12 g silica) using a gradient (DCM/MeOH from 10:0 to 9:1). The desired fractions were collected and evaporated under vacuum yielding intermediate 22″ (1.2 g, 82%) as an orange semi-crystalline solid.

### Step 4

To a mixture of intermediate 22″ (1.20 g, 5.47 mmol) in DCM (15 mL) was added MnO₂ (2.38, 27.3 mol). The reaction mixture was stirred at room temperature overnight after which it was filtered over a pad of Dicalite(Registered trademark). The filtrate was concentrated under vacuum to give the desired product 23″ as a white solid (858 mg, 93%), which was used as such for the next step.

### Step 5

Intermediate 23″ (480 mg, 2.84 mmol) was dissolved in acetone (14 mL) and water (4.7 mL) and the solution was cooled at 0-5° C. 2-Methyl-2-butene (3.0 mL, 28.4 mmol), sodium dihydrogenphosphate (511 mg, 4.1 mmol) and sodium chlorite (962 mg, 8.5 mmol) were then added. The suspension was stirred at 0-5° C. for 30 minutes and then at r.t. for 3 h. The acetone was roughly removed under reduced pressure. The resulting suspension was cooled to 0° C. and a 2 N HCl solution was added until the reaction mixture became a clear yellow solution. The mixture was extracted several times using a solution MeOH/DCM (1:4). The combined organic layers were evaporated yielding intermediate A24″ as a white solid (501 mg, 95%).

### Preparation of Intermediate A25″

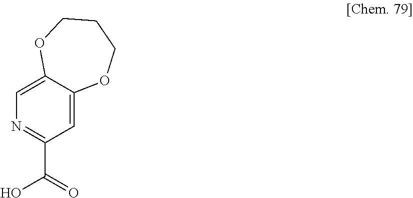 [SMILES: O=C(O)c1cc2c(cn1)OCCCO2]

Intermediate A25″ was prepared following the same synthesis route as described hereinabove for intermediate A24″, but using 1,3-dibromopropane.

### Preparation of Intermediate A30″

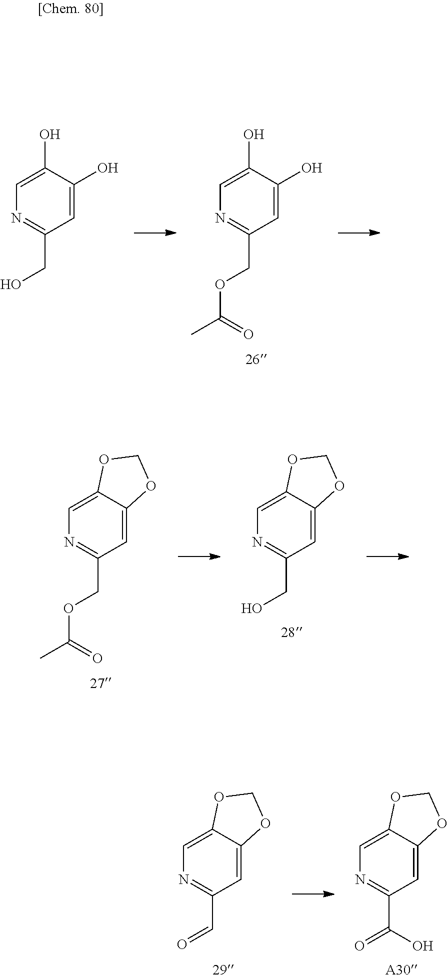

### Step 1

A mixture of 6-hydroxymethyl-3,4-pyridinediol (20 g, 65.9 mmol) in acetic acid (50 mL) was stirred and heated at 60° C. for 6 h. The mixture was then concentrated. Methyl tert-butyl ether (50 mL) was added and the mixture was stirred at room temperature for 0.5 hour. The precipitate was filtered, and dried in vacuo to give the product 26″ (5.0 g, 75%).

### Step 2

A mixture of intermediate 26″ (5.0 g, 20.6 mmol), chloroiodomethane (5.4 g, 30.8 mmol) and K₂CO₃ (6.0 g, 43.2 mmol) in DMF (120 mL) was stirred at 80° C. for 2 h. Next, the reaction was concentrated. Water (150 mL) was added and the mixture was extracted with ethyl acetate (3×100 mL). The organic layer was dried (Na₂SO₄), filtered and concentrated to give the crude product 27″ (2.0 g, 50%), which was used as such.

### Step 3

A mixture of intermediate 27″ (2.0 g, 10.2 mmol) in HCl (35.5% in water, 40 mL) and acetic acid (5 mL, 87.3 mmol) was stirred at 100° C. for 12 hours. Next, the mixture was concentrated. Water was added to the residue and the pH adjusted to 8 with NaHCO₃ (saturated aq. solution). The mixture was extracted with ethyl acetate (3×100 mL). The combined org. layers were dried (Na₂SO₄), filtered and concentrated to give the crude product 28″ (500 mg, 32%), which was used as such.

### Step 4

A mixture of intermediate 28″ (1.00 g, 6.53 mmol) and MnO₂ (2.84 g, 32.65 mmol) in DCM (50 mL) was stirred at room temperature for 16 hours. Next, the mixture was filtered through a Celite (Registered trademark) pad. The filtrate was concentrated to give the crude product 29″ (1.30 g, quantitative).

### Step 5

A mixture of intermediate 29″ (1.3 g 8.60 mmol) in acetone (2 mL) and water (10 mL) was stirred at room temperature. NaClO₂ (1.01 g, 11.18 mmol) was added and the mixture was stirred 5 min. Sulfamic acid (1.13 g, 11.61 mmol) was added and the mixture was stirred at room temperature for 2 hours. The formed precipitate was filtered, washed with water (10 mL) and dried in vacuo at 60° C. (12 hours) yielding intermediate A30″ (670 mg, 45%).

### Preparation of Intermediate A34″

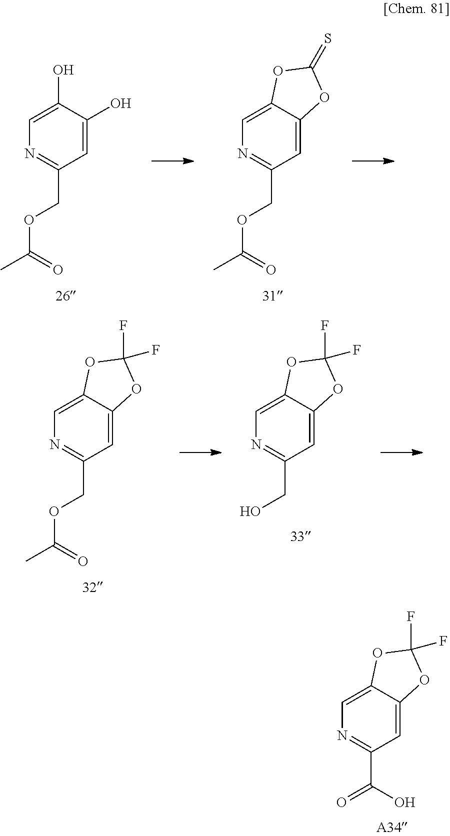 [SMILES: CC(=O)OCc1cc(O)c(O)cn1 ; CC(=O)OCc1cc2oc(=S)oc2cn1 ; CC(=O)OCc1cc2c(cn1)OC(F)(F)O2 ; OCc1cc2c(cn1)OC(F)(F)O2 ; O=C(O)c1cc2c(cn1)OC(F)(F)O2]

### Step 1

Thiophosgene (17 mL, 227 mmol) was added slowly to a suspension of intermediate 26″ (28 g, 151 mmol) and DMAP (37 g, 302 mmol) in DCM (0.8 L) stirred at 0° C. under N₂. During the addition, the formation of a light red precipitate was immediately observed. The reaction was allowed to warm to r.t. and stirred for 2 h, after which it was diluted with water. The organic layer was separated and the aqueous one extracted with DCM (3×100 mL). The combined organic layers were washed with brine, dried over MgSO₄, filtered and solvents were evaporated to yield a dark brown oil. The crude was purified by flash chromatography (SiO₂, EtOAc:DCM, 0:100 to 35:75). After evaporation of the product fractions, intermediate 31″ was obtained as an orange crystalline solid (27.5 g, 81%).

### Step 2

THF (70% in pyridine, 100 g, 3481 mmol) was added dropwise to a stirred solution of intermediate 31″ (16 g, 71.0 mmol) in DCM (322 mL) under nitrogen at −78° C. in a polypropylene vessel by using a plastic syringe. Caution: HF reacts vigorously with glassware at r.t., hence glassware needs to be avoided. Then 1,3-dibromo-5,5-dimethylhydantoin (61 g, 213 mmol) was subsequently added portionwise and the mixture was stirred at −78° C. for 20 min. Then the cooling bath was replaced by one with ice-NaCl and reaction was stirred for 1 h. The cooled mixture was quenched by careful addition of 50% NaOH solution until the pH became neutral. Then Na₂S₂O₃ (10% aq. solution, 40 mL) was added. The mixture was filtered to remove the white solid, which was washed with DCM. The filtrate was extracted with DCM (2×) The combined organic layers were washed with water, dried (MgSO₄), filtered and the solvents evaporated in vacuo. To remove the pyridine was residue was codistilled with toluene (50 mL). The crude product was purified by flash column chromatography (silica; EtOAc in heptane 0/100 to 30/70). The desired fractions were collected and concentrated in vacuo to yield intermediate 32″ as a pale yellow oil (15 g, 91%).

### Step 3

A mix of intermediate 32″ (10.0 g, 43.3 mmol) and K₂CO₃ (12 g, 86.5 mmol) in MeOH (228 mL) was heated to reflux for 1 h. Next the solvent was removed under reduced pressure. The residue was dissolved in DCM and water. The org. layer was separated and the aq. layer was extracted with DCM. The combined org. layers were dried with MgSO₄, filtered and the solvent was removed under reduced pressure. Caution: the product is volatile. Intermediate 33″ was obtained as a light yellow oil (6.7 g, 82%) and used in next step without further purification.

### Step 4

TEMPO (0.78 g, 4.96 mmol) was added to a mixture of intermediate 33″ (6.7 g, 35 mmol) in phosphate buffer (pH 7, 94 mL) and ACN (105 mL). The reaction was stirred at 35° C. Sodium chlorite (20 g, 177 mmol) in NaOCl (15% aq. solution, 58 mL, 141 mmol) and water (28 mL) were added simultaneously in three slow additions each 30 min. The resulting reaction mixture was stirred at 35° C. for 16 hours. The pH was then adjusted to 8 by addition of 1 N aq. NaOH solution. Aq. saturated Na₂S₂O₃ solution was added until the r.m. turned white, and stirring was continued for 30 min. The pH was adjusted to 4 by addition of a 1 N HCl solution and the solvent was evaporated under reduced pressure. Caution: pH 1 decomposes the molecule. The pH was now adjusted to 2 with 1 N HCl and the aqueous residue was extracted with EtOAc, dried (MgSO₄) and concentrated under reduced pressure. The product A34″ was triturated with DIPE:heptane (1:2) and obtained as a greenish white solid (5.7 g, 79%).

### Preparation of Intermediate A40″

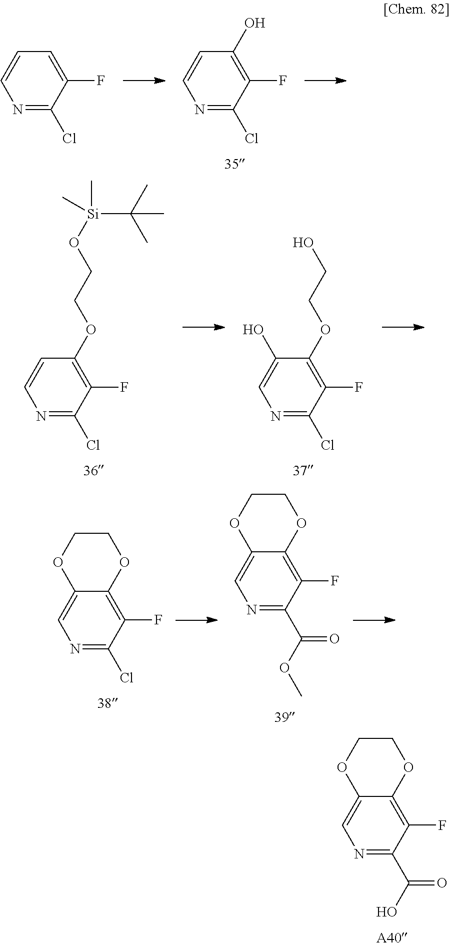

### Step 1

n-BuLi (2 M in cyclohexane, 6 mL, 12.0 mmol) was added to a solution of diisopropylamine (1.8 mL, 12.8 mmol) in dry THF (50 mL) at −78° C. Next, 2-chloro-3-fluoropyridine (1 mL, 10.058 mmol, CAS 17282-04-1) was added dropwise and the reaction mixture was stirred for 2 h at −78° C. Then, trimethylborate (2.4 mL, 21.3 mmol) was added dropwise and the mixture was stirred another 2 h. Then, peracetic acid (39% in acetic acid, 3.0 mL, 17.7 mmol) was added together with water (0.5 mL) and the mixture was allowed to warm to 0° C. and stirred for 1 h. The clear solution turned turbid. An aq. saturated Na₂S₂O₃ solution was added, and the org layer was separated. The aq. layer was extracted with EtOAc multiple times, the combined organic layers were dried over MgSO₄, filtered and concentrated. The residue was purified by flash column chromatography (120 g silica, redisep Gold, gradient DCM/MeOH 99:1 to 95:5). After concentration of the product fractions, intermediate 35″ was obtained as white solid (1.16 g, 78%).

### Step 2

Intermediate 35″ (620 mg, 4.20 mmol) was dissolved in DMF (12 mL), then (2-bromoethoxy)-tert-butyldimethylsilane (1.8 mL, 8.31 mmol) and K₂CO₃ (1.16 g, 8.39 mmol) were added and the mixture was stirred at 70° C. for 3 h. Then the reaction was cooled to R.T., diluted with diethylether and washed with water and brine. The org. layer was dried over MgSO₄, filtered and concentrated. Flash column chromatography (80 g silica, Redisep, heptane/EtOAc, gradient 1:0 to 8:2) delivered intermediate 36″ as a yellowish oil (1.26 g, 98%).

### Step 3

n-BuLi (2.0 M in cyclohexane, 2.5 mL, 5.0 mmol) was added to a solution of TMP (0.9 mL, 5.3 mmol) in dry THF (12 mL) at −78° C. Next, a precooled solution of intermediate 36″ (1.25 g, 4.1 mmol) in THF (5 mL) was added via a cannula and the reaction mixture was stirred for 20 h at −78° C. After 20 h, trimethylborate (1 mL, 8.9 mmol) was added dropwise and the mixture was stirred another 3 h. Then, peracetic acid (39% in acetic acid, 1.2 mL, 7.1 mmol) was added together with water (0.5 mL) and the mixture was allowed to warm to 0° C. and stirred for 1 h. The clear solution turned turbid. An aq. sat. Na₂S₂O₃ solution was added and the layers were separated. The aq. layer was extracted with EtOAc and the combined org. layers were dried over MgSO₄ and concentrated, delivering a yellow oil. This oil was dissolved in MeOH (27 mL) and HCl (37% in H₂O, 0.67 mL, 8.1 mmol) was added upon which the solution became light yellow. After 20 min of stirring, the r.m. was concentrated, redissolved in EtOAc washed with a brine solution. The organic layer was dried over MgSO₄ and concentrated, delivering a yellow oil which was purified by flash chromatography (40 g silica, Redisep, gradient DCM/MeOH/AcOH 99:1:0.5 to 94:6:0.5), delivering intermediate 37″ (500 mg, 50% yield, 85% pure).

### Step 4

Intermediate 37″ (95 mg, 0.46 mmol) was dissolved in THF (4 mL). Molecular sieves (3 A) were added, the solution was stirred for 15 min, then DIAD (0.15 mL, 0.76 mmol) was added dropwise. Next, a solution of TPP (150 mg, 0.57 mmol) in THF (4 mL) was added dropwise. The r.m. was stirred at r.t. for 4 h after which the r.m. was blown to dryness with nitrogen. The residue was triturated with DIPE. A white solid formed, which was filtered, together with the molecular sieves. The filtrate was concentrated and subjected to flash column chromatography (12 g silica, Redisep Gold, heptane/EtOAc, gradient 1:0 to 7:3) to give intermediate 38″ as a white fluffy powder (75 mg, 86%).

### Step 5

In a 75 mL stainless steel autoclave were added KOAc (294 mg, 3.00 mmol), Pd(OAc)₂ (22 mg, 0.10 mmol) and DPPP (84 mg, 0.20 mmol) to a solution of intermediate 38″ (167 mg, 0.89 mmol) in MeOH (10 mL). The autoclave was closed and pressurized to 70 bar CO gas, evacuated again, then backfilled with CO and the reaction was carried out for 20 hours at 140° C. Next, the reaction was cooled to r.t. and concentrated. The residue was purified by flash column chromatography (12 g silica, Redisep Gold, gradient: heptane to heptane:EtOAc 3:7), delivering intermediate 39″ as a white solid (109 mg, 0.51 mmol, 58% yield).

### Step 6

To a solution of intermediate 39″ (107 mg, 0.50 mmol) in THF (4 mL) and water (1 mL) was added LiOH.H₂O (30 mg, 0.73 mmol) at ambient temperature. After 2 h, the RM was concentrated and water was added. The solution was acidified with 1 N HCl to pH 3-4. DCM was added and the layers were separated. The aq. layer was extracted multiple times with DCM until no product remained, and the combined organic layers were dried over MgSO₄ and concentrated, delivering intermediate A40″ as white solid (70 mg, 70%).

### Preparation of Intermediate A47″

### Step 1

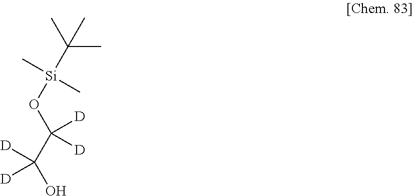

Intermediate 41″ was synthesized following a procedure described in literature (J. Org. Chem. 1986, 51, 3388-3390), starting from commercial ethylene-d₄ glycol [CAS 2219-51-4].

### Step 2

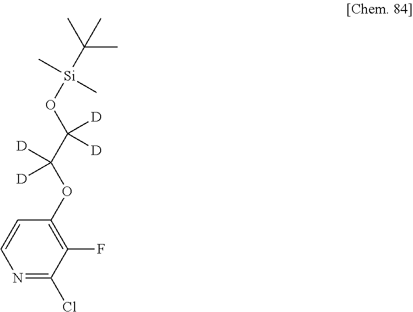 [SMILES: [2H]C([2H])(Oc1ccnc(Cl)c1F)C([2H])([2H])O[Si](C)(C)C(C)(C)C]

At 5° C. to a solution of intermediate 35″ (601 mg, 4.08 mmol), intermediate 41 (735 mg, 4.08 mmol) and TPP (1710 mg, 6.52 mmol) in THF (69 mL). DIAD (1.28 mL, 6.52 mmol) was dropwise added and the r.m. was stirred at r.t. for 1 h. Next, all volatiles were evaporated and the residue was dissolved in toluene and purified on a ISCO purification system (Silica, Redisep, 40 g, 15 min, gradient heptane/EtOAc from 100/0 to 70/30). Product fractions were collected and the solvent was removed under reduced pressure to afford intermediate 42″ (1200 mg, 95%).

### Step 3

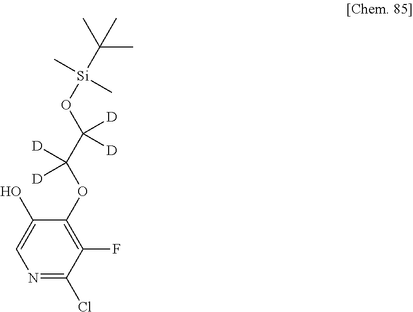 [SMILES: [2H]C([2H])(Oc1c(O)cnc(Cl)c1F)C([2H])([2H])O[Si](C)(C)C(C)(C)C]

n-BuLi (2.0 M in cyclohexane, 3.18 mL, 5.08 mmol) was added to a solution of TMP (0.92 mL, 5.38 mmol) in dry THF (9 mL) at −78° C. Next, a precooled solution of intermediate 42″ (1.33 g, 4.29 mmol) in THF (13 mL) was added via a cannula and the reaction mixture was stirred for 20 h at −78° C. After 20 h, trimethylborate (1.03 mL, 9.15 mmol) was added dropwise and the mixture was stirred another 2 h. Then, peracetic acid (39% in acetic acid, 1.2 mL, 7.08 mmol) was added together with water (0.5 mL) and the mixture was allowed to warm to 0° C. and stirred for 1 h. The clear solution turned turbid. A mixture of aq. saturated Na₂S₂O₃ solution was added, the layers were separated. The aq. layer was extracted with EtOAc, the combined organic layers were dried over MgSO₄ and concentrated, delivering intermediate 43″ as a yellow oil. The residue was purified on a ISCO purification system (Redisep, 40 g, 15 min, gradient heptane/EtOAc from 100/0 to 75/25). Product fractions were collected and the solvent was removed under reduced pressure to afford intermediate 43″ (1.00 g, 71%, 66:44 mixture with intermediate 42″), which was used as such in the next step.

### Step 4

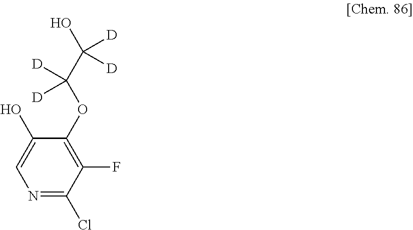 [SMILES: [2H]C([2H])(O)C([2H])([2H])Oc1c(O)cnc(Cl)c1F]

Intermediate 43″ was dissolved in MeOH (20 mL) and HCl (37% in water, 0.5 mL, 5.99 mmol) was dropwise added at r.t. The r.m. was stirred at r.t. for 30 min, after which the MeOH was removed under reduced pressure. The residue was neutralized with an aq. sat. NaHCO₃ solution and extracted with EtOAc. The combined org layers were dried over MgSO₄, filtered and the solvent was evaporated to afford a first crop of intermediate 44″. The aq. phase was further acidified by the addition of a 1 N HCl solution to pH 6-7 and the product was then extensively extracted (5×20 mL) with EtOAc until LCMS showed no remaining product in the aq. phase. The combined OL were dried over MgSO₄, filtered and the solvent was evaporated. The residue was purified by flash chromatography (40 g silica, Gold Redisep, gradient DCM/MeOH 100/0 to 97/3), delivering a second crop of intermediate 44″, and a mixed fraction which was further purified by preparative HPLC (Stationary phase: RP XBridge Prep C18 OBD-10 μm, 30×150 mm, Mobile phase: 0.25% NH₄HCO₃ solution in water, MeOH) yielding a third crop of intermediate 44″. The three crops were combined to give intermediate 44″ as a white solid (205 mg, 33%).

### Step 5

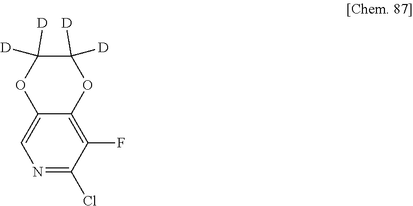 [SMILES: [2H]C1([2H])Oc2cnc(Cl)c(F)c2OC1([2H])[2H]]

Intermediate 45″ was synthesized following an analogous procedure to that described for the preparation of 38″ starting from 44″.

### Step 6

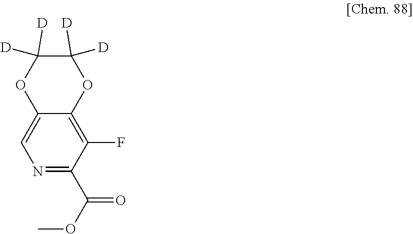 [SMILES: [2H]C1([2H])Oc2cnc(C(=O)OC)c(F)c2OC1([2H])[2H]]

Intermediate 46″ was synthesized following an analogous procedure to that described for the preparation of 39″ starting from 45″.

### Step 7

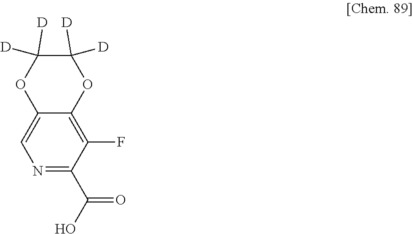 [SMILES: [2H]C1([2H])Oc2cnc(C(=O)O)c(F)c2OC1([2H])[2H]]

Intermediate A47″ was synthesized following an analogous procedure to that described for the preparation of 40″ starting from 46″.

### Preparation of Intermediate A50″

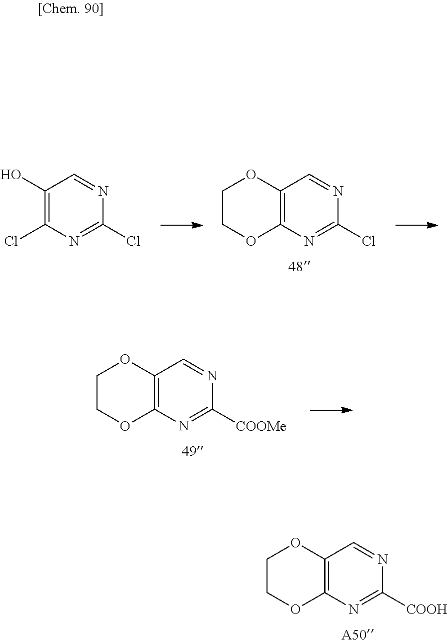 [SMILES: Oc1cnc(Cl)nc1Cl ; Clc1ncc2c(n1)OCCO2 ; COC(=O)c1ncc2c(n1)OCCO2 ; O=C(O)c1ncc2c(n1)OCCO2]

### Step 1

To a solution of 2,4-dichloropyrimidin-5-ol (3.30 g, 20 mmol) in DMF (66 mL) were added potassium carbonate (13.8 g, 100 mmol) and 2-bromoethan-1-ol (7.50 g, 60 mmol). After being stirred for 4 h at 100° C., the mixture was diluted with water and extracted with ethyl acetate. The combined organic layers were washed with water and evaporated. The crude product was purified by flash column chromatography (silica gel, 9:1-1:1 hexane/ethyl acetate) to give 48″ (490 mg, 2.84 mmol, 14%) as a white solid. ¹H NMR (400 MHz, CDCl₃) δ: 4.30-4.39 (m, 2H), 4.51-4.56 (m, 2H), 8.19 (s, 1H).

### Step 2

A solution of 48″ (490 mg, 2.84 mmol) and [1,1′-bis(diphenylphosphino)ferrocene]dichloropalladium(II) dichloromethane adduct (232 mg, 0.284 mmol) in methanol (10 mL, 247 mmol) and triethylamine (5 mL, 36.1 mmol) was stirred for 7 h at 110° C. under carbon monooxide (0.5-0.8 MPa). The mixture was evaporated and purified by flash column chromatography (silica gel, 1:1-0:100 hexane/ethyl acetate) to give 49″ (270 mg, 1.38 mmol, 49%) as a white solid. ¹H NMR (400 MHz, CDCl₃) δ: 4.01 (s, 3H), 4.37-4.42 (m, 2H), 4.55-4.60 (m, 2H), 8.36 (s, 1H).

### Step 3

To a suspension of 49″ (270 mg, 1.38 mmol) in methanol (5.4 mL) was added a solution of sodium hydroxide (1.38 mL, 2.75 mmol, 2 mol/L in water). After being stirred for 3 h at room temperature, the mixture was neutralized with a solution of hydrogen chloride (1.38 mL, 2.75 mmol, 2 mol/L in water) and evaporated to remove methanol. The suspension was filtered, and the collected solid was washed with water to give A50″ (188 mg, 1.03 mmol, 75% yield) as a white solid. ¹H NMR (400 MHz, DMSO-d₆) δ: 4.28-4.32 (m, 2H), 4.50-4.55 (m, 2H), 8.35 (s, 1H).

### Preparation of Intermediate A54″

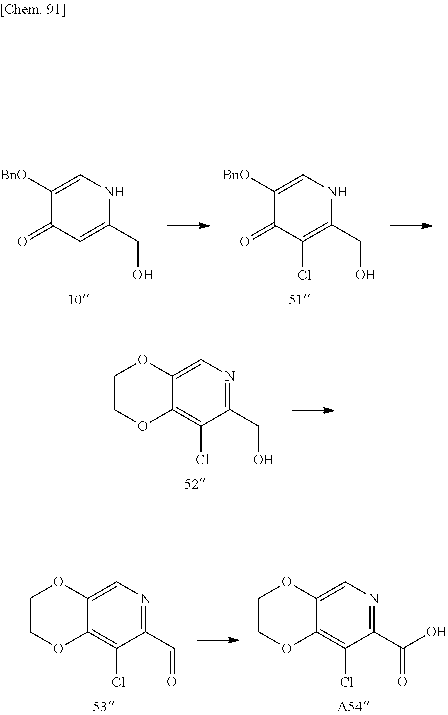 [SMILES: O=c1cc(CO)[nH]cc1OCc1ccccc1 ; O=c1c(OCc2ccccc2)c[nH]c(CO)c1Cl ; OCc1ncc2c(c1Cl)OCCO2 ; O=Cc1ncc2c(c1Cl)OCCO2 ; O=C(O)c1ncc2c(c1Cl)OCCO2]

### Step 1

To a suspension of Compound 10″ (4.77 g, 20.6 mmol) in chloroform (47.7 mL) was added NCS (3.31 g, 24.8 mmol). After being stirred for 2 hours at 70° C., the reaction mixture was cooled to room temperature and chloroform (48 mL) was added to the reaction mixture. The suspension was filtered to give Compound 51″ (5.97 g, 20.6 mmol, 100%) as a white solid. ¹H NMR (400 MHz, CDCl₃) δ: 3.15-3.70 (1H, br), 4.34 (1H, s), 5.03 (1H, 7.22-7.50 (6H, m).

### Step 2

To a suspension of Compound 51″ (5.97 g, 20.6 mmol) in THF (59.7 mL) were added an aqueous 2 mol/L sodium hydroxide (12.4 mL, 24.8 mmol) and 10 w/w % palladium on carbon (3 g). After being stirred for 3 hours at room temperature under 1 atm hydrogen. The reaction mixture was filtered through Celite (Registered trademark) pad. The filtrate was evaporated. To a suspension of the residue in DMF (59.7 mL) were added potassium carbonate (8.55 g, 61.9 mmol) and 1, 2-dibromoethane (2.67 mL, 30.9 mmol). The reaction mixture was stirred for 1 hour at 70° C. and for 3 hours at 90° C. To the reaction mixture was added toluene (100 mL), and the suspension was filtered. The filtrate was evaporated. The crude product was added to a silica gel column and eluted with hexane/EtOAc 50% to 100%. Collected fractions were evaporated to afford Compound 52″ (2.65 g, 13.1 mmol, 64%) as a mixture of yellow oil and solid. ¹H NMR (400 MHz, CDCl₃) δ: 4.14-4.18 (1H, br), 4.30-4.34 (2H, m), 4.45-4.48 (2H, m), 4.68-4.71 (2H, m), 8.09 (1H, s).

### Step 3

To a suspension of Compound 52″ (2.65 g, 13.1 mmol) in DCM (26.5 mL) was added manganese dioxide (15.0 g, 173 mmol). After being stirred for 1 hour at room temperature, the reaction mixture was filtered through Celite (Registered trademark) pad. The filtrate was evaporated. The crude product was added to a silica gel column and eluted with hexane/EtOAc 10% to 50%. Collected fractions were evaporated to afford Compound 53″ (1.03 g, 5.15 mmol, 39%) as a white solid. ¹H NMR (400 MHz, CDCl₃) δ: 4.39-4.43 (2H, m), 4.49-4.52 (2H, m), 8.39 (1H, s), 10.2 (1H, s).

### Step 4

To a solution of Compound 53″ (1.03 g, 5.15 mmol) in acetone (30.8 mL) and water (10.3 mL) were added sodium dihydrogen phosphate (927 mg, 7.73 mL), 2-methyl-2-butene (5.46 mL, 51.5 mmol) and sodium chlorite (1.75 g, 15.5 mmol) at 0° C. After being stirred for 1 hour at room temperature, aqueous 2 mol/L hydrochloric acid (7 mL) was added to the reaction mixture. The mixture was evaporated and cooled to 0° C. The suspension was filtered to give intermediate A54″ (433 mg, 2.01 mmol, 39%) as a white solid. ¹H NMR (400 MHz, DMSO-d₆) δ: 4.37-4.41 (2H, m), 4.48-4.52 (2H, m), 8.13 (1H, s).

### Preparation of Anilines of Formula (II-B)

### Preparation of Intermediate B1″

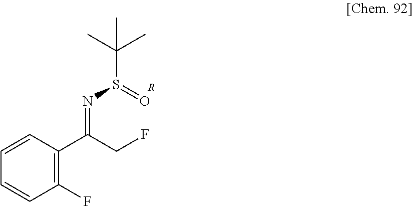

The synthesis of intermediate B1″ from 2-fluoro-1-(2-fluorophenyl)-ethanone has been described previously in, for instance, WO 2014065434.

### Preparation of Intermediate B2″

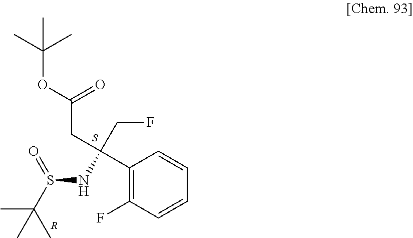

A mixture of zinc (252 g, 3856 mmol) and CuCl (38 g, 386 mmol) in THF (1.5 L) was stirred at reflux for 30 min. The heating bath was removed while maintaining vigorous stirring. The addition funnel was then charged with tert-butyl bromoacetate (188 g, 964 mmol) in THF (250 mL). This solution was slowly and carefully added dropwise under reflux was re-initiated. The addition was continued at a rate that maintained a controllable reflux. Once the addition was complete, the reaction mixture was stirred an addition 30 minutes at ambient temperature, then at 50° C. for 30 minutes. The reaction mixture was then cooled to 0° C., and intermediate B1″ (100 g, 386 mmol) in THF (250 mL) was added. The reaction mixture was further stirred at 0° C. for 4 hours. Next, the reaction mixture was filtered and the filtrate was washed with 2.5 M HCl (500 mL), sat. aq. NaHCO₃ (500 mL×2) and sat. aq. NaCl (1 L) after which it was dried over Na₂SO₄, filtered and concentrated under vacuum to afford the crude intermediate B2″ as the yellow oil (140 g, 97%).

### Preparation of Intermediate B3″

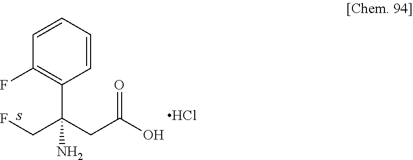

A mixture of intermediate B2″ (150 g, 399 mmol) in HCl (4 M in 1,4-dioxane, 1.07 L) was heated at 70° C. and stirred for 3 h. Next, the mixture was cooled to rt and the white precipitate filtered off and dried to afford intermediate B3″ (hydrochloric acid salt, 115 g, 100%).

### Preparation of Intermediate B4″

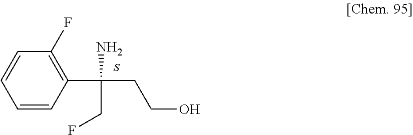

A mixture of intermediate B4″ (100 g, 347 mmol) in THF (500 mL) was stirred at 0° C. under N2. BH₃ (1 M in THF, 694 mL, 694 mmol) was added dropwise and the reaction was stirred at room temperature for 3 h. The resulting mixture was poured into sodium bicarbonate (80 g) and ice (300 g). Ethyl acetate (500 mL) was added and the mixture was stirred at room temperature. The mixture was extracted with ethyl acetate (600 mL×3). The organic layer was dried over Na₂SO₄, filtered and concentrated to afford the crude product. The residue was purified by column chromatography over silica gel (eluent: petroleum ether/ethyl acetate 30:1 to petroleum ether/ethyl acetate 5:1). The pure fractions were collected and the solvent was evaporated under vacuum to give the product B4″ as colorless oil (64 g, 91%).

### Preparation of Intermediate B5″

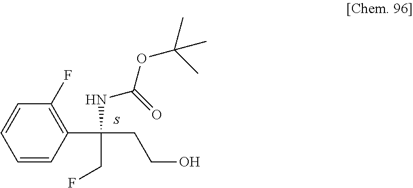

Di-tert-butyl dicarbonate (50 g, 229 mmol) was added to a solution of intermediate B4″ (41.5 g, 177 mmol) in 2-methyltetrahydrofuran (755 mL) and EtOH (30 mL). The reaction mixture was heated for 40 h at 63° C., then cooled to 20° C. The reaction mixture was washed with 100 mL water together with 10 mL NH₃ (25% in water). The aq. layer was extracted once more with 20 mL 2-methyl-tetrahydrofuran. The combined org. layers were dried (MgSO₄), filtered and concentrated in vacuo. The residue was purified by column chromatography (2 L glass filter filled with silica gel, eluent: 0-50% EtOAc in heptane), the product fractions were concentrated and dried providing intermediate B5″ (46 g, 86%).

### Preparation of Intermediate B6″

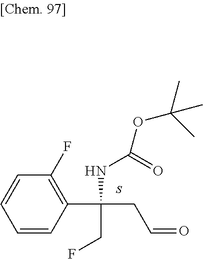

Method A″: To a solution of intermediate B5″ (47 g, 156 mmol) in DCM (500 mL) was added Dess-Martin periodinane (76 g, 176 mmol) in portions at 0° C. The resulting mixture was stirred at RT for 1 h. A combination of sat. aq. Na₂S₂O₃ and sat. aq. NaHCO₃-solution was added, together with water, and the mixture was stirred for 30 min. The org. layer was separated, the aq. layer was extracted with DCM. The combined organic layers were dried over MgSO₄ and concentrated. The residue was purified by column chromatography (2 L glass filter filled with silica gel, eluent: 0-20% EtOAc in heptane), delivering intermediate B6″ as a colorless viscous liquid (39 g, 84%).

Method B″: To a solution of trichloroisocyanuric acid (74 g, 318 mmol) and TEMPO (1.40 g, 8.96 mmol) in 300 mL DCM was added intermediate B5″ (90 g, 299 mmol) in 150 mL DCM dropwise at 0.5° C. The resulting mixture was stirred at 1.5° C. for 1 h. (TLC 30% EtOAc in heptane). Dicalite (Registered trademark) was added to the orange suspension, after stirring for 5 min it was filtered and washed with 200 mL DCM to give filtrate 1. A combination of 81 g Na₂S₂O₃ and 54 g Na₂CO₃ was dissolved in 500 mL water in an OptiMax (Registered trademark) Synthesis Workstation, Mettler Toledo). This mixture was cooled to 1° C., then filtrate 1 was added over 15 min at 1° C., and stirring was continued for 15 min. Dicalite (Registered trademark) was added and this suspension was filtered and washed with 200 mL DCM. This organic layer was washed with 90 g NaCl in 540 mL water. The layers were separated. The organic layer was dried over MgSO₄ and the dry organic layer was used as such in the next reaction step.

### Preparation of Intermediate B7″

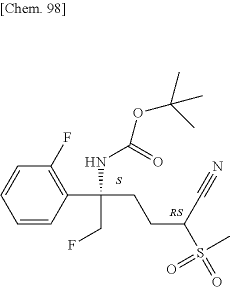

To a solution of intermediate B6″ obtained via method A″ (37.5 g, 125 mmol) in MeOH (500 mL) were added methylsulfonylacetonitrile (30 g, 244 mmol) and MgO (8 g, 198 mmol). The resulting mixture was stirred at 65° C. for 5 h, then at ambient temperature for 15 min. The suspension was mixed with Dicalite (Registered trademark), filtered and rinsed with 2-methyltetrahydrofuran. The obtained filtrate was concentrated to 1/10 volume, then 2-methyltetrahydrofuran (500 mL) was added. The reaction mixture was cooled to 0° C., and NaBH₄ (1.5 g, 39 mmol) was added. After 15 min, the Knoevenagel product was entirely reduced to intermediate B7. (TLC monitoring, silica gel, eluent: 30% EtOAc in heptane). The resulting suspension was mixed with Dicalite (Registered trademark), filtered and rinsed with 2-methyltetrahydrofuran. HCl (1 N, 120 mL) was added, followed by brine and 2-methyltetrahydrofuran. The org. layer was separated, the aq. layer was extracted with 2-methyltetrahydrofuran. The combined org. layers were dried over MgSO₄, filtered and concentrated. The residue was subjected to column chromatography (silica-filled glass filter (2 L), eluent: 20-30% EtOAc in heptane). The pure product fractions were concentrated delivering intermediate B7″ as yellow-white sticky oil (40 g, 79%).

### Preparation of Intermediate B8″

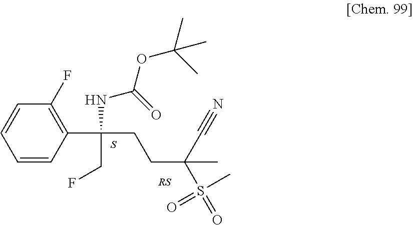

To a solution of intermediate B7″ (40 g, 99.4 mmol) in 2-methyltetrahydrofuran (500 mL) was added NaH (60% dispersion in mineral oil, 4.6 g, 115 mmol) at 0° C. (caution: exothermic reaction, hydrogen evolution). After 30 min of stirring, MeI (8 mL, 127 mmol) was added dropwise, keeping the temperature at 0° C. The resulting mixture was stirred at 0° C. for 30 min, after which TLC (silica gel, eluent: 40% EtOAc in heptane) showed full conversion. Sat. aq. NH₄Cl and water were added and the layers were separated. The aq. layer was extracted with 2-methyltetrahydrofuran. The combined organic layers were dried over MgSO₄, filtered and concentrated. The residue was purified by column chromatography (silica-filled glass filter, 2 L, eluent 20-30% EtOAc in heptane). The product fractions were combined, delivering intermediate B8″ as yellow-white foam (37 g, 89%).

### Preparation of Intermediate B9″

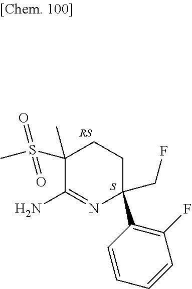

TFA (8.7 g, 76.3 mmol) was added to a solution of intermediate B8″ (18 g, 43.2 mmol) in toluene (450 mL) in a Hastelloy (Registered trademark) reactor of 500 mL. The reactor was sealed and the mixture stirred for 10 h with an internal temperature of 115° C. Next, the reaction mixture was cooled to rt, washed with aq. sat. NaHCO₃ together with aq. sat. Na₂CO₃. The organic layer was separated, the aq. layer was once more extracted with toluene. The combined organic layers were dried (MgSO₄), filtered and concentrated. The residue was purified with flash column chromatography using (330 g silica gel, eluent: DCM/(7 N NH₃ in MeOH), gradient from 100:0 to 97:3). The product fractions were collected and evaporated providing intermediate B9″ as a foamy solid (13.6 g, 99%).

### Preparation of Intermediates B10a″ and B10b″

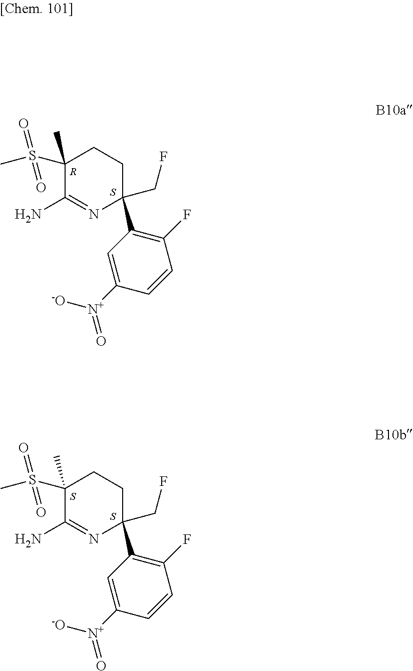

Intermediate B9″ 35.2 g, 111 mmol) was dissolved in TFA (200 mL, 2613 mmol) and then cooled to 0° C. H2504 (28 mL, 525 mmol) was added at 0° C. Then potassium nitrate (12 g, 119 mmol) was added slowly keeping the temperature below 3° C. The reaction was finished after the addition was complete. The mixture was poured onto a mixture of ice (1 kg), DCM (1 L) and NH₃ (25% in water). Next, 200 mL Na₂CO₃ aq. sat. sol. was added. The organic layer was separated, and the water layer was extracted once more with 500 mL DCM. The combined organic layers were dried (MgSO₄), filtered and evaporated giving a foamy solid (40 g). A separation of diastereomers was performed via preparative SFC (stationary phase: Diacel Chiralpak (Registered trademark) AD 20×250 mm, mobile phase: CO₂, iPrOH+0.4 iPrNH₂) yielding intermediate B10b″ (15 g, 37%) and intermediate B10a″ 16 g, 40%).

### Preparation of Intermediate B11″

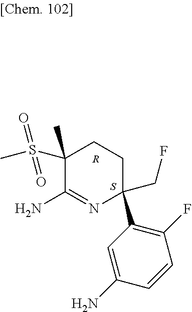

Intermediate B10a″ 3.7 g, 10.2 mmol) was dissolved in MeOH (100 mL), water (50 mL) and THF (100 mL), then Fe (4.4 g, 78.8 mmol) and NH₄Cl (5.8 g, 108 mmol) were added. The reaction mixture was stirred at 63° C. for 1 h. Then the mixture was cooled and diluted with DCM. Next, NaHCO₃ aq. sat. solution and Dicalite (Registered trademark) were added. The mixture was filtered and the filter cake washed with DCM. The organic layer of the filtrate was separated, dried (MgSO₄), filtered and evaporated. The resulting sticky foam was dissolved in DCM (10 mL) and DIPE (10 mL) was added. This solution was concentrated leaving a solid foam, which was scratched to provide intermediate B11″ as a fine powdery solid (3.4 g, 100%).

### Preparation of Intermediate B12″

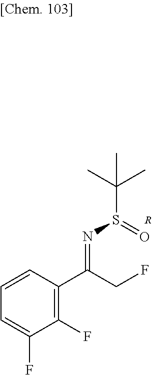

The synthesis of intermediate B12″ has been described previously in WO 2016075064.

### Preparation of Intermediate B13″

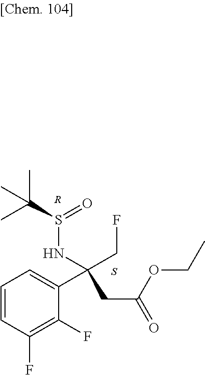

In a three-necked flask, fitted with a thermometer, a reflux condenser and an addition funnel, a suspension of zinc (28.3 g, 432 mmol) and CuCl (4.3 g, 43.3 mmol) in THF (140 mL) was stirred at reflux for 30 min. The heating bath was then removed while maintaining vigorous stirring and the addition funnel was charged with ethyl bromoacetate (12.0 mL, 108 mmol) in THF (30 mL). This solution was added dropwise until reflux was re-initiated. The addition was continued at a rate that maintained a controllable reflux. After the addition, the reaction mixture was stirred for 30 min at ambient temperature, then for 30 min at 50° C. The mixture was cooled to 0° C., and a solution of intermediate B12″ (12.0 g, 43.3 mmol) in THF (20 mL) was added dropwise. The reaction mixture was stirred at 0° C. for 2 hours. Next, the mixture was filtered over a pad of Celite (Registered trademark) and the filter cake was washed with diethyl ether. The filtrate was washed with 0.25 M aqueous HCl, followed by washings with sat. aq. NaHCO₃ (2×) and brine. The organic layer was dried over MgSO₄, and concentrated in vacuo. The residue was purified by flash column chromatography over silica gel (eluent: DCM). The product containing fractions were concentrated, delivering an orange residue, which was subjected again to flash column chromatography over silica gel (eluent: 0-30% EtOAc in heptane). The product containing fractions were collected and concentrated, delivering the product B13″ as yellow oil (13 g, 82%).

### Preparation of Intermediate B14″ [Chem. 105]

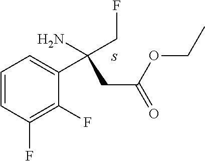

To a solution of intermediate B13″(8 g, 21.9 mmol) in MeOH (70 mL) was added HCl (70 mL, 4 M in dioxane). The mixture was stirred at ambient temperature for 1 h, then concentrated in vacuo. The solid residue was redissolved in EtOAc, and basified with sat. aq. Na₂CO₃. The layers were separated, the organic layer was dried over MgSO₄ and concentrated, yielding intermediate B14″ (4.0 g, 70%) as yellow oil.

### Preparation of Intermediate B15″ [Chem. 106]

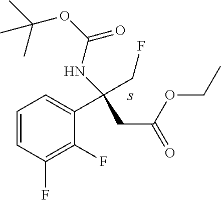

A solution of intermediate B14″ (7 g, 26.8 mmol) and di-tert-butyl dicarbonate (11.7 g, 53.6 mmol) in MeOH (83 mL) was stirred at 50° C. overnight. Afterwards, the reaction mixture was concentrated and subjected to flash column chromatography over silica gel (0 to 50% EtOAc in heptane). The product containing fractions were concentrated in vacuo, affording intermediate B15″ (5 g, 52%).

### Preparation of Intermediate B16″

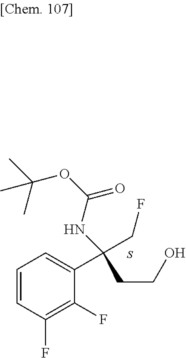

Lithium borohydride (13.8 mL, 27.6 mmol, 2 M solution in THF) was added dropwise at 0° C. to a solution of intermediate B15″ (5.0 g, 13.8 mmol) in THF (150 mL). The reaction was stirred at ambient temperature for 6 hours. Next, it was cooled to 0° C. and a 10% aq. NH₄C1-solution was added dropwise. The mixture was diluted with EtOAc and the layers were separated. The aqueous one was extracted with EtOAc. The combined organic layers were dried over MgSO₄ and concentrated in vacuo. The residue was purified by flash column chromatography over silica gel (eluent: 0 to 100% EtOAc in heptane). The product containing fractions were collected and dried in vacuo, resulting in intermediate B16″ (4.0 g, 91%).

### Preparation of Intermediate B17″ [Chem. 108]

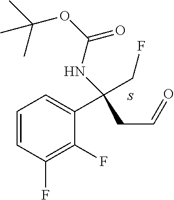

To a solution of intermediate B16″ (4.0 g, 12.5 mmol) in DCM (350 mL) was added Dess-Martin periodinane (8.3 g, 19.6 mmol) in portions at 0° C. The resulting mixture was stirred at RT for 30 min. A combination of sat. aq. Na₂S₂O₃ and sat. aq. NaHCO₃-solution was added, and the mixture was stirred for 30 min. The org. layer was separated, the aq. layer was extracted with DCM. The combined organic layers were dried over MgSO₄ and concentrated. The residue was purified by flash column chromatography over silica gel (eluent: 0-100% EtOAc in heptane), delivering intermediate B17″ as a colorless viscous liquid (3.5 g, 88%).

### Preparation of Intermediate B18″

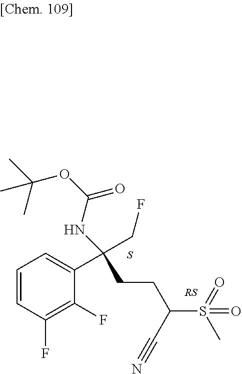

To a solution of intermediate B17″ 3.5 g, 11 mmol) in MeOH (56 mL) was added 2-methyl-sulfonyl acetonitrile (2.6 g, 22 mmol) and MgO (0.5 g, 13.2 mmol). The resulting mixture was stirred at 60° C. for 20 hours. The reaction was cooled down to room temperature and filtered over Celite (Registered trademark). The filtrate was concentrated and dissolved in THF (130 mL). The solution was cooled to 0° C. and sodium borohydride (0.42 g, 11 mmol) was added. The reaction mixture was stirred for 30 min, followed by neutralization with aq. HCl (1 M). The mixture was diluted with DCM and a sat. aq. NaHCO₃ solution was added. The layers were separated and the organic one was dried over MgSO₄ and concentrated. The residue was purified by flash column chromatography over silica gel (0-30% EtOAc in heptane), delivering intermediate B18″ (3.2 g, 69%).

### Preparation of Intermediate B19″ [Chem. 110]

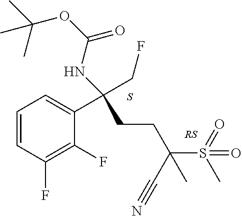

To a solution of intermediate B18″ 3.2 g, 7.6 mmol) in THF (110 mL) was added NaH (550 mg, 13.7 mmol, 60% dispersion in mineral oil) at 10° C. The mixture was stirred at that temperature for 15 min, then iodomethane (0.52 mL, 8.4 mmol) was added. The mixture was stirred for 45 min, after which water and EtOAc were added. The organic layer was isolated, dried over MgSO₄ and concentrated in vacuo. The residue was purified by flash column chromatography over silica gel (eluent: 0-40% EtOAc in heptane). The product containing fractions were concentrated, affording intermediate B19″ (2.2 g, 67%)

### Preparation of Intermediate B20″

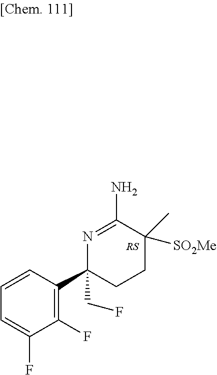 [SMILES: CC1(S(C)(=O)=O)CC[C@@](CF)(c2cccc(F)c2F)N=C1N]

Intermediate B19″ (2.2 g, 5.1 mmol) was stirred in formic acid (28 mL) at room temperature for 20 hours, then for 5 hours at 90° C. The reaction mixture was cooled down to room temperature, diluted with EtOAc and basified to pH 10 by addition of aq. sat. Na₂CO₃. The organic layer was separated, dried (MgSO₄) and concentrated in vacuo. The residue was purified by flash column chromatography over silica gel (eluent: 0-3% (7 N NH₃ in MeOH) in DCM), resulting in intermediate B20″ (0.97 g, 57%).

### Preparation of Intermediate B21a″ and B21b″

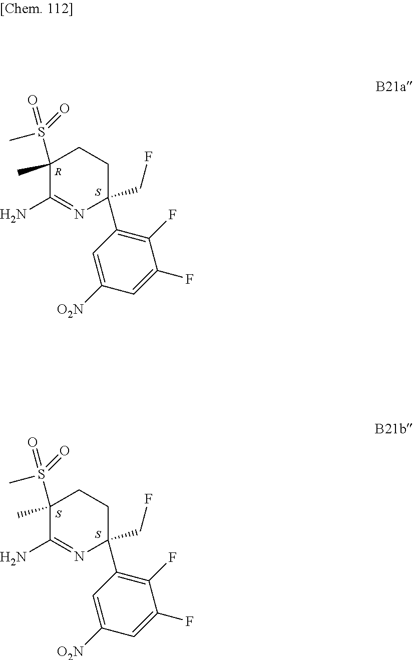

To a solution of intermediate B20″ (100 mg, 0.293 mmol) in TFA (3 mL, 39.2 mmol) was added sulfuric acid (0.1 mL, 1.88 mmol) at 0° C. Next, potassium nitrate (32 mg, 0.313 mmol) was added portion wise and the mixture was stirred at 0° C. for 150 min and then at rt overnight. Then, the reaction was poured on a mixture of 5 mL ice, 15 mL DCM, 5 mL NH₃ (25%) and 5 mL Na₂CO₃ sat. aq. solution. The layers were separated and the aqueous one was extracted with DCM. The combined organic layers were dried over MgSO₄, filtered and concentrated, to afford a light yellow oil. A purification by flash column chromatography was performed (12 g silica) using as eluent heptane/EtOAc from 1:0 to 4:6. Two fractions were collected and evaporated, providing intermediate B21a″ (35 mg, 31%) and intermediate B21b″ (54 mg, 49%) both as a transparent glass.

### Preparation of Intermediate B22″

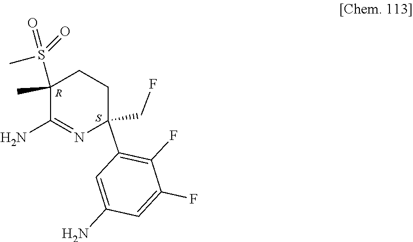

Intermediate B21a″ (20 mg, 0.053 mmol) was dissolved in MeOH (1 mL), water (0.5 mL) and THF (1 mL), then Fe (22 mg, 0.394 mmol) and NH₄Cl (30 mg, 0.561 mmol) were added. Nitrogen was bubbled through the mixture for 5 min. The reaction mixture was then stirred at 65° C. for 3.5 h. Next, the mixture was cooled to rt and diluted with DCM and NaHCO₃ sat. aq. solution and filtered over a path of Dicalite (Registered trademark). The filtrate cake was washed with DCM. The organic layer was separated and the aqueous one extracted once with DCM. The combined organic layers were dried (MgSO₄), filtered and evaporated to dryness, providing intermediate B22″ as a yellow foam (27 mg, 100%).

### Preparation of Intermediate B23″

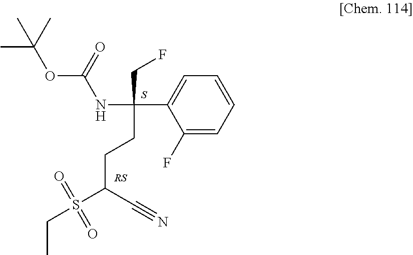

Intermediate B6″ (84 g, 281 mmol) obtained via method B″ as a DCM solution (677 mL) and 2-(ethanesulfonyl)acetonitrile (50 g, 339 mmol) was added at 20° C. in an EasyMax(Registered trademark) Advanced Synthesis Workstation (Mettler-Toledo), then DBU (0.84 mL, 5.61 mmol) was added. The reaction mixture was stirred overnight at 20° C. Then acetic acid (1.61 mL, 28 mmol) was added at 20° C., and the reaction mixture was stirred for 90 min. The reaction mixture was then concentrated. THF (677 mL) was added, and the reaction mixture was concentrated to 600 mL after which 2-propanol (85 mL) was added. This solution was cooled to 0° C. Sodium borohydride (6.37, 168 mmol) was added in portions to the reaction mixture over 20 min at 10° C. and then it was stirred for another 30 min at rt. Subsequently HCl (1 M in water, 196 mL) was added at 0° C., then the layers were separated and the organic layer was washed with a solution of 90 g NaCl in 540 mL water. The organic layer was separated, dried (MgSO₄), filtered and evaporated. The residue was purified by chromatography using a 4 liter glass fritted filter filled with silica gel, using heptane/EtOAc going from 100:0 to 60:40 as eluent. The product fractions were collected and evaporated providing intermediate B23″ (102 g, 87%).

### Preparation of Intermediate B24″

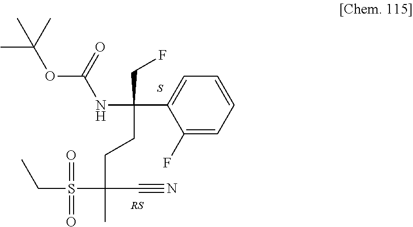

Intermediate B23″ (45 g, 108 mmol) was dissolved in anhydrous THF (563 mL) under N2 atmosphere at 10° C. NaH (60% dispersion in mineral oil, 5.3 g, 132 mmol) was added over 20 min, stirred for 20 min at 10° C. (OptiMax(Registered trademark) Synthesis Workstation, Mettler Toledo) and then MeI (8.2 mL, 132 mmol) was added over 20 min. Stirring was continued at 10° C. for 0.5 h. The reaction was carefully quenched with water (100 mL) and diluted with EtOAc. Brine was added (100 mL). The OL was separated and the aqueous one was extracted with EtOAc. The combined OL were dried with MgSO₄, filtered and the solvent was evaporated under reduced pressure. The crude residue was used in the next step (47 g, quantitative).

### Preparation of Intermediate B25a and B25b

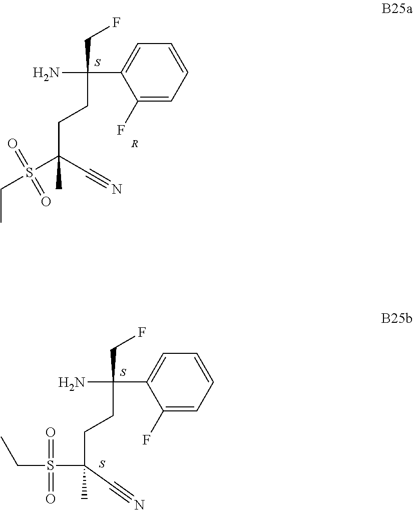

Intermediate B24″ (15.5 g, 36 mmol) was dissolved in 90 mL THF and stirred at rt. Then HCl (6 N in water, 30 mL, 180 mmol) was added (slight exotherm). The RM was stirred at 55° C. for 2 h after which it was cooled to rt. Toluene (50 mL) was added and the product was extracted in the acidic water layer. The OL was washed with water (50 mL). The combined water layers were neutralized with 50% aq. NaOH and the product was extracted with THF (2×50 mL). The combined OL were dried (MgSO₄), filtered and concentrated. Then 70 mL 2-propanol was added and the mixture was concentrated to approx. 40 mL. Then 70 mL DIPE was added and the mixture stirred at rt for 5 min, then at 0° for 1 h. A precipitate formed which was filtered, washed and dried which contains mainly B25a (6.2 g, 51%). The filtrate was concentrated giving a residue containing mainly B25b (4.5 g, 38%).

### Preparation of Intermediate 26″

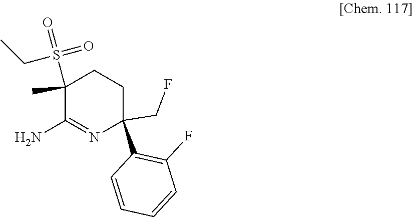 [SMILES: CCS(=O)(=O)[C@]1(C)CC[C@@](CF)(c2ccccc2F)N=C1N]

TFA (2.7 mL, 35.4 mmol) was added to intermediate B25a (13.0 g, 39.3 mmol) in 149 mL toluene. This reaction mixture was stirred and refluxed for 1 h after which the solvent was removed by evaporation. The residue was taken up in ethyl acetate and washed with Na₂CO₃ sat. solution, the org. layer was dried (MgSO₄), filtered and evaporated. The residue was taken up in a mixture of 20 mL DIPE and 3 mL 2-propanol and heated until homogeneous. The resulting solution was cooled to 0° C. and stirred, after which a precipitation occurred. The solid was filtered off, washed with some DIPE and dried (10.7 g, 82%).

### Preparation of Intermediate B27″

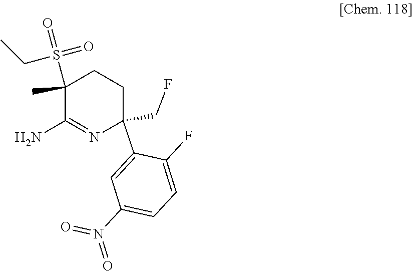 [SMILES: CCS(=O)(=O)[C@]1(C)CC[C@@](CF)(c2cc([N+](=O)[O-])ccc2F)N=C1N]

Intermediate B26″ (17 g, 51.4 mmol) was dissolved in 140 mL TFA and then cooled to 0° C. H₂SO₄ (15 mL, 281 mmol) was added at 0° C. Then KNO₃ (5.3 g, 52.4 mmol) was added slowly keeping the temperature below 3° C. The reaction was finished after complete addition and poured onto a mixture of 0.7 kg ice, 0.7 L DCM and 240 mL 25% aq. NH₃. The organic layer was separated, the water layer was extracted with 200 mL DCM. The combined organic layers were dried (MgSO₄), filtered and evaporated. The residue was triturated in 60 mL 2-propanol, heated and cooled while stirring. The solid was filtered, washed and dried (18 g, 92%).

### Preparation of Intermediate B28″

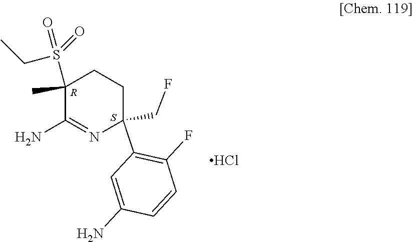

Intermediate B27″ (40 g, 107 mmol) was dissolved in a mixture of 600 mL MeOH, 300 mL water and 600 mL THF. Then iron (48 g, 859 mmol) and NH₄Cl (66 g, 1234 mmol) were added. The reaction mixture was stirred at 65° C. for 5 h. After 2 h, more iron (24 g, 430 mmol) and NH₄Cl (17 g, 309 mmol) were added. Then the reaction mixture was cooled to rt and diluted with DCM and NaHCO₃ aq. sat. solution, after which Dicalite (Registered trademark) was added. The mixture was filtered and the filter washed with DCM. The organic layer was separated, dried (MgSO₄), filtered and evaporated. The residue was dissolved in ethyl acetate, HCl (6 N in 2-propanol, 20 mL, 120 mmol) was added after which a solid formed. The solid was filtered, washed with DIPE and dried in vacuum at 40° C. (38 g, 93%).

### Preparation of Intermediate B29″

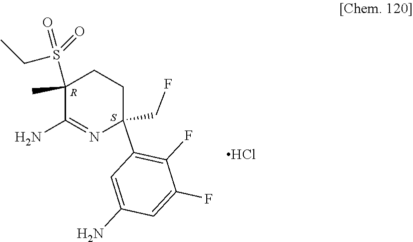

Intermediate B29″ was prepared in a similar way as intermediate B28″ but starting from intermediate B12″ instead of intermediate B1″.

### Preparation of the Final Compounds

### Example 1″

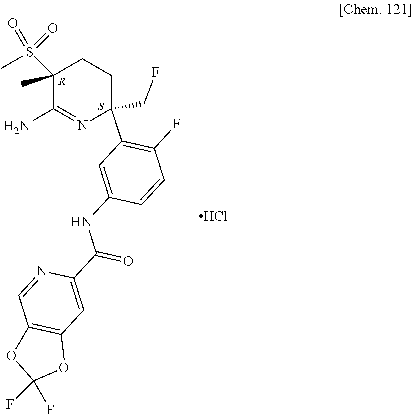

Intermediate B11″ (1.00 g, 3.02 mmol) was dissolved in MeOH (100 mL) at r.t. under N2. HCl (6 N in 2-propanol, 3.02 mL, 18.11 mmol) was added and the mixture was stirred for 5 min. Then, intermediate A34″ (705 mg, 3.47 mmol) and EDCI (1.16 g, 6.04 mmol) were added and the mixture was stirred at r.t. for 50 min. Additional intermediate A34″ (357 mg, 1.74 mmol) and EDCI (0.29 g, 1.51 mmol) were added and the mixture stirred one more hour. More intermediate A34 (123 mg, 0.604 mmol) and EDCI (0.16 g, 0.604 mmol) were added and the mixture stirred one more hour. Next, the mixture was evaporated to dryness and taken up in DCM (100 mL) and aq. sat. bicarbonate solution (40 mL). The organic layer was separated and the aqueous layer extracted with DCM (2×40 mL). The combined organic layers were dried over MgSO₄, filtered and concentrated under vacuum. The residue was purified by flash column chromatography (80 g silica gel, eluent DCM/(7 N NH₃ in MeOH), gradient 100:0 to 97:3), yielding compound II-6 (free base, 1390 mg, 89%) as a white solid. The solid was suspended in 2-propanol, excess HCl (6 N in 2-propanol) was added and the solvent evaporated (repeated once). The resulting solid was then triturated in 2-propanol, filtered and dried 3 h in vacuo at 75° C., yielding compound II-7 (HCl salt, 1260 mg, 75%) as a white powder.

### Example 2″

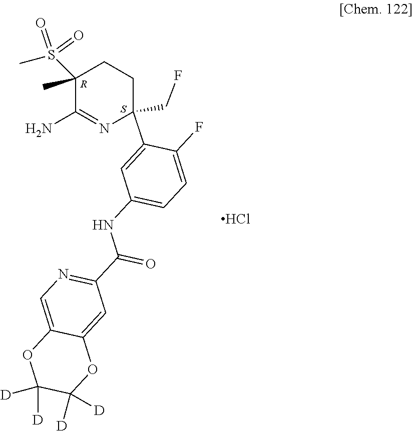

Intermediate B11″ (500 mg, 1.51 mmol) was dissolved in MeOH (50 mL) at r.t. under N2. HCl (6 N in 2-propanol, 1.51 mL, 9.05 mmol) was added and the mixture was stirred for 5 min. Then, intermediate A24″ (523 mg, 1.81 mmol) and EDCI (376 mg, 1.96 mmol) were added and the mixture was stirred at r.t. for 1.5 h. More EDCI (144 mg, 0.75 mmol) and intermediate A24″ (218 mg, 0.75 mmol) and stirred another 2 h. The mixture was evaporated to dryness and taken up in DCM (50 mL) and saturated aq. NaHCO₃ solution (50 mL). The organic layer was separated and the aqueous one extracted with DCM (2×100 mL). The combined organic layers were dried over MgSO₄, filtered and concentrated under vacuum. A purification by flash column chromatography was performed (40 g silica gel) using a gradient (DCM/(NH₃ in MeOH), 100:0 to 97:3). Evaporation of the product fractions yielded compound II-2 as a yellowish oil, which was dissolved in isopropanol. HCl (6 N in isopropanol, 5 mL) was added and the solvent evaporated in vacuo; this operation was repeated twice. The solid was then triturated in isopropanol and filtered. The resulting solid was lyophilized to afford compound II-3 (HCl salt, 427 mg, 53%) as a white solid.

### Example 3″

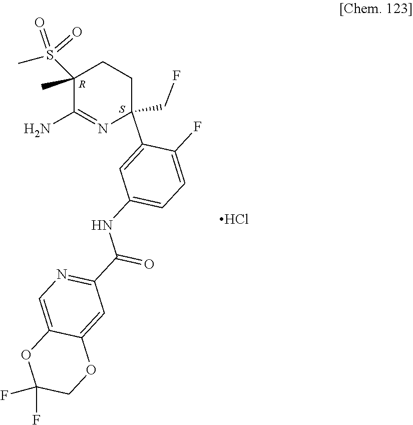

Intermediate B11″ (500 mg, 1.51 mmol) was dissolved in MeOH (50 mL) at r.t. under N2. HCl (6 N in 2-propanol, 1.51 mL, 9.05 mmol) was added and the mixture was stirred for 5 min. Then, intermediate A9″ (377 mg, 1.74 mmol) and EDCI (1.16 g, 6.04 mmol) were added and the mixture was stirred at r.t. for 50 min. More EDCI (144 mg, 0.75 mmol) and intermediate A9″ (218 mg, 0.75 mmol) were added and the mixture further stirred 1 h. The mixture was evaporated to dryness and taken up in DCM (100 mL) and saturated aq. NaHCO₃ solution (40 mL). The organic layer was separated and the aqueous one extracted with DCM (2×80 mL). The combined organic layers were dried over MgSO₄, filtered and concentrated under vacuum. A purification by flash column chromatography was performed (80 g silica gel, gradient DCM/(7 N NH₃ in MeOH), 100:0 to 95:5), yielding compound II-9 (930 mg, yield 116%) as a white foam. This foam was suspended in isopropanol, HCl (6 N in 2-propanol) was added and the solvent evaporated; this operation was repeated twice. The solid was then triturated in 2-propanol, filtered and dried in vacuo overnight at 75° C. yielding compound II-10 (HCl salt, 614 mg, yield 72%) as a white powder.

### Example 4″

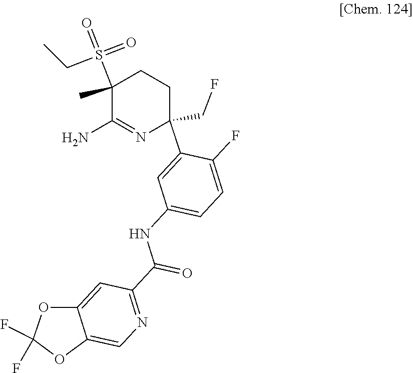 [SMILES: CCS(=O)(=O)[C@]1(C)CC[C@@](CF)(c2cc(NC(=O)c3cc4c(cn3)OC(F)(F)O4)ccc2F)N=C1N]

Intermediate B28″ (mono HCl salt, 12.0 g, 31.4 mmol) was dissolved in 280 mL MeOH at rt. Then, intermediate A34″ (6.4 g, 31.5 mmol) and EDCI (8.0 g, 41.7 mmol) were added and the mixture was stirred at rt for 20 min. The mixture was concentrated under vacuum and the residue was taken up in ethyl acetate (200 mL), stirred for 1 hour and then decanted. 50 mL EtOAc was added to the slurry, stirred for 5 minutes, decanted again and the obtained semi-solid slurry was taken up in DCM and an aq. sat. Na₂CO₃ solution. The organic layer was separated and the aqueous one extracted with DCM. The combined organic layers were dried over MgSO₄, filtered and concentrated under vacuum. The residue was purified by flash column chromatography (330 g silica gel, gradient: DCM/NH₃ (7 N in MeOH), from 100/0 up to 96/4). The desired product fractions were collected and evaporated.

Free base monohydrate: The residue was taken up in 2-propanol and the product precipitated with H₂O addition. After stirring for 30 minutes the solid was filtered, washed with DIPE and dried in vacuo at 50° C. giving compound II-20 (13.5 g, 78%).

Mono HCl salt: The residue was taken up in 20 mL ethyl acetate, then 0.8 mL of HCl (6 N in 2-propanol) was added, some solid occurred, the mixture was warmed up to 65° C., then cooled back to 20° C. The resulting solid was filtered, washed with ethyl acetate and dried for 2 days at 40° C. in the vacuum oven and one more night at 55° C. in the vacuum oven providing compound II-21.

### Example 5″

The compound II-29 was prepared according to the following synthetic route.

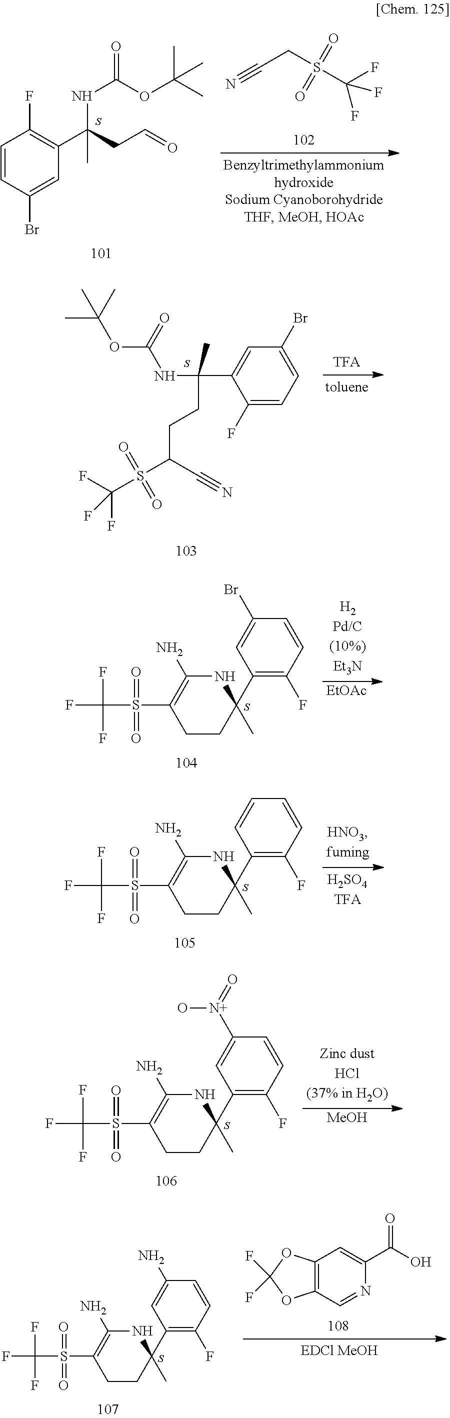

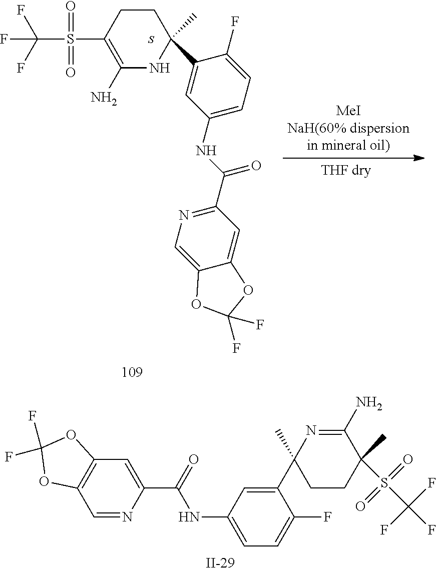

### Example 6″

The compound II-35 was prepared according to the following synthetic route.

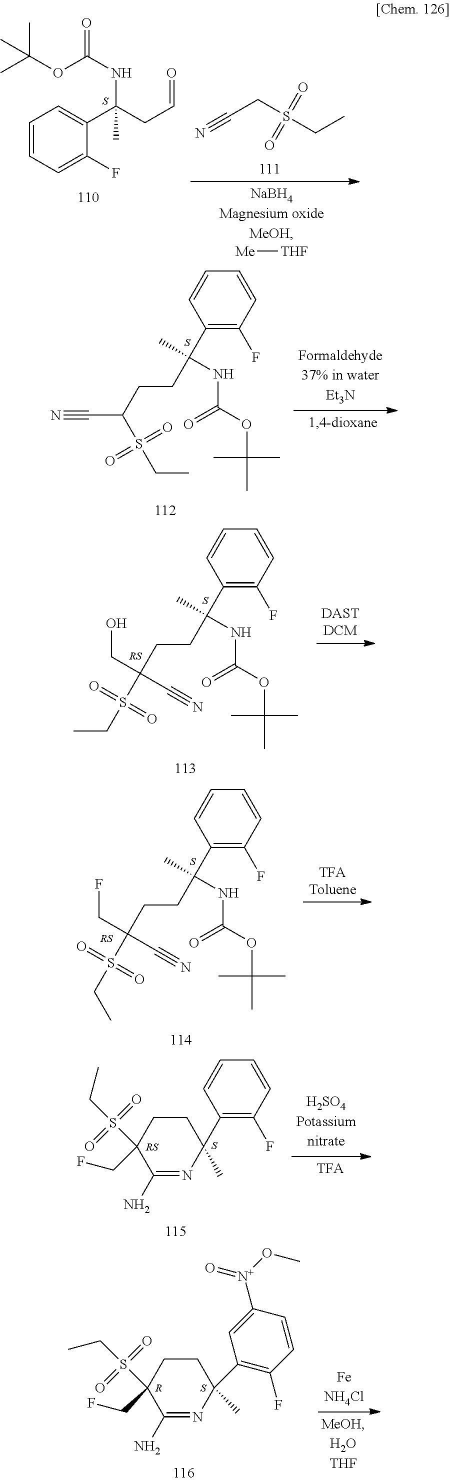

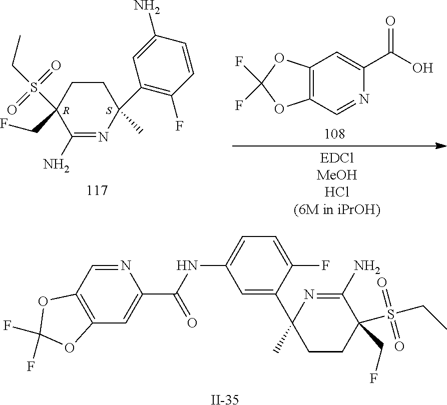

The following Table lists the compounds that were prepared by analogy to one of the above Examples. In case no salt form is indicated, the compound was obtained as a free base. ‘Co. No.’ means compound number. “cPr” means cyclopropyl.

TABLE 19

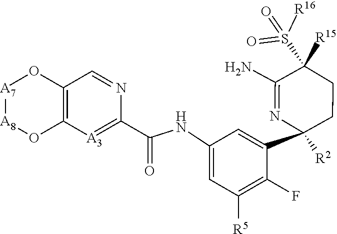

| | | | | | | | Stereo- | Salt |
|---|---|---|---|---|---|---|---|---|
| Co. No. | A₃ | R¹⁶ | R¹⁵ | R² | R⁵ | A₇—A₈ | chemistry | form |
| II-1 | CH | Me | Me | CH₂F | H | CH₂—CH₂ | (2S,5R) | |
| II-2 | CH | Me | Me | CH₂F | H | CD₂—CD₂ | (2S,5R) | |
| II-3 | CH | Me | Me | CH₂F | H | CD₂—CD₂ | (2S,5R) | HCl |
| II-4 | CH | Me | Me | CH₂F | H | CH₂ | (2S,5R) | |
| II-5 | CH | Me | Me | CH₂F | H | CH₂—CH₂—CH₂ | (2S,5R) | |
| II-6 | CH | Me | Me | CH₂F | H | CF₂ | (2S,5R) | |
| II-7 | CH | Me | Me | CH₂F | H | CF₂ | (2S,5R) | HCl |
| II-8 | CF | Me | Me | CH₂F | H | CH₂—CH₂ | (2S,5R) | |
| II-9 | CH | Me | Me | CH₂F | H | CF₂—CH₂ | (2S,5R) | |
| II-10 | CH | Me | Me | CH₂F | H | CF₂—CH₂ | (2S,5R) | HCl |
| II-11 | CH | Me | Me | CH₂F | H | CH₂—CHF (S) | (2S,5R) | |
| II-12 | CF | Me | Me | CH₂F | H | CD₂—CD₂ | (2S,5R) | |
| II-13 | CF | Me | Me | CH₂F | H | CF₂ | (2S,5R) | |
| II-14 | CF | Me | Me | CH₂F | H | CF₂—CH₂ | (2S,5R) | |
| II-15 | CMe | Me | Me | CH₂F | H | CF₂—CH₂ | (2S,5R) | |
| II-16 | CH | Me | Me | CH₂F | F | CD₂—CD₂ | (2S,5R) | |
| II-17 | CH | Me | Me | CH₂F | F | CF₂—CH₂ | (2S,5R) | |
| II-18 | CH | Me | Me | CH₂F | F | CF₂ | (2S,5R) | |
| II-19 | CH | Et | Me | CH₂F | H | CF₂—CH₂ | (2S,5R) | |
| II-20 | CH | Et | Me | CH₂F | H | CF₂ | (2S,5R) | |
| II-21 | CH | Et | Me | CH₂F | H | CF₂ | (2S,5R) | HCl |
| II-22 | N | Me | Me | CH₂F | F | CH₂CH₂ | (2S,5R) | |
| II-23 | CH | Et | Me | CH₂F | F | CF₂ | (2S,5R) | HCl |
| II-24 | CH | Et | Me | CH₂F | F | CD₂—CD₂ | (2S,5R) | |
| II-25 | CCl | Me | Me | CH₂F | H | CF₂ | (2S,5R) | |
| II-26 | CCl | Me | Me | CH₂F | H | CH₂—CH₂ | (2S,5R) | |

TABLE 20

| | | | | | | | | |
|---|---|---|---|---|---|---|---|---|
| | | | | | | | Stereo- | Salt |
| Co. No. | A₃ | R¹⁶ | R¹⁵ | R² | R⁵ | A₇-A₈ | chemistry | form |

| | | | | | | | |
|---|---|---|---|---|---|---|---|
| II-27 | CH | cPr | Me | CH₂F | H | CF₂CH₁ | (2S,5R) |
| II-28 | CH | iPr | Me | CH₂F | H | CF₂CH₂ | (2S,5R) |
| II-29 | CH | CF₃ | Me | Me | H | CF₂ | (2S,5R) |
| II-30 | CH | Me | CH₂F | Me | H | CD₂—CD₂ | (2S,5R) |
| II-31 | CH | Me | CH₂F | Me | H | CF₂—CH₂ | (2S,5R) |
| II-32 | CH | Me | CH₂F | Me | H | CF₂ | (2S,5R) |
| II-33 | CH | Et | CH₂F | Me | H | CD₂—CD₂ | (2S,5R) |
| II-34 | CH | Et | CH₂F | Me | H | CF₂—CH₂ | (2S,5R) |
| II-35 | CH | Et | CH₂F | Me | H | CF₂ | (2S,5R) |

### LC-MS (Liquid Chromatography/Mass Spectrometry)

### LC-MS General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified otherwise in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻, etc.). For molecules with multiple isotopic patterns (e.g. Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method codes (Flow expressed in mL/min; column temperature (T) in ° C.; Run time in minutes)

TABLE 21

| Method code | Instrument | Column | Mobile phase | Gradient | $\frac{Flow}{Col T}$ | Run time |
|---|---|---|---|---|---|---|
| A2 | Waters: Acquity ® UPLC ®- DAD and SQD | Waters: BEH C18 (1.7 μm, 2.1*50 mm) | A: 10 mM CH3COONH4 in 95% H2O + 5% CH3CN B: CH3CN | From 95% A to 5% A in 1.3 min, held for 0.7 min. | $\frac{0.8}{55}$ | 2 |
| B2 | Waters: Acquity ® UPLC ®- DAD and SQD | Waters: HSS T3 (1.8 μm, 2.1*100 mm) | A: 10 mM CH3COONH4 in 95% H2O + 5% CH3CN B: CH3CN | From 100% A to 5% A in 2.10 min, to 0% A in 0.90 min, to 5% A in 0.5 min | $\frac{0.7}{55}$ | 3.5 |
| C2 | Waters UPLC | Waters: XBridge (Registered Trade mark) C18 (5 μm, i.d. 4.6 × 50 mm) | A: 0.1% formic acid solution; B: 0.1% formic acid in acetonitrile | Linear gradient of 10% to 100% solvent B for 3 min, next 100% solvent B maintained for 1 min | $\frac{3}{ }$ | 4 |
| D2 | Shimadzu UFLC | Shimadzu: Shim-pack XR-ODS (2.2 μm, i.d. 50 x 3.0 mm) | A: 0.1% formic acid-containing aqueous solution; B 0.1% formic acid-containing acetonitrile solution | linear gradient from 10% to 100% solvent [B] for 3 min and 100% solvent [B] for 1 min | $\frac{3}{50}$ | 4 |

### Melting Points

Values are either peak values or melt ranges, and are obtained with experimental uncertainties that are commonly associated with this analytical method.

For a number of compounds, melting points were determined with a DSC823e (Mettler-Toledo) apparatus. Melting points were measured with a temperature gradient of 10° C./minute. Maximum temperature was 300° C.

Analytical data—melting point (m.p.) and LC/MS: Rt means retention time (in minutes), [M+H]⁺ means the protonated mass of the compound, [M−H]⁻ means the deprotonated mass of the compound, method refers to the method used for (LC)MS. For some compounds, the exact mass was determined.

TABLE 22

| | | | MW | | | |
|---|---|---|---|---|---|---|
| Co. no. | m.p. (° C.) | Rt | (theor) | [M + H]⁺ | [M − H]⁻ | method |
| II-1 | | 1.66 | 494 | 495 | 493 | B2 |
| II-2 | | 0.94 | 498 | 499 | 497 | A2 |
| II-3 | | 1.67 | 498 | 499 | 497 | B2 |
| II-4 | | 0.83 | 480 | 481 | 479 | A2 |
| II-5 | | 0.88 | 508 | 509 | 507 | A2 |
| II-6 | 190.5 | 1.08 | 516 | 517 | 515 | A2 |
| II-7 | 279.7 | 0.96 | 516 | 517 | 515 | A2 |
| II-8 | | 0.81 | 512 | 513 | 511 | A2 |
| II-9 | | 0.93 | 530 | 531 | 529 | A2 |
| II-10 | 264.1 | 0.93 | 530 | 531 | 529 | A2 |
| II-11 | | 0.87 | 512 | 513 | 511 | A2 |
| II-12 | | 0.82 | 516 | 517 | 515 | A2 |
| II-16 | | 1.76 | 516 | 517 | 515 | B2 |
| II-17 | | 1.89 | 548 | 549 | 547 | B2 |
| II-18 | 195 | 1.96 | 534 | 535 | 533 | B2 |
| II-19 | | 0.98 | 544 | 545 | 544 | A2 |
| II-20 | | 1.02 | 530 | 531 | 529 | A2 |
| II-21 | 271.8 | 1.02 | 530 | 531 | 529 | A2 |
| II-22 | | 0.76 | 513 | 514 | 512 | A2 |
| II-23 | | 1.09 | 548 | 549 | 547 | A2 |
| II-24 | | 0.89 | 512 | 513 | 511 | A2 |
| II-25 | | 1.47 | 550 | 551 | | C2 |
| II-26 | | 1.12 | 529 | 530 | | D2 |
| II-27 | | 0.99 | 556 | 557 | 555 | A2 |
| II-28 | | 1.03 | 558 | 559 | 557 | A2 |
| II-29 | | 2.24 | 552 | 553 | 551 | B2 |
| II-30 | | 0.87 | 498 | 499 | 497 | A2 |
| II-31 | | 0.96 | 530 | 531 | 529 | A2 |
| II-32 | | 1 | 516 | 517 | 515 | A2 |
| II-33 | | 1.82 | 512 | 513 | 511 | B2 |
| II-34 | | 1.01 | 544 | 545 | 543 | A2 |
| II-35 | | 1.04 | 530 | 531 | 529 | A2 |

### SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Analytical SFC-MS Methods (Flow expressed in mL/min; column temperature (T) in ° C.; Run time in minutes, Backpressure (BPR) in bars.

TABLE 23

| Method code | column | mobile phase | gradient | $\frac{Flow}{Col T}$ | $\frac{\begin{matrix}Run \\ Time\end{matrix}}{BPR}$ |
|---|---|---|---|---|---|
| Method 1 AD_iPrOH_G2A | Daicel Chiralpak | A: CO2 B: | 10%-50% B in 6 min, | $\frac{2.5}{40}$ | $\frac{9.5}{130}$ |
| 1P1BSFC3 | (Registered | iPrOH + 0.2% | hold 3.5 min | | |
| | trademark) | iPrNH₂ | | | |
| | AD3 column | | | | |
| | (3.0 μm, 150 × | | | | |
| | 4.6 mm) | | | | |
| Method 2 AS_EtOH_G2A1 | Daicel Chiralpak | A: CO2 B: | 5% B hold 6 min, to | $\frac{2.5}{40}$ | $\frac{9.5}{130}$ |
| P1BSFC2 | (Registered | EtOH + 0.2% | 50% in 1 | | |
| | trademark) | iPrNH2 | min hold 2.5 | | |
| | AS3 column | | min | | |
| | (3.0 μm, 150 × | | | | |
| | 4.6 mm) | | | | |

Analytical SFC data—Rt means retention time (in minutes), [M+H]⁺ means the protonated mass of the compound, method refers to the method used for (SFC)MS analysis of enantiomerically pure compounds.

TABLE 24

| Co. no. | Method | Rt | [M + H]⁺ |
|---|---|---|---|
| II-1 | 2 | 3.7 | 495 |
| II-16 | 2 | 7.4 | 517 |
| II-17 | 1 | 5.03 | 549 |
| II-18 | 1 | 5.45 | 535 |

### NMR

For a number of compounds, ¹H NMR spectra were recorded on a Bruker DPX-400 spectrometer operating at 400 MHz, on a Bruker DPX-360 operating at 360 MHz, or on a Bruker Advance 400 spectrometer operating at 400 MHz, or on a Bruker Avance 600 spectrometer operating at 600 MHz, using CHLOROFORM-d (deuterated chloroform, CDCl₃) or DMSO-d₆ (deuterated DMSO, dimethyl-d6 sulfoxide) or BENZENE-d6 (deuterated benzene, C₆D₆) or ACETONE-d₆ (deuterated acetone, (CD₃)₂CO) as solvents. Chemical shifts (δ) are reported in parts per million (ppm) relative to tetramethylsilane (TMS), which was used as internal standard.

TABLE 25

| Co. No. | ¹H NMR result |
|---|---|
| II-1 | ¹H NMR (360 MHz, DMSO-d₆) δ ppm 1.37-1.52 (m, 1 H) 1.55 (s, 3 H) 1.81- |
| | 1.95 (m, 1 H) 2.11-2.26 (m, 1 H) 2.30-2.41 (m, 1 H) 3.08 (s, 3 H) 4.32- |
| | 4.66 (m, 6 H) 6.01 (br s, 2 H) 7.10-7.17 (m, 1 H) 7.58 (s, 1 H) 7.76-7.81 (m, |
| | 2 H) 8.25 (s, 1 H) 10.49 (s, 1 H) |
| II-2 | ¹H NMR (400 MHz, CHLOROFORM-d) δ ppm 1.66 (s, 3 H) 1.75-1.88 (m, |
| | 1 H) 1.96-2.06 (m, 1 H) 2.54-2.67 (m, 2 H) 3.04 (s, 3 H) 4.41 (br dd, |
| | J = 47.8, 8.7 Hz, 1 H) 4.94 (br dd, J = 47.8, 8.7 Hz, 1 H) 5.45 (br s, 2 H) 7.06 |
| | (dd, J = 11.6, 8.7 Hz, 1 H) 7.72 (dd, J = 6.7, 2.6 Hz, 1 H) 7.80 (s, 1 H) 7.91 (dt, |
| | J = 8.3, 3.6 Hz, 1 H) 8.13 (s, 1 H) 9.85 (br s, 1 H) |
| II-3 | ¹H NMR (360 MHz, DMSO-d₆) δ ppm 1.57-1.70 (m, 1 H) 1.74 (s, 1 H) 1.75- |
| | 1.86 (m, 2 H) 2.06-2.18 (m, 1 H) 2.35-2.46 (m, 1 H) 2.51-2.54 (m, 1 H) |
| | 3.35 (s, 3 H) 4.69 (br dd, J = 47.2, 9.9 Hz, 1 H) 4.98 (dd, J = 46.5, 10.2 Hz, 1 |
| | H) 7.33 (dd, J = 12.1, 9.1 Hz, 1 H)7.61 (s, 1 H) 7.80 (dd, J = 7.3, 2.6 Hz, 1 H) |
| | 8.02-8.11 (m, 1 H) 8.28 (s, 1 H) 9.33 (br s, 1 H) 9.53 (br s, 1 H) 10.77 (s, 1 |
| | H) 10.82 (brs, 1 H) |
| II-4 | ¹H NMR (400 MHz, CHLOROFORM-d) δ ppm 1.67 (s, 3 H) 1.78-1.87 (m, |
| | 1 H) 1.96-2.10 (m, 1 H) 2.54-2.67 (m, 2 H) 3.04 (s, 3 H) 4.41 (br dd, |
| | J = 47.0, 8.5 Hz, 1 H) 4.94 (br dd, J = 47.9, 8.9 Hz, 1 H) 5.45 (br s, 2 H) 6.17 (s, |
| | 2H) 7.06 (dd, J = 11.4, 8.8 Hz, 1 H) 7.72 (dd, J = 6.8, 2.6 Hz, 1 H) 7.79 (s, 1 H) |
| | 7.90 (dt, J = 8.7, 3.5 Hz, 1 H) 8.06 (s, 1 H) 9.82 (br s, 1 H) |
| II-5 | ¹H NMR (360 MHz, CHLOROFORM-d) δ ppm 1.66 (s, 3 H) 1.76-1.86 (m, |
| | 1 H) 1.96-2.05 (m, 1 H) 2.25-2.37 (m, 2 H) 2.50-2.67 (m, 2 H) 3.04 (s, 3 |
| | H) 4.29-4.52 (m, 5 H) 4.82-5.06 (m, 1 H) 5.50 (br s, 2 H) 7.05 (dd, J = 11.3, |
| | 8.8 Hz, 1 H) 7.74 (dd, J = 7.0, 2.6 Hz, 1 H) 7.81 (s, 1 H) 7.88 (ddd, J = 8.8, 4.0, |
| | 2.9 Hz, 1 H) 8.17 (s, 1 H) 9.84 (br s, 1 H) |
| II-6 | ¹H NMR (360 MHz, CHLOROFORM-d) δ ppm 1.67 (s, 3 H) 1.78-1.88 (m, |
| | 1 H) 1.96-2.06 (m, 1 H) 2.55-2.69 (m, 2 H) 3.05 (s, 3 H) 4.42 (br dd, |
| | J = 47.0, 9.0 Hz, 1 H) 4.94 (dd, J = 47.8, 8.2 Hz, 1 H) 5.48 (br s, 2 H) 7.09 (dd, |
| | J = 11.5, 9.0 Hz, 1 H) 7.75 (dd, J = 6.8, 2.7 Hz, 1 H) 7.90 (dt, J = 8.8, 3.5 Hz, 1 |
| | H) 8.10 (s, 1 H) 8.38 (s, 1 H) 9.79 (br s, 1 H) |

TABLE 26

| | |
|---|---|
| II-7 | ¹H NMR (400 MHz, DMSO-d₆) δ ppm 1.54-1.70 (m, 1 H) 1.77 (s, 3 H) 2.03- |
| | 2.17 (m, 1 H) 2.32-2.43 (m, 1 H) 2.49-2.53 (m, 1 H) 3.31 (s, 3 H) 4.66 (br |
| | dd, J = 47.3, 10.3 Hz, 1 H) 4.95 (br dd, J = 46.1, 10.0 Hz, 1 H) 7.32 (dd, |
| | J = 12.1, 9.0 Hz, 1 H) 7.78 (dd, J = 7.5, 2.4 Hz, 1 H) 7.97-8.07 (m, 1 H) 8.18 |
| | (s, 1 H) 8.79 (s, 1 H) 9.42 (br s, 1 H) 9.51 (br s, 1 H) 10.88 (br s, 2 H) |
| II-8 | ¹H NMR (400 MHz, DMSO-d₆) δ ppm 1.51-1.62 (m, 1 H) 1.65 (s, 3 H) 1.94- |
| | 2.07 (m, 1 H) 2.23-2.36 (m, 1 H) 2.42-2.53 (m, 1 H) 3.19 (s, 3 H) 4.50- |
| | 4.63 (m, 5 H) 4.65-4.76 (m, 1 H) 6.11 (br s, 2 H) 7.24 (dd, J = 11.8, 8.7 Hz, 1 |
| | H) 7.80-7.90 (m, 2 H) 8.25 (s, 1 H) 10.53 (br s, 1 H) |
| II-9 | ¹H NMR (400 MHz, DMSO-d₆) δ ppm 1.42-1.53 (m, 1 H) 1.56 (s, 3 H) 1.85- |
| | 1.98 (m, 1 H) 2.14-2.29 (m, 1 H) 2.32-2.48 (m, 1 H) 3.10 (s, 3 H) 4.44- |
| | 4.66 (m, 2 H) 4.86 (t, J = 6.5 Hz, 2 H) 6.01 (br s, 2 H) 7.16 (dd, J = 11.7, 8.6 |
| | Hz, 1 H) 7.78-7.87 (m, 3 H) 8.60 (s, 1 H) 10.60 (br s, 1 H) |
| II-10 | ¹H NMR (400 MHz, DMSO-d₆) δ ppm 1.59-1.72 (m, 1 H) 1.81 (s, 3 H) 2.08- |
| | 2.17 (m, 1 H) 2.41 (td, J = 14.2, 2.9 Hz, 1 H) 2.52-2.56 (m, 1 H) 3.35 (s, 3 |
| | H) 4.57-5.08 (m, 2 H) 4.87 (t, J = 6.5 Hz, 2 H) 7.35 (dd, J = 12.1, 8.8 Hz, 1 H) |
| | 7.81 (dd, J = 7.5, 2.4 Hz, 1 H) 7.85 (s, 1 H) 8.03-8.14 (m, 1 H) 8.62 (s, 1 H) |
| | 9.43 (s, 1 H) 9.54 (s, 1 H) 10.87 (s, 1 H) 10.89 (s, 1 H) |
| II-11 | ¹H-NMR (CDCl3) δ: 1.67 (3H, s), 1.75-1.87 (1H, m), 1.95-2.06 (1H, m), 2.53- |
| | 2.67 (2H, m), 3.04 (3H, s), 4.09-4.21 (1H, m), 4.32-4.49 (1H, m), 4.51-4.59 |
| | (1H, m), 4.85-5.02 (1H, m), 6.14 (1H, d, J = 52.0 Hz), 7.06 (1H, dd, J = 11.4, |
| | 8.7 Hz), 7.71 (1H, dd, J = 6.9, 2.9 Hz), 7.88-7.91 (1H, m), 7.92 (1H, s), 8.24 |
| | (1H, s), 9.81 (1H, s) |
| II-12 | ¹H NMR (400 MHz, DMSO-d₆) δ ppm 1.46 (ddd, J = 14.8, 11.1, 3.5 Hz, 1 H) |
| | 1.54 (s, 3 H) 1.83-1.95 (m, 1 H) 2.13-2.24 (m, 1 H) 2.33-2.41 (m, 1 H) |
| | 3.08 (s, 3 H) 4.41-4.67 (m, 2 H) 5.99 (br s, 2 H) 7.13 (dd, J = 12.0, 8.7 Hz, 1 |
| | H) 7.63-7.81 (m, 2 H) 8.14 (s, 1 H) 10.41 (br s, 1 H) |
| II-16 | ¹H NMR (400 MHz, CHLOROFORM-d) δ ppm 1.66 (s, 3 H), 1.77-1.89 (m, |
| | 1 H), 1.92-2.06 (m, 1 H), 2.54-2.70 (m, 2 H), 3.03 (s, 3 H), 4.27-5.00 (m, 2 |
| | H), 7.32-7.44 (m. 1 H), 7.79 (s, 1 H), 7.99-8.10 (m, 1 H), 8.13 (s, 1 H), 9.87 |
| | (s, 1 H). |

TABLE 27

| | |
|---|---|
| II-17 | ¹H NMR (400 MHz, CHLOROFORM-d) δ ppm 1.67 (s, 3 H) 1.77-1.89 (m, |
| | 1 H) 1.95-2.06 (m, 1 H) 2.55-2.70 (m, 2 H) 3.04 (s, 3 H) 4.30-4.51 (m, 3 |
| | H) 4.91 (br dd, J = 47.2, 8.5 Hz, 1 H) 5.47 (br s, 2 H) 7.39-7.43 (m, 1 H) 7.95 |
| | (s, 1 H) 8.03 (ddd, J = 11.7, 6.8, 2.6 Hz, 1 H) 8.33 (s, 1 H) 9.83 (br s, 1 H) |
| II-18 | ¹H NMR (400 MHz, CHLOROFORM-d) δ ppm 1.66 (s, 3 H), 1.77-1.89 (m, |
| | 1 H), 1.92-2.06 (m, 1 H), 2.54-2.70 (m, 2 H), 3.03 (s, 3 H), 4.27-5.00 (m, 2 |
| | H), 7.32-7.44 (m, 1 H), 7.79 (s, 1 H), 7.99-8.10 (m, 1 H), 8.13 (s, 1 H), 9.87 |
| | (s, 1 H) |
| II-19 | ¹H NMR (400 MHz, CDCl₃) δ ppm 1.47 (t, J = 7.53 Hz, 3 H) 1.67 (s, 3 H) 1.73- |
| | 1.82 (m, 1 H) 1.96-2.10 (m, 1 H) 2.50-2.64 (m, 2 H) 3.20 (q, J = 7.46 Hz, 2 |
| | H) 4.41 (dd, J = 48.01, 8.95 Hz, 1 H) 4.41 (t, J = 5.90 Hz, 2 H) 4.91 (dd, |
| | J = 48.42, 9.36 Hz, 1 H) 5.47 (br s, 2 H) 7.06 (dd, J = 11.39, 8.95 Hz, 1 H) 7.72 |
| | (dd, J = 6.71, 2.64 Hz, 1 H) 7.85-7.90 (m, 1 H) 7.95 (s, 1 H) 8.31 (s, 1 H) 9.80 |
| | (s, 1 H) |
| II-20 | ¹H NMR (400 MHz, CDCl₃) δ ppm 1.48 (t, J = 7.53 Hz, 3 H) 1.68 (s, 3 H) 1.73- |
| | 1.84 (m, 1 H) 1.97-2.09 (m, 1 H) 2.52-2.64 (m, 2 H) 3.20 (q, J = 7.46 Hz, 2 |
| | H) 4.41 (dd, J = 47.20, 8.54 Hz, 1 H) 4.91 (dd, J = 48.83, 8.54 Hz, 1 H) 5.44 (br |
| | s, 2 H) 7.07 (dd, J = 11.39, 8.95 Hz, 1 H) 7.72 (dd, J = 6.71, 2.64 Hz, 1 H) 7.84- |
| | 7.92 (m, 1 H) 8.09 (d, J = 0.81 Hz, 1 H) 8.37 (s, 1 H) 9.77 (s, 1 H) |
| II-21 | ¹H NMR (400 MHz, DMSO-d₆) δ ppm 1.38 (t, J = 7.37 Hz, 3 H) 1.73- 1.83 |
| | (m, 1 H) 1.82 (s, 3 H) 2.18-2.26 (m, 1 H) 2.41-2.50 (m, 1 H) 2.54-2.62 (m, |
| | 1 H) 3.44-3.53 (m, 2 H) 4.76 (ddd, J = 47.32, 9.90, 1.10 Hz, 1 H) 4.97 (dd, |
| | J = 46.44, 9.90 Hz, 1 H) 7.31 (dd, J = 12.32, 9.02 Hz, 1 H) 7.87 (dd, J = 7.48, |
| | 2.64 Hz, 1 H) 7.99 (ddd, J = 8.80, 4.18, 2.64 Hz, 1 H) 8.16(s, 1 H) 8.76 (s, 1 |
| | H) 9.31 (br s, 2 H) 10.58 (s, 1 H) 10.90 (br s, 1 H) |
| II-22 | ¹H NMR (400 MHz, DMSO-d₆) δ ppm 1.63-1.74 (m, 1 H), 1.80 (s, 3 H), |
| | 2.09-2.19 (m, 1 H), 2.32-2.46 (m, 2 H), 3.35 (s, 3 H), 4.40-4.65 (m, 4 H), |
| | 4.64-5.11 (m, 2 H), 7.66 (br d, J = 6.1 Hz, 1 H), 8.06-8.24 (m, 1 H), 8.48 (s, |
| | 1 H), 9.26 (br s, 1 H), 9.56 (br s, 1 H), 10.80 (s, 1 H), 10.81 (s, 1 H), 10.96 (s, |
| | 1 H) |
| II-23 | ¹H NMR (400 MHz, DMSO-d₆) δ ppm 1.29 (br t, J = 7.26 Hz, 3 H) 1.46-1.58 |
| | (m, 1 H) 1.56 (s, 3 H) 1.88-2.02 (m, 1 H) 2.15-2.28 (m, 1 H) 2.34-2.46 (m, |
| | 1 H) 3.16-3.27 (m, 2 H) 4.40-4.72 (m, 2 H) 6.05 (br s, 2 H) 7.68-7.77 (m, |
| | 1 H) 7.95 (br dd, J = 9.79, 6.71 Hz, 1 H) 8.21 (s, 1 H) 8.81 (s, 1 H) 10.86 (br s, |
| | 1H) |

TABLE 28

| | |
|---|---|
| II-24 | ¹H NMR (400 MHz, CDCl₃) δ ppm 1.47 (t, J = 7.53 Hz, 3 H) 1.67 (s, 3 H) 1.72- |
| | 1.82 (m, 1 H) 1.98-2.09 (m, 1 H) 2.51-2.63 (m, 2 H) 3.20 (q, J = 7.46 Hz, 2 |
| | H) 4.40 (dd, J = 47.61, 8.54 Hz, 1 H) 4.91 (dd, J = 48.42, 8.54 Hz, 1 H) 5.46 (br |
| | s, 2 H) 7.04 (dd, J = 11.60, 8.75 Hz, 1 H) 7.70 (dd, J = 6.51, 2.85 Hz, 1 H) 7.79 |
| | (s, 1 H) 7.85-7.92 (m, 1 H) 8.11 (s, 1 H) 9.84 (s, 1 H) |
| II-25 | ¹H NMR (400 MHz, CDCl₃) δ ppm 1.66 (s, 3H), 1.78-1.87 (m, 1H), 1.95-2.06 |
| | (m, 1H), 2.53-2.70 (m, 2H), 3.04 (s, 3H), 4.40 (dd, J = 47.0, 8.5 Hz, 1H), 4.93 |
| | (dd, J = 47.2, 9.0 Hz, 1H), 7.08 (dd, J = 11.4, 8.8 Hz, 1H), 7.61 (dd, J = 6.7, |
| | 2.8 Hz, 1H), 7.98 (ddd, J = 7.2, 4.2, 3.0 Hz, 1H), 8.31 (s, 1H), 9.68 (s, 1H). |
| II-26 | ¹H NMR (400 MHz, CDCl₃) δ ppm 1.66 (s, 3H), 1.76-1.88 (m, 1H), 1.96-2.08 |
| | (m, 1H), 2.53-2.69 (m, 2H), 3.04 (s, 3H), 4.37-4.40 (m, 2H), 4.39 (dd, J = |
| | 47.5, 8.9 Hz, 1H), 4.49-4.52 (m, 2H), 4.94 (dd, J = 47.5, 8.8 Hz, 1H), 7.05 |
| | (dd, J = 11.7, 8.9 Hz, 1H), 7.58 (dd, J = 6.7, 2.8 Hz, 1H), 8.02 (ddd, J = 8.8, |
| | 4.1, 2.9 Hz, 1H), 8.10 (s, 1H), 9.83 (s, 1H). |

TABLE 29

| | |
|---|---|
| II-29 | 1H NMR (400 MHz, CHLOROFORM-d) δ ppm 1.60 (s, 3 H), 1.84 (s, 3 H), |
| | 1.86-1.93 (m, 1 H), 1.95-2.07 (m, 1 H), 2.28-2.41 (m, 1 H), 2.55-2.69 (m, |
| | 1 H), 4.82-5.36 (m, 2 H), 6.96-7.16 (m, 1 H), 7.60-7.73 (m, 2 H), 7.67 (dd, |
| | J = 7.0, 2.9 Hz, 1 H), 7.73-7.85 (m, 1 H), 8.08 (s, 1 H), 8.34 (s, 1 H), 9.74 (s, 1 |
| | H) |
| II-30 | 1H NMR (400 MHz, DMSO-d6) δ ppm 1.51 (s, 3 H), 1.65-1.85 (m, 1 H), |
| | 1.90-2.19 (m, 2 H), 2.33-2.47 (m, 1 H), 3.10-3.26 (m, 3 H), 4.72-4.92 (m, |
| | 1 H), 4.97-5.24 (m, 1 H), 5.75 (s, 2 H), 7.12 (br dd, J = 11.6, 8.9 Hz, 1 H), |
| | 7.59 (s, 1 H), 7.70 (dd, J = 7.5, 2.6 Hz, 1 H), 7.75-7.86 (m, 1 H), 8.25 (s, 1 H), |
| | 10.39 (brs, 1 H) |
| II-31 | 1H NMR (400 MHz, DMSO-d6) δ ppm 1.51 (s, 3 H), 1.64-1.84 (m, 1 H), |
| | 1.90-2.18 (m, 2 H), 2.34-2.47 (m, 1 H), 3.18 (br s, 3 H), 4.72-4.91 (m, 3 |
| | H), 4.95-5.28 (m, 1 H), 5.83 (br s, 1 H), 7.14 (br dd, J = 11.8, 8.9 Hz, 1 H), |
| | 7.73 (dd, J = 7.5, 2.6 Hz, 1 H), 7.77-7.82 (m, 1 H), 7.83 (s, 1 H), 8.60 (s, 1 H), |
| | 10.54 (brs, 1 H) |
| II-32 | 1H NMR (400 MHz, DMSO-d6) δ ppm 1.55 (s, 3 H), 1.68-1.82 (m, 1 H), |
| | 2.01-2.22 (m, 2 H), 2.39-2.48 (m, 1 H), 3.22 (s, 3 H), 4.73-5.22 (m, 2 H), |
| | 5.49-7.00 (m, 1 H), 7.17 (dd, J = 11.9, 8.8 Hz, 1 H), 7.74-7.85 (m, 2 H), 8.20 |
| | (s, 1 H), 8.80 (s, 1 H), 10.64 (br s, 1 H) |
| II-33 | 1H NMR (400 MHz, DMSO-d6) δ ppm 1.29 (br t, J = 7.4 Hz, 3 H), 1.51 (s, 3 |
| | H), 1.63-1.86 (m, 1 H), 1.90-2.18 (m, 2 H), 2.33-2.47 (m, 1 H), 3.32-3.45 |
| | (m, 2 H), 4.64-5.29 (m, 2 H), 5.84 (br s, 1 H), 7.12 (br dd, J = 11.6, 8.9 Hz, 1 |
| | H), 7.59 (s, 1 H), 7.70 (dd, J = 7.5, 2.4 Hz, 1 H), 7.75-7.85 (m, 1 H), 8.25 (s, 1 |
| | H), 10.39 (br s, 1 H) |
| II-34 | 1H NMR (400 MHz, DMSO-d6, 80° C.) δ ppm 1.32 (t, J = 7.4 Hz, 4 H), 1.54 |
| | (d, J = 1.3 Hz, 3 H), 1.73-1.89 (m, 1 H), 1.99-2.11 (m, 1 H), 2.12-2.22 (m, 1 |
| | H), 2.38-2.48 (m, 1 H), 3.25-3.45 (m, 2 H), 4.71-4.90 (m, 4 H), 4.92-5.15 |
| | (m, 1 H), 5.28-6.45 (m, 1 H), 7.11 (dd, J = 12.0, 8.7 Hz, 1 H), 7.72-7.81 (m, |
| | 2 H), 7.81 (s, 1 H), 8.55 (s, 1 H), 10.29 (br s, 1 H) |
| II-35 | 1H NMR (400 MHz, CHLOROFORM-d) δ ppm 1.44-1.57 (m, 3 H), 1.65 (s, |
| | 3 H), 1.98-2.15 (m, 2 H), 2.26-2.39 (m, 1 H), 2.39-2.54 (m, 1 H), 3.09- |
| | 3.35 (m, 2 H), 4.55-5.53 (m, 3 H), 7.06 (br dd, J = 11.3, 8.9 Hz, 1 H), 7.61- |
| | 7.71 (m, 1 H), 7.77 (br dd, J = 5.5, 2.9 Hz, 1 H), 8.09 (s, 1 H), 8.35 (s, 1 H), |
| | 9.75 (br s, 1 H) |

### CHN Determinations

For a compound, amount of Carbon, Hydrogen and Nitrogen (CHN) in (% w/w) was determined by Dynamic Flash Combustion.

TABLE 30

| Co. No. | C | H | N |
|---|---|---|---|
| II-6 | 48.5 | 3.8 | 10.85 |
| II-21 | 47.2 | 4.1 | 9.6 |

### Optical Rotations:

Optical rotations were measured on a Perkin-Elmer 341 polarimeter with a sodium lamp and reported as follows: [α]° (λ, c g/100 ml, solvent, T° C.).

[α]_{λ}^{T}=(100α)/(l×c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation, the concentration and solvent are always provided in parentheses after the rotation. The rotation is reported using degrees and no units of concentration are given (it is assumed to be g/100 ml).

TABLE 31

| Co. No. | Result |
|---|---|
| II-6 | +81.9° (589 nm, c 0.536 w/v %, DMF, 20° C.) |

Test Examples for the compounds of the present invention are mentioned below.

### Pharmacological Examples

The compounds provided in the present invention are inhibitors of the beta-site APP-cleaving enzyme 1 (BACE1). Inhibition of BACE1, an aspartic protease, is believed to be relevant for treatment of Alzheimer's Disease (AD). The production and accumulation of beta-amyloid peptides (Abeta) from the beta-amyloid precursor protein (APP) is believed to play a key role in the onset and progression of AD. Abeta is produced from the amyloid precursor protein (APP) by sequential cleavage at the N- and C-termini of the Abeta domain by beta-site APP-cleaving enzyme 1 and gamma-secretase, respectively.

Compounds of Formula (IA), (IB), or (IC) are expected to have their effect selectively at BACE1 versus BACE2 by virtue of their ability to selectively bind to BACE1 versus BACE2 and inhibit the BACE1 versus BACE2 enzymatic activity. The behaviour of such inhibitors is tested using a biochemical competitive radioligand binding assay, a biochemical Fluorescence Resonance Energy Transfer (FRET) based assay and a cellular αLisa assay described below, which are suitable for the identification of such compounds.

### Test Example 1-1: BACE1 and BACE2 Biochemical Competitive Radioligand Binding Assay

To explore the BACE1 versus BACE2 enzyme selectivity, the binding affinity (Ki) to the respective purified enzymes was determined in a competitive radioligand binding assay, i.e. in competition with a tritiated non-selective BACE1/BACE2 inhibitor.

Briefly in test tubes, compounds of interest were combined with the radioligand and the BACE1 or BACE2-containing HEK 293 derived membrane. The competitive binding reaction was performed at pH 6.2 and incubated at room temperature until the equilibrium was reached. Afterwards free radioligand was separated from bound radioligand by filtration with a Brandell 96 harvester. The filter was washed 4 times with washing buffer and the filter sheets were punched into scintillation vials. Ultima Gold scintillation cocktail was added and samples were shaken. The day after, the vials were counted in a Tricarb scintillation counter to obtain the disintegrations per minute (dpm) of the bound radioligand.

Calculating the % CTL=(sample/HC)*100, with HC being the high control, i.e. total binding of radioligand, allowed to fit curves through the data points of the different doses of test compound. The pIC₅₀ or IC₅₀ was calculated and could be converted to Kᵢ by the formula Kᵢ=IC₅₀/(1+([RL]/K_{d})), with [RL] being the used concentration of radioligand and K_{d} the determinated dissociation constant of the radioligand-membrane complex.

### Test Example 1-2: BACE1 and BACE2 Biochemical Competitive Radioligand Binding Assay

To explore the BACE1 versus BACE2 enzyme selectivity, the binding affinity (Ki) to the respective purified enzymes was determined in a competitive radioligand binding assay, i.e. in competition with a tritiated non-selective BACE1/BACE 2 inhibitor.

Briefly in test tubes, compounds of interest are combined with the radioligand and the BACE1 or BACE2-containing HEK 293 derived membrane. The competitive binding reaction is performed at pH 6.2 and incubated at room temperature until the equilibrium is reached. Afterwards free radioligand is separated from bound radioligand by filtration with a Brandell 96 harvester. The filter is washed 4 times with washing buffer and the filter sheets are punched into scintillation vials. Ultima Gold scintillation cocktail is added and samples are shaken. The day after, the vials are counted in a Tricarb scintillation counter to obtain the disintegrations per minute (dpm) of the bound radioligand.

Calculating the % Inhibition=100−[(sample−LC)/(HC−LC))*100], with HC being the high control, i.e. total binding of radioligand and LC representing the non-specific binding measured in the presence of 10 μM of 3-[(1S)-4-[isobutyl(2-morpholinoethyl)amino]-1-isopropyl-butyl]-6-phenoxy-4H-pyrido[3,4-d]pyrimidin-2-amine, a known BACE inhibitor, allows to fit curves through the data points of the different doses of test compound. The pIC₅₀ or IC₅₀ is calculated and can be converted to Kᵢ by the formula Kᵢ=IC₅₀/(1+([RL]/K_{d})), with [RL] being the used concentration of radioligand and K_{d} the determined dissociation constant of the radioligand-membrane complex.

The following exemplified compounds were tested essentially as described above and exhibited the following binding affinity:

TABLE 32

| | Binding | Binding |
|---|---|---|
| | BACE1 | BACE2 |
| Co. No. | Kᵢ (nM) | Kᵢ (nM) |
| II-1 | 0.37 | 104 |
| II-2 | 0.18 | 95 |
| II-3 | 0.26 | 128 |
| II-4 | 0.18 | 11.4 |
| II-5 | 4.34 | 454 |
| II-6 | 0.63 | 103 |
| II-7 | 0.47 | 79 |
| II-8 | 0.17 | 152 |
| II-9 | ND | ND |
| II-10 | 0.08 | 20 |
| II-11 | ND | ND |
| II-12 | ND | ND |
| II-13 | 0.77 | 56 |
| II-14 | 0.17 | 23 |
| II-15 | ND | ND |
| II-16 | 0.49 | 434 |
| II-17 | 0.16 | 29 |
| II-18 | ND | ND |
| II-19 | 0.12 | 15 |
| II-20 | 0.21 | 68 |
| II-21 | ND | ND |
| II-22 | 0.37 | 104 |
| II-23 | 0.19 | 104 |
| II-24 | 0.14 | 63 |
| II-25 | 1.34 | 53 |
| II-26 | 0.44 | 220 |
| II-27 | 0.26 | 18 |
| II-28 | 0.42 | 91 |
| II-29 | 0.4 | 11 |
| II-30 | ND | ND |
| II-31 | ND | ND |
| II-32 | ND | ND |
| II-33 | ND | ND |
| II-34 | ND | ND |
| II-35 | ND | ND |

ND means not determined.

### Test Example 2-1

### BACE1 Biochemical FRET Based Assay

This assay is a Fluorescence Resonance Energy Transfer Assay (FRET) based assay. The substrate for this assay is an APP derived 13 amino acids peptide that contains the ‘Swedish’ Lys-Met/Asn-Leu mutation of the amyloid precursor protein (APP) beta-site secretase cleavage site. This substrate also contains two fluorophores: (7-methoxycoumarin-4-yl) acetic acid (Mca) is a fluorescent donor with excitation wavelength at 320 nm and emission at 405 nm and 2,4-dinitrophenol (Dnp) is a proprietary quencher acceptor. The distance between those two groups has been selected so that upon light excitation, the donor fluorescence energy is significantly quenched by the acceptor, through resonance energy transfer. Upon cleavage by BACE1, the fluorophore Mca is separated from the quenching group Dnp, restoring the full fluorescence yield of the donor. The increase in fluorescence is linearly related to the rate of proteolysis.

Briefly in a 384-well format recombinant BACE1 protein in a final concentration of 0.04 μg/mL is incubated for 450 minutes at room temperature with 20 μm substrate in incubation buffer (final concentrations: 33.3 mM Citrate buffer pH 5.0, 0.033% PEG, 3% DMSO) in the absence or presence of compound. Next the amount of proteolysis is directly measured by fluorescence measurement at T=0′-120′ and T=450′ (excitation at 320 nm and emission at 405 nm). Results are expressed in RFU (Relative Fluorescence Units), as difference between T450 and Tx (Tx is chosen depending on the reaction speed between 0 and 120 minutes.).

A best-fit curve is fitted by a minimum sum of squares method to the plot of % Controlmin versus compound concentration. From this an IC₅₀ value (inhibitory concentration causing 50% inhibition of activity) can be obtained.

$$\begin{matrix}LC = Median of the low control values \\ = L ow control \text{:} Reaction without enzyme \\ HC = Median of the High control values \\ = High Control \text{:} Reaction with enzymes\end{matrix}$$

$$\% Effect = 100 \cdot [ ( sample \cdot LC ) / ( HC \cdot LC ) * 100 ]$$

$$\% Control = ( sample / HC ) * 100$$

$$\% Controlmin = ( sample \cdot LC ) / ( HC \cdot LC ) * 100$$

A compound of the formula (IA), (IB), or (IC) has BACE1 inhibiting activity, and it is sufficient that the compound can inhibit the BACE1 receptor.

Specifically, by the protocol above shown, IC50 is preferably 5000 nM or less, more preferably 1000 nM or less, further preferably 100 nM or less.

### (2) BACE2 Biochemical FRET Based Assay

This assay is a Fluorescence Resonance Energy Transfer Assay (FRET) based assay. The substrate for this assay contains the ‘Swedish’ Lys-Met/Asn-Leu mutation of the amyloid precursor protein (APP) beta-secretase cleavage site. This substrate also contains two fluorophores: (7-methoxycoumarin-4-yl) acetic acid (Mca) is a fluorescent donor with excitation wavelength at 320 nm and emission at 405 nm and 2,4-dinitrophenol (Dnp) is a proprietary quencher acceptor. The distance between those two groups has been selected so that upon light excitation, the donor fluorescence energy is significantly quenched by the acceptor, through resonance energy transfer. Upon cleavage by the beta-secretase, the fluorophore Mca is separated from the quenching group Dnp, restoring the full fluorescence yield of the donor. The increase in fluorescence is linearly related to the rate of proteolysis. Briefly in a 384-well format recombinant BACE2 protein in a final concentration of 0.4 μg/mL was incubated for 450 minutes at room temperature with 10 μM substrate in incubation buffer (final concentrations: 33.3 mM Citrate buffer pH 5.0, 0.033% PEG, 2% DMSO) in the absence or presence of compound. Next the amount of proteolysis was directly measured by fluorescence measurement at T=0 and T=450 (excitation at 320 nm and emission at 405 nm). Results were expressed in RFU (Relative Fluorescence Units), as difference between T450 and TO.

A best-fit curve was fitted by a minimum sum of squares method to the plot of % Controlmin versus compound concentration. From this an IC₅₀ value (inhibitory concentration causing 50% inhibition of activity) can be obtained.

$$\begin{matrix}LC = Median of the low control values \\ = L ow control \text{:} Reaction without enzyme \\ HC = Median of the High control values \\ = High Control \text{:} Reaction with enzymes\end{matrix}$$

$$\% Effect = 100 \cdot [ ( sample \cdot LC ) / ( HC \cdot LC ) * 100 ]$$

$$\% Control = ( sample / HC ) * 100$$

$$\% Controlmin = ( sample \cdot LC ) / ( HC \cdot LC ) * 100$$

The following exemplified compounds were tested essentially as described above and exhibited the following activity:

TABLE 33

| | BACE1 | BACE2 | |
|---|---|---|---|
| | IC50 | IC50 | |
| No | (nM) | (nM) | selectivity |
| I-001 | 1.2 | 72.0 | 60.0 |
| I-002 | 9.6 | 427.0 | 44.7 |
| I-003 | 2.8 | 112.0 | 40.7 |
| I-004 | 13.2 | 513.0 | 38.9 |
| I-005 | 2.0 | 77.6 | 38.0 |
| I-006 | 15.5 | 447.0 | 28.8 |
| I-007 | 2.1 | 61.7 | 28.8 |
| I-008 | 9.6 | 219.0 | 22.9 |
| I-009 | 1.5 | 32.4 | 21.5 |
| I-010 | 6.9 | 135.0 | 19.5 |
| I-011 | 1.2 | 18.6 | 15.9 |
| I-012 | 2.9 | 53.7 | 18.7 |
| I-013 | 3.2 | 55.0 | 17.4 |
| I-014 | 4.1 | 57.5 | 14.1 |
| I-015 | 1.3 | 13.5 | 10.5 |
| I-020 | 3.2 | 209.0 | 64.5 |
| I-021 | 5.4 | 123.0 | 22.9 |

TABLE 34

| | BACE1 | BACE2 | |
|---|---|---|---|
| | IC50 | IC50 | |
| No. | (nM) | (nM) | Selectivity |
| I-032 | 89.1 | 10000 | 112 |
| I-033 | 6.92 | 3090 | 447 |
| I-035 | 2.20 | 661 | 301 |
| I-036 | 13.2 | 2190 | 166 |
| I-037 | 20.9 | 5500 | 263 |
| I-039 | 2.24 | 490 | 218 |
| I-040 | 7.94 | 2040 | 257 |
| I-043 | 9.77 | 2290 | 234 |
| I-044 | 14.5 | 1910 | 131 |
| I-046 | 8.32 | 2190 | 263 |
| I-049 | 3.60 | 490 | 136 |
| I-050 | 5.37 | 1070 | 199 |
| I-053 | 10.0 | 2000 | 200 |
| I-054 | 9.33 | 6030 | 646 |
| I-057 | 2.57 | 1000 | 389 |

### Test Example 2-2

### BACE1 Biochemical FRET Based Assay

This assay is a Fluorescence Resonance Energy Transfer Assay (FRET) based assay. The substrate for this assay is an APP derived 13 amino acids peptide that contains the ‘Swedish’ Lys-Met/Asn-Leu mutation of the amyloid precursor protein (APP) beta-site secretase cleavage site. This substrate also contains two fluorophores: (7-methoxycoumarin-4-yl) acetic acid (Mca) is a fluorescent donor with excitation wavelength at 320 nm and emission at 405 nm and 2,4-dinitrophenol (Dnp) is a proprietary quencher acceptor. The distance between those two groups has been selected so that upon light excitation, the donor fluorescence energy is significantly quenched by the acceptor, through resonance energy transfer. Upon cleavage by BACE1, the fluorophore Mca is separated from the quenching group Dnp, restoring the full fluorescence yield of the donor. The increase in fluorescence is linearly related to the rate of proteolysis.

Briefly in a 384-well format recombinant BACE1 protein in a final concentration of 0.04 μg/mL is incubated for 450 minutes at room temperature with 20 μm substrate in incubation buffer (final concentrations: 33.3 mM Citrate buffer pH 5.0, 0.033% PEG, 3% DMSO) in the absence or presence of compound. Next the amount of proteolysis is directly measured by fluorescence measurement at T=0′-120′ and T=450′ (excitation at 320 nm and emission at 405 nm). Results are expressed in RFU (Relative Fluorescence Units), as difference between T450 and Tx (Tx is chosen depending on the reaction speed between 0 and 120 minutes.).

Data are analysed using Screener (Registered trademark) (Genedata, Switzerland). Data were normalized and % inhibition=100−[(sample−LC)/(HC−LC))*100], was plotted versus the log concentration of the test compound. Curves were analysed using non-linear regression analysis and IC50 values (inhibitory concentration causing 50% inhibition of activity) were derived from individual curves.

LC=Median of the low control values

=Low control: Reaction without enzyme

HC=Median of the High control values

=High Control: Reaction with enzyme

% Inhibition=100−[(sample−LC)/(HC−LC)*100]

The following exemplified compounds were tested essentially as described above and exhibited the following activity:

TABLE 35

| | BACE1 Biochemical FRET |
|---|---|
| | based assay - Method 1 |
| Co. No. | PIC₅₀ |
| II-1 | 8.63 |
| II-2 | 8.55 |
| II-3 | 8.4 |
| II-4 | 8.63 |
| II-5 | 7.55 |
| II-6 | 8.41 |
| II-7 | 8.34 |
| II-8 | 8.62 |
| II-9 | 8.77 |
| II-10 | 8.86 |
| II-11 | 8.19 |
| II-12 | 8.39 |
| II-13 | 8.01 |
| II-14 | 8.79 |
| II-15 | 7.93 |
| II-16 | 8.61 |
| II-17 | 8.94 |
| II-18 | 8.49 |
| II-19 | 8.8 |
| II-20 | 8.6 |
| II-21 | 8.67 |
| II-22 | 8.38 |
| II-23 | 8.53 |
| II-24 | 8.75 |
| II-25 | 8.05 |
| II-26 | 8.13 |
| II-27 | 8.68 |
| II-28 | 8.53 |
| II-29 | 8.23 |
| II-30 | 8.06 |
| II-31 | 8.27 |
| II-32 | 7.75 |
| II-33 | 8.31 |
| II-34 | 8.49 |
| II-35 | 8.27 |

ND means not determined.

### Test Example 2-3

### BACE1 Cellular Assay in SKNBE2 Cells

In two αLisa assays the levels of Abeta 1-42 or Abeta total produced and secreted into the medium of human neuroblastoma SKNBE2 cells are quantified. The assays are based on the human neuroblastoma SKNBE2 expressing the wild type Amyloid Precursor Protein (hAPP695). The compounds are diluted and added to these cells, incubated for 18 hours and then measurements of Abeta 1-42 or Abeta total are taken. Abeta 1-42 or Abeta total are measured by sandwich αLisa using biotinylated antibody AbN/25 attached to streptavidin coated donor beads and antibody cAb42/26 or Ab 4G8 conjugated acceptor beads for the detection of Abeta 1-42 or Abeta total respectively. In the presence of Abeta 1-42 or Abeta total, the beads come into close proximity. The excitation of the donor beads provokes the release of singlet oxygen molecules that trigger a cascade of energy transfer in the acceptor beads, resulting in light emission. Light emission is measured after 1 h incubation (excitation at 650 nm and emission at 615 nm).

Data are analysed using Screener (Registered trademark) (Genedata, Switzerland). Data were normalized and % inhibition=100−[(sample−LC)/(HC−LC))*100], was plotted versus the log concentration of the test compound. Curves were analysed using non-linear regression analysis and IC50 values (inhibitory concentration causing 50% inhibition of activity) were derived from individual curves.

LC=Median of the low control values
=Low control: Reaction without enzyme

HC=Median of the High control values
=High Control: Reaction with enzyme

% Inhibition=100−[(sample−LC)/(HC−LC)*100]

The following exemplified compounds were tested essentially as described above and exhibited the following activity:

TABLE 36

| | Cellular αLisa assay in SKNBE2 cells |
|---|---|
| | Abeta 42 |
| Co. No. | pIC₅₀ |
| II-1 | 8.53 |
| II-2 | 8.47 |
| II-3 | 8.56 |
| II-4 | 7.96 |
| II-5 | 7.12 |
| II-6 | 8.19 |
| II-7 | 8.53 |
| II-8 | 8.8 |
| II-9 | 8.89 |
| II-10 | 8.89 |
| II-11 | 8.45 |
| II-12 | 8.59 |
| II-13 | 7.84 |
| II-14 | 8.24 |
| II-15 | 7.64 |
| II-16 | 8.19 |
| II-17 | 8.81 |
| II-18 | 8.35 |
| II-19 | 8.64 |
| II-20 | 8.36 |
| II-21 | 8.54 |
| II-22 | 7.71 |
| II-23 | 8.35 |
| II-24 | 8.54 |
| II-25 | 7.51 |
| II-26 | 7.81 |
| II-27 | 8.95 |
| II-28 | 8.53 |
| II-29 | 8.03 |
| II-30 | 7.95 |
| II-31 | 8.17 |
| II-32 | 7.77 |
| II-33 | 8.28 |
| II-34 | 8.48 |
| II-35 | 8.01 |

### Test Example 2-4

### BACE2 Biochemical FRET Based Assay

This assay is a Fluorescence Resonance Energy Transfer Assay (FRET) based assay. The substrate for this assay contains the ‘Swedish’ Lys-Met/Asn-Leu mutation of the amyloid precursor protein (APP) beta-secretase cleavage site. This substrate also contains two fluorophores: (7-methoxycoumarin-4-yl) acetic acid (Mca) is a fluorescent donor with excitation wavelength at 320 nm and emission at 405 nm and 2,4-dinitrophenol (Dnp) is a proprietary quencher acceptor. The distance between those two groups has been selected so that upon light excitation, the donor fluorescence energy is significantly quenched by the acceptor, through resonance energy transfer. Upon cleavage by the beta-secretase, the fluorophore Mca is separated from the quenching group Dnp, restoring the full fluorescence yield of the donor. The increase in fluorescence is linearly related to the rate of proteolysis.

Briefly in a 384-well format recombinant BACE2 protein in a final concentration of 0.4 μg/mL is incubated for 450 minutes at room temperature with 10 μM substrate in incubation buffer (final concentrations: 33.3 mM Citrate buffer pH 5.0, 0.033% PEG, 2% DMSO) in the absence or presence of compound. Next the amount of proteolysis is directly measured by fluorescence measurement at T=0 and T=450 (excitation at 320 nm and emission at 405 nm). Results are expressed in RFU (Relative Fluorescence Units), as difference between T450 and TO.

Data are analysed using Screener (Registered trademark) (Genedata, Switzerland). Data were normalized and % inhibition=100−[(sample−LC)/(HC−LC))*100], was plotted versus the log concentration of the test compound. Curves were analysed using non-linear regression analysis and IC50 values (inhibitory concentration causing 50% inhibition of activity) were derived from individual curves.

LC=Median of the low control values

=Low control: Reaction without enzyme

HC=Median of the High control values

=High Control: Reaction with enzyme

% Inhibition=100−[(sample−LC)/(HC−LC)*100]

The following exemplified compounds were tested essentially as described above and exhibited the following activity:

TABLE 37

| | BACE2 Biochemical FRET |
|---|---|
| Co. No. | based assay pIC₅₀ |
| II-1 | 6.47 |
| II-2 | 6.64 |
| II-3 | 6.39 |
| II-4 | 7.33 |
| II-5 | 5.94 |
| II-6 | 6.83 |
| II-7 | 6.75 |
| II-8 | 6.51 |
| II-9 | 7.08 |
| II-10 | 7.18 |
| II-11 | 6.48 |
| II-12 | 6.43 |
| II-13 | 6.45 |
| II-14 | 6.75 |
| II-15 | 6.48 |
| II-16 | 6.24 |
| II-17 | 7.16 |
| II-18 | 6.62 |
| II-19 | 7.11 |
| II-20 | 6.73 |
| II-21 | 6.69 |
| II-22 | 6.37 |
| II-23 | 6.59 |
| II-24 | 6.5 |
| II-25 | 6.48 |
| II-26 | 6.07 |
| II-27 | 7.28 |
| II-28 | 6.76 |
| II-29 | 6.77 |
| II-30 | 6.01 |
| II-31 | 6.6 |
| II-32 | 6.29 |
| II-33 | 6.3 |
| II-34 | 6.81 |
| II-35 | 6.53 |

### Test Example 2-5

### BACE2 Cellular Assay in MING Cells

Cellular BACE2 activity was measured by determination of the level of secreted TMEM27 into the medium of MIN6 cells using an MSD platform. The assay is based on the mouse insulinoma MIN6 cells expressing Flag-V5-TMEM27-HA. When BACE2 cleaves TMEM27 the N-terminal part of TMEM27 with the V5-Flag tag will be shed into the medium, and the amount of this cleaved product is measured with the MSD assay.

Briefly in a 96-well format the cells are incubated for 24 hours in the presence of the compound followed by the measurement of secreted TMEM27 in the medium via MSD using FLAG-L5 coating antibody and a V5 detection antibody. When TMEM27 is captured on the electrode surface of the multi-array microplate the detection antibody, conjugated with the sulfo-Tag™, is in close proximity of the surface, it generates a light via a series of reduction and oxidation reactions. The intensity of the emitted light is measured with the MSD imager to provide a quantitative measure for the analytes in the sample.

Data are analysed using Screener (Registered trademark) (Genedata, Switzerland). Data were normalized and % inhibition=100−[(sample−LC)/(HC−LC))*100], was plotted versus the log concentration of the test compound. Curves were analysed using non-linear regression analysis and IC₅₀ values (inhibitory concentration causing 50% inhibition of activity) were derived from individual curves.

LC=Median of the low control values

=Low control: Reaction without enzyme

HC=Median of the High control values

=High Control: Reaction with enzyme

% Inhibition=100−[(sample−LC)/(HC−LC)*100]

The following exemplified compounds were tested essentially as described above and exhibited the following activity:

TABLE 38

| | Cellular MSD assay in Min6 cells |
|---|---|
| | TV1EV127 |
| Co. No. | pIC₅₀ |
| II-1 | 6.23 |
| II-2 | 6.60 |
| II-3 | 6.49 |
| II-4 | 7.09 |
| II-5 | 5.83 |
| II-6 | 6.76 |
| II-7 | 6.86 |
| II-8 | 6.42 |
| II-9 | ND |
| II-10 | 7.14 |
| II-11 | 6.44 |
| II-12 | 6.18 |
| II-13 | 6.13 |
| II-14 | 6.70 |
| II-15 | 6.37 |
| II-16 | 5.78 |
| II-17 | 6.47 |
| II-18 | ND |
| II-19 | 6.82 |
| II-20 | 6.51 |
| II-21 | ND |
| II-22 | 6.06 |
| II-24 | 6.52 |
| II-25 | 6.18 |
| II-26 | 5.72 |
| II-27 | ND |
| II-28 | ND |
| II-29 | ND |
| II-30 | ND |
| II-31 | ND |
| II-32 | ND |
| II-33 | ND |
| II-34 | 6.53 |
| II-35 | 6.21 |

ND means not determined.

### Test Example 3-1: Lowering Effect on the Brain 6 Amyloid in Rats

Compound of the present invention is suspended in 0.5% methylcellulose, the final concentration is adjusted to 2 mg/mL, and this is orally administered to male Crl:SD rat (7 to 9 weeks old) at 1 to 30 mg/kg. In a vehicle control group, only 0.5% methylcellulose is administered, and an administration test is performed at 3 to 8 animals per group. A brain is isolated 3 hours after administration, a cerebral hemisphere is isolated, a weight thereof is measured, the hemisphere is rapidly frozen in liquid nitrogen, and stored at −80° C. until extraction date. The frozen cerebral hemisphere is transferred to a homogenizer manufactured by Teflon (Registered trademark) under ice cooling, a 5-fold volume of a weight of an extraction buffer (containing 1% CHAPS ({3-[(3-chloroamidopropyl)dimethylammonio]-1-propanesulfonate}), 20 mmol/L Tris-HCl (pH 8.0), 150 mmol/L NaCl, Complete (Roche) protease inhibitor) is added, up and down movement is repeated, and this is homogenized to solubilize for 2 minutes. The suspension is transferred to a centrifugation tube, allowed to stand on an ice for 3 hours or more and, thereafter centrifuged at 200,000×g, 4° C. for 20 minutes. After centrifugation, the supernatant is transferred to an ELISA plate (product No. 294-62501, Wako Junyaku Kogyo) for measuring ß amyloid 40. ELISA measurement is performed according to the attached instruction. The lowering effect is calculated as a ratio compared to the brain ß amyloid 40 level of vehicle control group of each test.

### Test Example 3-2: Lowering Effect on the Brain ß Amyloid in Mice

Compound of the present invention was dissolved in 20% hydroxyl-beta-cyclodextrin, the final concentration was adjusted to 2 mg/mL, and this was orally administered to male Crl:CD1 (ICR) mouse (6 to 8 weeks old) at 1 to 30 mg/kg. In a vehicle control group, only 20% hydroxyl-beta-cyclodextrin was administered, and an administration test was performed at 3 to 6 animals per group. A brain was isolated 1 to 6 hours after administration, a cerebral hemisphere was isolated, a weight thereof was measured, the hemisphere was rapidly frozen in liquid nitrogen, and stored at −80° C. until extraction date.

The frozen cerebral hemisphere was transferred to a homogenize tube containing ceramic beads in a 8-fold volume of a weight of an extraction buffer (containing 0.4% DEA (diethylamine), 50 mmol/L NaCl, Complete protease inhibitor (Roche)) and incubated on an ice for 20 minutes. Thereafter, the homogenization was done using MP BIO FastPrep(Registered trademark)-24 with Lysing matrix D 1.4 mm ceramic beads (20 seconds at 6 m/s). Then, the tube spins down for 1 minute, the supernatant was transferred to a centrifugation tube, and centrifuged at 221,000×g, 4° C. for 50 minutes. After centrifugation, the supernatant was transferred to Nunc Maxisorp (Registered trademark) plate (Thermo Fisher Scientific) coating with antibody against N-terminal of 6 amyloid for measuring total 6 amyloid, and the plate was incubated overnight at 4° C. The plate was washed with TBS-T (Tris buffered saline containing 0.05% Triton X-100), and HRP-conjugated 4G8 dissolved in PBS (pH 7.4) containing 0.1% casein was added in the plate and incubated at 4° C. for 1 hour. After it was washed with TBS-T, SuperSignal ELISA Pico Chemiluminescent Substrate (Thermo Scientific) was added in the plate. Then, the chemi-luminescence counting was measured by ARVO (Registered trademark) MX 1420 Multilabel Counter (Perkin Elmer) as soon as possible. The lowering effect was calculated as a ratio compared to the brain total ß amyloid level of vehicle control group of each test.

### Test Example 4-1: CYP3A4 Fluorescent MBI Test

The CYP3A4 fluorescent MBI test is a test of investigating enhancement of CYP3A4 inhibition of a compound by a metabolism reaction. 7-benzyloxytrifluoromethylcoumarin (7-BFC) is debenzylated by the CYP3A4 enzyme (enzyme expressed in Escherichia coli) and 7-hydroxytrifluoromethylcoumarin (7-HFC) is produced as a fluorescing metabolite. The test is performed using 7-HFC production reaction as a marker reaction.

The reaction conditions are as follows: substrate, 5.6 μmol/L 7-BFC; pre-reaction time, 0 or 30 minutes; substrate reaction time, 15 minutes; reaction temperature, 25° C. (room temperature); CYP3A4 content (expressed in Escherichia coli), 62.5 pmol/mL at pre-reaction time, 6.25 pmol/mL (10-fold dilution) at reaction time; concentrations of the compound of the present invention, 0.625, 1.25, 2.5, 5, 10, 20 μmol/L (6 points).

An enzyme in a K-Pi buffer (pH 7.4) and a compound of the present invention solution as a pre-reaction solution are added to a 96-well plate at the composition of the pre-reaction. A part of pre-reaction solution is transferred to another 96-well plate, and diluted 10-fold by a substrate in a K-Pi buffer. NADPH as a co-factor is added in order to initiate a marker reaction (without preincubation). After a predetermined time of the marker reaction, acetonitrile/0.5 mol/L Tris (trishydroxyaminomethane)=4/1 (v/v) solution is added in order to terminate the marker reaction. On the other hand, NADPH is also added to a remaining pre-reaction solution in order to initiate a pre-reaction (with preincubation). After a predetermined time of a pre-raction, a part is transferred to another 96-well plate, and diluted 10-fold by a substrate in a K-Pi buffer in order to initiate the marker reaction. After a predetermined time of the marker reaction, acetonitrile/0.5 mol/L Tris (trishydroxyaminomethane)=4/1 (v/v) solution is added in order to terminate the marker reaction. Fluorescent values of 7-HFC as a metabolite are measured in each index reaction plate with a fluorescent plate reader (Ex=420 nm, Em=535 nm).

The sample adding DMSO to a reaction system instead of compound of the present invention solution is adopted as a control (100%) because DMSO is used as a solvent to dissolve a compound of the present invention. Remaining activity (%) is calculated at each concentration of the compound of the present invention added as the solution, and IC₅₀ value is calculated by reverse-presumption using a logistic model with a concentration and an inhibition rate. When a difference subtracting IC₅₀ value with preincubation from that without preincubation is 5 μM or more, this is defined as positive (+). When the difference is 3 μM or less, this is defined as negative (−).

### Test Example 4-2: CYP3A4(MDZ) MBI Test

CYP3A4(MDZ) MBI test is a test of investigating mechanism based inhibition (MBI) potential on CYP3A4 inhibition of a compound. CYP3A4 inhibition is evaluated using 1-hydroxylation reaction of midazolam (MDZ) by pooled human liver microsomes as a marker reaction.

The reaction conditions were as follows: substrate, 10 μmol/L MDZ; pre-reaction time, 0 or 30 minutes; substrate reaction time, 2 minutes; reaction temperature, 37° C.; protein content of pooled human liver microsomes, 0.5 mg/mL at pre-reaction time, 0.05 pmg/mL (at 10-fold dilution) at reaction time; concentrations of the compound of the present invention, 1, 5, 10, 20 μmol/L (4 points).

Pooled human liver microsomes in a K-Pi buffer (pH 7.4) and a compound of the present invention solution as a pre-reaction solution were added to a 96-well plate at the composition of the pre-reaction. A part of pre-reaction solution was transferred to another 96-well plate, and diluted 10-fold by a substrate in a K-Pi buffer. NADPH as a co-factor was added to initiate the marker reaction (without preincubation). After a predetermined time of the marker reaction, methanol/acetonitrile=1/1 (v/v) solution was added in order to terminate the marker reaction. On the other hand, NADPH was also added to a remaining pre-reaction solution in order to initiate a pre-reaction (with preincubation). After a predetermined time of a pre-reaction, a part was transferred to another 96-well plate, and diluted 10-fold by a substrate in a K-Pi buffer in order to initiate the marker reaction. After a predetermined time of the marker reaction, methanol/acetonitrile=1/1 (v/v) solution is added in order to terminate the marker reaction. After centrifuged at 3000 rpm for 15 minutes, 1-hydroxymidazolam in the supernatant is quantified by LC/MS/MS.

The sample adding DMSO to a reaction system instead of compound of the present invention solution was adopted as a control (100%) because DMSO is used as a solvent to dissolve a compound of the present invention. Remaining activity (%) was calculated at each concentration of the compound of the present invention added as the solution, and IC₅₀ value was calculated by reverse-presumption using a logistic model with a concentration and an inhibition rate. Shifted IC value was calculated as “IC value without preincubation (0 minutes)/IC value with preincubation (30 minutes)”. When a shifted IC value was 1.5 or more, this was defined as positive. When a shifted IC value was less than 1.1, this was defined as negative.

TABLE 39

| No. | MBI MDZ |
|---|---|
| I-011 | Negative |
| I-012 | Negative |
| I-024 | Negative |
| I-026 | Negative |
| I-027 | Negative |
| I-035 | Negative |
| I-036 | Negative |
| I-046 | Negative |
| I-049 | Negative |

### Test Example 5: CYP Inhibition Test

The CYP inhibition test is a test to assess the inhibitory effect of a compound of the present invention towards typical substrate metabolism reactions on CYP enzymes in human liver microsomes. The marker reactions on human main five CYP enzymes (CYP1A2, 2C9, 2C19, 2D6, and 3A4) were used as follows; 7-ethoxyresorufin O-deethylation (CYP1A2), tolbutamide methyl-hydroxylation (CYP2C9), mephenytoin 4′-hydroxylation (CYP2C19), dextromethorphan 0-demethylation (CYP2D6), and terfenadine hydroxylation (CYP3A4). The commercially available pooled human liver microsomes were used as an enzyme resource.

The reaction conditions were as follows: substrate, 0.5 μmol/L ethoxyresorufin (CYP1A2), 100 μmol/L tolbutamide (CYP2C9), 50 μmol/L S-mephenytoin (CYP2C19), 5 μmol/L dextromethorphan (CYP2D6), 1 μmol/L terfenadine (CYP3A4); reaction time, 15 minutes; reaction temperature, 37° C.; enzyme, pooled human liver microsomes 0.2 mg protein/mL; concentrations of the compound of the present invention, 1, 5, 10, 20 μmol/L (4 points).

Five kinds of substrates, human liver microsomes, and a compound solution of the present invention in 50 mmol/L Hepes buffer were added to a 96-well plate at the composition as described above as a reaction solution. NADPH as a cofactor was added to this 96-well plate in order to initiate marker reactions. After the incubation at 37° C. for 15 minutes, a methanol/acetonitrile=1/1 (v/v) solution was added in order to terminate the marker reactions. After the centrifugation at 3000 rpm for 15 minutes, resorufin (CYP1A2 metabolite) in the supernatant was quantified by a fluorescent plate reader or LC/MS/MS, and hydroxytolbutamide (CYP2C9 metabolite), 4′-hydroxymephenytoin (CYP2C19 metabolite), dextrorphan (CYP2D6 metabolite), and terfenadine alcohol metabolite (CYP3A4 metabolite) in the supernatant were quantified by LC/MS/MS.

The sample adding DMSO to a reaction system instead of compound of the present invention solution was adopted as a control (100%) because DMSO was used as a solvent to dissolve a compound of the present invention. Remaining activity (%) was calculated at each concentration of a compound of the present invention, and IC50 value was calculated by reverse presumption using a logistic model with a concentration and an inhibition rate.

### Test Example 6: Fluctuation Ames Test

Each 20 μL of freeze-stored Salmonella typhimurium (TA98 and TA100 strain) is inoculated in 10 mL of liquid nutrient medium (2.5% Oxoid nutrient broth No. 2), and the cultures are incubated at 37° C. under shaking for 10 hours. 7.70 to 8.00 mL of TA98 culture is centrifuged (2000×g, 10 minutes) to remove medium, and the bacteria is suspended in 7.70 mL of Micro F buffer (K₂HPO₄: 3.5 g/L, KH₂PO₄: 1 g/L, (NH₄)₂SO₄: 1 g/L, trisodium citrate dihydrate: 0.25 g/L, MgSO₄-7H₂O: 0.1 g/L), and the suspension is added to 120 mL of Exposure medium (Micro F buffer containing Biotin: 8 μg/mL, histidine: 0.2 μg/mL, glucose: 8 mg/mL). 3.10 to 3.42 mL of TA100 culture is added to 130 mL of Exposure medium to prepare the test bacterial solution. 588 μL of the test bacterial solution (or mixed solution of 498 μL of the test bacterial solution and 90 μL of the S9 mix in the case with metabolic activation system) are mixed with each 12 μL of the following solution: DMSO solution of the compound of the present invention (several stage dilution from maximum dose 50 mg/mL at 2 to 3-fold ratio); DMSO as negative control; 50 μg/mL of 4-nitroquinoline-1-oxide DMSO solution as positive control for TA98 without metabolic activation system; 0.25 μg/mL of 2-(2-furyl)-3-(5-nitro-2-furyl)acrylamide DMSO solution as positive control for TA100 without metabolic activation system; 40 μg/mL of 2-aminoanthracene DMSO solution as positive control for TA98 with metabolic activation system; or 20 μg/mL of 2-aminoanthracene DMSO solution as positive control for TA100 with metabolic activation system. A mixed solution is incubated at 37° C. under shaking for 90 minutes. 460 μL of the bacterial solution exposed to the compound of the present invention is mixed with 2300 μL of Indicator medium (Micro F buffer containing biotin: 8 μg/mL, histidine 0.2 μg/mL, glucose: 8 mg/mL, Bromo Cresol Purple: 37.5 μg/mL), each 50 μL is dispensed into 48 wells/dose in the microwell plates, and is subjected to stationary cultivation at 37° C. for 3 days. A well containing the bacteria, which has obtained the ability of proliferation by mutation in the gene coding amino acid (histidine) synthetase, turns the color from purple to yellow due to pH change. The number of the yellow wells among the 48 total wells per dose is counted, and evaluate the mutagenicity by comparing with the negative control group. (−) means that mutagenicity is negative and (+) means positive.

### Test Example 7: Solubility Test

The solubility of each compound of the present invention was determined under 1% DMSO addition conditions. A 10 mmol/L solution of the compound was prepared with DMSO, and 2 μL of the compound of the present invention solution was added, respectively, to 198 μL of JP 1st fluid (water was added to 2.0 g of sodium chloride and 7.0 mL of hydrochloric acid to reach 1000 mL) and JP 2nd fluid (1 volume of water was added to 1 volume of the solution which 3.40 g of potassium dihydrogen phosphate and 3.55 g of anhydrous disodium hydrogen phosphate dissolve in water to reach 1000 mL). The mixture was left standing for 16 hours at 25° C. or shaken for 1 hour at room temperature, and the mixture was vacuum-filtered. The filtrate was ten or one hundred-fold diluted with methanol/water=1/1 (v/v) or MeCN/MeOH/H₂O(=1/1/2), and the compound concentration in the filtrate was measured with LC/MS or solid phase extraction (SPE)/MS by the absolute calibration method.

### Test Example 8: Metabolic Stability Test

Using a commercially available pooled human liver microsomes, a compound of the present invention was reacted for a constant time, a remaining rate was calculated by comparing a reacted sample and an unreacted sample, thereby, a degree of metabolism in liver was assessed.

A reaction was performed (oxidative reaction) at 37° C. for 0 minute or 30 minutes in the presence of 1 mmol/L NADPH in 0.2 mL of a buffer (50 mmol/L Tris-HCl pH 7.4, 150 mmol/L potassium chloride, 10 mmol/L magnesium chloride) containing 0.5 mg protein/mL of human liver microsomes. After the reaction, 50 μL of the reaction solution was added to 100 μL of a methanol/acetonitrile=1/1 (v/v), mixed and centrifuged at 3000 rpm for 15 minutes. The compound of the present invention in the supernatant was quantified by LC/MS/MS or solid phase extraction (SPE)/MS, and a remaining amount of the compound of the present invention after the reaction was calculated, letting a compound amount at 0 minute reaction time to be 100%.

### Test Example 9: hERG Test

For the purpose of assessing risk of an electrocardiogram QT interval prolongation, effects on delayed rectifier K+ current (I_{Kr}), which plays an important role in the ventricular repolarization process of the compound of the present invention, was studied using CHO cells expressing human ether-a-go-go related gene (hERG) channel.

A cell was retained at a membrane potential of −80 mV by whole cell patch clamp method using an automated patch clamp system (QPatch; Sophion Bioscience A/S). After application of leak potential at −50 mV, I_{Kr} induced by depolarization pulse stimulation at +20 mV for 2 seconds and, further, repolarization pulse stimulation at −50 mV for 2 seconds was recorded.

After the generated current was stabilized, extracellular solution (NaCl: 145 mmol/L, KCl: 4 mmol/L, CaCl₂: 2 mmol/L MgCl₂: 1 mmol/L, 1 mmol/L, HEPES(4-(2-hydroxyethyl)-1-piperazineethanesulfonic acid: 10 mmol/L, glucose: 10 mmol/L pH=7.4) in which the compound of the present invention have been dissolved at an objective concentration was applied to the cell under the room temperature condition for 10 minutes. From the recording I_{Kr}, an absolute value of the tail peak current was measured based on the current value at the resting membrane potential using an analysis software (QPatch assay software; Sophion Bioscience A/S). Further, the % inhibition relative to the tail peak current before application of the compound of the present invention was calculated, and compared with the vehicle-applied group (0.1% dimethyl sulfoxide solution) to assess influence of the compound of the present invention on I_{Kr}.

### Test Example 10: Powder Solubility Test

Appropriate amounts of the compound of the present invention are put into appropriate containers. 200 μL of JP 1^{st} fluid (water is added to 2.0 g of sodium chloride and 7.0 mL of hydrochloric acid to reach 1000 mL), 200 μL of JP 2^{nd} fluid (1 volume of water is added to 1 volume of the solution which 3.40 g of potassium dihydrogen phosphate and 3.55 g of anhydrous disodium hydrogen phosphate dissolve in water to reach 1000 mL), 200 μL of fasted state simulated intestinal fluid (FaSSIF), and 200 μL of fed state simulated intestinal fluid (FeSSIF) are added to the respective containers. When total amount of the compound of the present invention is dissolved after the addition of the test fluid, the compound is added as appropriate. The containers are sealed, and shaken for 1 and/or 24 hours at 37° C. The mixtures are filtered, and 100 μL of methanol is added to each of the filtrate (100 μL) so that the filtrates are two-fold diluted. The dilution ratio may be changed if necessary. After confirming that there is no bubbles and precipitates in the diluted solution, the containers are sealed and shaken. Quantification is performed by HPLC with an absolute calibration method.

### Test Example 11: Pharmacokinetic Study

Materials and Methods for Studies on Oral Absorption

(1) Animal: mouse or rat
(2) Breeding conditions: mouse or rat was allowed free access to the tap water and the solid food.
(3) Dose and grouping: orally or intravenously administered at a predetermined dose; grouping was as follows (Dose depends on the compound)
Oral administration: approximately 1 to 30 mg/kg (n=2 to 3)
Intravenous administration: approximately 0.5 to 10 mg/kg (n=2 to 3)
(4) Dosing formulation: for oral administration, in a solution or a suspension state; for intravenous administration, in a solubilized state
(5) Dosing method: in oral administration, forcedly administer using a syringe attached a flexible feeding tube; in intravenous administration, administer from caudal vein using a syringe attached with a needle.
(6) Evaluation items: blood was collected at the scheduled time, and the plasma concentration of the compound of the present invention was measured by LC/MS/MS
(7) Statistical analysis: regarding the transition of the plasma concentration of the compound of the present invention, the area under the plasma concentration-time curve (AUC) was calculated by trapezoidal method, and the bioavailability (BA) of the compound of the present invention was calculated from the AUCs of the oral administration group and intravenous administration group.

### Test Example 12: Brain Distribution Studies

Compound of the present invention was intravenously administered to a rat at approximately 0.5 mg/mL/kg dosage. 30 minutes later, all blood was drawn from the abdominal aorta under isoflurane anesthesia for death from exsanguination.

The brain was enucleated and 20 to 25% of homogenate thereof was prepared with distilled water.

The obtained blood was used as plasma after centrifuging. The control plasma was added to the brain sample at 1:1. The control brain homogenate was added to the plasma sample at 1:1. Each sample was measured using LC/MS/MS. The obtained area ratio (a brain/plasma) was used for the brain Kp value.

### Test Example 13: Ames Test

Ames test is performed by using Salmonellas (Salmonella typhimurium) TA 98, TA100, TA1535 and TA1537 and Escherichia coli WP2uvrA as test strains with or without metabolic activation in the pre-incubation method to check the presence or absence of gene mutagenicity of compounds of the present invention.

### Test Example 14: P-gp Substrate Test

### 1. Cell Line:

a. MDR1/LLC-PK1 (Becton Dickinson)

b. LLC-PK1 (Becton Dickinson)

### 2. Reference Substrates:

a. Digoxin (2 μM)

Methods and Procedures

1. MDR1 expressing LLC-PK1 cells and its parent cells were routinely cultured in Medium A (Medium 199 (Invitrogen) supplemented with 10% FBS (Invitrogen), gentamycin (0.05 mg/mL, Invitrogen) and hygromycin B (100 μg/mL, Invitrogen)) at 37° C. under 5% CO2/95% O2 gasses. For the transport experiments, these cells were seeded on Transwell (Registered trademark) insert (96-well, pore size: 0.4 μm, Coaster) at a density of 1.4×10⁴ cells/insert and added Medium B (Medium 199 supplemented with 10% FBS and gentamycin at 0.05 mg/mL) to the feeder tray. These cells were incubated in a CO₂ incubator (5% CO2/95% O2 gasses, 37° C.) and replace apical and basolateral culture medium every 48-72 hr after seeding. These cells were used between 4 and 6 days after seeding.

2. The medium in the culture insert seeded with MDR1 expressing cells or parent cells were removed by aspiration and rinsed by HBSS. The apical side (140 μL) or basolateral side (175 μL) was replaced with transport buffer containing reference substrates and the present invention and then an aliquot (50 μL) of transport buffer in the donor side was collected to estimate initial concentration of reference substrate and the present invention. After incubation for designed time at 37° C., an aliquot (50 μL) of transport buffer in the donor and receiver side were collected. Assay was performed by duplicate or triplicate.

3. Reference substrate and the present invention in the aliquot was quantified by LC/MS/MS.

Calculations

Permeated amounts across monolayers of MDR1 expressing and parent cells were determined, and permeation coefficients (Pe) were calculated using Excel 2003 from the following equitation:

Pe (cm/sec)=Permeated amount (pmol)/area of cell membrane (cm²)/initial concentration (nM)/incubation time (sec)

Where, permeated amount was calculated from permeation concentration (nM, concentration of the receiver side) of the substance after incubation for the defined time (sec) multiplied by volume (mL) and area of cell membrane was used 0.1433 (cm2).

The efflux ratio was calculated using the following equation:

Efflux Ratio=Basolateral-to-Apical Pe/Apical-to-Basolateral Pe

The net flux was calculated using the following equation:

Net flux=Efflux Ratio in MDR1 expressing cells/Efflux Ratio in parent cells

### Test Example 15: Inhibitory Effects on P-Gp Transport

Materials

### 1. Cell Line:

a. MDR1/LLC-PK1 (Becton Dickinson)

b. LLC-PK1 (Becton Dickinson)

### 2. Reference Substrates:

a. [³H]Digoxin (1 μM)

b. [¹⁴C]Mannitol (1 μM)

### 3. Reference Inhibitor:

Verapamil (1 μM)

Methods and Procedures

1. MDR1 expressing LLC-PK1 cells and its parent cells are routinely cultured in Medium A (Medium 199 (Invitrogen) supplemented with 10% FBS (Invitrogen), gentamycin (0.05 mg/mL, Invitrogen) and hygromycin B (100 μg/mL, Invitrogen)) at 37° C. under 5% CO_{2/95}% 02 gasses. For the transport experiments, these cells are seeded on Transwell (Registered trademark) insert (96-well, pore size: 0.4 μm, Coaster) at a density of 1.4×10⁴ cells/insert and added Medium B (Medium 199 supplemented with 10% FBS and gentamycin at 0.05 mg/mL) to the feeder tray. These cells are incubated in a CO₂ incubator (5% CO₂/95% O₂ gasses, 37° C.) and replace apical and basolateral culture medium every 48-72 hr after seeding. These cells are used between 6 and 9 days after seeding.

2. The medium in the culture insert seeded with MDR1 expressing cells or parent cells are removed by aspiration and rinsed by HBSS. The apical side (150 μL) or basolateral side (200 μL) is replaced with transport buffer containing reference substrates with or without the compound of the present invention and then an aliquot (50 μL) of transport buffer in the donor side is collected to estimate initial concentration of reference substrate. After incubation for designed time at 37° C., an aliquot (50 μL) of transport buffer in the donor and receiver side are collected. Assay is performed by triplicate.

3. An aliquot (50 μL) of the transport buffer is mixed with 5 mL of a scintillation cocktail, and the radioactivity is measured using a liquid scintillation counter.

Calculations

Permeated amounts across monolayers of MDR1 expressing and parent cells are determined, and permeation coefficients (Pe) are calculated using Excel 2003 from the following equitation:

Pe (cm/sec)=Permeated amount (pmol)/area of cell membrane (cm²)/initial concentration (nM)/incubation time (sec)

Where, permeated amount is calculated from permeation concentration (nM, concentration of the receiver side) of the substance after incubation for the defined time (sec) multiplied by volume (mL) and area of cell membrane is used 0.33 (cm²). The efflux ratio will be calculated using the following equation:

Efflux Ratio=Basolateral-to-Apical Pe/Apical-to-Basolateral Pe

The net flux is calculated using the following equation:

Net flux=Efflux Ratio in MDR1 expressing cells/Efflux Ratio in parent cells

The percent of control is calculated as the net efflux ratio of reference compounds in the presence of the compound of the present invention to that in the absence of the compound of the present invention.

IC₅₀ values are calculated using the curve-fitting program XLfit.

### Test Example 16: P-Gp Substrate Test Using mdr1a/1b (−/−) B6 Mice

Materials

Animal: mdr1a/1b (−/−) B6 mice (KO mouse) or C57BL/6J mice (Wild mouse)

Methods and Procedures

1. Animals may be fed prior to dosing of the compounds of the present invention.

2. The compounds of the present invention are dosed to three animals for each time point and blood and brain samples are removed at selected time points (e.g. 15 min, 30 min, 1 hr, 2 hr, 4 hr, 6 hr, 8 hr, or 24 hr) after dosing. Blood (0.3-0.7 mL) is collected via trunk blood collection with syringe containing anticoagulants (EDTA and heparin). Blood and tissue (e.g. brain) samples are immediately placed on melting ice.

3. Blood samples are centrifuged (1780×g for 10 minutes) for cell removal to obtain plasma. Then, plasma samples are transferred to a clean tube and stored in a −70° C. freezer until analysis.

4. Tissue (e.g. brain) samples are homogenized at a 1:3 ratio of tissue weight to ml of stilled water and transferred to a clean tube and stored in a −70° C. freezer until analysis.

5. Plasma and tissue (e.g. brain) samples are prepared using protein precipitation and analyzed by LC/MS/MS. The analytical method is calibrated by including a standard curve constructed with blank plasma or brain samples and known quantities of analyte. Quality control samples are included to monitor the accuracy and precision of the methodology.

6. Plasma and brain concentration values (ng/mL and ng/g) are introduced into an appropriate mathematical tool used for calculating the pharmacokinetic parameters. A common platform is the WinNonlin (Registered trademark) pharmacokinetic software modeling program.

Calculations

Kp; Tissue to Plasma concentration ratio

Kp ratio=Kp in KO mouse/Kp in Wild mouse

KO/Wild ratio of AUC Tissue/AUC Plasma={AUC Tissue/AUC Plasma (KO mouse)}/{AUC Tissue/AUC Plasma (Wild mouse)}

### Test Example 17: Anesthetized Guinea Pig Cardiovascular Study

Animal species: Guinea pig (Slc: Hartley, 4-5 weeks old, male), N=4

### Study Design:

Dosage: 3, 10, and 30 mg/kg (in principle)

(The compounds of the present invention are administered cumulatively)

### Formulation:

Composition of Vehicle; Dimethylacetamide (DMA): Polyethylene glycol 400 (PEG400): Distilled water (D.W.)=1:7:2 (in principle).

The compounds of the present invention are dissolved with DMA and then added PEG400 and D.W. Finally, 1.5, 5, and 15 mg/mL solutions are prepared.

### Dosing Route and Schedule:

Intravenous infusion for 10 min (2 mL/kg).

0 to 10 min: 3 mg/kg, 30 to 40 min: 10 mg/kg, 60 to 70 min: 30 mg/kg

Vehicle is administered by the same schedule as the above.

### Group Composition:

Vehicle group and the compound of the present invention group (4 guinea pigs per group).

### Evaluation Method:

### Evaluation Items:

Mean blood pressure [mmHg], Heart rate (derived from blood pressure waveform [beats/min]), QTc (ms), and Toxicokinetics.

### Experimental Procedure:

Guinea pigs are anesthetized by urethane (1.4 g/kg, i.p.), and inserted polyethylene tubes into carotid artery (for measuring blood pressure and sampling blood) and jugular vein (for infusion test compounds). Electrodes are attached subcutaneously (Lead 2). Blood pressure, heart rate and electrocardiogram (ECG) are measured using PowerLab (Registered trademark) system (ADInstruments).

### Toxicokinetics:

Approximately 0.3 mL of blood (approximately 120 μL as plasma) is drawn from carotid artery with a syringe containing heparin sodium and cooled with ice immediately at each evaluation point. Plasma samples are obtained by centrifugation (4° C., 10000 rpm, 9300×g, 2 minutes). The procedure for separation of plasma is conducted on ice or at 4° C. The obtained plasma (TK samples) is stored in a deep freezer (set temperature: −80° C.).

Analysis methods: Mean blood pressure and heart rate are averaged a 30-second period at each evaluation time point. ECG parameters (QT interval [ms] and QTc are derived as the average waveform of a 10-second consecutive beats in the evaluation time points. QTc [Fridericia's formula; QTc=QT/(RR)1/3)] is calculated using the PowerLab (Registered trademark) system. The incidence of arrhythmia is visually evaluated for all ECG recordings (from 0.5 hours before dosing to end of experiment) for all four animals.

### Evaluation Time Points:

Before (pre dosing), and 10, 25, 40, 55, 70, and 85 min after the first dosing.

### Data Analysis of QTc:

Percentage changes (%) in QTc from the pre-dose value are calculated (the pre-dose value is regarded as 100%). Relative QTc is compared with vehicle value at the same evaluation point.

### Test Example 18-1: Pharmacology in the Beagle Dog

Test compounds were tested to evaluate the effect on the beta-amyloid profile in cerebrospinal fluid (CSF) of dogs after a single dose, in combination with pharmacokinetic (PK) follow up and limited safety evaluation.

In the case of compounds shown below, two beagle dogs (1 male, 1 female) were dosed with vehicle (1 mL/kg of an aqueous solution of 20% cyclodextrin) and 4 beagle dogs (2 males and 2 females) were dosed with test compound at the doses indicated in the following Table in an aqueous 20% cyclodextrin solution with a concentration in mg/mL identical to the dose given in mg/kg) on an empty stomach.

CSF was taken in conscious animals directly from the lateral ventricle via a cannula which was screwed in the skull and covered with subcutaneous tissue and skin, before and at 4, 8, 25 and 49 hours after dosing. Eight hours after dosing the animals got access to their regular meal for 30 minutes. Blood was taken for PK follow up (0.5, 1, 2, 4, 8, 25 and 49 hours) and serum samples for biochemistry were taken before and at 8 and 25 h after dosing. The CSF samples were used for measurement of Abeta 1-37, Abeta 1-38, Abeta 1-40 and Abeta 1-42. The results are summarized in the Table below:

TABLE 40

| | % Decrease in | % Decrease in | % Decrease in | |
|---|---|---|---|---|
| | Abeta 1-42 at | Abeta 1-42 at | Abeta 1-42 at | |
| | 8 h post dosing | 25 h post dosing | 49 h post dosing | |
| | compared to | compared to | compared to | Dose |
| No. | own baseline | own baseline | own baseline | (mg/kg) |
| I-011 | −54 | −37 | NR | 5 |
| I-011 | −74 | −77 | −30 | 10 |
| I-024 | −40 | NR | NR | 0.63 |
| I-024 | −66 | NR | NR | 2.5 |
| I-035 | −52 | NR | NR | 2.5 |
| I-035 | −63 | −27 | NR | 5 |
| I-035 | −82 | −28 | NR | 10 |
| I-053 | −31 | NR | NR | 0.63 |
| I-053 | −50 | −27 | NR | 2.5 |

% decrease indicated at 8 h and at last time point at which relevant decrease (>20% decrease) was observed.

### Test Example 18-2: Pharmacology in the Beagle Dog

Test compounds were tested to evaluate the effect on the beta-amyloid profile in cerebrospinal fluid (CSF) of dogs after a single dose, in combination with pharmacokinetic (PK) follow up and limited safety evaluation.

For each of compound II-6 or II-10, four beagle dogs (2 males, 2 females) were dosed with vehicle (1 mL/kg of an aqueous solution of 20% cyclodextrin) and 12 beagle dogs (2 males and 2 females per dosage group) were dosed with test compounds as follows:

TABLE 41

| Compound | Dosage |
|---|---|
| II-6 | 0.16, 0.31, and 0.63 mg/kg in 0.16, 0.31, and 0.63 |
| | mg/mL of an aqueous 20% cyclodextrin solution, on |
| | an empty stomach |
| II-10 | 0.16, 0.63, 1.25 and 2.5 mg/kg in 0.16, 0.63, 1.25 and |
| | 2.5 mg/kg of an aqueous 20% cyclodextrin solution, |
| | on an empty stomach |

In the case of compounds II-2, II-3 and II-18, two beagle dogs (1 male, 1 female) were dosed with vehicle (1 mL/kg of an aqueous solution of 20% cyclodextrin) and 4 beagle dogs (2 males and 2 females) were dosed with test compound (II-2, II-3, or II-18) at the doses indicated in the following Table in an aqueous 20% cyclodextrin solution with a concentration in mg/mL identical to the dose given in mg/kg) on an empty stomach.

CSF was taken in conscious animals directly from the lateral ventricle via a cannula which was screwed in the skull and covered with subcutaneous tissue and skin, before and at 4, 8, 25 and 49 hours after dosing. Eight hours after dosing the animals got access to their regular meal for 30 minutes. Blood was taken for PK follow up (0.5, 1, 2, 4, 8, 25 and 49 hours) and serum samples for biochemistry were taken before and at 8 and 25 h after dosing. The CSF samples were used for measurement of Abeta 1-37, Abeta 1-38, Abeta 1-40 and Abeta 1-42. The results are summarized in the Table below:

TABLE 42

| | | % Decrease in | | |
|---|---|---|---|---|
| | % Decrease in | Abeta 1-42 at | % Decrease in | |
| | Abeta 1-42 at | 24 h^{(a)} or 25 h^{(b)} | Abeta 1-42 at | |
| | 8 h post dosing | post dosing | 49 h post dosing | |
| | compared to | compared to | compared to | Dose |
| Co. No. | own baseline | own baseline | own baseline | (mg/kg) |
| II-2 | 33 | | | 0.63 |
| II-3 | 61 | | | 5 |
| II-6 | 51 | 31 | | 0.16 |
| | 69 | | 22 | 0.31 |
| | 68 | 62 | | 0.63 |
| II-10 | 37 | | | 0.16 |
| | 68 | 21 | | 0.63 |
| | 73 | 27 | | 1.25 |
| | 76 | 28 | | 2.5 |
| II-18 | 53 | 27 | | 0.63 |
| II-20 | 51 | 30 | 25 | 0.16 |
| | 72 | 70 | 52 | 0.63 |
| | 73 | 76 | 58 | 1 |
| | 86 | 91 | 85 | 3.75 |

% decrease indicated at 8 h and at last time point at which relevant decrease (>20% decrease) was observed.

### Test Example 19: Dansyl GSH Trapping Test

Dansyl glutathione (glutathione) trapping is a test of investigating reactive metabolites.

The reaction conditions were as follows: substrate, 50 μmol/L the compounds of the present invention; trapping reagent, 0.1 mmol/L dansyl GSH; protein content of pooled human liver microsomes, 1 mg/mL; pre-reaction time, 5 minutes; reaction time, 60 minutes; reaction temperature, 37° C.

Pooled human liver microsomes and a solution of the compound of the present invention in K-Pi buffer (pH 7.4) as a pre-reaction solution were added to a 96-well plate at the composition of the pre-reaction. NADPH as a cofactor was added to initiate a reaction. After a predetermined time of a reaction, a part is transferred to another 96-well plate, and a solution of acetonitrile including 5 mmol/L dithiothreitol was added to stop the reaction. After centrifuged at 3000 rpm for 15 minutes, fluorescence peak area of the dansyl GSH trapped metabolites was quantified by HPLC with fluorescence detection.

### Test Example 20: [¹⁴C]-KCN Trapping Test

[¹⁴C]-potassium cyanide (KCN) trapping is a test of investigating reactive metabolites.

The reaction conditions were as follows: substrate, 10 or 50 μmol/L the compounds of the present invention; trapping reagent, 1 mmol/L [¹⁴C]-KCN (11.7 μCi/tube); protein content of pooled human liver microsomes, 1 mg/mL; pre-reaction time, 5 minutes; reaction time, 60 minutes; reaction temperature, 37° C.

Pooled human liver microsomes and a solution of the compound of the present invention in K-Pi buffer (pH 7.4) as a pre-reaction solution were added to a 96-well plate at the composition of the pre-reaction. NADPH as a cofactor was added to initiate a reaction. After a predetermined time, the metabolic reactions were terminated and [¹⁴C]-KCN trapped metabolites were extracted to 100 μL methanol solutions by spin-column. Radio peak area of the [¹⁴C]-KCN trapped metabolites is quantified by Radio-HPLC system.

### Formulation Examples

The following Formulation Examples are only exemplified and not intended to limit the scope of the present invention.

### Formulation Example 1: Tablet

| | |
|---|---|
| Compound of the present invention | 15 mg |
| Lactose | 15 mg |
| Calcium stearate | 3 mg |

All of the above ingredients except for calcium stearate are uniformly mixed. Then the mixture is crushed, granulated and dried to obtain a suitable size of granules. Then, calcium stearate is added to the granules. Finally, tableting is performed under a compression force.

### Formulation Example 2: Capsules

| | |
|---|---|
| Compound of the present invention | 10 mg |
| Magnesium stearate | 10 mg |
| Lactose | 80 mg |

The above ingredients are mixed uniformly to obtain powders or fine granules, and then the obtained mixture is filled in capsules.

### Formulation Example 3: Granules

| | |
|---|---|
| Compound of the present invention | 30 g |
| Lactose | 265 g |
| Magnesium stearate | 5 g |

After the above ingredients are mixed uniformly, the mixture is compressed. The compressed matters are crushed, granulated and sieved to obtain suitable size of granules.

### Formulation Example 4: Orally Disintegrated Tablets

The compounds of the present invention and crystalline cellulose are mixed, granulated and tablets are made to give orally disintegrated tablets.

### Formulation Example 5: Dry Syrups

The compounds of the present invention and lactose are mixed, crushed, granulated and sieved to give suitable sizes of dry syrups.

### Formulation Example 6: Injections

The compounds of the present invention and phosphate buffer are mixed to give injection.

### Formulation Example 7: Infusions

The compounds of the present invention and phosphate buffer are mixed to give injection.

### Formulation Example 8: Inhalations

The compound of the present invention and lactose are mixed and crushed finely to give inhalations.

### Formulation Example 9: Ointments

The compounds of the present invention and petrolatum are mixed to give ointments.

### Formulation Example 10: Patches

The compounds of the present invention and base such as adhesive plaster or the like are mixed to give patches.

### INDUSTRIAL APPLICABILITY

The compounds of the present invention can be a medicament useful as an agent for treating or preventing a disease induced by production, secretion and/or deposition of amyloid ß proteins.

1. A compound of Formula (IA), (IB) or (IC): 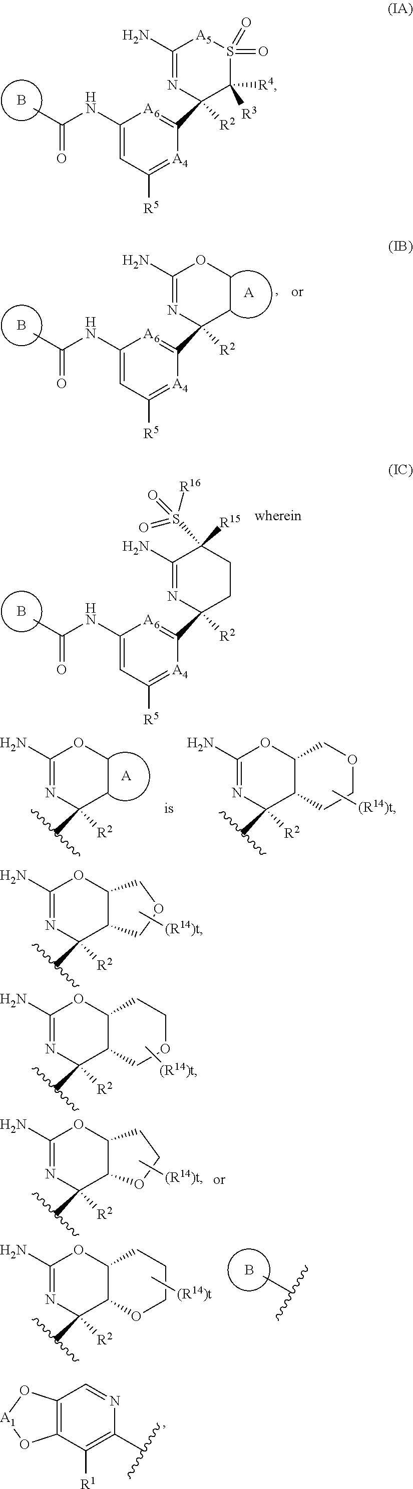 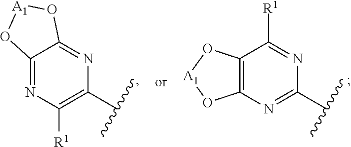
-A₁- is alkylene optionally substituted with one or more halogen;
R¹ is a hydrogen atom, halogen, alkyl, haloalkyl or amino;
R² is substituted or unsubstituted alkyl;
R³ and R⁴ are each independently a hydrogen atom, halogen, alkyl or haloalkyl;
R⁵ is a hydrogen atom or halogen;
A₄ is N or CR⁶ wherein R⁶ is a hydrogen atom, halogen or substituted or unsubstituted alkyl;
A₆ is CR¹⁸ or N;
R¹⁸ is a hydrogen atom;
A₄ and A₆ are not simultaneously both N;
A₅ is NR⁷ or CR⁸R⁹;
R⁷ is substituted or unsubstituted alkyl;
R⁸ and R⁹ are each independently a hydrogen atom, halogen, alkyl or haloalkyl;
R², R³R⁴, R⁸ and R⁹ may be any one of (i) to (iv):
(i) R² and one of R³ and R⁴ may be taken together with the carbon atoms to which they are attached to form a carbocycle or a heterocycle;
(ii) one of R³ and R⁴ and one of R⁸ and R⁹ may form alkylene wherein each carbon atom in the alkylene may be replaced with an oxygen atom or a nitrogen atom; the carbon atom(s) in the alkylene is each independently substituted with one or more group(s) selected from R^{a}; and the nitrogen atom(s) in the alkylene is each substituted with one or more group(s) selected from R^{b};
R^{a} is a hydrogen atom, halogen, hydroxy, cyano, or substituted or unsubstituted alkyl;
R^{b} is a hydrogen atom or substituted or unsubstituted alkyl;
(iii) R³ and R⁴ may be taken together with the carbon atom to which they are attached to form a carbocycle or a heterocycle;
(iv) R⁸ and R⁹ may be taken together with the carbon atom to which they are attached to form a carbocycle or a heterocycle;
R¹⁴ is each independently alkyl optionally substituted with one or more group(s) selected from halogen, cyano, alkyloxy, haloalkyloxy, and non-aromatic carbocyclyl; or heteroaryl optionally substituted with one or more alkyl;
two R¹⁴s attached to a same carbon atom may be taken together with the carbon atom to which they are attached to form a 3- to 5-membered non-aromatic carbocycle optionally substituted with one or more group(s) selected from halogen, alkyl and haloalkyl;
t is an integer from 0 to 3;
R¹⁵ is alkyl optionally substituted with one or more group(s) selected from halogen; and
R¹⁶ is substituted or unsubstituted alkyl or non-aromatic carbocyclyl;
or a pharmaceutically acceptable salt thereof.

2. The compound according to claim 1, wherein the compound is represented by the Formula (IA-2): 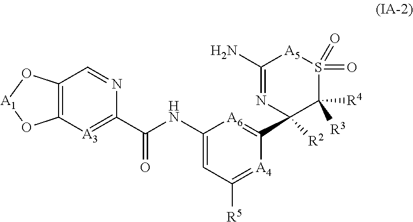
wherein
A₃ is N or CR¹; and the other symbols are the same as defined in claim 1,
or a pharmaceutically acceptable salt thereof.

3. The compound according to claim 1, wherein -A₁- is selected from the group consisting of
(i) —CH₂—,
(ii) —CH₂—CH₂—,
(iii) —CH₂—CH₂—CH₂—,
(iv) —CD₂-,
(v) —CD₂-CD₂-,
(vi) —CD₂-CD₂-CD₂-,
(vii) —CF₂—,
(viii) —CF₂—CH₂—,
(ix) —CH₂—CF₂—,
(x) —CF₂—CH₂—CH₂—,
(xi) —CH₂—CF₂—CH₂—,
(xii) —CH₂—CH₂—CF₂—,
(xiii) —CHF—,
(xiv) —CHF—CH₂—,
(xv) —CH₂—CHF—,
(xvi) —CHF—CH₂—CH₂—,
(xvii) —CH₂—CHF—CH₂—,
(xviii) —CH₂—CH₂—CHF—,
(xix) —CH(Me)-,
(xx) —CH(Me)-CH₂—,
(xxi) —CH₂—CH(Me)-.
(xxii) —CH(Me)-CH₂—CH₂—
(xxiii) —CH₂—CH(Me)-CH₂—, and
(xxiv) —CH₂—CH₂—CH(Me)-;
or a pharmaceutically acceptable salt thereof.

4. The compound according to claim 1, wherein -A₁- is selected from the group consisting of
(iv) —CD₂-,
(v) —CD₂-CD₂-,
(vi) —CD₂-CD₂-CD₂-,
(vii) —CF₂—,
(viii) —CF₂—CH₂—,
(ix) —CH₂—CF₂—,
(x) —CF₂—CH₂—CH₂—,
(xi) —CH₂—CF₂—CH₂—,
(xii) —CH₂—CH₂—CF₂—,
(xiii) —CHF—,
(xiv) —CHF—CH₂—,
(xv) —CH₂—CHF—,
(xvi) —CHF—CH₂—CH₂—,
(xvii) —CH₂—CHF—CH₂—, and
(xviii) —CH₂—CH₂—CHF—,
or a pharmaceutically acceptable salt thereof.

5. The compound according to claim 1, wherein 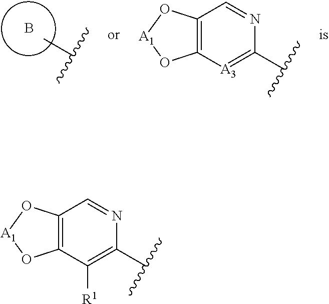
wherein R¹ is a hydrogen atom, fluoro, chloro, or methyl; or a pharmaceutically acceptable salt thereof.

6. The compound according to claim 1, wherein 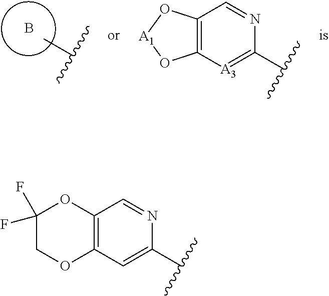
or a pharmaceutically acceptable salt thereof.

7. The compound according to claim 1, wherein R² is methyl optionally substituted with fluoro; or a pharmaceutically acceptable salt thereof.

8. The compound according to claim 1, wherein R³ and R⁴ are a hydrogen atom; or a pharmaceutically acceptable salt thereof.

9. The compound according to claim 1, wherein R⁵ is a hydrogen atom; or a pharmaceutically acceptable salt thereof.

10. The compound according to claim 1, wherein A₄ is CR⁶ wherein R⁶ is halogen; or a pharmaceutically acceptable salt thereof.

11. The compound according to claim 1, wherein A₅ is NR⁷; or a pharmaceutically acceptable salt thereof.

12. The compound according to claim 11, wherein R⁷ is methyl; or a pharmaceutically acceptable salt thereof.

13. The compound according to claim 1, wherein A₅ is CR⁸R⁹, wherein R⁸ and R⁹ are methyl; or a pharmaceutically acceptable salt thereof.

14. The compound according to claim 1 selected from the group consisting of Compound I-001, I-004, I-009, I-011, I-012, I-023, I-024, I-026, I-027, I-029, I-035, and I-043; or a pharmaceutically acceptable salt thereof.

15. A pharmaceutical composition comprising the compound according to claim 1, or a pharmaceutically acceptable salt thereof.

16. The pharmaceutical composition according to claim 15 for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia.

17-18. (canceled)

19. A method for inhibiting BACE1 activity comprising administering the compound according to claim 1, or a pharmaceutically acceptable salt thereof.

20. A method for treating or preventing Alzheimer dementia, mild cognitive impairment or prodromal Alzheimer's disease, for preventing the progression of Alzheimer dementia, mild cognitive impairment, or prodromal Alzheimer's disease, or for preventing the progression in a patient asymptomatic at risk for Alzheimer dementia comprising administering the compound according to claim 1, or a pharmaceutically acceptable salt thereof.

21. A BACE1 inhibitor comprising the compound according to claim 1, or a pharmaceutically acceptable salt thereof.
